(19) United States
(12) Patent Application Publication
(10) Pub. No.: US 2025/0002469 A1
(43) Pub. Date: Jan. 2, 2025

(54) ORGANIC COMPOUND AND ORGANIC LIGHT-EMITTING DEVICE COMPRISING SAME

(71) Applicant: P&H TECH Co., Ltd, Yongin-si (KR)

(72) Inventors: Seo-Yong HYUN, Yongin-si (KR); Seung Jae LEE, Yongin-si (KR); Gwan-hee PARK, Yongin-si (KR)

(73) Assignee: P&H TECH Co., Ltd, Yongin-si (KR)

(21) Appl. No.: 18/290,866

(22) PCT Filed: Jul. 19, 2022
(86) PCT No.: PCT/KR2022/010512

§ 371 Date: Jan. 22, 2024

(30) Foreign Application Priority Data
Jul. 22, 2021 (KR) 10-2021-0096200
Jul. 19, 2022 (KR) 10-2022-0088676

(51) Int. Cl. C07D 307/91; C07B 59/00; C07D 333/76; C07D 405/12; C07D 407/12; C07D 409/12; H10K 85/60

(52) U.S. Cl. CPC C07D 307/91; C07B 59/004; C07D 333/76; C07D 405/12; C07D 407/12; C07D 409/12; H10K 85/615; H10K 85/633; H10K 85/654; H10K 85/6572; H10K 85/6574; H10K 85/6576; C07B 2200/05

(57) ABSTRACT

The present invention relates to: an organic compound which is used for an organic layer, such as a hole transport layer or an electron blocking layer, in an organic light-emitting device; and an organic light-emitting device comprising same. When the organic compound according to the present invention is used for an organic layer in a device, improved luminous characteristics of the device can be implemented in terms of driving at low voltage, excellent luminous efficiency, and the like, and thus the present invention can be effectively used for industrial applications of various display and lighting devices.

### TECHNICAL FIELD

The present invention relates to a compound that is employed to an organic light-emitting device, and more particularly, to an organic compound that is employed as a material for an organic layer in an organic light-emitting device and an organic light-emitting device with improved luminescent properties such as low-voltage driving of the device, excellent luminous efficiency, etc. by employing the organic compound.

### BACKGROUND ART

The organic light-emitting device may be formed even on a transparent substrate, and may be driven at a low voltage of 10 V or less compared to a plasma display panel or an inorganic electroluminescence (EL) display. In addition, the device consumes relatively little power and has good color representation. The device may display three colors of green, blue, and red, and thus has recently become a subject of intense interest as a next-generation display device.

However, in order for such an organic light-emitting device to exhibit the aforementioned characteristics, the materials constituting an organic layer in the device, such as hole injecting materials, hole transport materials, light-emitting materials, electron transport materials, and electron injecting materials, are prerequisites for the support by stable and efficient materials. However, the development of a stable and efficient organic layer material for an organic light-emitting device has not yet been sufficiently made.

Thus, further improvements in terms of efficiency and life characteristics are required for good stability, high efficiency, long lifetime, and large size of organic light-emitting devices. Particularly, there is a strong need to develop materials constituting each organic layer of organic light-emitting devices.

In this regard, recently, research has been actively conducted to improve the conductivity (mobility) of conventional organic materials with respect to a material for a hole transport layer in the structure of the organic light-emitting device.

### DISCLOSURE

### Technical Problem

An aspect of the present invention intends to provide a novel organic compound that is employed to organic layers such as a hole transport layer, an electron blocking layer, etc. in an organic light-emitting device to implement excellent luminescent properties such as low-voltage driving of the device, improved luminous efficiency, etc., and an organic light-emitting device including the same.

### Technical Solution

An aspect of the present invention provides an organic compound represented by Formula 1 below and an organic light-emitting device in which the organic compound is included in organic layers such as a hole transport layer, an electron blocking layer, etc. in the device.

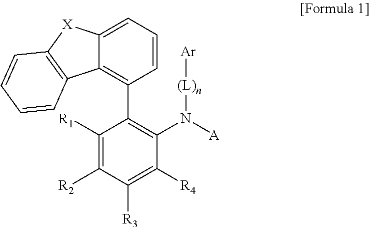

The characteristic structure of Formula I above and the definitions of compounds implemented thereby, X, R₁ to R₄, L, Ar, and A will be described below.

### Advantageous Effects

According to the present invention, when an organic compound is employed as a material for an organic layer such as a hole transport layer, an electron blocking layer, etc. in an organic light-emitting device, it is possible to implement luminescent properties such as low-voltage driving of the device, excellent luminous efficiency, etc., and thus can be usefully used in various display devices.

### BEST MODE

Hereinafter, the present invention will be described in more detail.

The present invention relates to an organic compound represented by Formula 1 below capable of achieving luminescent properties such as low-voltage driving of the device, excellent luminous efficiency, etc. of an organic light-emitting device. Structurally, it is characterized that (iii) an amine structure is introduced into position 1 of (ii) dibenzofuran (thiophene) through (ii) an ortho-linked phenylene, and the amine structure consists of a (spiro) fluorenyl group (A) and an aryl (heteroaryl) group (Ar) excluding a fluorenyl group. Through the structural characteristics, the organic compound is employed to a hole transport layer, an electron blocking layer, etc. in the organic light-emitting device to improve a low-voltage driving property of the device and a luminous efficiency property.

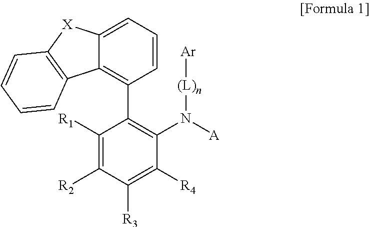

In Formula I above,
X is O or S,
R₁ to R₄ are the same as or different from each other, and are each independently hydrogen or deuterium,
L is a single bond or selected from a substituted or unsubstituted arylene group (however, excluding fluorenylene group) having 6 to 30 carbon atoms and a substituted or unsubstituted heteroarylene group having 3 to 30 carbon atoms, n is an integer of 0 to 2, and when the n is 2, a plurality of L are the same as or different from each other,
Ar is selected from a substituted or unsubstituted alkyl group having 1 to 20 carbon atoms, a substituted or unsubstituted cycloalkyl group having 3 to 20 carbon atoms, a substituted or unsubstituted aryl group (however, excluding a fluorenyl group) having 6 to 30 carbon atoms, and a substituted or unsubstituted heteroaryl group having 3 to 30 carbon atoms, and
A is represented by Structural Formula 1 or 2 below as a (spiro) fluorenyl structure.

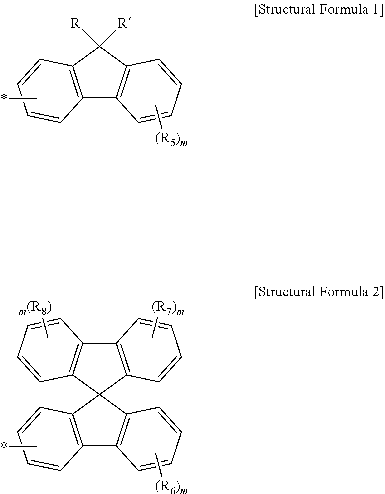

In Structural Formula 1 or 2 above,
R and R′ are each independently alkyl groups having 1 to 7 carbon atoms, and
R₅ to R₈ are the same as or different from each other, and are each independently selected from hydrogen, deuterium, a cyano group, a halogen group, a substituted or unsubstituted alkyl group having 1 to 20 carbon atoms, a substituted or unsubstituted cycloalkyl group having 3 to 20 carbon atoms, a substituted or unsubstituted aryl group having 6 to 30 carbon atoms and a substituted or unsubstituted heteroaryl group having 3 to 30 carbon atoms, and m is each an integer of 0 to 4, and when each m is 2 or more, a plurality of R₅ to R₈ are the same as or different from each other.

Meanwhile, in the definitions of L, Ar and R₅ to R₈ above, the ‘substituted or unsubstituted’ means substitution of L, Ar and R₅ to R₈ above with one or at least two substituents selected from the group consisting of deuterium, a halogen group, a cyano group, a nitro group, a hydroxy group, an alkyl group, a halogenated alkyl group, a deuterated alkyl group, a cycloalkyl group, a heterocycloalkyl group, an alkoxy group, a halogenated alkoxy group, a deuterated alkoxy group, an amine group, an aryl group, a heteroaryl group, an alkylsilyl group and an arylsilyl group, substitution with a substituent to which two or more of the substituents are linked, or having no substituent.

For specific examples, the substituted arylene group means that a phenyl group, a biphenyl group, a naphthalene group, a fluorenyl group, a pyrenyl group, a phenanthrenyl group, a perylene group, a tetracenyl group, and an anthracenyl group are substituted with other substituents.

In addition, the substituted heteroaryl group means that a pyridyl group, a thiophenyl group, a triazine group, a quinoline group, a phenanthroline group, an imidazole group, a thiazole group, an oxazole group, a carbazole group and a condensate heteroring group thereof, for example, a benzquinoline group, a benzimidazole group, a benzoxazole group, a benzthiazole group, a benzcarbazole group, a dibenzothiophenyl group, and a dibenzofuran group are substituted with other substituents.

In an embodiment of the present invention, examples of the substituents will be described in detail below, but are not limited thereto.

In an embodiment of the present invention, the alkyl groups may be straight or branched. The number of carbon atoms in the alkyl groups is not particularly limited but is preferably from 1 to 20. Specific examples of the alkyl groups include, but are not limited to, methyl, ethyl, propyl, n-propyl, isopropyl, butyl, n-butyl, isobutyl, tert-butyl, sec-butyl, 1-methylbutyl, 1-ethylbutyl, pentyl, n-pentyl, isopentyl, neopentyl, tert-pentyl, hexyl, n-hexyl, 1-methylpentyl, 2-methylpentyl, 4-methyl-2-pentyl, 3,3-dimethylbutyl, 2-ethylbutyl, heptyl, n-heptyl, 1-methylhexyl, cyclopentylmethyl, cyclohexylmethyl, octyl, n-octyl, tert-octyl, 1-methylheptyl, 2-ethylhexyl, 2-propylpentyl, n-nonyl, 2,2-dimethylheptyl, 1-ethylpropyl, 1,1-dimethylpropyl, isohexyl, 2-methylpentyl, 4-methylhexyl, and 5-methylhexyl groups.

In an embodiment of the present invention, the alkoxy groups may be straight or branched. The number of carbon atoms in the alkoxy groups is not particularly limited but is preferably from 1 to 20 as long as steric hindrance is avoided. Specific examples of the alkoxy groups include, but are not limited to, methoxy, ethoxy, n-propoxy, isopropoxy, i-propyloxy, n-butoxy, isobutoxy, tert-butoxy, sec-butoxy, n-pentyloxy, neopentyloxy, isopentyloxy, n-hexyloxy, 3,3-dimethylbutyloxy, 2-ethylbutyloxy, n-octyloxy, n-nonyloxy, n-decyloxy, benzyloxy, and p-methylbenzyloxy groups.

In an embodiment of the present invention, the deuterated alkyl group or alkoxy group and the halogenated alkyl group or alkoxy group mean an alkyl group or alkoxy group in which the above alkyl group or alkoxy group is substituted with deuterium or a halogen group.

In an embodiment of the present invention, the aryl groups may be monocyclic or polycyclic. The number of carbon atoms in the aryl groups is not particularly limited but is preferably from 6 to 30. Examples of the monocyclic aryl groups include phenyl, biphenyl, terphenyl, and stilbene groups but the scope of the present invention is not limited thereto. Examples of the polycyclic aryl groups include naphthyl, anthracenyl, phenanthrenyl, pyrenyl, perylenyl, tetracenyl, chrysenyl, fluorenyl, acenaphathcenyl, triphenylene, and fluoranthrene groups, but the scope of the present invention is not limited thereto.

In an embodiment of the present invention, the heteroaryl groups refer to heterocyclic groups containing heteroatoms selected from O, N, and S. The number of carbon atoms is not particularly limited, but preferably from 2 to 30. In an embodiment of the present invention, specific examples thereof include, but are not limited to, thiophene, furan, pyrrole, imidazole, thiazole, oxazole, oxadiazole, triazole, pyridyl, bipyridyl, pyrimidyl, triazine, triazole, acridyl, pyridazine, pyrazinyl, quinolinyl, quinazoline, quinoxalinyl, phthalazinyl, pyridopyrimidinyl, pyridopyrazinyl, pyrazinopyrazinyl, isoquinoline, indole, carbazole, benzoxazole, benzimidazole, benzothiazole, benzocarbazole, benzothiophene, dibenzothiophene, benzofuranyl, dibenzofuranyl, phenanthroline, thiazolyl, isoxazolyl, oxadiazolyl, thiadiazolyl, benzothiazolyl, phenothiazinyl, phenoxazine, and phenothiazine groups.

In an embodiment of the present invention, the silyl group is an unsubstituted silyl group or a silyl group substituted with an alkyl group, an aryl group, and the like, and specific examples of the silyl group include trimethylsilyl, triethylsilyl, triphenylsilyl, trimethoxysilyl, dimethoxyphenylsilyl, diphenylmethylsilyl, diphenylvinylsilyl, methylcyclobutylsilyl, dimethylfurylsilyl, and the like, but are not limited thereto.

Specific examples of the halogen groups as substituents used in an embodiment of the present invention include fluorine (F), chlorine (CI), and bromine (Br).

In an embodiment of the present invention, the cycloalkyl group refers to a monocyclic, polycyclic and spiro alkyl radical, includes the same, and preferably contains a cyclic carbon atom having 3 to 20 carbon atoms, and includes cyclopropyl, cyclopentyl, cyclohexyl, bicycloheptyl, spirodecyl, spiroundecyl, adamantyl, and the like, and the cycloalkyl group may be arbitrarily substituted.

In an embodiment of the present invention, the heterocycloalkyl group refers to an aromatic or non-aromatic cyclic radical containing one or more heteroatoms, and includes the same, and one or more heteroatoms are selected from among O, S, N, P, B, Si, and Se, preferably O, N or S, and specifically, in the case of including N, the one or more heteroatoms may be aziridine, pyrrolidine, piperidine, azepane, azocane, and the like.

In the present invention, the amine group may be —NH₂, an alkylamine group, an arylamine group, an arylheteroarylamine group, etc., the arylamine group refers to amine substituted with an aryl group, the alkylamine group refers to amine substituted with an alkyl group, and the arylheteroarylamine group refers to amine substituted with aryl and heteroaryl groups. Examples of the arylamine group include a substituted or unsubstituted monoarylamine group, a substituted or unsubstituted diarylamine group, or a substituted or unsubstituted triarylamine group. The aryl group and the heteroaryl group in the arylamine group and the arylheteroarylamine group may be a monocyclic aryl group, a monocyclic heteroaryl group, a polycyclic aryl group, or a polycyclic heteroaryl group, and the arylamine group and the arylheteroarylamine group including two or more aryl groups and heteroaryl groups may include a monocyclic aryl group (heteroaryl group), a polycyclic aryl group (heteroaryl group), or both a monocyclic aryl group (heteroaryl group) and a polycyclic aryl group (heteroaryl group). In addition, the aryl group and the heteroaryl group in the arylamine group and the arylheteroarylamine group may be selected from examples of the above-mentioned aryl group and heteroaryl group.

The organic compound represented by Formula I above according to the present invention may be used in various organic layers in the organic light-emitting device due to its structural specificity, and may be preferably used in a hole transport layer or an electron blocking layer.

Preferred specific examples of the organic compound represented by Formula I according to the present invention include the following compounds, but are not limited thereto.

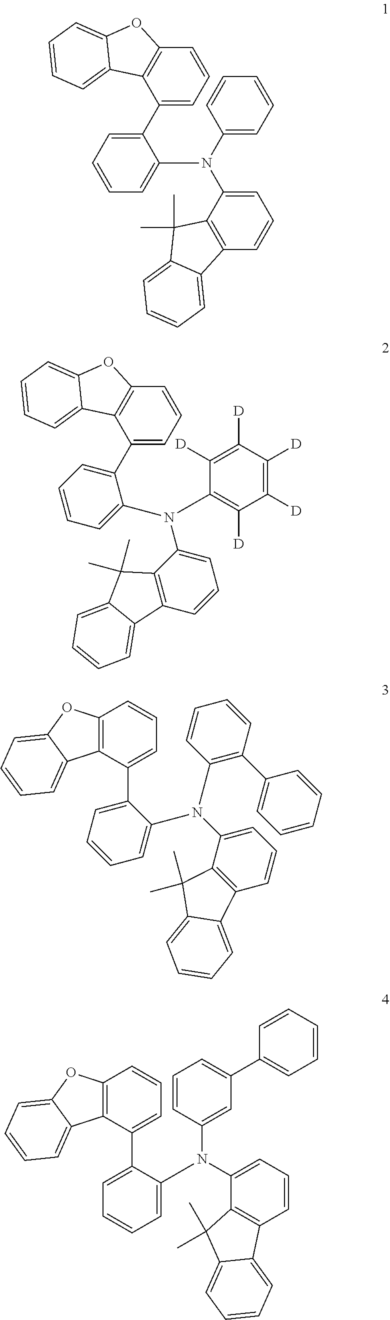

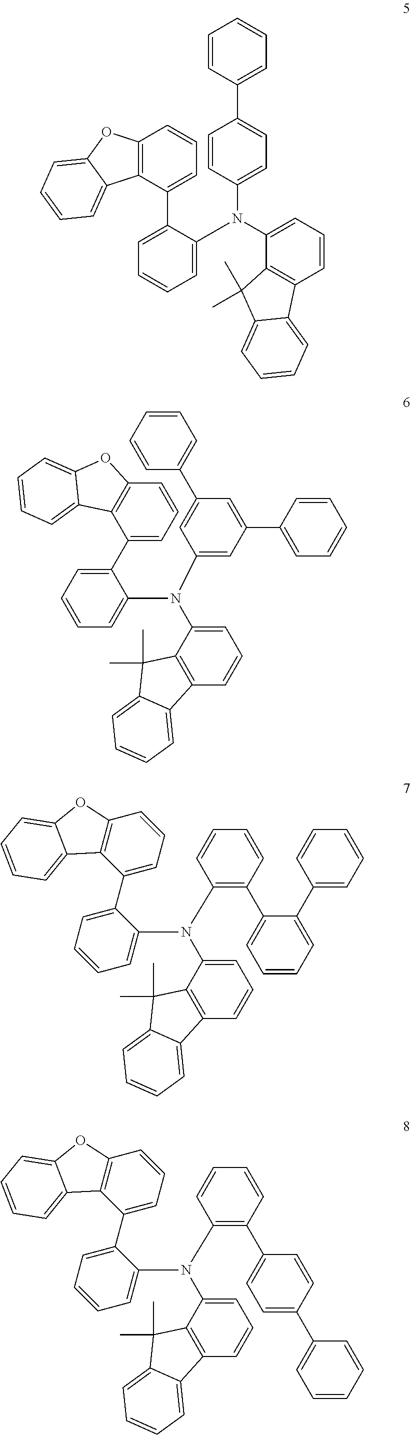 [SMILES: CC1(C)c2ccccc2-c2cccc(N(c3ccc(-c4ccccc4)cc3)c3ccccc3-c3cccc4oc5ccccc5c34)c21 ; CC1(C)c2ccccc2-c2cccc(N(c3cc(-c4ccccc4)cc(-c4ccccc4)c3)c3ccccc3-c3cccc4oc5ccccc5c34)c21 ; CC1(C)c2ccccc2-c2cccc(N(c3ccccc3-c3ccccc3-c3ccccc3)c3ccccc3-c3cccc4oc5ccccc5c34)c21 ; CC1(C)c2ccccc2-c2cccc(N(c3ccccc3-c3ccc(-c4ccccc4)cc3)c3ccccc3-c3cccc4oc5ccccc5c34)c21]

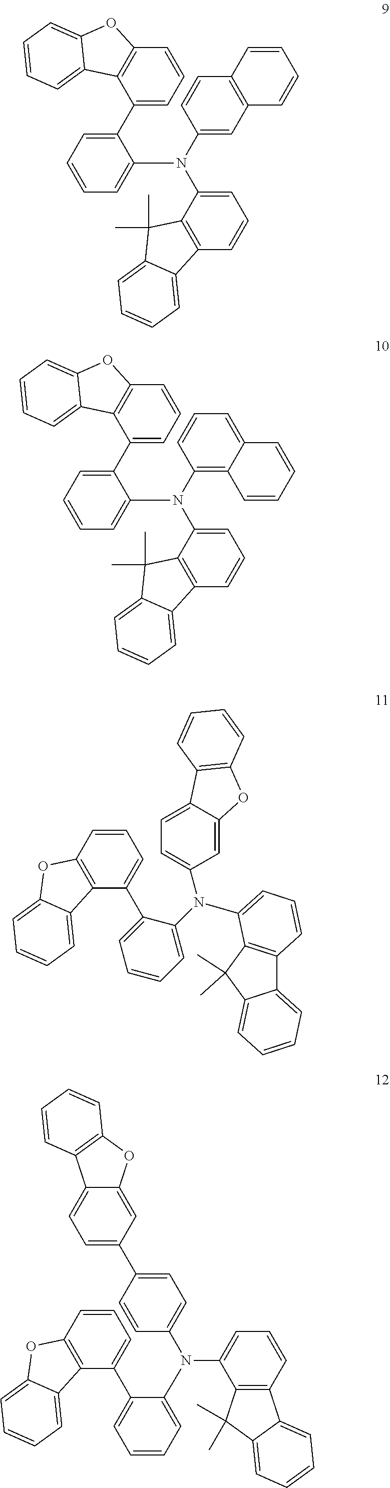 [SMILES: CC1(C)c2ccccc2-c2cccc(N(c3ccc4ccccc4c3)c3ccccc3-c3cccc4oc5ccccc5c34)c21 ; CC1(C)c2ccccc2-c2cccc(N(c3ccccc3-c3cccc4oc5ccccc5c34)c3cccc4ccccc34)c21 ; CC1(C)c2ccccc2-c2cccc(N(c3ccc4c(c3)oc3ccccc34)c3ccccc3-c3cccc4oc5ccccc5c34)c21 ; CC1(C)c2ccccc2-c2cccc(N(c3ccc(-c4ccc5c(c4)oc4ccccc45)cc3)c3ccccc3-c3cccc4oc5ccccc5c34)c21]

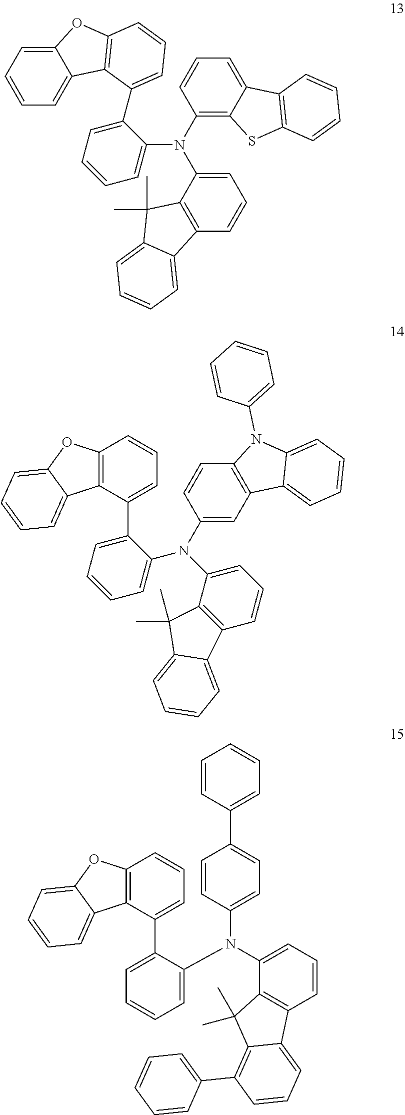

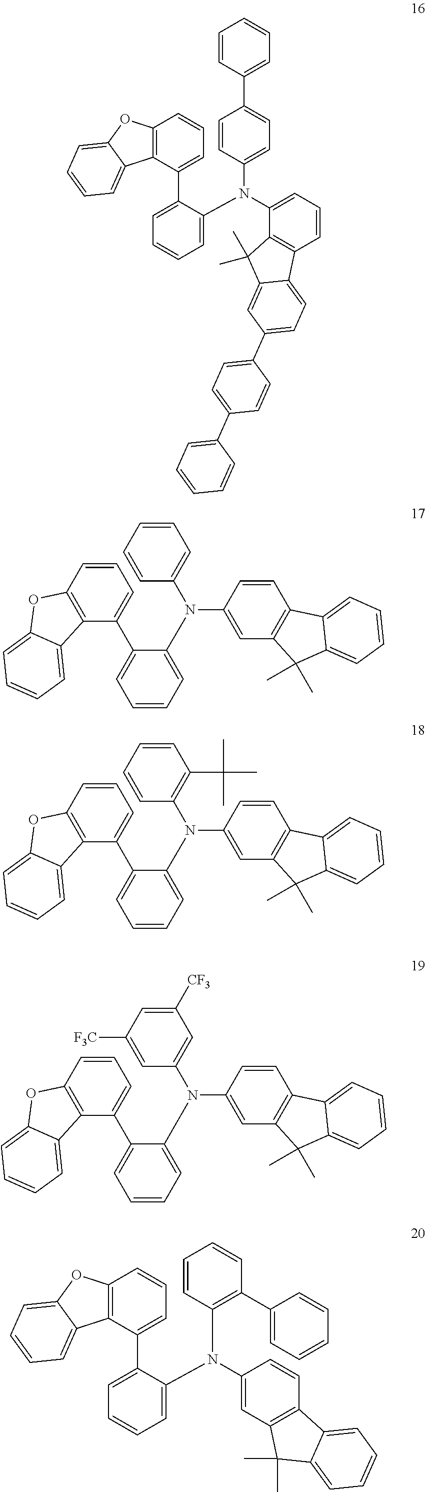

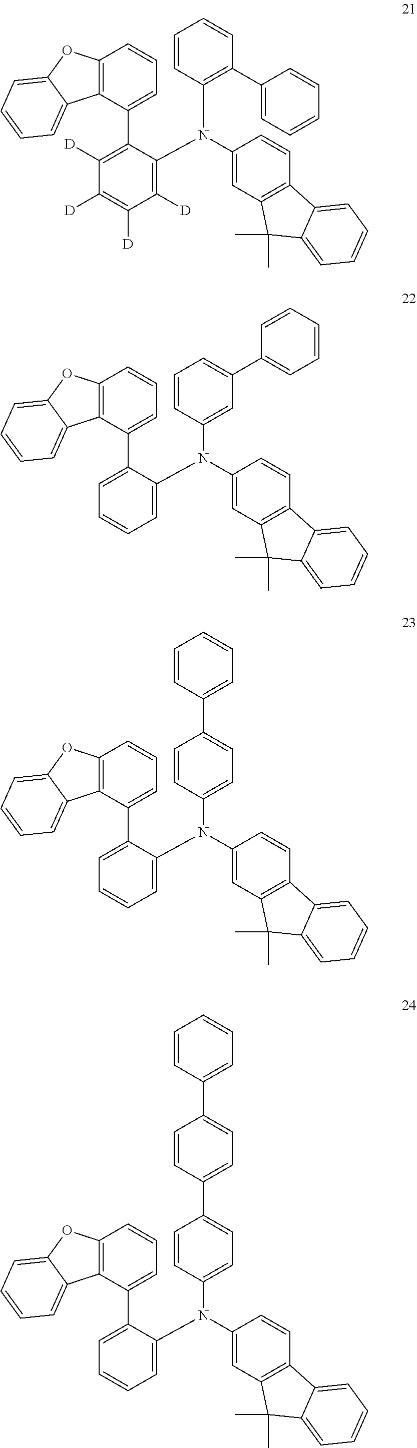

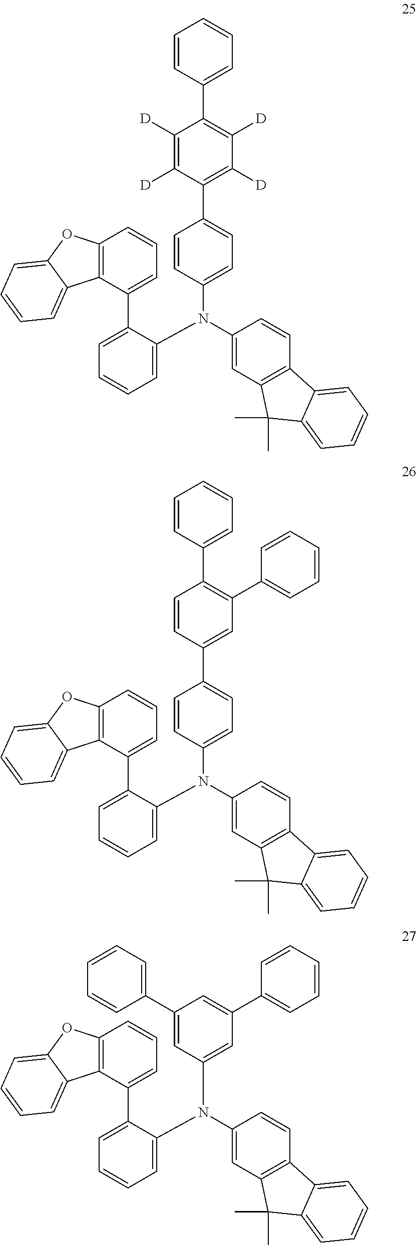

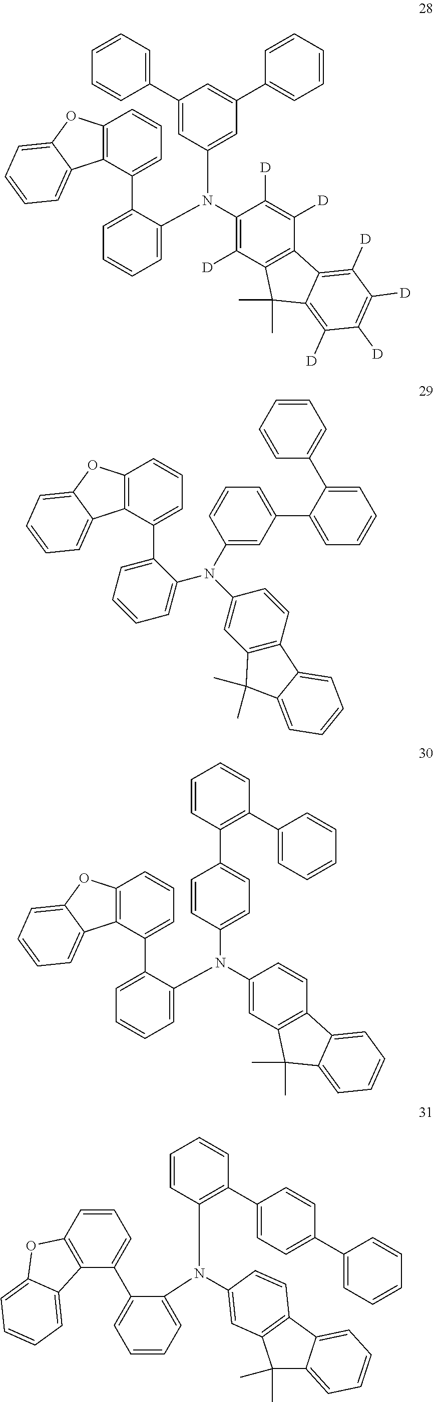

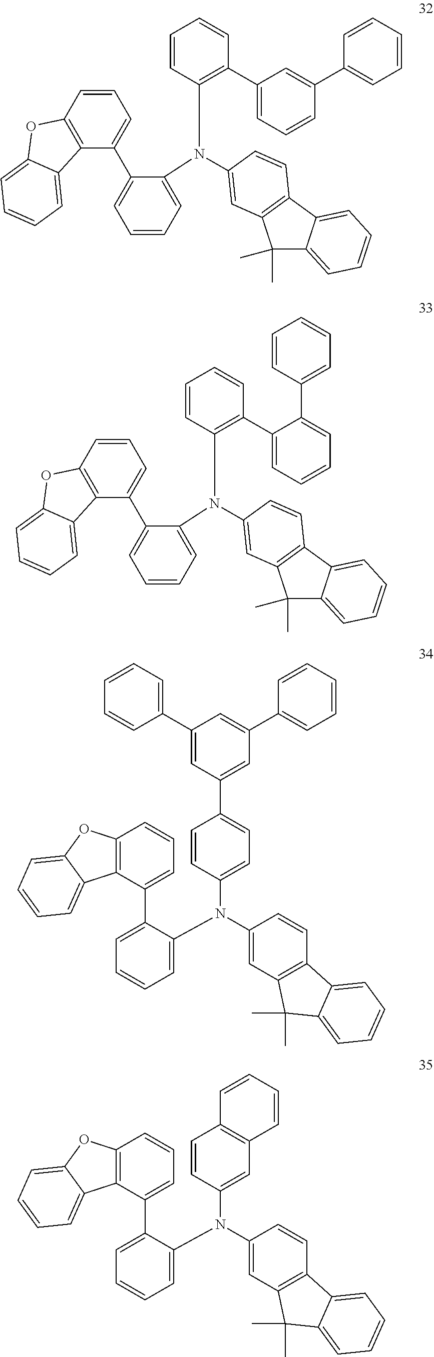

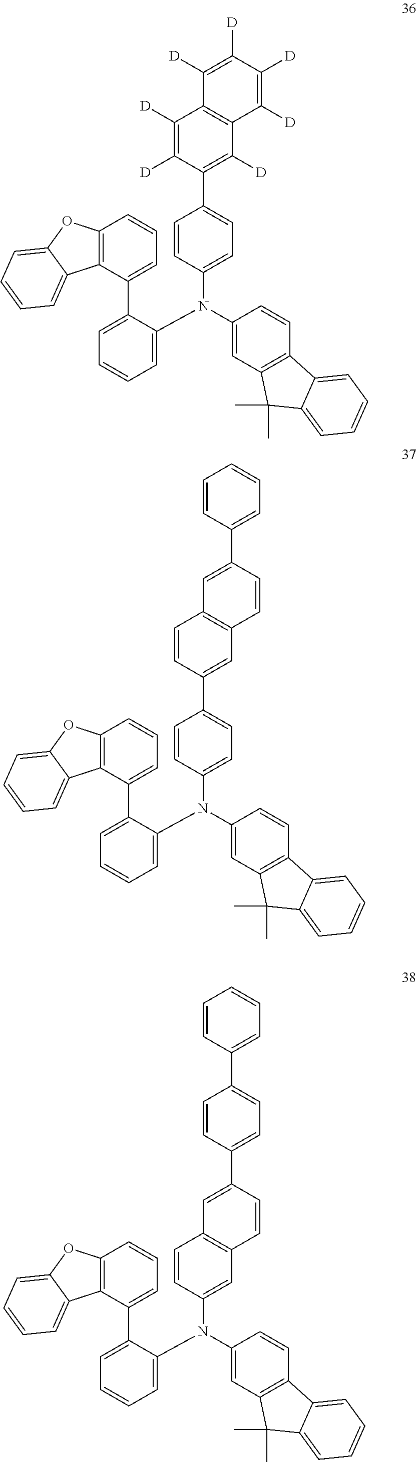

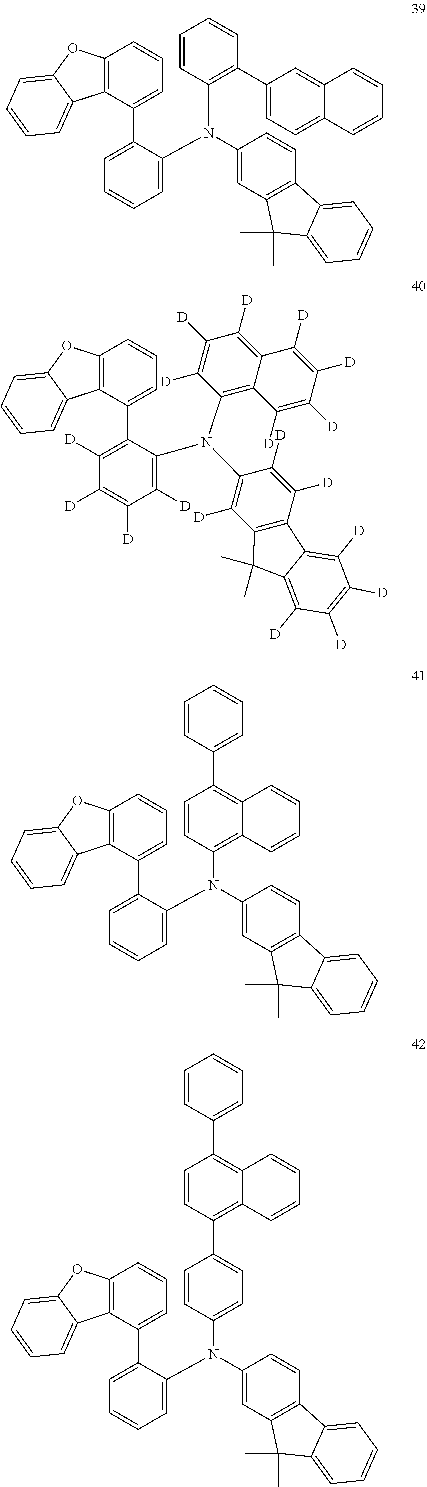

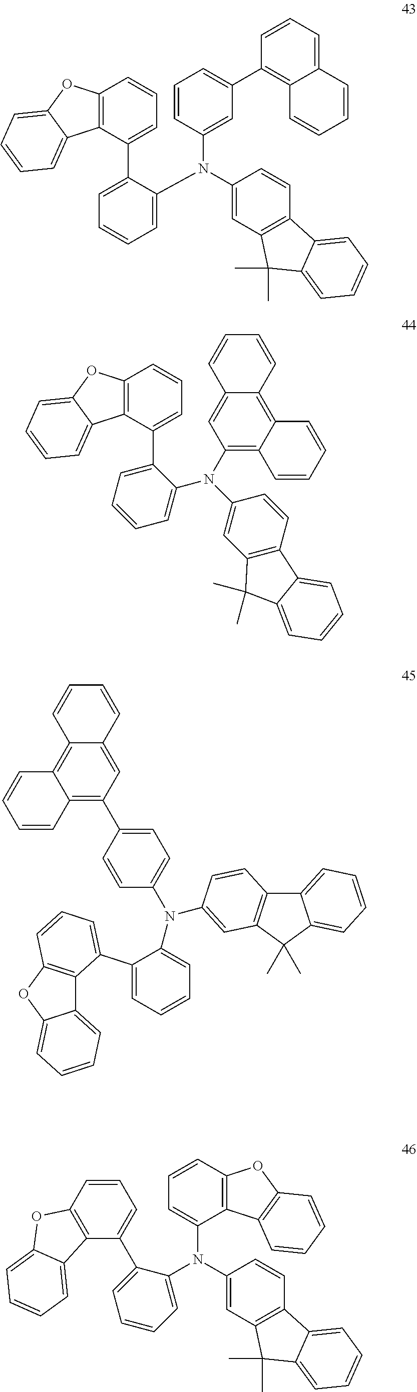

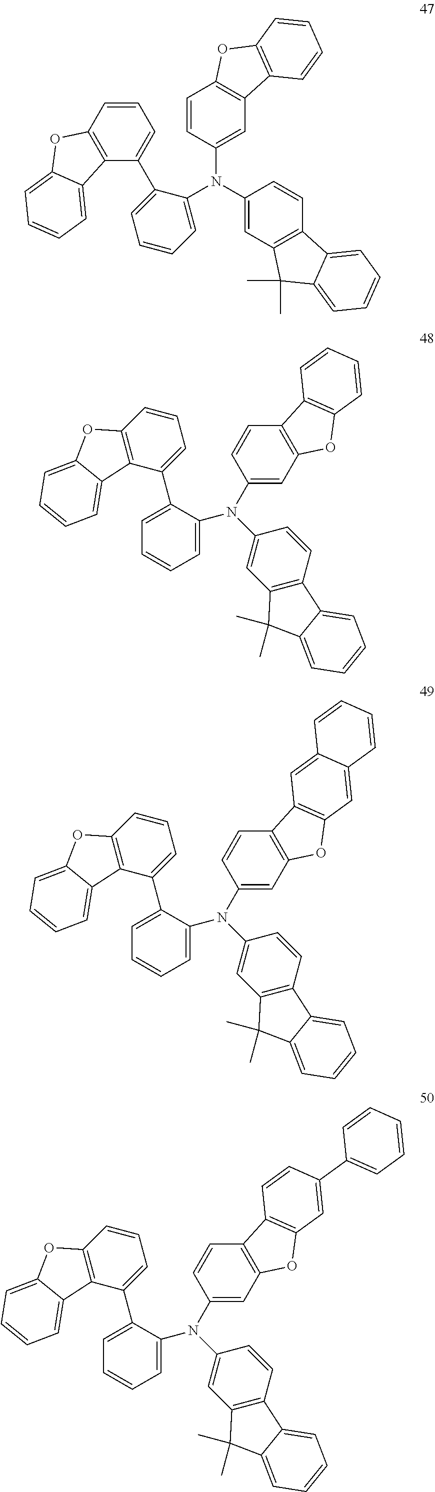

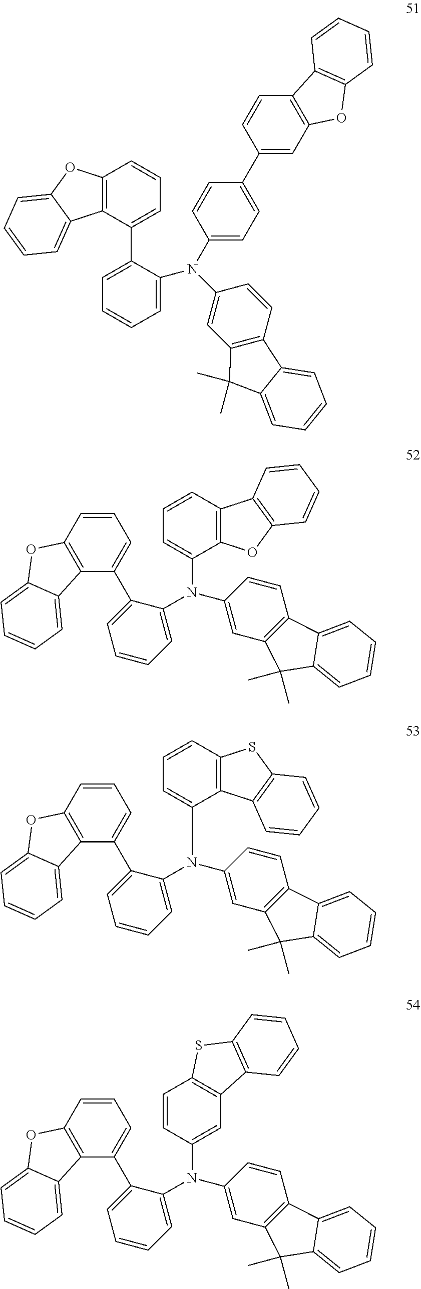

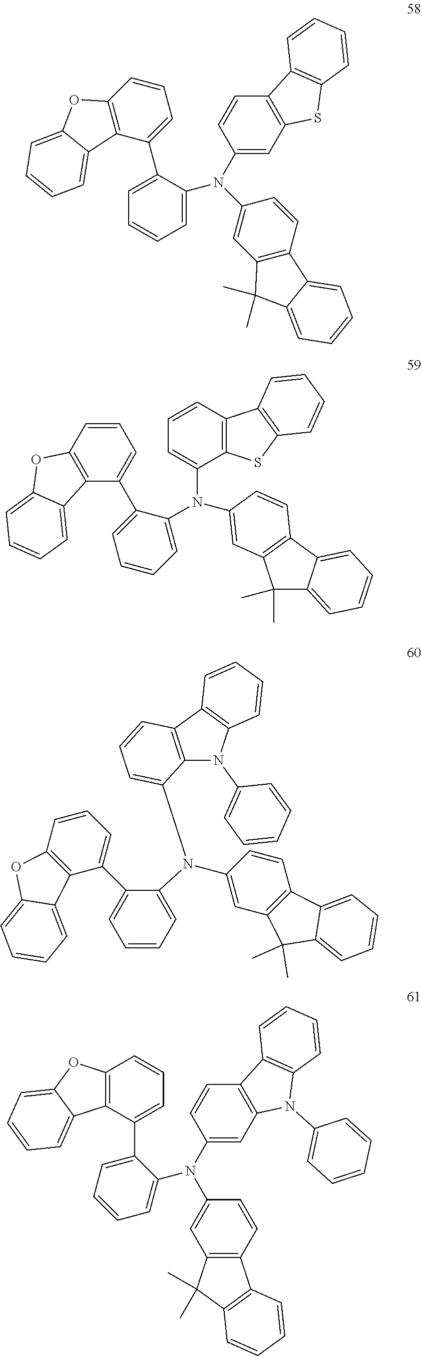

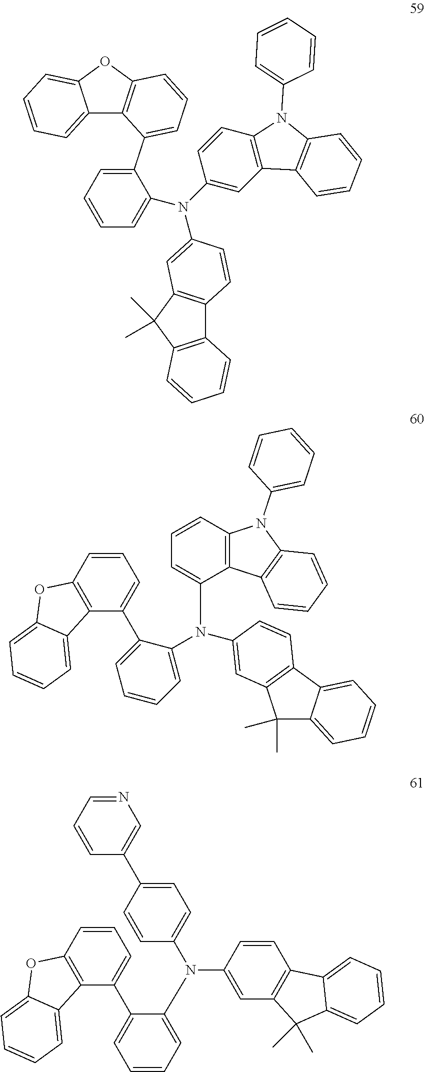 [SMILES: CC1(C)c2ccccc2-c2ccc(N(c3ccc4c(c3)c3ccccc3n4-c3ccccc3)c3ccccc3-c3cccc4oc5ccccc5c34)cc21 ; CC1(C)c2ccccc2-c2ccc(N(c3ccccc3-c3cccc4oc5ccccc5c34)c3cccc4c3c3ccccc3n4-c3ccccc3)cc21 ; CC1(C)c2ccccc2-c2ccc(N(c3ccc(-c4cccnc4)cc3)c3ccccc3-c3cccc4oc5ccccc5c34)cc21]

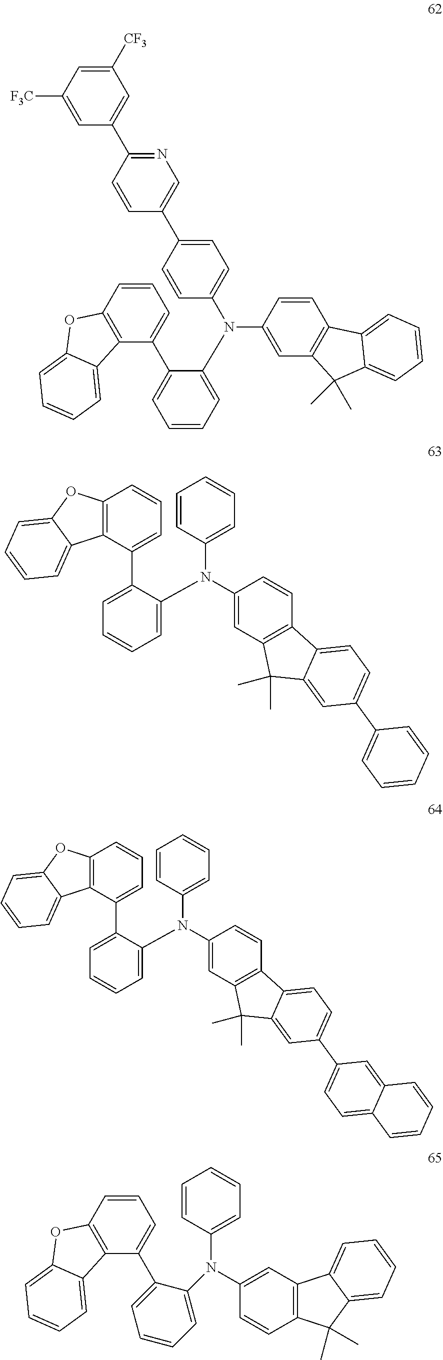 [SMILES: CC1(C)c2ccccc2-c2ccc(N(c3ccc(-c4ccc(-c5cc(C(F)(F)F)cc(C(F)(F)F)c5)nc4)cc3)c3ccccc3-c3cccc4oc5ccccc5c34)cc21 ; CC1(C)c2cc(-c3ccccc3)ccc2-c2ccc(N(c3ccccc3)c3ccccc3-c3cccc4oc5ccccc5c34)cc21 ; CC1(C)c2cc(-c3ccc4ccccc4c3)ccc2-c2ccc(N(c3ccccc3)c3ccccc3-c3cccc4oc5ccccc5c34)cc21 ; CC1(C)c2ccccc2-c2cc(N(c3ccccc3)c3ccccc3-c3cccc4oc5ccccc5c34)ccc21]

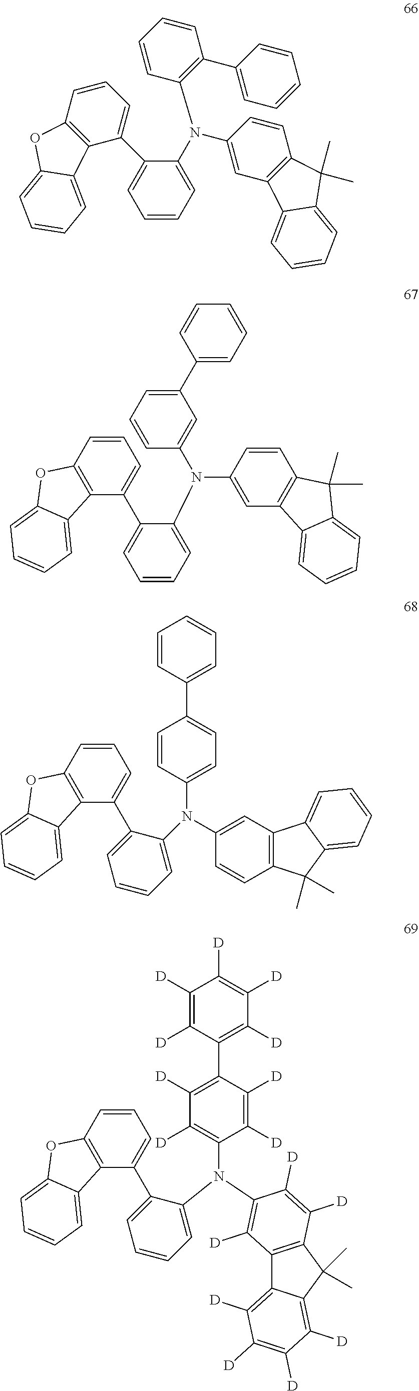

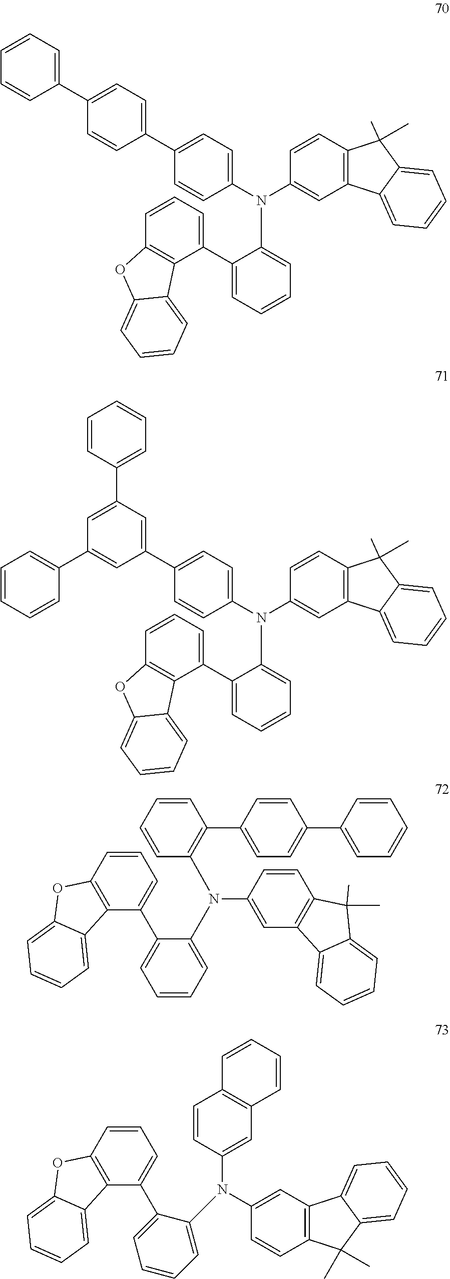

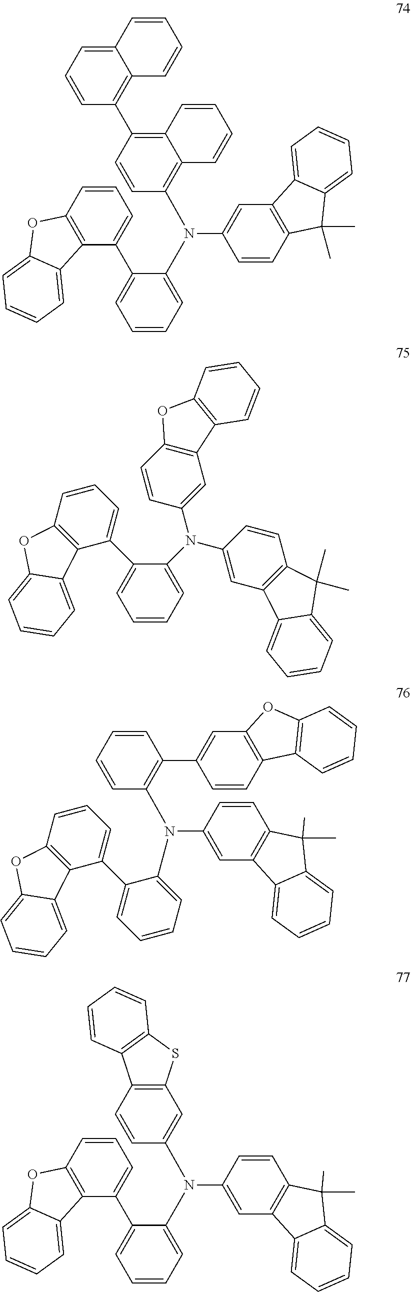

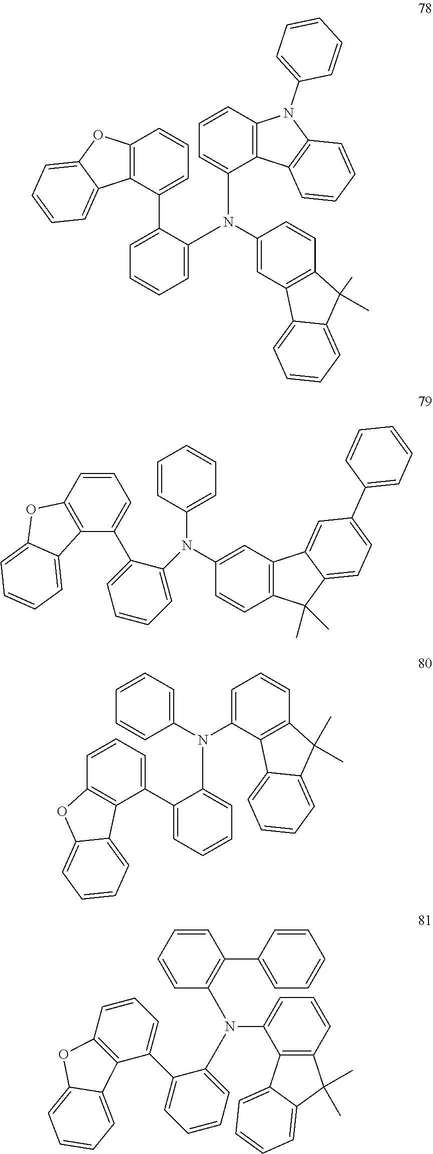 [SMILES: CC1(C)c2ccccc2-c2cc(N(c3ccccc3-c3cccc4oc5ccccc5c34)c3cccc4c3c3ccccc3n4-c3ccccc3)ccc21 ; CC1(C)c2ccc(-c3ccccc3)cc2-c2cc(N(c3ccccc3)c3ccccc3-c3cccc4oc5ccccc5c34)ccc21 ; CC1(C)c2ccccc2-c2c(N(c3ccccc3)c3ccccc3-c3cccc4oc5ccccc5c34)cccc21 ; CC1(C)c2ccccc2-c2c(N(c3ccccc3-c3ccccc3)c3ccccc3-c3cccc4oc5ccccc5c34)cccc21]

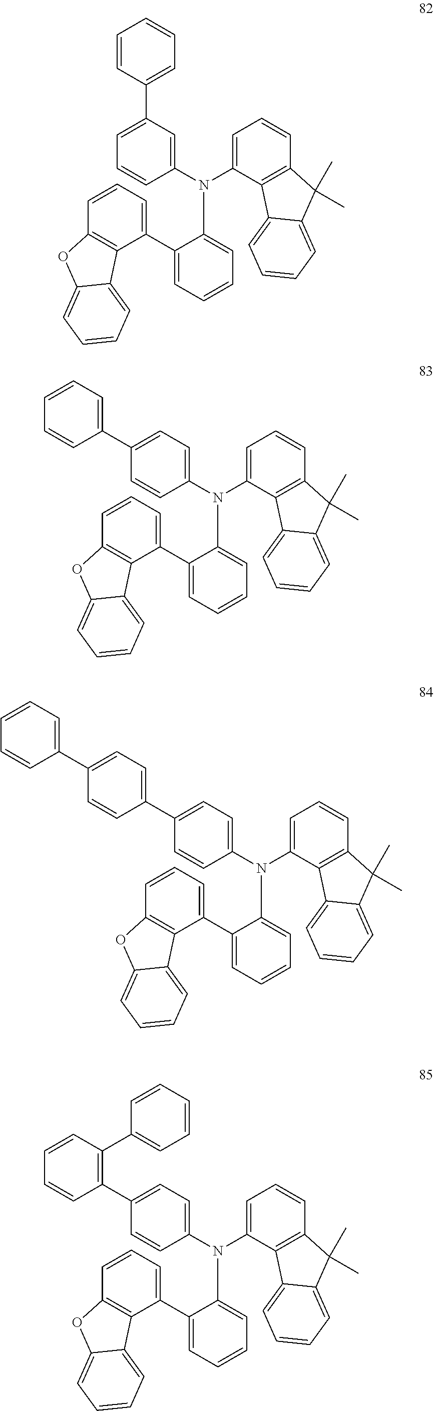

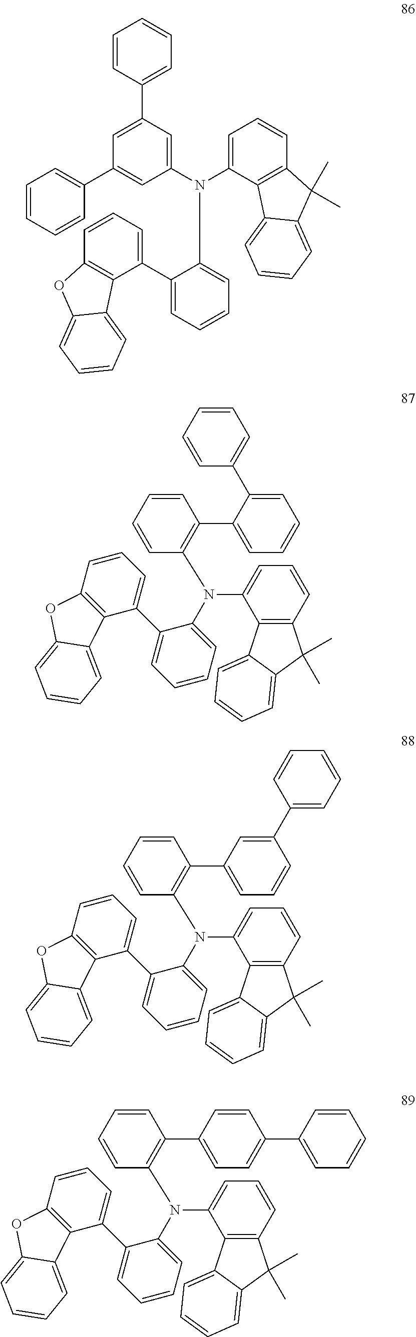

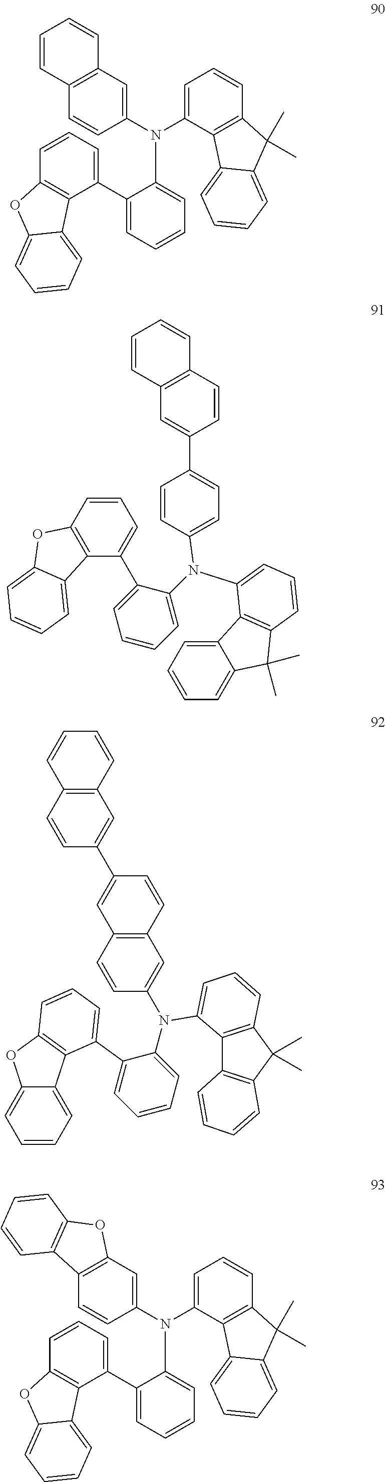 [SMILES: CC1(C)c2ccccc2-c2c(N(c3ccc4ccccc4c3)c3ccccc3-c3cccc4oc5ccccc5c34)cccc21 ; CC1(C)c2ccccc2-c2c(N(c3ccc(-c4ccc5ccccc5c4)cc3)c3ccccc3-c3cccc4oc5ccccc5c34)cccc21 ; CC1(C)c2ccccc2-c2c(N(c3ccc4cc(-c5ccc6ccccc6c5)ccc4c3)c3ccccc3-c3cccc4oc5ccccc5c34)cccc21 ; CC1(C)c2ccccc2-c2c(N(c3ccc4c(c3)oc3ccccc34)c3ccccc3-c3cccc4oc5ccccc5c34)cccc21]

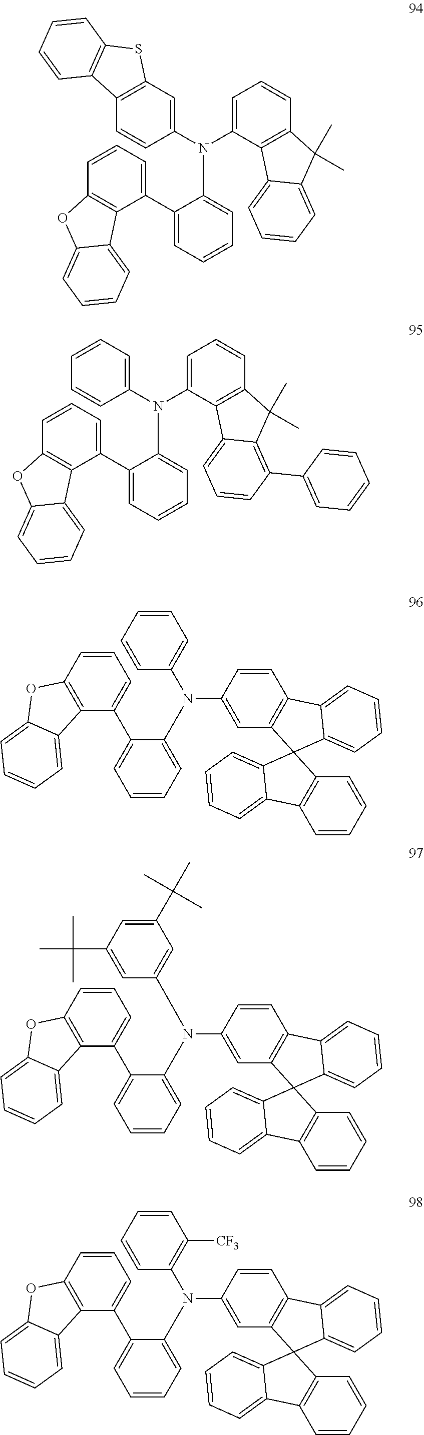

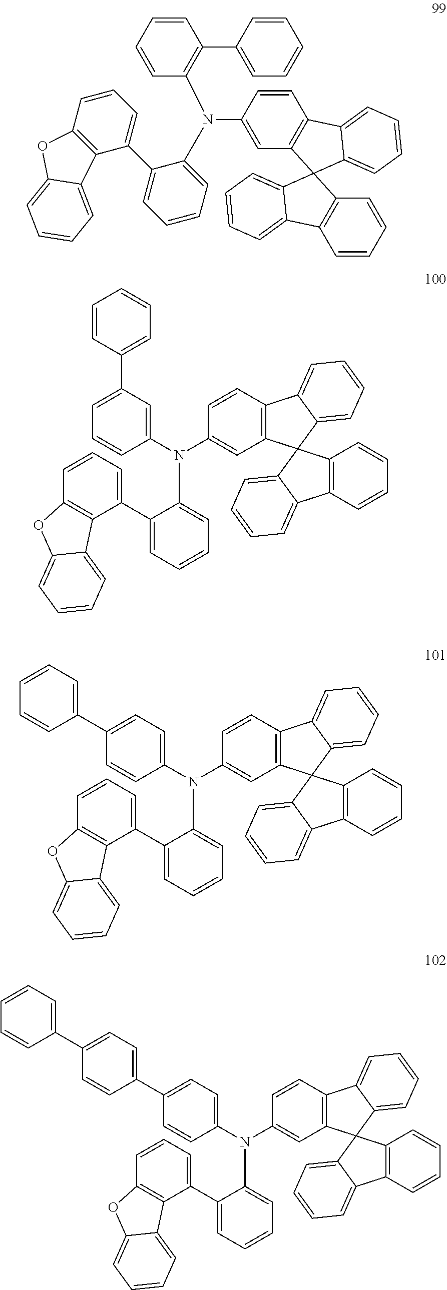 [SMILES: c1ccc(-c2ccccc2N(c2ccc3c(c2)C2(c4ccccc4-c4ccccc42)c2ccccc2-3)c2ccccc2-c2cccc3oc4ccccc4c23)cc1 ; c1ccc(-c2cccc(N(c3ccc4c(c3)C3(c5ccccc5-c5ccccc53)c3ccccc3-4)c3ccccc3-c3cccc4oc5ccccc5c34)c2)cc1 ; c1ccc(-c2ccc(N(c3ccc4c(c3)C3(c5ccccc5-c5ccccc53)c3ccccc3-4)c3ccccc3-c3cccc4oc5ccccc5c34)cc2)cc1 ; c1ccc(-c2ccc(-c3ccc(N(c4ccc5c(c4)C4(c6ccccc6-c6ccccc64)c4ccccc4-5)c4ccccc4-c4cccc5oc6ccccc6c45)cc3)cc2)cc1]

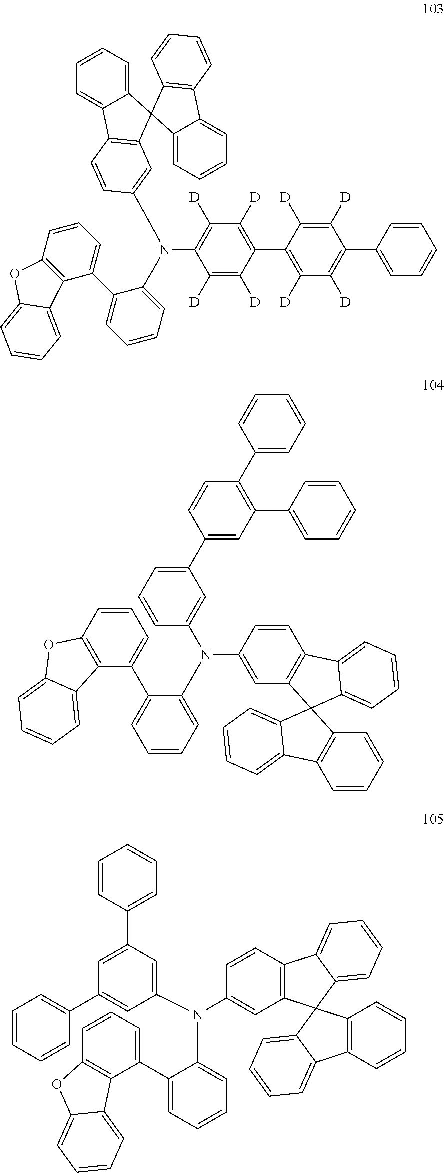 [SMILES: [2H]c1c([2H])c(-c2c([2H])c([2H])c(N(c3ccc4c(c3)C3(c5ccccc5-c5ccccc53)c3ccccc3-4)c3ccccc3-c3cccc4oc5ccccc5c34)c([2H])c2[2H])c([2H])c([2H])c1-c1ccccc1 ; c1ccc(-c2ccc(-c3cccc(N(c4ccc5c(c4)C4(c6ccccc6-c6ccccc64)c4ccccc4-5)c4ccccc4-c4cccc5oc6ccccc6c45)c3)cc2-c2ccccc2)cc1 ; c1ccc(-c2cc(-c3ccccc3)cc(N(c3ccc4c(c3)C3(c5ccccc5-c5ccccc53)c3ccccc3-4)c3ccccc3-c3cccc4oc5ccccc5c34)c2)cc1]

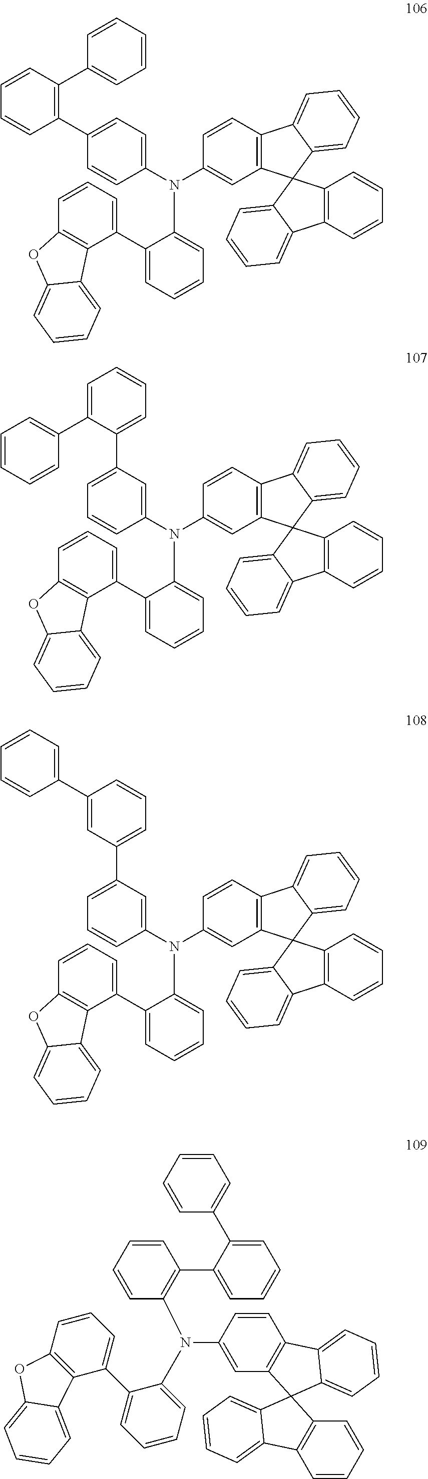

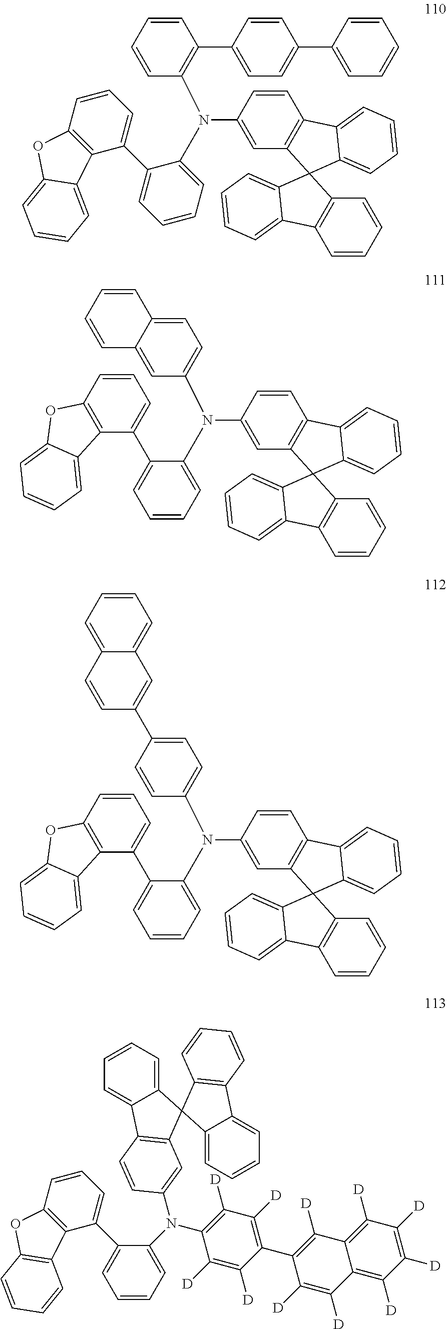 [SMILES: c1ccc(-c2ccc(-c3ccccc3N(c3ccc4c(c3)C3(c5ccccc5-c5ccccc53)c3ccccc3-4)c3ccccc3-c3cccc4oc5ccccc5c34)cc2)cc1 ; c1ccc(N(c2ccc3c(c2)C2(c4ccccc4-c4ccccc42)c2ccccc2-3)c2ccc3ccccc3c2)c(-c2cccc3oc4ccccc4c23)c1 ; c1ccc(N(c2ccc(-c3ccc4ccccc4c3)cc2)c2ccc3c(c2)C2(c4ccccc4-c4ccccc42)c2ccccc2-3)c(-c2cccc3oc4ccccc4c23)c1 ; [2H]c1c([2H])c(N(c2ccc3c(c2)C2(c4ccccc4-c4ccccc42)c2ccccc2-3)c2ccccc2-c2cccc3oc4ccccc4c23)c([2H])c([2H])c1-c1c([2H])c([2H])c2c([2H])c([2H])c([2H])c([2H])c2c1[2H]]

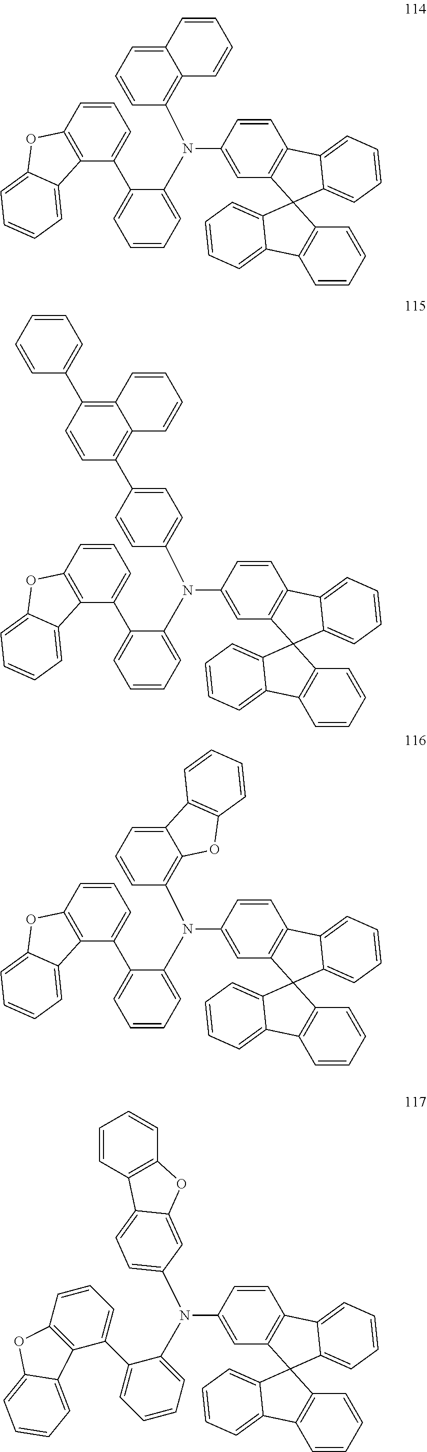 [SMILES: c1ccc(N(c2ccc3c(c2)C2(c4ccccc4-c4ccccc42)c2ccccc2-3)c2cccc3ccccc23)c(-c2cccc3oc4ccccc4c23)c1 ; c1ccc(-c2ccc(-c3ccc(N(c4ccc5c(c4)C4(c6ccccc6-c6ccccc64)c4ccccc4-5)c4ccccc4-c4cccc5oc6ccccc6c45)cc3)c3ccccc23)cc1 ; c1ccc(N(c2ccc3c(c2)C2(c4ccccc4-c4ccccc42)c2ccccc2-3)c2cccc3c2oc2ccccc23)c(-c2cccc3oc4ccccc4c23)c1 ; c1ccc(N(c2ccc3c(c2)C2(c4ccccc4-c4ccccc42)c2ccccc2-3)c2ccc3c(c2)oc2ccccc23)c(-c2cccc3oc4ccccc4c23)c1]

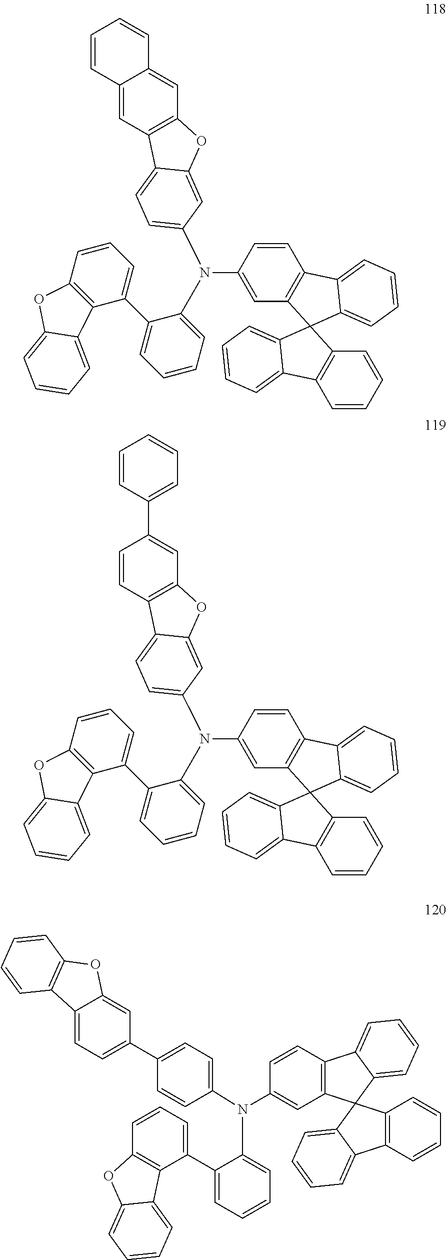 [SMILES: c1ccc(N(c2ccc3c(c2)C2(c4ccccc4-c4ccccc42)c2ccccc2-3)c2ccc3c(c2)oc2cc4ccccc4cc23)c(-c2cccc3oc4ccccc4c23)c1 ; c1ccc(-c2ccc3c(c2)oc2cc(N(c4ccc5c(c4)C4(c6ccccc6-c6ccccc64)c4ccccc4-5)c4ccccc4-c4cccc5oc6ccccc6c45)ccc23)cc1 ; c1ccc(N(c2ccc(-c3ccc4c(c3)oc3ccccc34)cc2)c2ccc3c(c2)C2(c4ccccc4-c4ccccc42)c2ccccc2-3)c(-c2cccc3oc4ccccc4c23)c1]

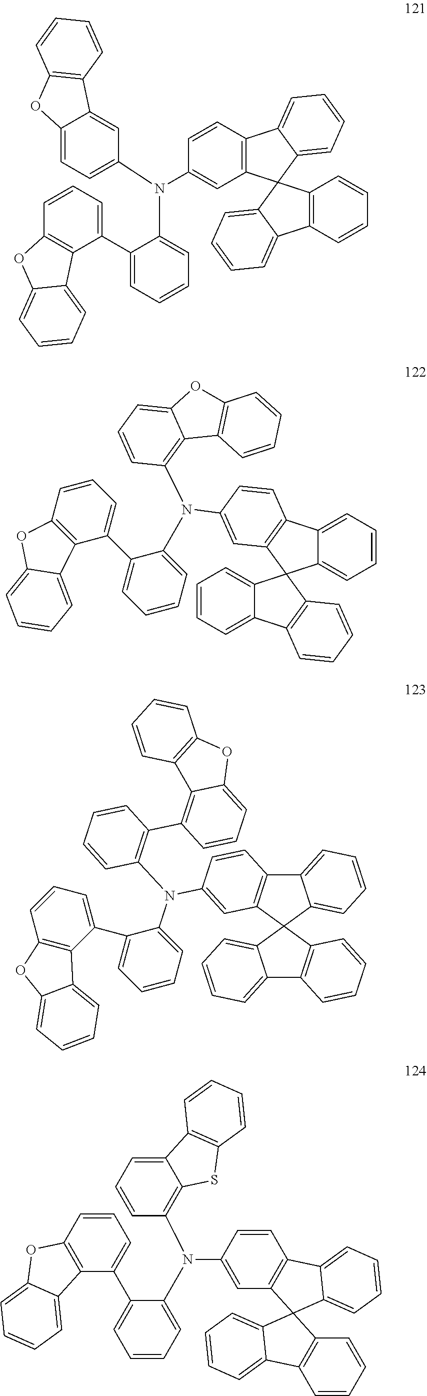 [SMILES: c1ccc(N(c2ccc3c(c2)C2(c4ccccc4-c4ccccc42)c2ccccc2-3)c2ccc3oc4ccccc4c3c2)c(-c2cccc3oc4ccccc4c23)c1 ; c1ccc(N(c2ccc3c(c2)C2(c4ccccc4-c4ccccc42)c2ccccc2-3)c2cccc3oc4ccccc4c23)c(-c2cccc3oc4ccccc4c23)c1 ; c1ccc(N(c2ccc3c(c2)C2(c4ccccc4-c4ccccc42)c2ccccc2-3)c2ccccc2-c2cccc3oc4ccccc4c23)c(-c2cccc3oc4ccccc4c23)c1 ; c1ccc(N(c2ccc3c(c2)C2(c4ccccc4-c4ccccc42)c2ccccc2-3)c2cccc3c2sc2ccccc23)c(-c2cccc3oc4ccccc4c23)c1]

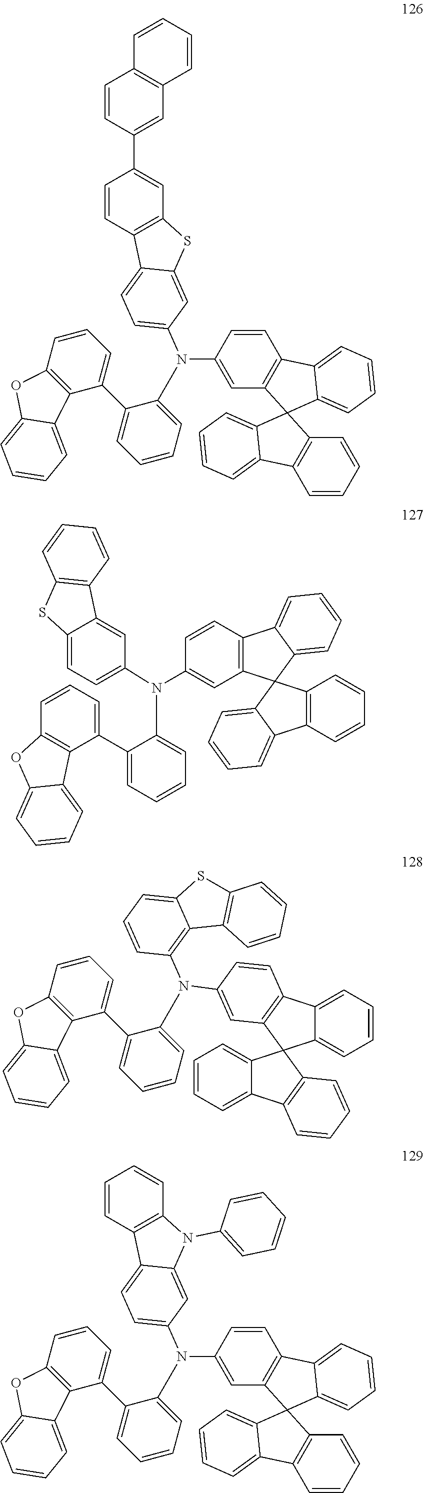 [SMILES: c1ccc(N(c2ccc3c(c2)C2(c4ccccc4-c4ccccc42)c2ccccc2-3)c2ccc3c(c2)sc2cc(-c4ccc5ccccc5c4)ccc23)c(-c2cccc3oc4ccccc4c23)c1 ; c1ccc(N(c2ccc3c(c2)C2(c4ccccc4-c4ccccc42)c2ccccc2-3)c2ccc3sc4ccccc4c3c2)c(-c2cccc3oc4ccccc4c23)c1 ; c1ccc(N(c2ccc3c(c2)C2(c4ccccc4-c4ccccc42)c2ccccc2-3)c2cccc3sc4ccccc4c23)c(-c2cccc3oc4ccccc4c23)c1 ; c1ccc(-n2c3ccccc3c3ccc(N(c4ccc5c(c4)C4(c6ccccc6-c6ccccc64)c4ccccc4-5)c4ccccc4-c4cccc5oc6ccccc6c45)cc32)cc1]

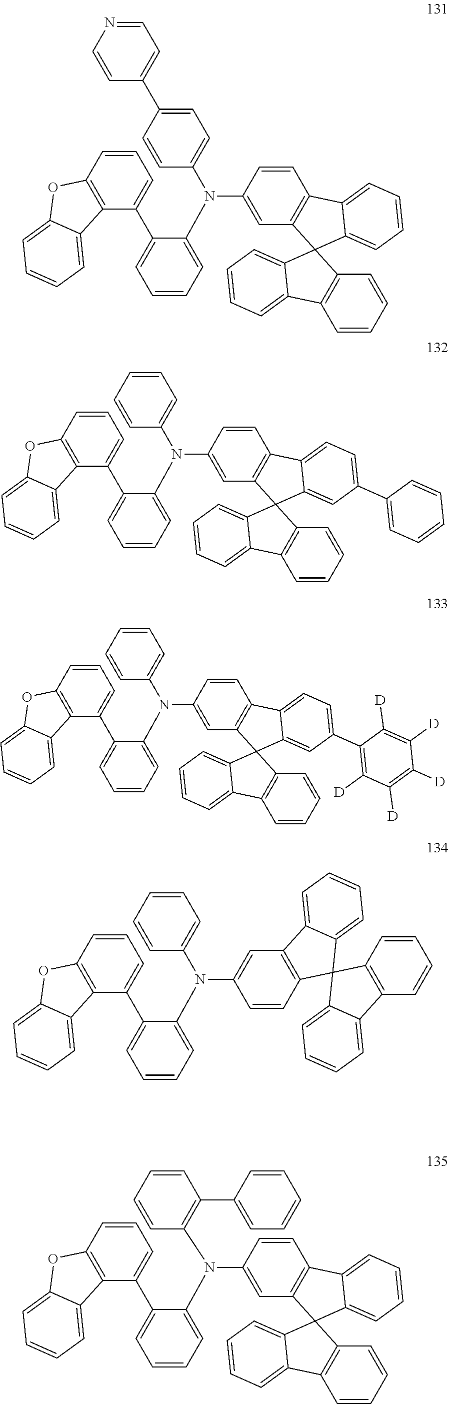 [SMILES: c1ccc(N(c2ccc(-c3ccncc3)cc2)c2ccc3c(c2)C2(c4ccccc4-c4ccccc42)c2ccccc2-3)c(-c2cccc3oc4ccccc4c23)c1 ; c1ccc(-c2ccc3c(c2)C2(c4ccccc4-c4ccccc42)c2cc(N(c4ccccc4)c4ccccc4-c4cccc5oc6ccccc6c45)ccc2-3)cc1 ; [2H]c1c([2H])c([2H])c(-c2ccc3c(c2)C2(c4ccccc4-c4ccccc42)c2cc(N(c4ccccc4)c4ccccc4-c4cccc5oc6ccccc6c45)ccc2-3)c([2H])c1[2H] ; c1ccc(N(c2ccc3c(c2)-c2ccccc2C32c3ccccc3-c3ccccc32)c2ccccc2-c2cccc3oc4ccccc4c23)cc1 ; c1ccc(-c2ccccc2N(c2ccc3c(c2)C2(c4ccccc4-c4ccccc42)c2ccccc2-3)c2ccccc2-c2cccc3oc4ccccc4c23)cc1]

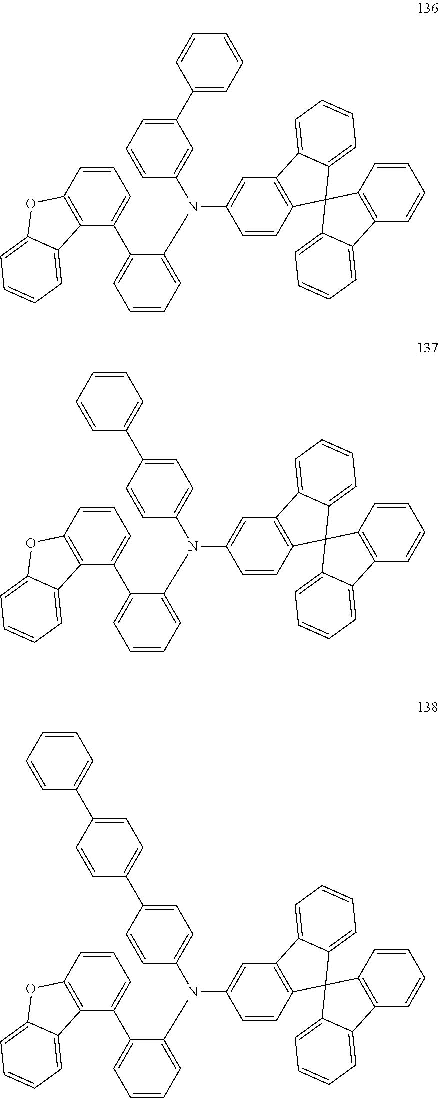 [SMILES: c1ccc(-c2cccc(N(c3ccc4c(c3)-c3ccccc3C43c4ccccc4-c4ccccc43)c3ccccc3-c3cccc4oc5ccccc5c34)c2)cc1 ; c1ccc(-c2ccc(N(c3ccc4c(c3)-c3ccccc3C43c4ccccc4-c4ccccc43)c3ccccc3-c3cccc4oc5ccccc5c34)cc2)cc1 ; c1ccc(-c2ccc(-c3ccc(N(c4ccc5c(c4)-c4ccccc4C54c5ccccc5-c5ccccc54)c4ccccc4-c4cccc5oc6ccccc6c45)cc3)cc2)cc1]

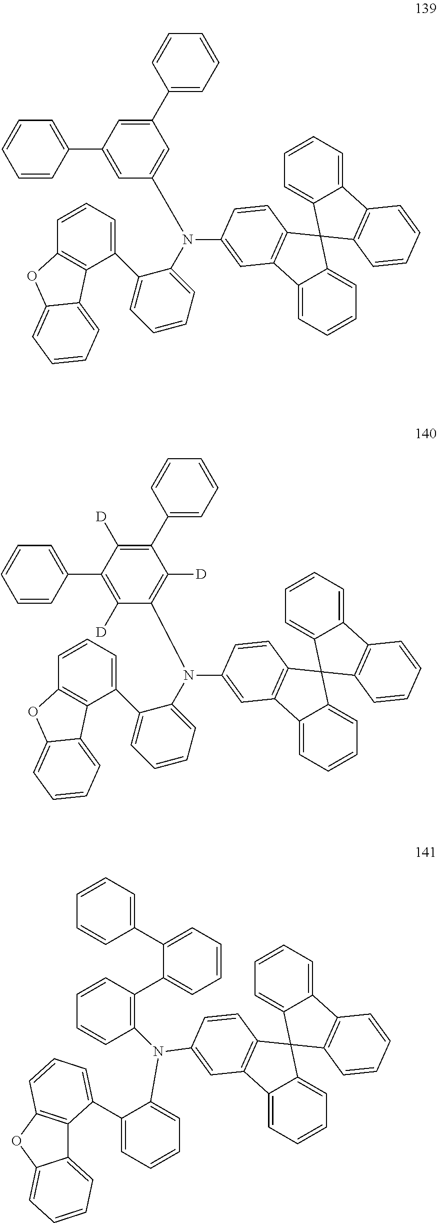 [SMILES: c1ccc(-c2cc(-c3ccccc3)cc(N(c3ccc4c(c3)-c3ccccc3C43c4ccccc4-c4ccccc43)c3ccccc3-c3cccc4oc5ccccc5c34)c2)cc1 ; [2H]c1c(-c2ccccc2)c([2H])c(N(c2ccc3c(c2)-c2ccccc2C32c3ccccc3-c3ccccc32)c2ccccc2-c2cccc3oc4ccccc4c23)c([2H])c1-c1ccccc1 ; c1ccc(-c2ccccc2-c2ccccc2N(c2ccc3c(c2)-c2ccccc2C32c3ccccc3-c3ccccc32)c2ccccc2-c2cccc3oc4ccccc4c23)cc1]

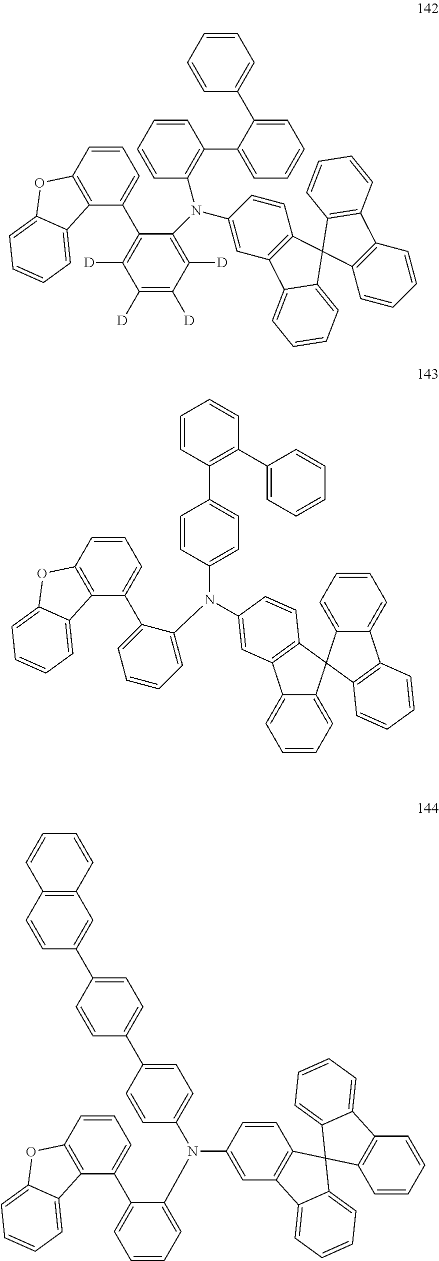 [SMILES: [2H]c1c([2H])c([2H])c(N(c2ccc3c(c2)-c2ccccc2C32c3ccccc3-c3ccccc32)c2ccccc2-c2ccccc2-c2ccccc2)c(-c2cccc3oc4ccccc4c23)c1[2H] ; c1ccc(-c2ccccc2-c2ccc(N(c3ccc4c(c3)-c3ccccc3C43c4ccccc4-c4ccccc43)c3ccccc3-c3cccc4oc5ccccc5c34)cc2)cc1 ; c1ccc(N(c2ccc(-c3ccc(-c4ccc5ccccc5c4)cc3)cc2)c2ccc3c(c2)-c2ccccc2C32c3ccccc3-c3ccccc32)c(-c2cccc3oc4ccccc4c23)c1]

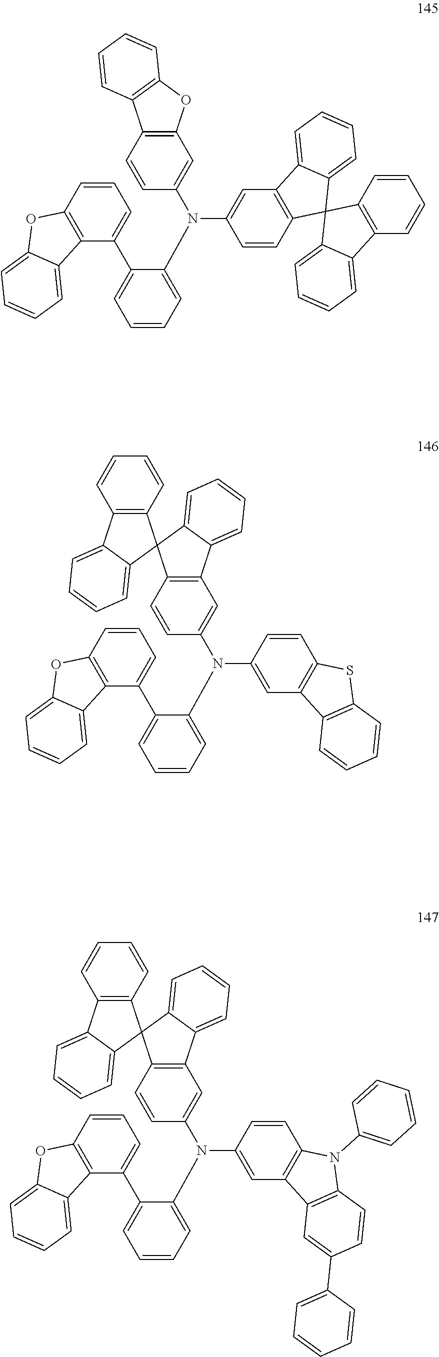 [SMILES: c1ccc(N(c2ccc3c(c2)-c2ccccc2C32c3ccccc3-c3ccccc32)c2ccc3c(c2)oc2ccccc23)c(-c2cccc3oc4ccccc4c23)c1 ; c1ccc(N(c2ccc3c(c2)-c2ccccc2C32c3ccccc3-c3ccccc32)c2ccc3sc4ccccc4c3c2)c(-c2cccc3oc4ccccc4c23)c1 ; c1ccc(-c2ccc3c(c2)c2cc(N(c4ccc5c(c4)-c4ccccc4C54c5ccccc5-c5ccccc54)c4ccccc4-c4cccc5oc6ccccc6c45)ccc2n3-c2ccccc2)cc1]

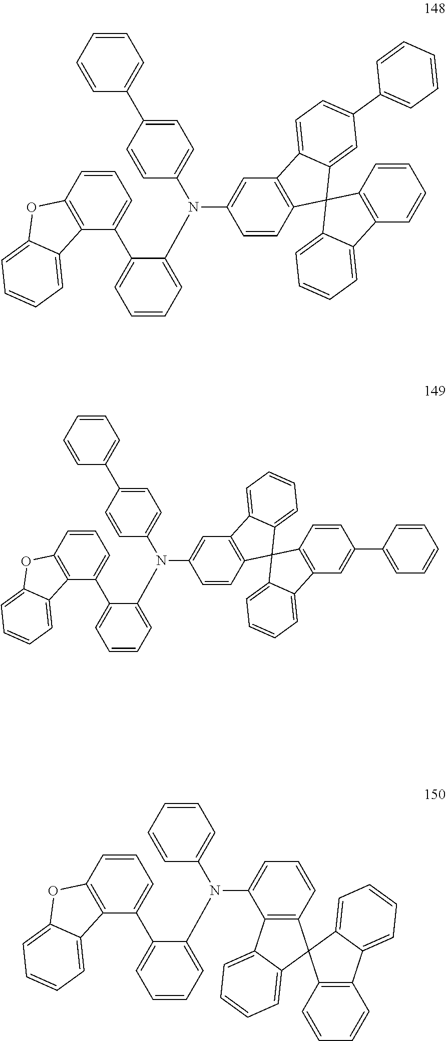 [SMILES: c1ccc(-c2ccc(N(c3ccc4c(c3)-c3ccc(-c5ccccc5)cc3C43c4ccccc4-c4ccccc43)c3ccccc3-c3cccc4oc5ccccc5c34)cc2)cc1 ; c1ccc(-c2ccc(N(c3ccc4c(c3)-c3ccccc3C43c4ccccc4-c4cc(-c5ccccc5)ccc43)c3ccccc3-c3cccc4oc5ccccc5c34)cc2)cc1 ; c1ccc(N(c2ccccc2-c2cccc3oc4ccccc4c23)c2cccc3c2-c2ccccc2C32c3ccccc3-c3ccccc32)cc1]

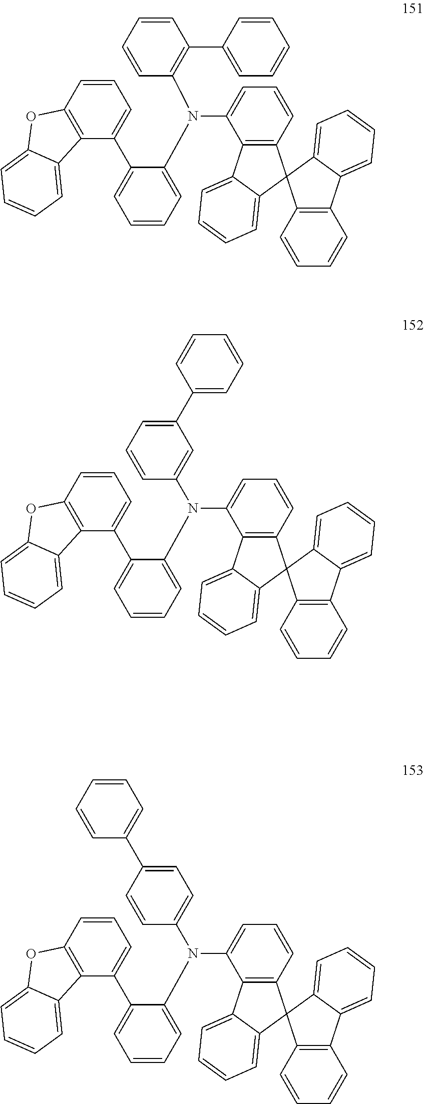 [SMILES: c1ccc(-c2ccccc2N(c2ccccc2-c2cccc3oc4ccccc4c23)c2cccc3c2-c2ccccc2C32c3ccccc3-c3ccccc32)cc1 ; c1ccc(-c2cccc(N(c3ccccc3-c3cccc4oc5ccccc5c34)c3cccc4c3-c3ccccc3C43c4ccccc4-c4ccccc43)c2)cc1 ; c1ccc(-c2ccc(N(c3ccccc3-c3cccc4oc5ccccc5c34)c3cccc4c3-c3ccccc3C43c4ccccc4-c4ccccc43)cc2)cc1]

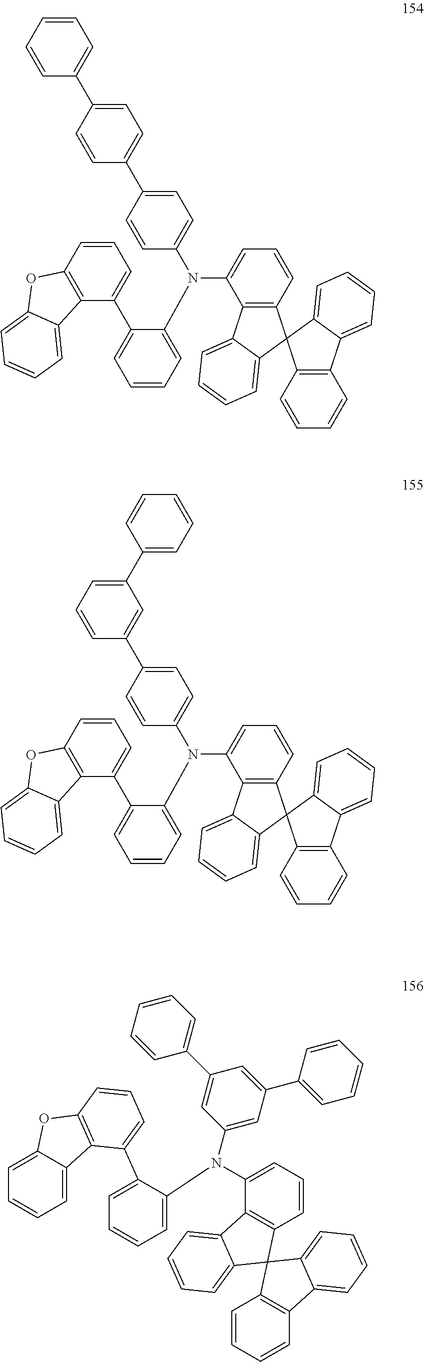 [SMILES: c1ccc(-c2ccc(-c3ccc(N(c4ccccc4-c4cccc5oc6ccccc6c45)c4cccc5c4-c4ccccc4C54c5ccccc5-c5ccccc54)cc3)cc2)cc1 ; c1ccc(-c2cccc(-c3ccc(N(c4ccccc4-c4cccc5oc6ccccc6c45)c4cccc5c4-c4ccccc4C54c5ccccc5-c5ccccc54)cc3)c2)cc1 ; c1ccc(-c2cc(-c3ccccc3)cc(N(c3ccccc3-c3cccc4oc5ccccc5c34)c3cccc4c3-c3ccccc3C43c4ccccc4-c4ccccc43)c2)cc1]

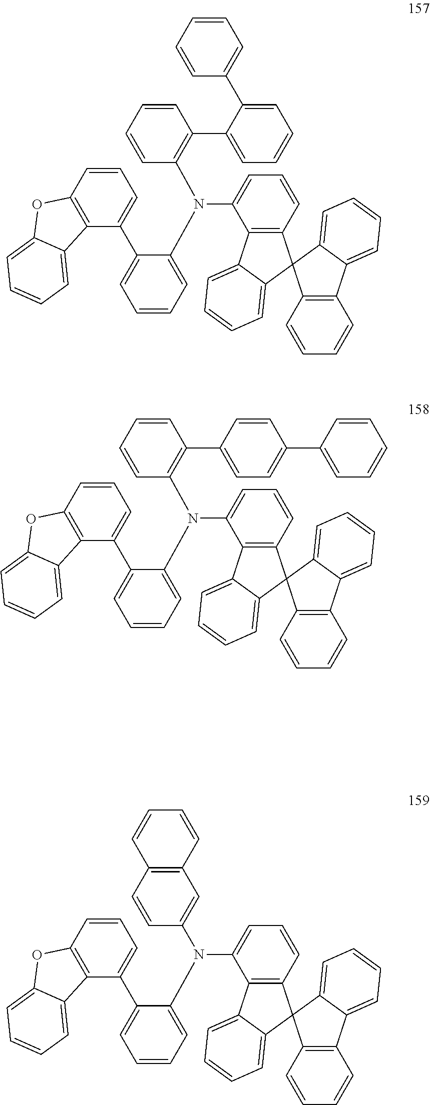 [SMILES: c1ccc(-c2ccccc2-c2ccccc2N(c2ccccc2-c2cccc3oc4ccccc4c23)c2cccc3c2-c2ccccc2C32c3ccccc3-c3ccccc32)cc1 ; c1ccc(-c2ccc(-c3ccccc3N(c3ccccc3-c3cccc4oc5ccccc5c34)c3cccc4c3-c3ccccc3C43c4ccccc4-c4ccccc43)cc2)cc1 ; c1ccc(N(c2ccc3ccccc3c2)c2cccc3c2-c2ccccc2C32c3ccccc3-c3ccccc32)c(-c2cccc3oc4ccccc4c23)c1]

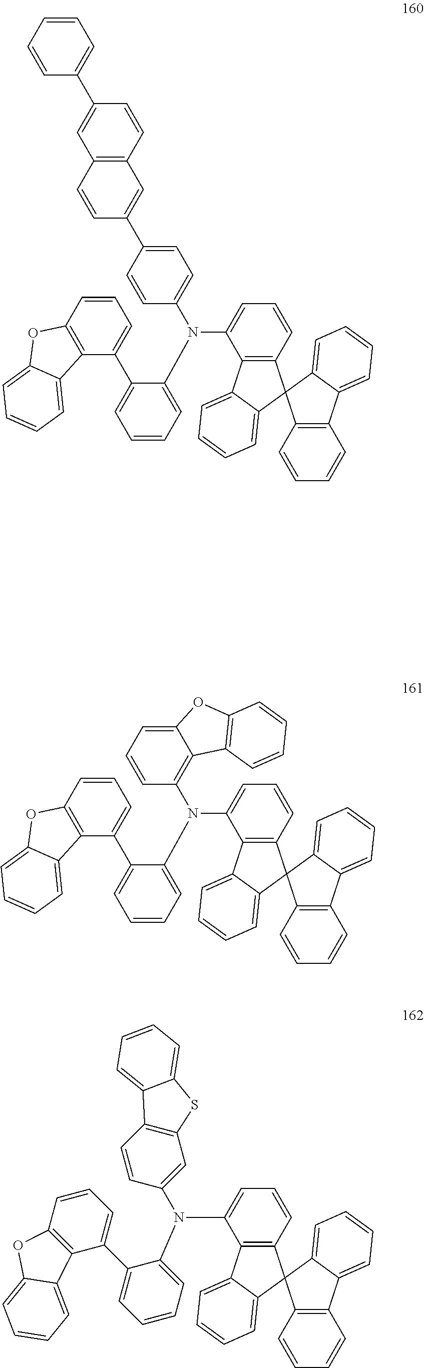 [SMILES: c1ccc(-c2ccc3cc(-c4ccc(N(c5ccccc5-c5cccc6oc7ccccc7c56)c5cccc6c5-c5ccccc5C65c6ccccc6-c6ccccc65)cc4)ccc3c2)cc1 ; c1ccc(N(c2cccc3c2-c2ccccc2C32c3ccccc3-c3ccccc32)c2cccc3oc4ccccc4c23)c(-c2cccc3oc4ccccc4c23)c1 ; c1ccc(N(c2ccc3c(c2)sc2ccccc23)c2cccc3c2-c2ccccc2C32c3ccccc3-c3ccccc32)c(-c2cccc3oc4ccccc4c23)c1]

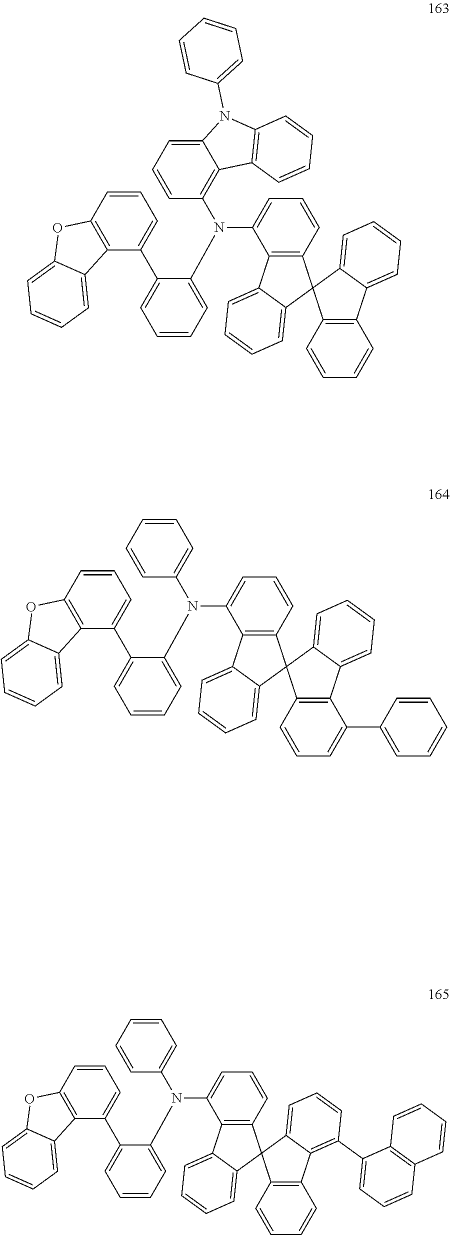 [SMILES: c1ccc(-n2c3ccccc3c3c(N(c4ccccc4-c4cccc5oc6ccccc6c45)c4cccc5c4-c4ccccc4C54c5ccccc5-c5ccccc54)cccc32)cc1 ; c1ccc(-c2cccc3c2-c2ccccc2C32c3ccccc3-c3c(N(c4ccccc4)c4ccccc4-c4cccc5oc6ccccc6c45)cccc32)cc1 ; c1ccc(N(c2ccccc2-c2cccc3oc4ccccc4c23)c2cccc3c2-c2ccccc2C32c3ccccc3-c3c(-c4cccc5ccccc45)cccc32)cc1]

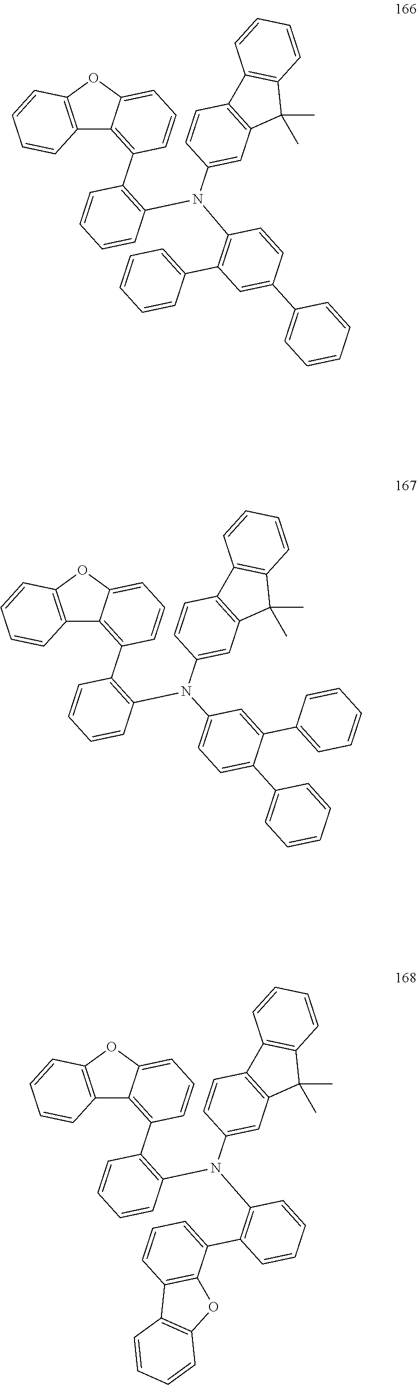 [SMILES: CC1(C)c2ccccc2-c2ccc(N(c3ccc(-c4ccccc4)cc3-c3ccccc3)c3ccccc3-c3cccc4oc5ccccc5c34)cc21 ; CC1(C)c2ccccc2-c2ccc(N(c3ccc(-c4ccccc4)c(-c4ccccc4)c3)c3ccccc3-c3cccc4oc5ccccc5c34)cc21 ; CC1(C)c2ccccc2-c2ccc(N(c3ccccc3-c3cccc4c3oc3ccccc34)c3ccccc3-c3cccc4oc5ccccc5c34)cc21]

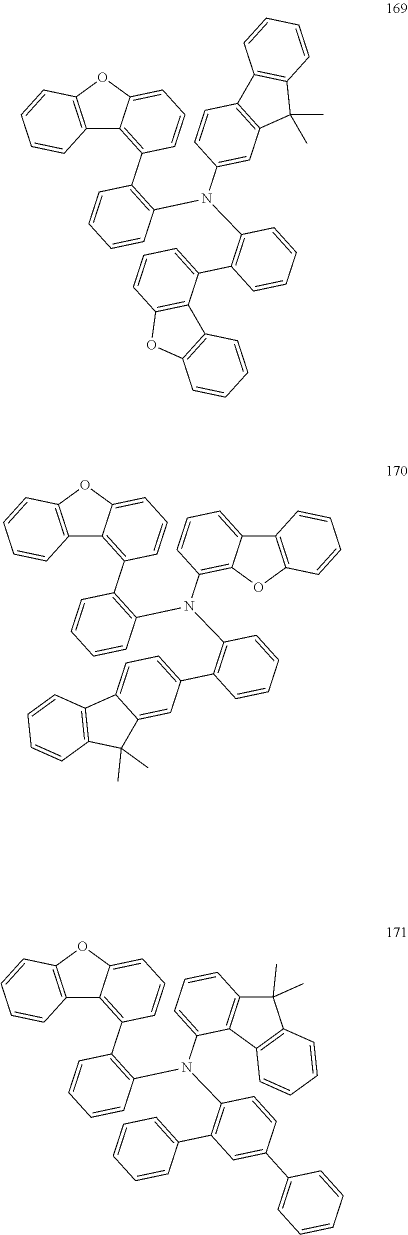 [SMILES: CC1(C)c2ccccc2-c2ccc(N(c3ccccc3-c3cccc4oc5ccccc5c34)c3ccccc3-c3cccc4oc5ccccc5c34)cc21 ; CC1(C)c2ccccc2-c2ccc(-c3ccccc3N(c3ccccc3-c3cccc4oc5ccccc5c34)c3cccc4c3oc3ccccc34)cc21 ; CC1(C)c2ccccc2-c2c(N(c3ccc(-c4ccccc4)cc3-c3ccccc3)c3ccccc3-c3cccc4oc5ccccc5c34)cccc21]

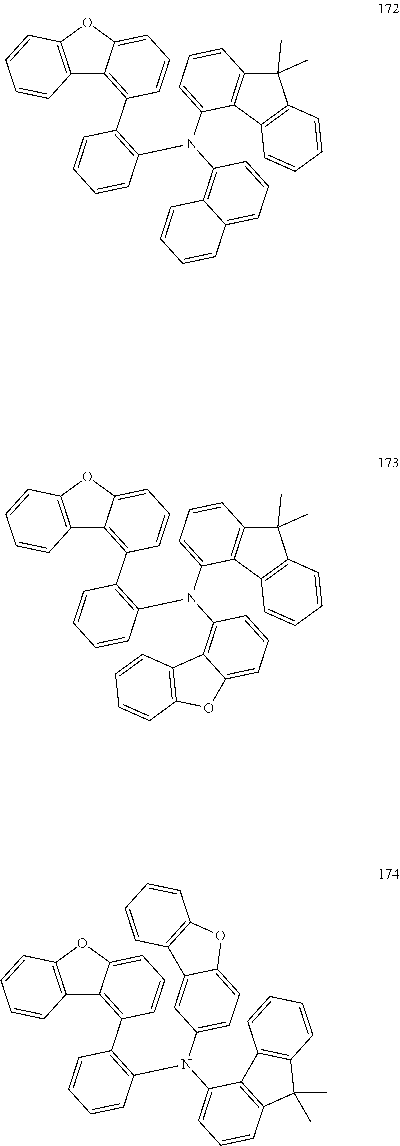 [SMILES: CC1(C)c2ccccc2-c2c(N(c3ccccc3-c3cccc4oc5ccccc5c34)c3cccc4ccccc34)cccc21 ; CC1(C)c2ccccc2-c2c(N(c3ccccc3-c3cccc4oc5ccccc5c34)c3cccc4oc5ccccc5c34)cccc21 ; CC1(C)c2ccccc2-c2c(N(c3ccc4oc5ccccc5c4c3)c3ccccc3-c3cccc4oc5ccccc5c34)cccc21]

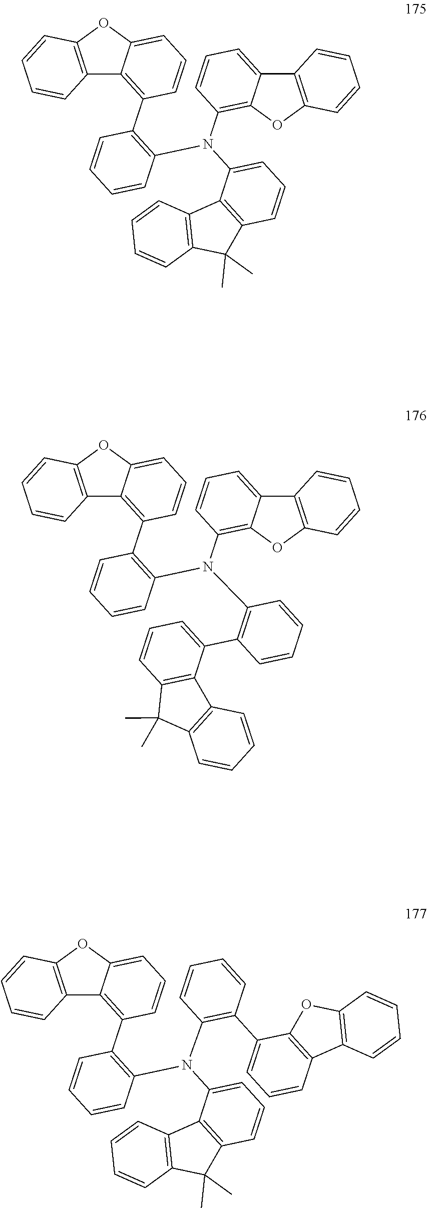 [SMILES: CC1(C)c2ccccc2-c2c(N(c3ccccc3-c3cccc4oc5ccccc5c34)c3cccc4c3oc3ccccc34)cccc21 ; CC1(C)c2ccccc2-c2c(-c3ccccc3N(c3ccccc3-c3cccc4oc5ccccc5c34)c3cccc4c3oc3ccccc34)cccc21 ; CC1(C)c2ccccc2-c2c(N(c3ccccc3-c3cccc4c3oc3ccccc34)c3ccccc3-c3cccc4oc5ccccc5c34)cccc21]

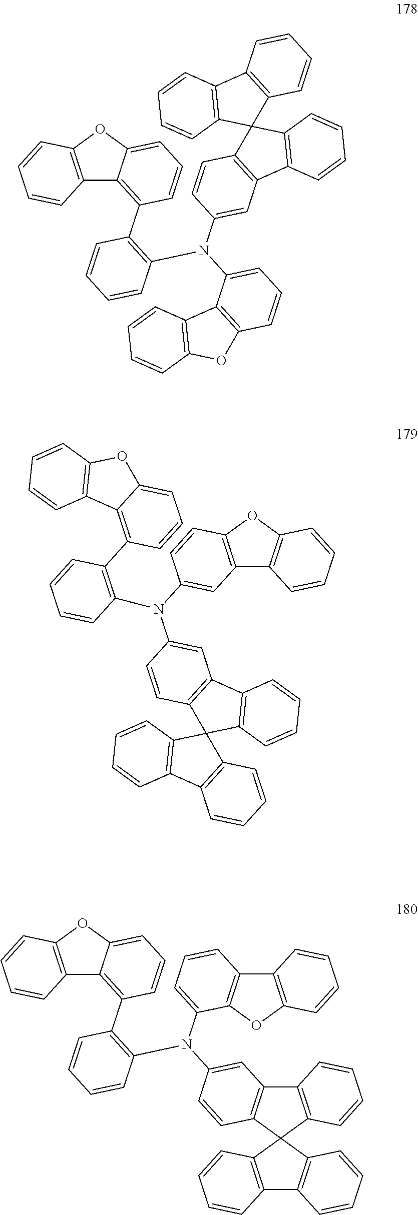 [SMILES: c1ccc(N(c2ccc3c(c2)-c2ccccc2C32c3ccccc3-c3ccccc32)c2cccc3oc4ccccc4c23)c(-c2cccc3oc4ccccc4c23)c1 ; c1ccc(N(c2ccc3c(c2)-c2ccccc2C32c3ccccc3-c3ccccc32)c2ccc3oc4ccccc4c3c2)c(-c2cccc3oc4ccccc4c23)c1 ; c1ccc(N(c2ccc3c(c2)-c2ccccc2C32c3ccccc3-c3ccccc32)c2cccc3c2oc2ccccc23)c(-c2cccc3oc4ccccc4c23)c1]

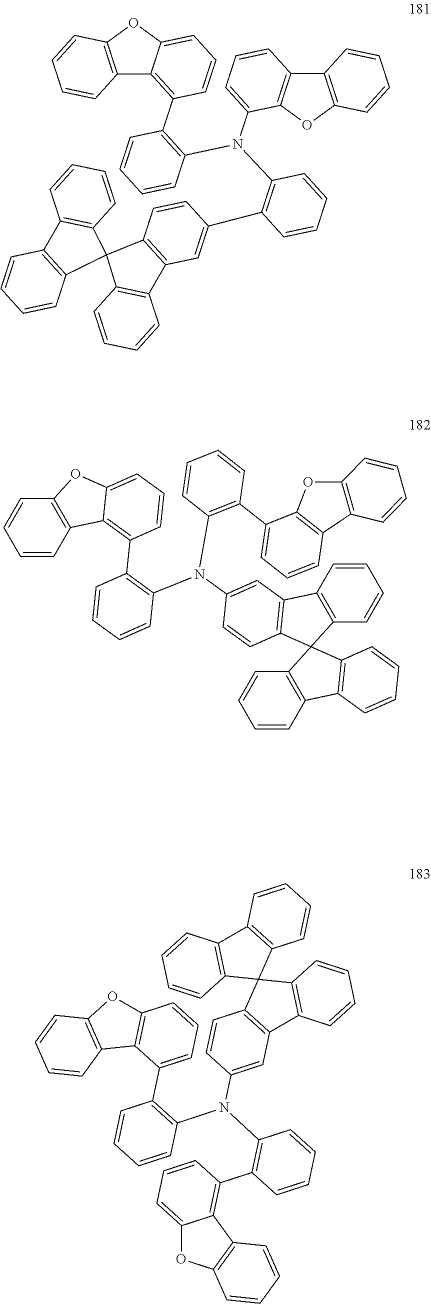 [SMILES: c1ccc(N(c2ccccc2-c2cccc3oc4ccccc4c23)c2cccc3c2oc2ccccc23)c(-c2ccc3c(c2)-c2ccccc2C32c3ccccc3-c3ccccc32)c1 ; c1ccc(N(c2ccc3c(c2)-c2ccccc2C32c3ccccc3-c3ccccc32)c2ccccc2-c2cccc3oc4ccccc4c23)c(-c2cccc3c2oc2ccccc23)c1 ; c1ccc(N(c2ccc3c(c2)-c2ccccc2C32c3ccccc3-c3ccccc32)c2ccccc2-c2cccc3oc4ccccc4c23)c(-c2cccc3oc4ccccc4c23)c1]

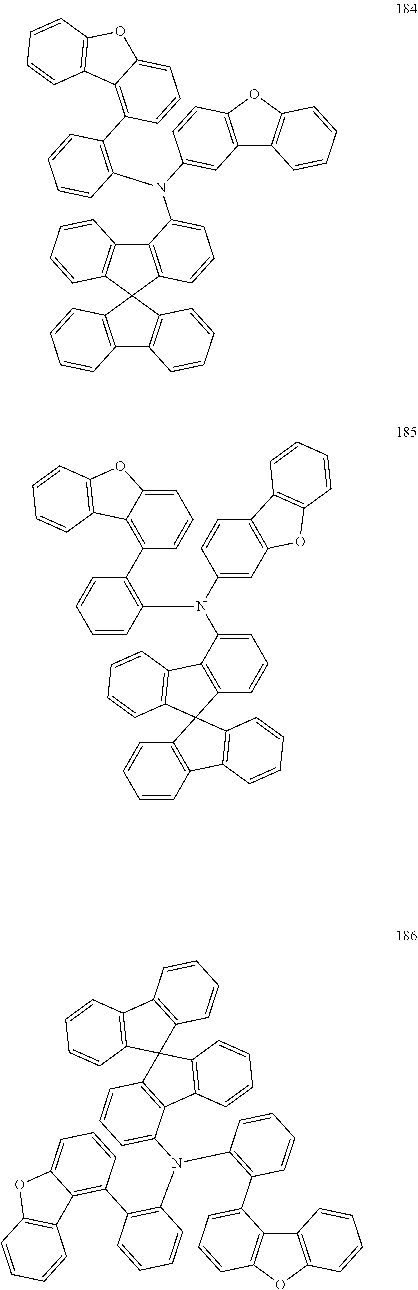 [SMILES: c1ccc(N(c2ccc3oc4ccccc4c3c2)c2cccc3c2-c2ccccc2C32c3ccccc3-c3ccccc32)c(-c2cccc3oc4ccccc4c23)c1 ; c1ccc(N(c2ccc3c(c2)oc2ccccc23)c2cccc3c2-c2ccccc2C32c3ccccc3-c3ccccc32)c(-c2cccc3oc4ccccc4c23)c1 ; c1ccc(N(c2ccccc2-c2cccc3oc4ccccc4c23)c2cccc3c2-c2ccccc2C32c3ccccc3-c3ccccc32)c(-c2cccc3oc4ccccc4c23)c1]

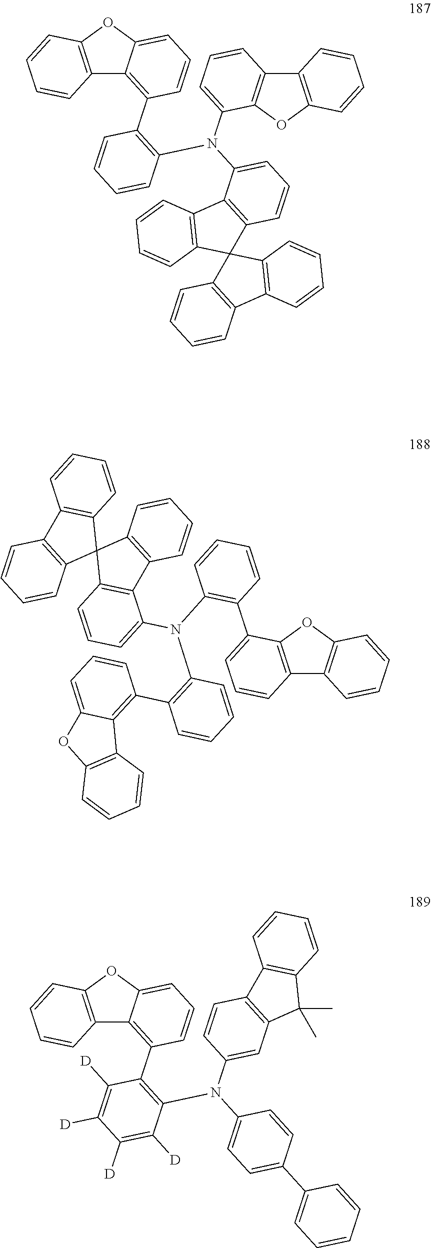 [SMILES: c1ccc(N(c2cccc3c2-c2ccccc2C32c3ccccc3-c3ccccc32)c2cccc3c2oc2ccccc23)c(-c2cccc3oc4ccccc4c23)c1 ; c1ccc(N(c2ccccc2-c2cccc3oc4ccccc4c23)c2cccc3c2-c2ccccc2C32c3ccccc3-c3ccccc32)c(-c2cccc3c2oc2ccccc23)c1 ; [2H]c1c([2H])c([2H])c(N(c2ccc(-c3ccccc3)cc2)c2ccc3c(c2)C(C)(C)c2ccccc2-3)c(-c2cccc3oc4ccccc4c23)c1[2H]]

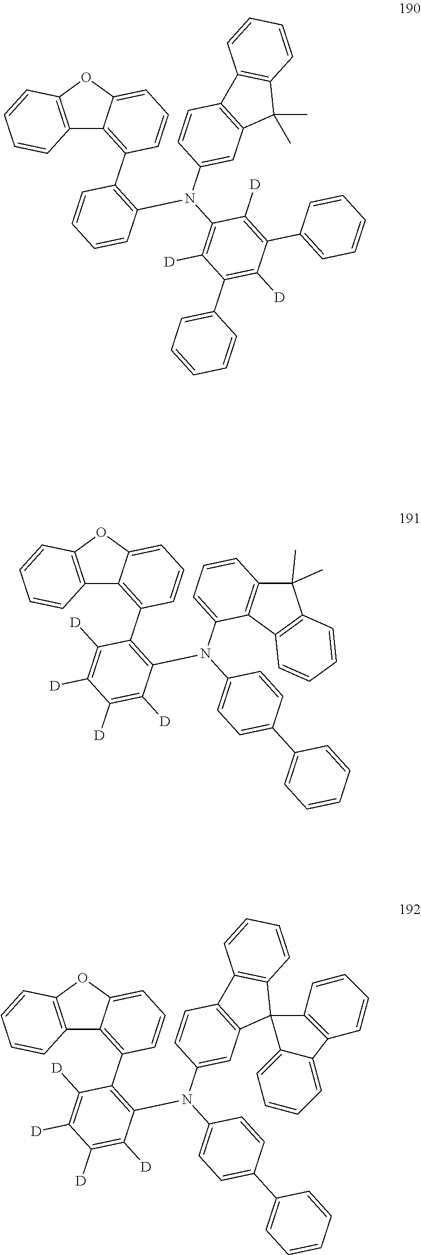 [SMILES: [2H]c1c(-c2ccccc2)c([2H])c(N(c2ccc3c(c2)C(C)(C)c2ccccc2-3)c2ccccc2-c2cccc3oc4ccccc4c23)c([2H])c1-c1ccccc1 ; [2H]c1c([2H])c([2H])c(N(c2ccc(-c3ccccc3)cc2)c2cccc3c2-c2ccccc2C3(C)C)c(-c2cccc3oc4ccccc4c23)c1[2H] ; [2H]c1c([2H])c([2H])c(N(c2ccc(-c3ccccc3)cc2)c2ccc3c(c2)C2(c4ccccc4-c4ccccc42)c2ccccc2-3)c(-c2cccc3oc4ccccc4c23)c1[2H]]

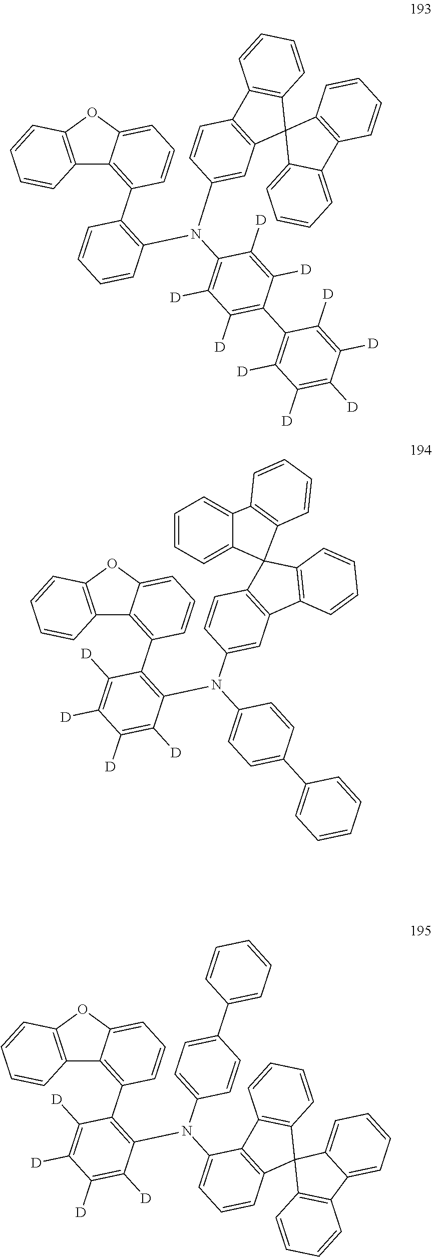

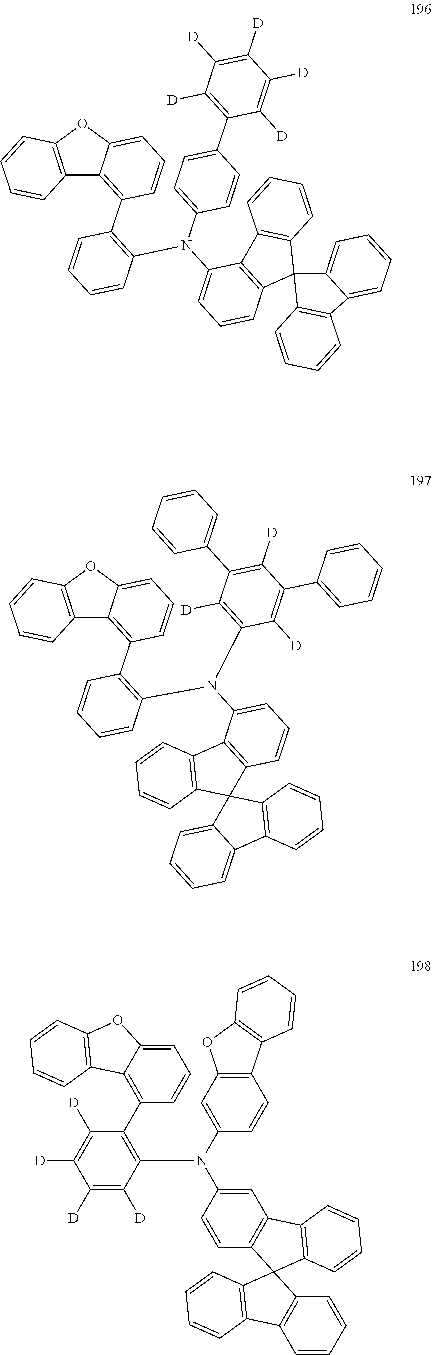 [SMILES: [2H]c1c([2H])c([2H])c(-c2ccc(N(c3ccccc3-c3cccc4oc5ccccc5c34)c3cccc4c3-c3ccccc3C43c4ccccc4-c4ccccc43)cc2)c([2H])c1[2H] ; [2H]c1c(-c2ccccc2)c([2H])c(N(c2ccccc2-c2cccc3oc4ccccc4c23)c2cccc3c2-c2ccccc2C32c3ccccc3-c3ccccc32)c([2H])c1-c1ccccc1 ; [2H]c1c([2H])c([2H])c(N(c2ccc3c(c2)-c2ccccc2C32c3ccccc3-c3ccccc32)c2ccc3c(c2)oc2ccccc23)c(-c2cccc3oc4ccccc4c23)c1[2H]]

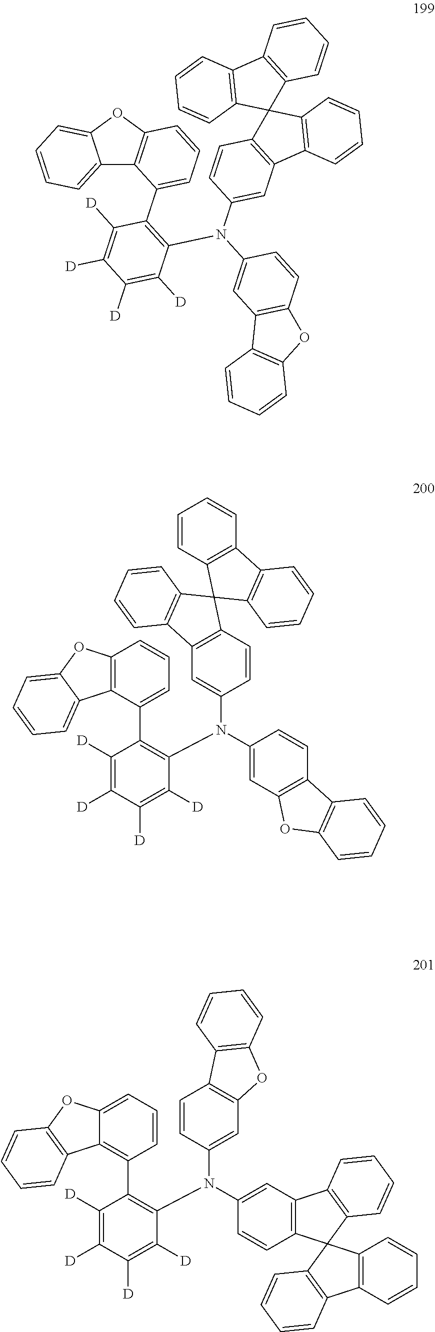 [SMILES: [2H]c1c([2H])c([2H])c(N(c2ccc3c(c2)-c2ccccc2C32c3ccccc3-c3ccccc32)c2ccc3oc4ccccc4c3c2)c(-c2cccc3oc4ccccc4c23)c1[2H] ; [2H]c1c([2H])c([2H])c(N(c2ccc3c(c2)-c2ccccc2C32c3ccccc3-c3ccccc32)c2ccc3c(c2)oc2ccccc23)c(-c2cccc3oc4ccccc4c23)c1[2H] ; [2H]c1c([2H])c([2H])c(N(c2ccc3c(c2)-c2ccccc2C32c3ccccc3-c3ccccc32)c2ccc3c(c2)oc2ccccc23)c(-c2cccc3oc4ccccc4c23)c1[2H]]

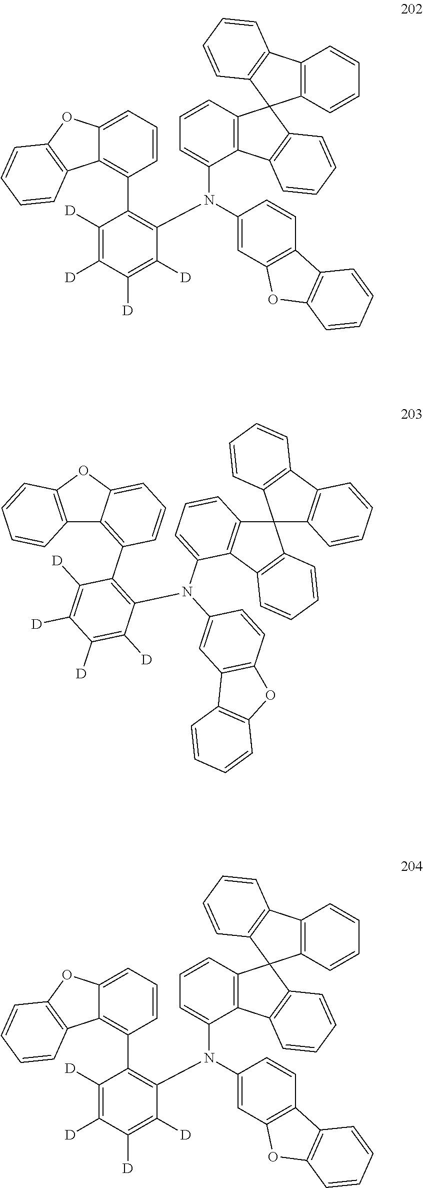 [SMILES: [2H]c1c([2H])c([2H])c(N(c2ccc3c(c2)oc2ccccc23)c2cccc3c2-c2ccccc2C32c3ccccc3-c3ccccc32)c(-c2cccc3oc4ccccc4c23)c1[2H] ; [2H]c1c([2H])c([2H])c(N(c2ccc3oc4ccccc4c3c2)c2cccc3c2-c2ccccc2C32c3ccccc3-c3ccccc32)c(-c2cccc3oc4ccccc4c23)c1[2H] ; [2H]c1c([2H])c([2H])c(N(c2ccc3c(c2)oc2ccccc23)c2cccc3c2-c2ccccc2C32c3ccccc3-c3ccccc32)c(-c2cccc3oc4ccccc4c23)c1[2H]]

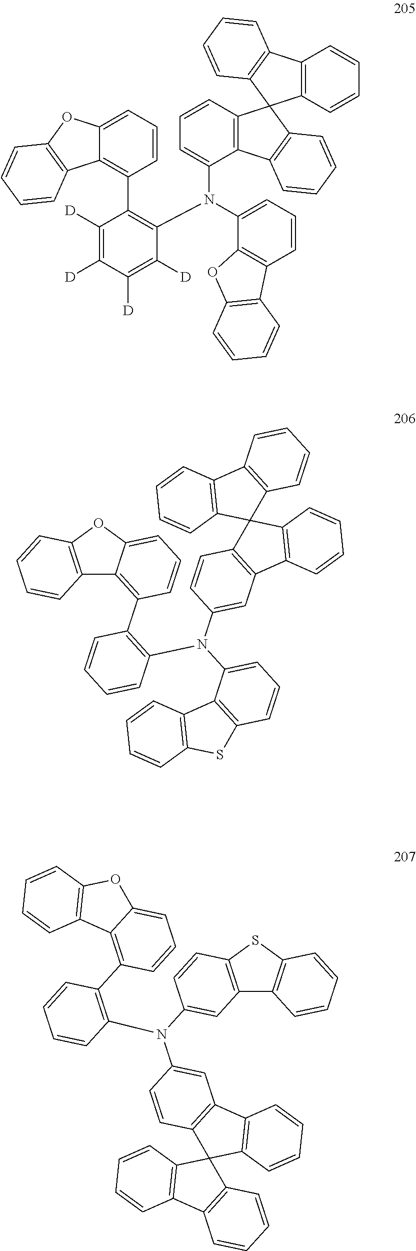 [SMILES: [2H]c1c([2H])c([2H])c(N(c2cccc3c2-c2ccccc2C32c3ccccc3-c3ccccc32)c2cccc3c2oc2ccccc23)c(-c2cccc3oc4ccccc4c23)c1[2H] ; c1ccc(N(c2ccc3c(c2)-c2ccccc2C32c3ccccc3-c3ccccc32)c2cccc3sc4ccccc4c23)c(-c2cccc3oc4ccccc4c23)c1 ; c1ccc(N(c2ccc3c(c2)-c2ccccc2C32c3ccccc3-c3ccccc32)c2ccc3sc4ccccc4c3c2)c(-c2cccc3oc4ccccc4c23)c1]

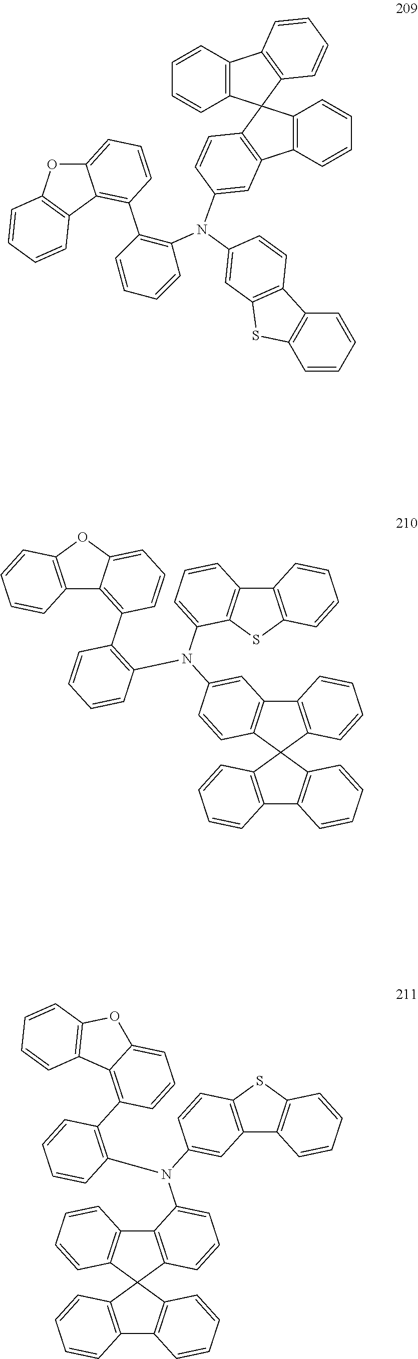 [SMILES: c1ccc(N(c2ccc3c(c2)-c2ccccc2C32c3ccccc3-c3ccccc32)c2ccc3c(c2)sc2ccccc23)c(-c2cccc3oc4ccccc4c23)c1 ; c1ccc(N(c2ccc3c(c2)-c2ccccc2C32c3ccccc3-c3ccccc32)c2cccc3c2sc2ccccc23)c(-c2cccc3oc4ccccc4c23)c1 ; c1ccc(N(c2ccc3sc4ccccc4c3c2)c2cccc3c2-c2ccccc2C32c3ccccc3-c3ccccc32)c(-c2cccc3oc4ccccc4c23)c1]

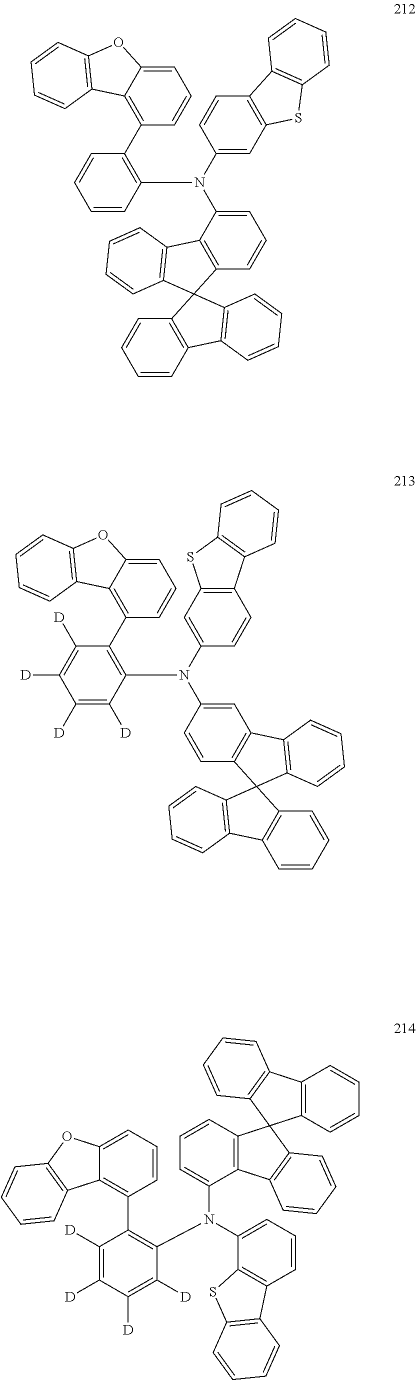 [SMILES: c1ccc(N(c2ccc3c(c2)sc2ccccc23)c2cccc3c2-c2ccccc2C32c3ccccc3-c3ccccc32)c(-c2cccc3oc4ccccc4c23)c1 ; [2H]c1c([2H])c([2H])c(N(c2ccc3c(c2)-c2ccccc2C32c3ccccc3-c3ccccc32)c2ccc3c(c2)sc2ccccc23)c(-c2cccc3oc4ccccc4c23)c1[2H] ; [2H]c1c([2H])c([2H])c(N(c2cccc3c2-c2ccccc2C32c3ccccc3-c3ccccc32)c2cccc3c2sc2ccccc23)c(-c2cccc3oc4ccccc4c23)c1[2H]]

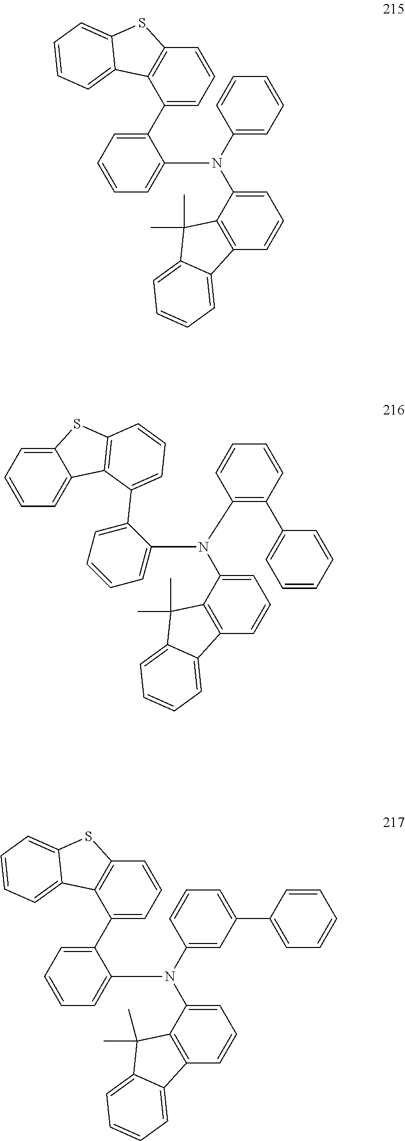 [SMILES: CC1(C)c2ccccc2-c2cccc(N(c3ccccc3)c3ccccc3-c3cccc4sc5ccccc5c34)c21 ; CC1(C)c2ccccc2-c2cccc(N(c3ccccc3-c3ccccc3)c3ccccc3-c3cccc4sc5ccccc5c34)c21 ; CC1(C)c2ccccc2-c2cccc(N(c3cccc(-c4ccccc4)c3)c3ccccc3-c3cccc4sc5ccccc5c34)c21]

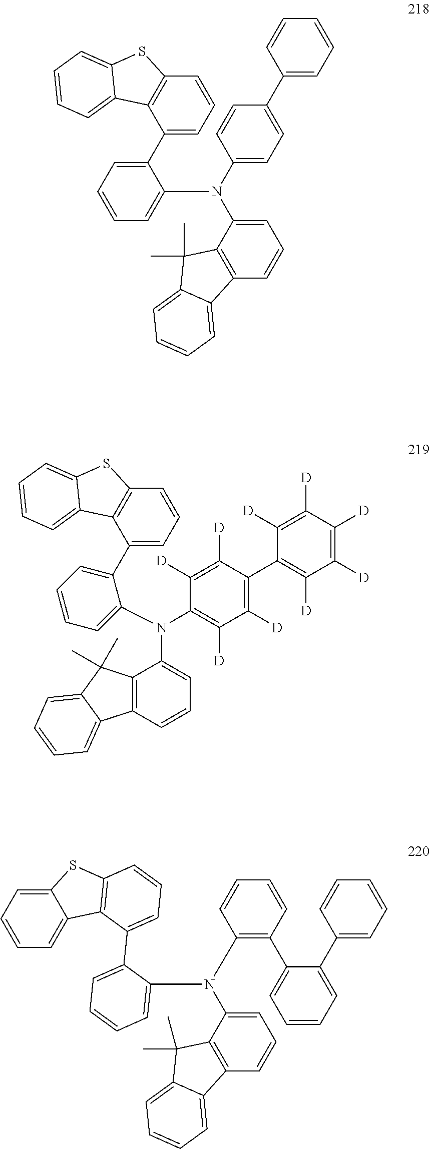 [SMILES: CC1(C)c2ccccc2-c2cccc(N(c3ccc(-c4ccccc4)cc3)c3ccccc3-c3cccc4sc5ccccc5c34)c21 ; [2H]c1c([2H])c([2H])c(-c2c([2H])c([2H])c(N(c3ccccc3-c3cccc4sc5ccccc5c34)c3cccc4c3C(C)(C)c3ccccc3-4)c([2H])c2[2H])c([2H])c1[2H] ; CC1(C)c2ccccc2-c2cccc(N(c3ccccc3-c3ccccc3-c3ccccc3)c3ccccc3-c3cccc4sc5ccccc5c34)c21]

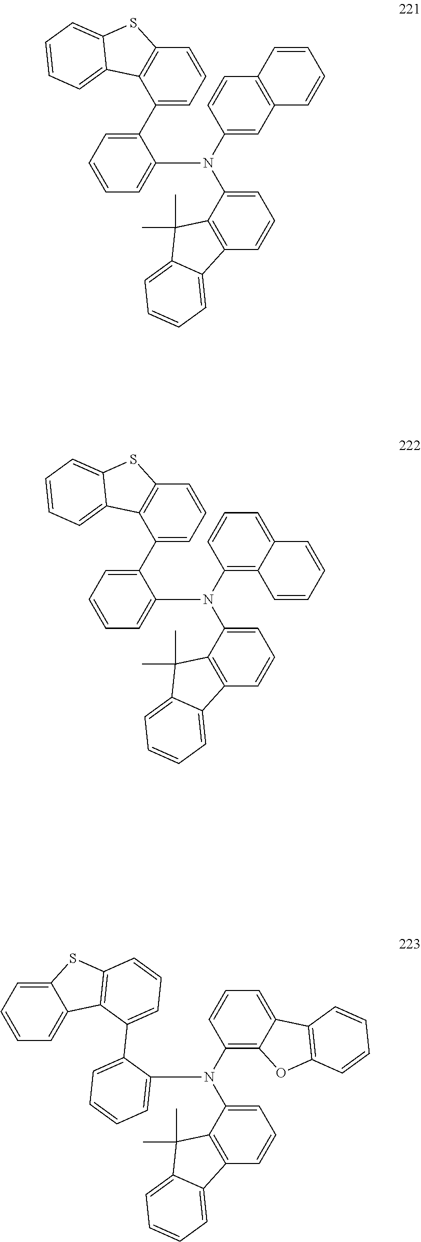 [SMILES: CC1(C)c2ccccc2-c2cccc(N(c3ccc4ccccc4c3)c3ccccc3-c3cccc4sc5ccccc5c34)c21 ; CC1(C)c2ccccc2-c2cccc(N(c3ccccc3-c3cccc4sc5ccccc5c34)c3cccc4ccccc34)c21 ; CC1(C)c2ccccc2-c2cccc(N(c3ccccc3-c3cccc4sc5ccccc5c34)c3cccc4c3oc3ccccc34)c21]

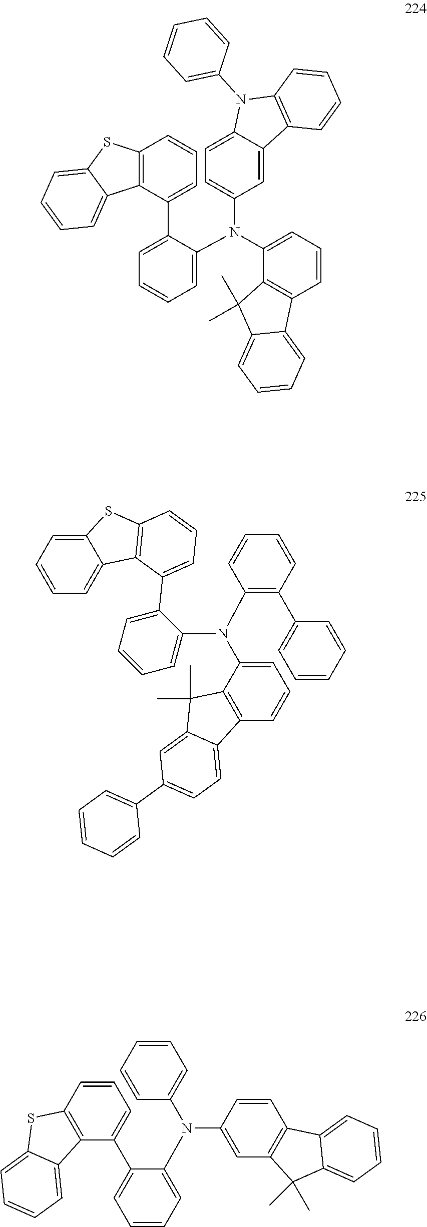 [SMILES: CC1(C)c2ccccc2-c2cccc(N(c3ccc4c(c3)c3ccccc3n4-c3ccccc3)c3ccccc3-c3cccc4sc5ccccc5c34)c21 ; CC1(C)c2cc(-c3ccccc3)ccc2-c2cccc(N(c3ccccc3-c3ccccc3)c3ccccc3-c3cccc4sc5ccccc5c34)c21 ; CC1(C)c2ccccc2-c2ccc(N(c3ccccc3)c3ccccc3-c3cccc4sc5ccccc5c34)cc21]

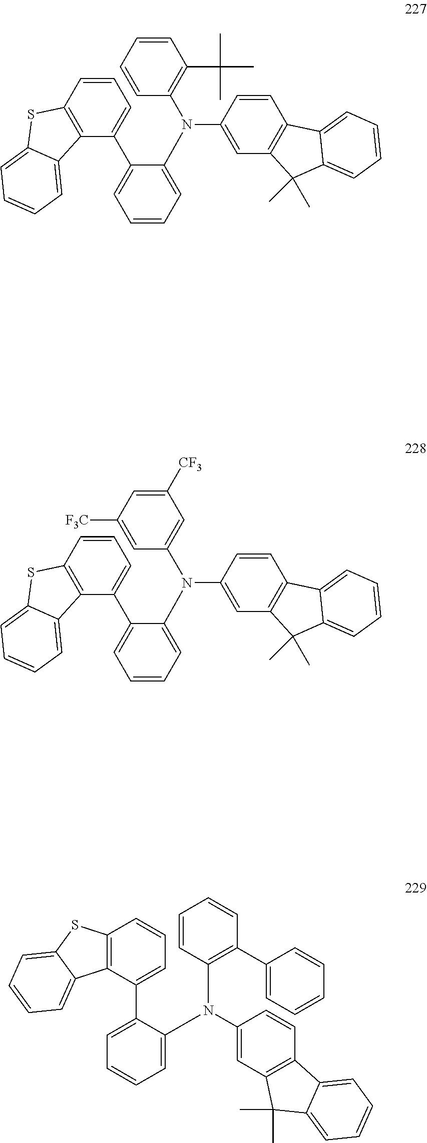 [SMILES: CC(C)(C)c1ccccc1N(c1ccc2c(c1)C(C)(C)c1ccccc1-2)c1ccccc1-c1cccc2sc3ccccc3c12 ; CC1(C)c2ccccc2-c2ccc(N(c3cc(C(F)(F)F)cc(C(F)(F)F)c3)c3ccccc3-c3cccc4sc5ccccc5c34)cc21 ; CC1(C)c2ccccc2-c2ccc(N(c3ccccc3-c3ccccc3)c3ccccc3-c3cccc4sc5ccccc5c34)cc21]

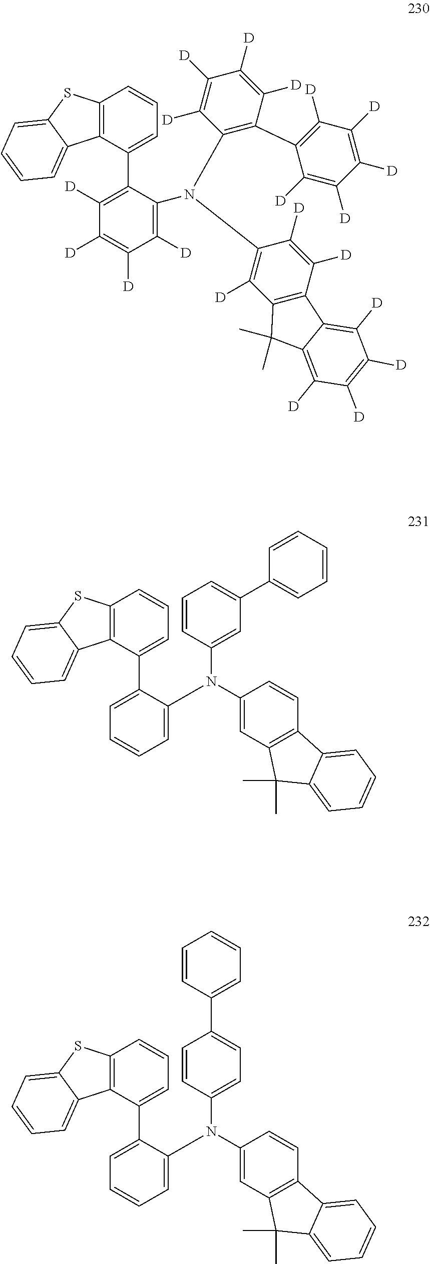 [SMILES: [2H]c1c([2H])c([2H])c(-c2c([2H])c([2H])c([2H])c([2H])c2N(c2c([2H])c([2H])c3c(c2[2H])C(C)(C)c2c([2H])c([2H])c([2H])c([2H])c2-3)c2c([2H])c([2H])c([2H])c([2H])c2-c2cccc3sc4ccccc4c23)c([2H])c1[2H] ; CC1(C)c2ccccc2-c2ccc(N(c3cccc(-c4ccccc4)c3)c3ccccc3-c3cccc4sc5ccccc5c34)cc21 ; CC1(C)c2ccccc2-c2ccc(N(c3ccc(-c4ccccc4)cc3)c3ccccc3-c3cccc4sc5ccccc5c34)cc21]

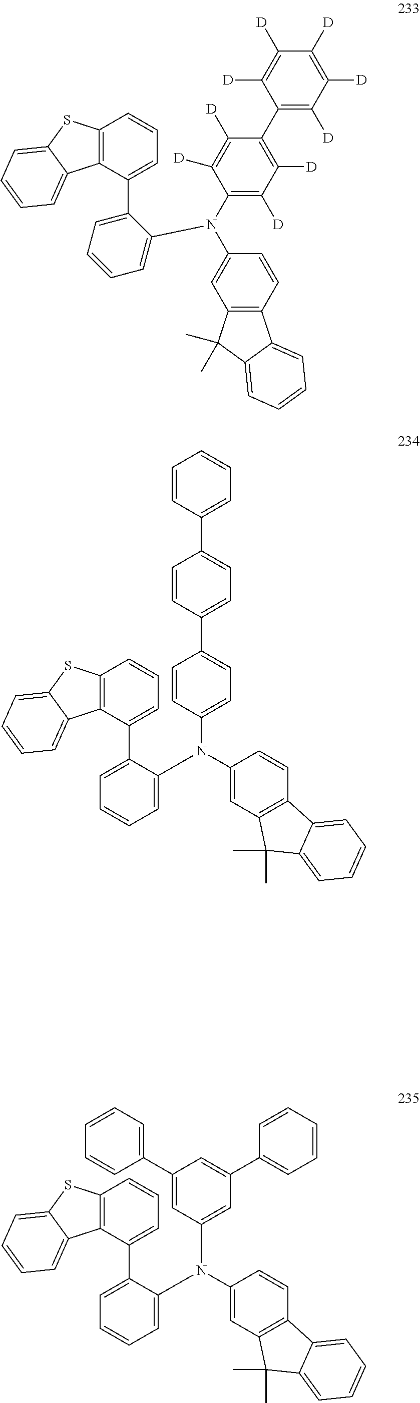 [SMILES: [2H]c1c([2H])c([2H])c(-c2c([2H])c([2H])c(N(c3ccc4c(c3)C(C)(C)c3ccccc3-4)c3ccccc3-c3cccc4sc5ccccc5c34)c([2H])c2[2H])c([2H])c1[2H] ; CC1(C)c2ccccc2-c2ccc(N(c3ccc(-c4ccc(-c5ccccc5)cc4)cc3)c3ccccc3-c3cccc4sc5ccccc5c34)cc21 ; CC1(C)c2ccccc2-c2ccc(N(c3cc(-c4ccccc4)cc(-c4ccccc4)c3)c3ccccc3-c3cccc4sc5ccccc5c34)cc21]

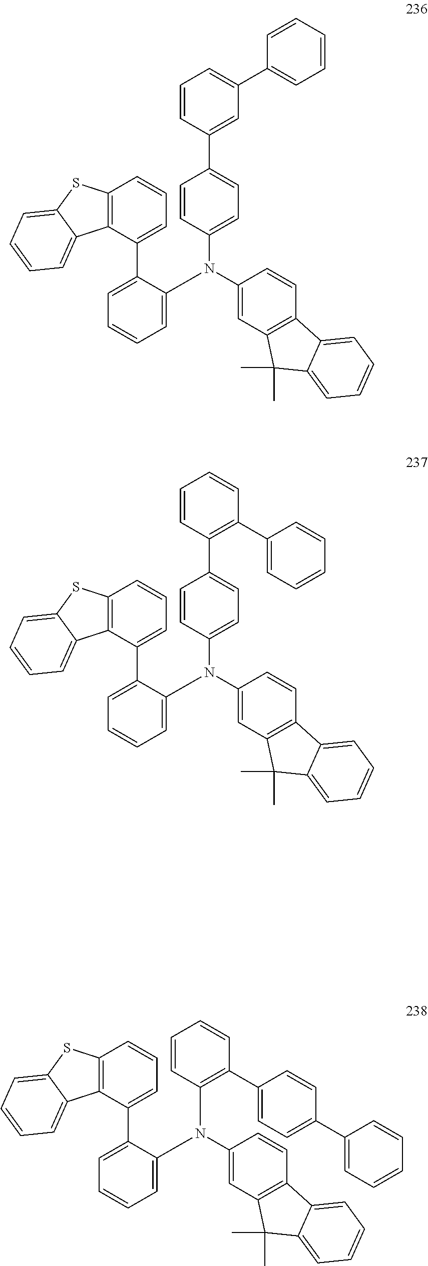 [SMILES: CC1(C)c2ccccc2-c2ccc(N(c3ccc(-c4cccc(-c5ccccc5)c4)cc3)c3ccccc3-c3cccc4sc5ccccc5c34)cc21 ; CC1(C)c2ccccc2-c2ccc(N(c3ccc(-c4ccccc4-c4ccccc4)cc3)c3ccccc3-c3cccc4sc5ccccc5c34)cc21 ; CC1(C)c2ccccc2-c2ccc(N(c3ccccc3-c3ccc(-c4ccccc4)cc3)c3ccccc3-c3cccc4sc5ccccc5c34)cc21]

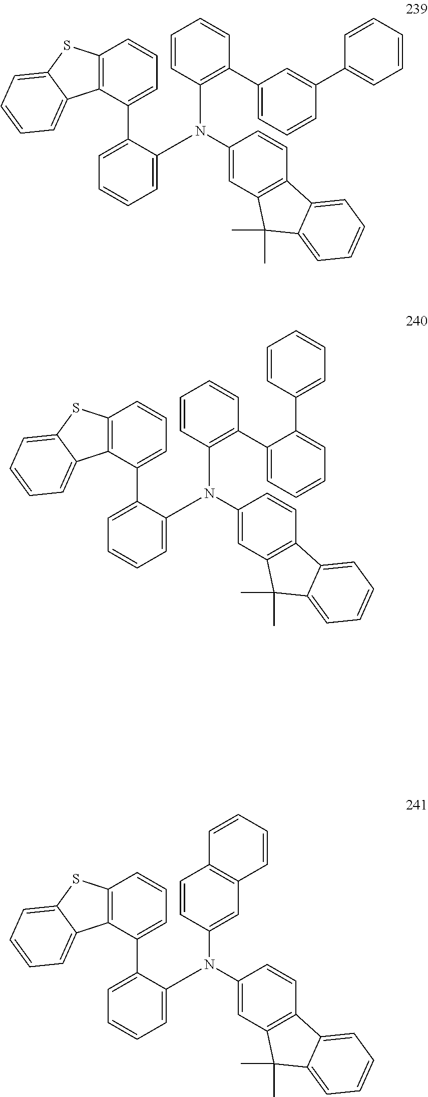 [SMILES: CC1(C)c2ccccc2-c2ccc(N(c3ccccc3-c3cccc(-c4ccccc4)c3)c3ccccc3-c3cccc4sc5ccccc5c34)cc21 ; CC1(C)c2ccccc2-c2ccc(N(c3ccccc3-c3ccccc3-c3ccccc3)c3ccccc3-c3cccc4sc5ccccc5c34)cc21 ; CC1(C)c2ccccc2-c2ccc(N(c3ccc4ccccc4c3)c3ccccc3-c3cccc4sc5ccccc5c34)cc21]

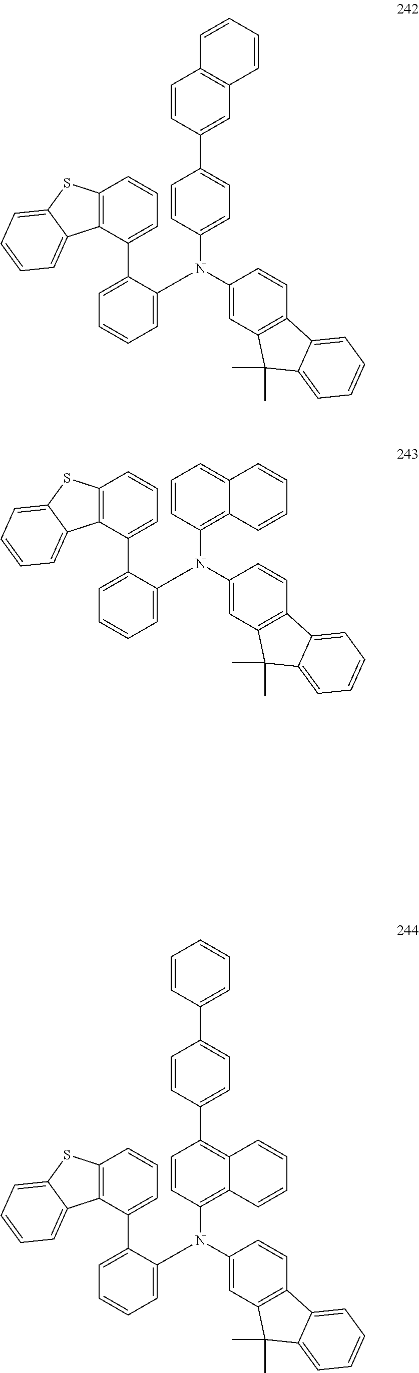 [SMILES: CC1(C)c2ccccc2-c2ccc(N(c3ccc(-c4ccc5ccccc5c4)cc3)c3ccccc3-c3cccc4sc5ccccc5c34)cc21 ; CC1(C)c2ccccc2-c2ccc(N(c3ccccc3-c3cccc4sc5ccccc5c34)c3cccc4ccccc34)cc21 ; CC1(C)c2ccccc2-c2ccc(N(c3ccccc3-c3cccc4sc5ccccc5c34)c3ccc(-c4ccc(-c5ccccc5)cc4)c4ccccc34)cc21]

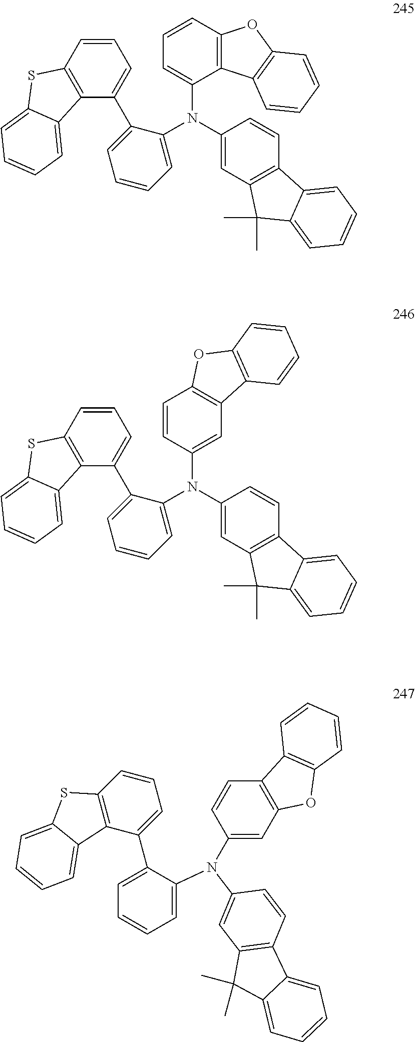 [SMILES: CC1(C)c2ccccc2-c2ccc(N(c3ccccc3-c3cccc4sc5ccccc5c34)c3cccc4oc5ccccc5c34)cc21 ; CC1(C)c2ccccc2-c2ccc(N(c3ccc4oc5ccccc5c4c3)c3ccccc3-c3cccc4sc5ccccc5c34)cc21 ; CC1(C)c2ccccc2-c2ccc(N(c3ccc4c(c3)oc3ccccc34)c3ccccc3-c3cccc4sc5ccccc5c34)cc21]

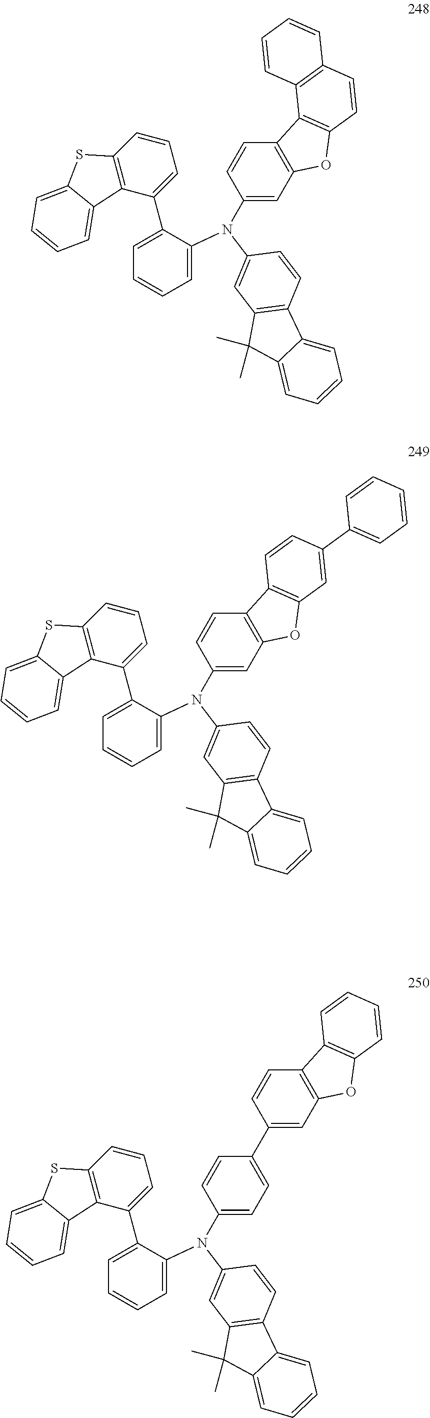 [SMILES: CC1(C)c2ccccc2-c2ccc(N(c3ccc4c(c3)oc3ccc5ccccc5c34)c3ccccc3-c3cccc4sc5ccccc5c34)cc21 ; CC1(C)c2ccccc2-c2ccc(N(c3ccc4c(c3)oc3cc(-c5ccccc5)ccc34)c3ccccc3-c3cccc4sc5ccccc5c34)cc21 ; CC1(C)c2ccccc2-c2ccc(N(c3ccc(-c4ccc5c(c4)oc4ccccc45)cc3)c3ccccc3-c3cccc4sc5ccccc5c34)cc21]

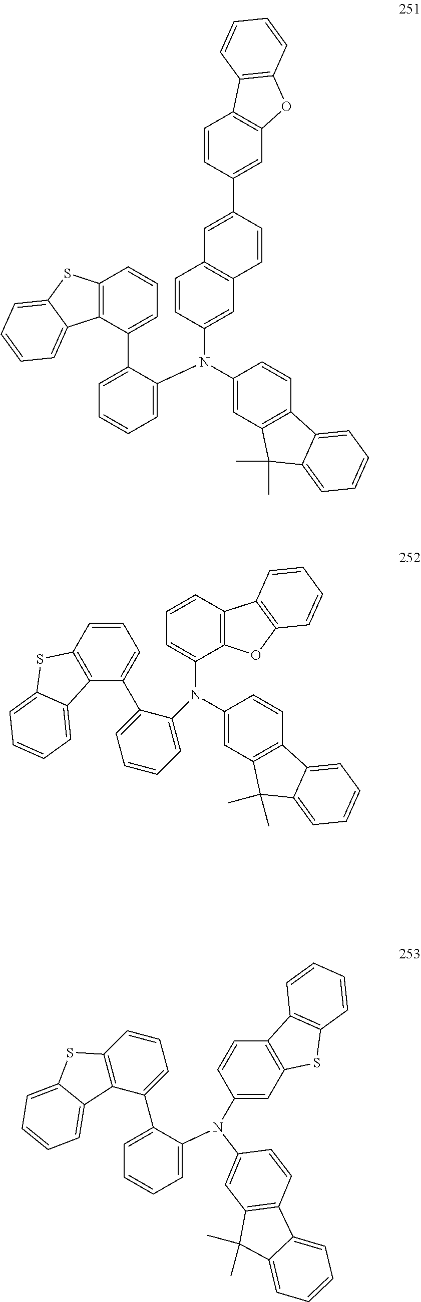 [SMILES: CC1(C)c2ccccc2-c2ccc(N(c3ccc4cc(-c5ccc6c(c5)oc5ccccc56)ccc4c3)c3ccccc3-c3cccc4sc5ccccc5c34)cc21 ; CC1(C)c2ccccc2-c2ccc(N(c3ccccc3-c3cccc4sc5ccccc5c34)c3cccc4c3oc3ccccc34)cc21 ; CC1(C)c2ccccc2-c2ccc(N(c3ccc4c(c3)sc3ccccc34)c3ccccc3-c3cccc4sc5ccccc5c34)cc21]

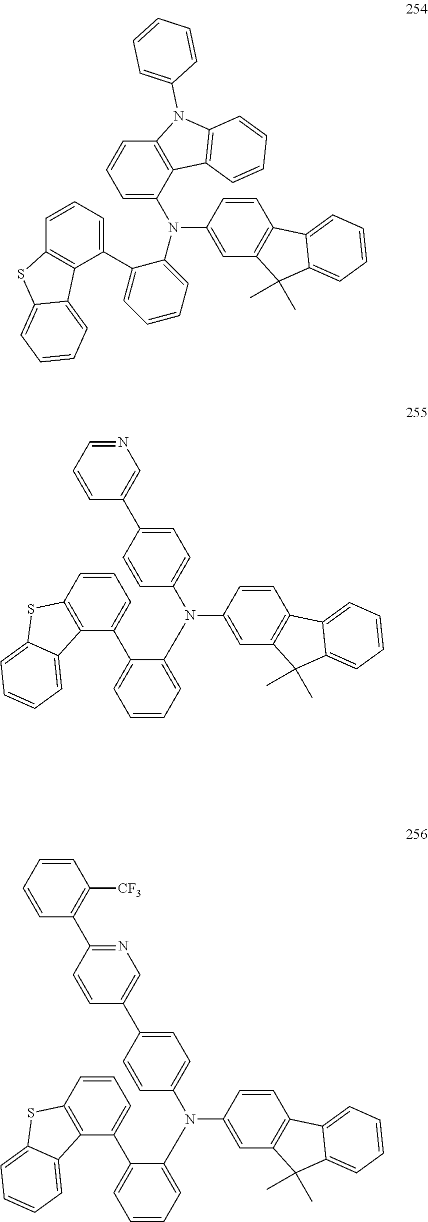 [SMILES: CC1(C)c2ccccc2-c2ccc(N(c3ccccc3-c3cccc4sc5ccccc5c34)c3cccc4c3c3ccccc3n4-c3ccccc3)cc21 ; CC1(C)c2ccccc2-c2ccc(N(c3ccc(-c4cccnc4)cc3)c3ccccc3-c3cccc4sc5ccccc5c34)cc21 ; CC1(C)c2ccccc2-c2ccc(N(c3ccc(-c4ccc(-c5ccccc5C(F)(F)F)nc4)cc3)c3ccccc3-c3cccc4sc5ccccc5c34)cc21]

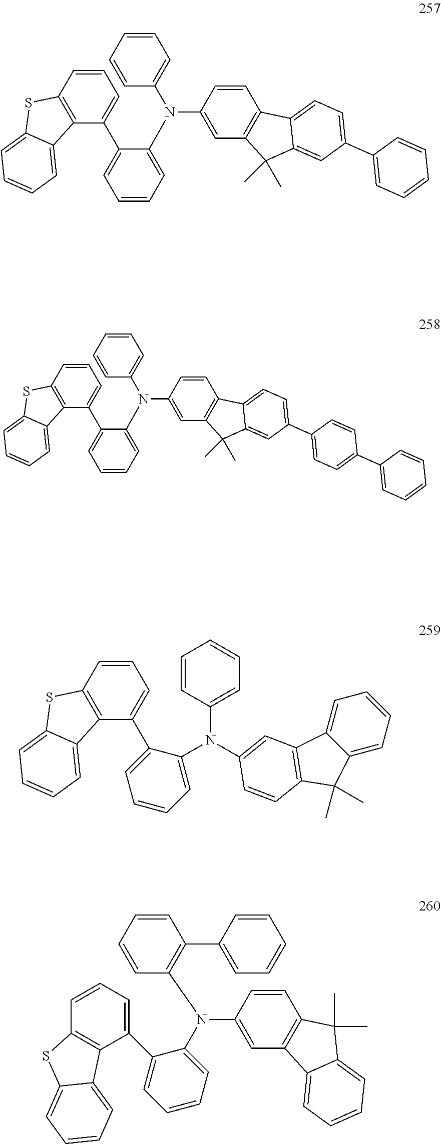

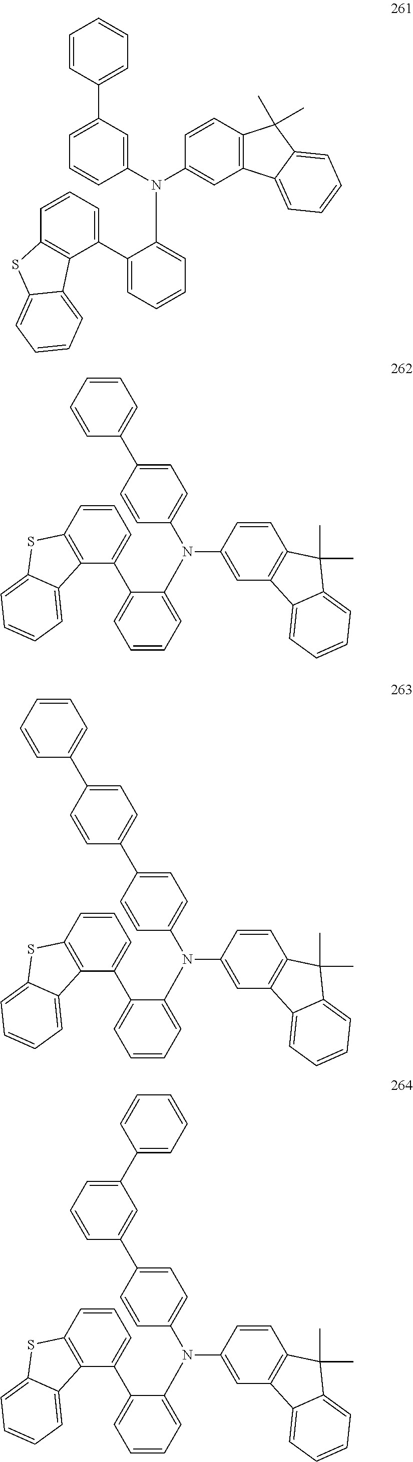

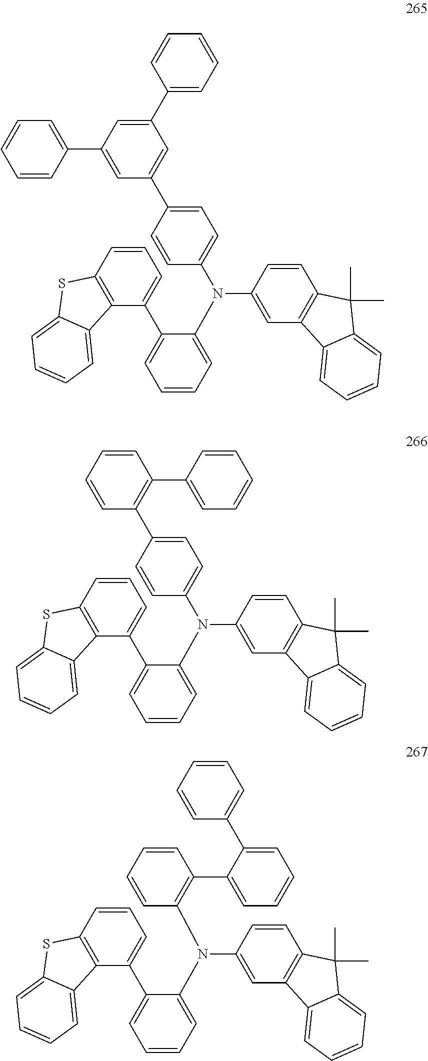

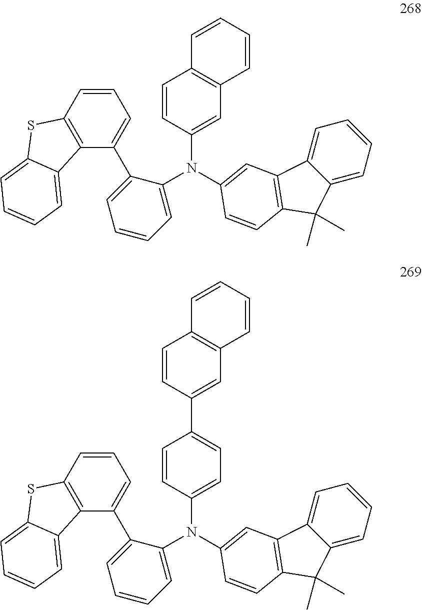

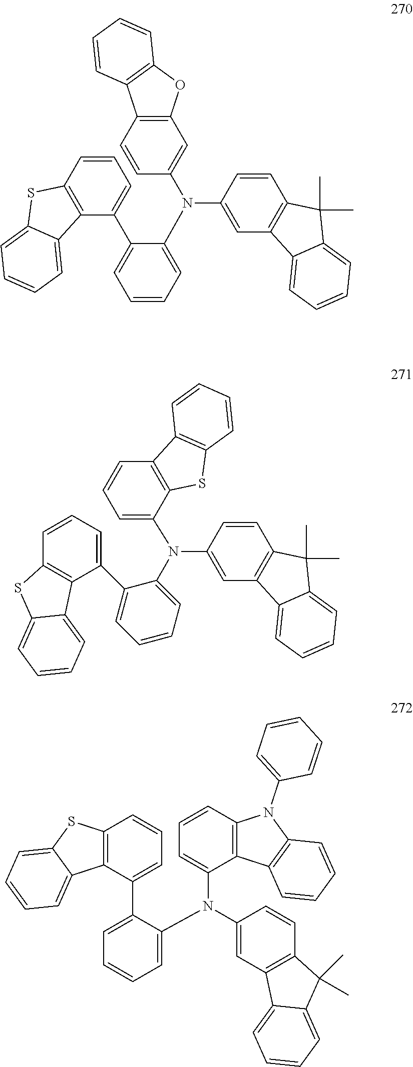 [SMILES: CC1(C)c2ccccc2-c2cc(N(c3ccc4c(c3)oc3ccccc34)c3ccccc3-c3cccc4sc5ccccc5c34)ccc21 ; CC1(C)c2ccccc2-c2cc(N(c3ccccc3-c3cccc4sc5ccccc5c34)c3cccc4c3sc3ccccc34)ccc21 ; CC1(C)c2ccccc2-c2cc(N(c3ccccc3-c3cccc4sc5ccccc5c34)c3cccc4c3c3ccccc3n4-c3ccccc3)ccc21]

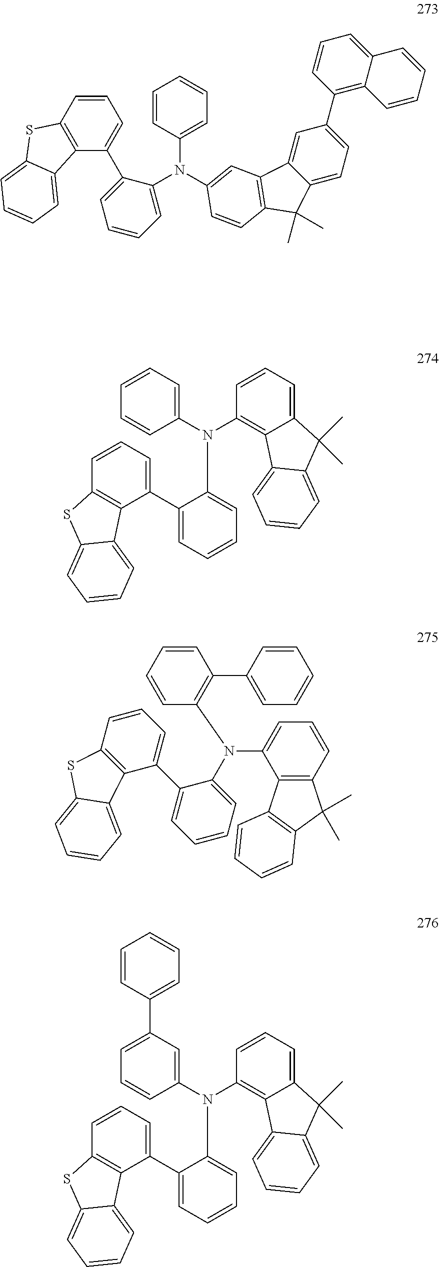

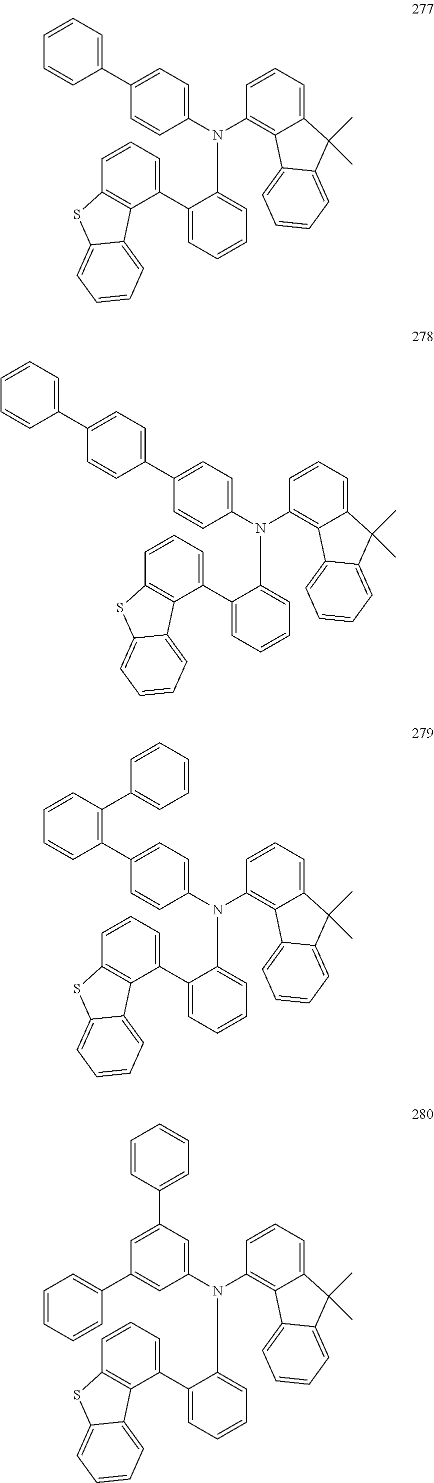 [SMILES: CC1(C)c2ccccc2-c2c(N(c3ccc(-c4ccccc4)cc3)c3ccccc3-c3cccc4sc5ccccc5c34)cccc21 ; CC1(C)c2ccccc2-c2c(N(c3ccc(-c4ccc(-c5ccccc5)cc4)cc3)c3ccccc3-c3cccc4sc5ccccc5c34)cccc21 ; CC1(C)c2ccccc2-c2c(N(c3ccc(-c4ccccc4-c4ccccc4)cc3)c3ccccc3-c3cccc4sc5ccccc5c34)cccc21 ; CC1(C)c2ccccc2-c2c(N(c3cc(-c4ccccc4)cc(-c4ccccc4)c3)c3ccccc3-c3cccc4sc5ccccc5c34)cccc21]

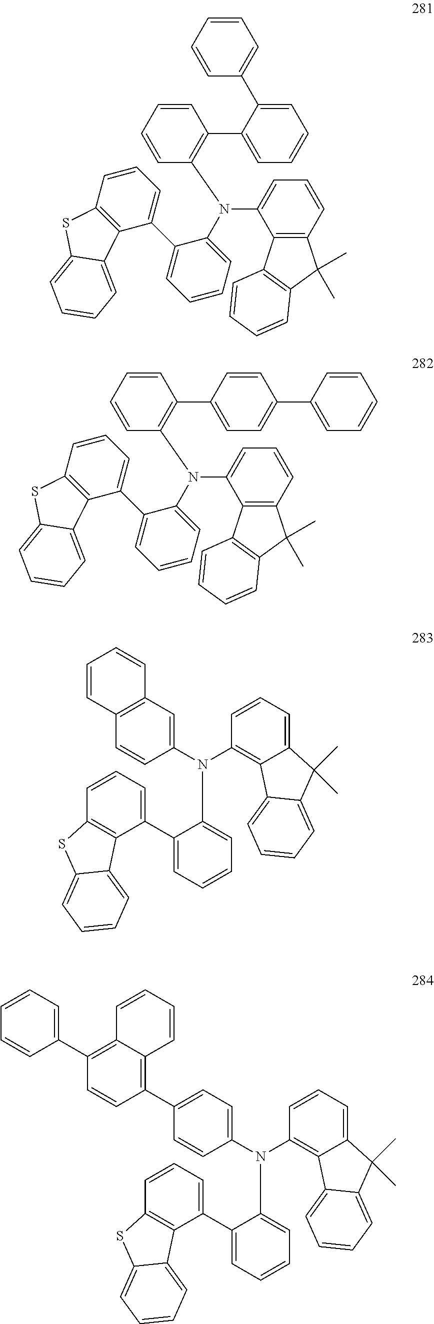

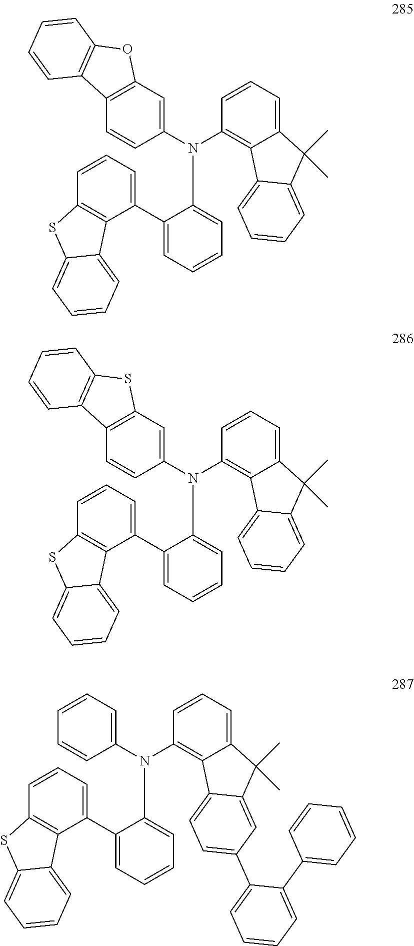

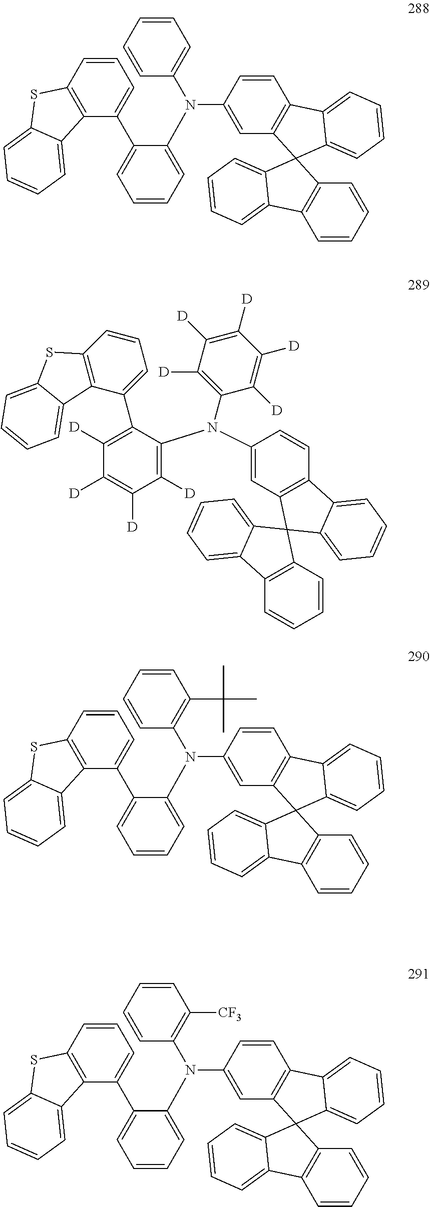 [SMILES: c1ccc(N(c2ccc3c(c2)C2(c4ccccc4-c4ccccc42)c2ccccc2-3)c2ccccc2-c2cccc3sc4ccccc4c23)cc1 ; [2H]c1c([2H])c([2H])c(N(c2ccc3c(c2)C2(c4ccccc4-c4ccccc42)c2ccccc2-3)c2c([2H])c([2H])c([2H])c([2H])c2-c2cccc3sc4ccccc4c23)c([2H])c1[2H] ; CC(C)(C)c1ccccc1N(c1ccc2c(c1)C1(c3ccccc3-c3ccccc31)c1ccccc1-2)c1ccccc1-c1cccc2sc3ccccc3c12 ; FC(F)(F)c1ccccc1N(c1ccc2c(c1)C1(c3ccccc3-c3ccccc31)c1ccccc1-2)c1ccccc1-c1cccc2sc3ccccc3c12]

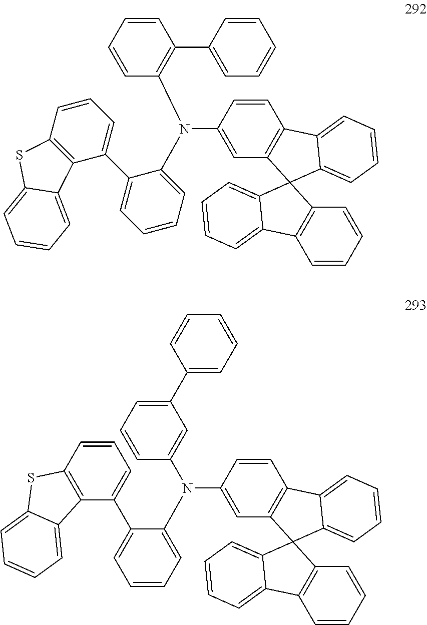 [SMILES: c1ccc(-c2ccccc2N(c2ccc3c(c2)C2(c4ccccc4-c4ccccc42)c2ccccc2-3)c2ccccc2-c2cccc3sc4ccccc4c23)cc1 ; c1ccc(-c2cccc(N(c3ccc4c(c3)C3(c5ccccc5-c5ccccc53)c3ccccc3-4)c3ccccc3-c3cccc4sc5ccccc5c34)c2)cc1]

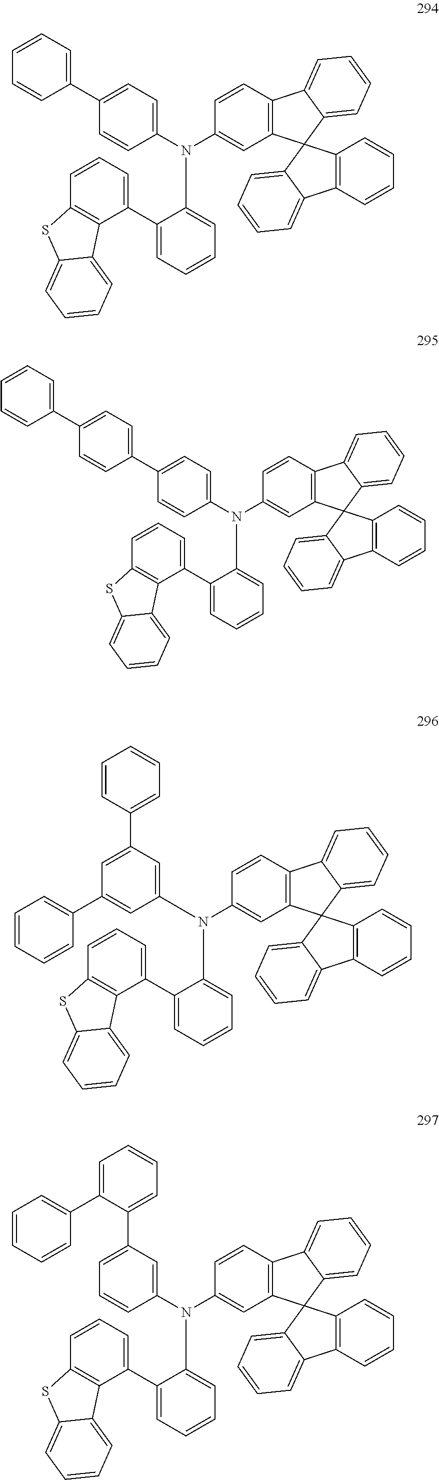 [SMILES: c1ccc(-c2ccc(N(c3ccc4c(c3)C3(c5ccccc5-c5ccccc53)c3ccccc3-4)c3ccccc3-c3cccc4sc5ccccc5c34)cc2)cc1 ; c1ccc(-c2ccc(-c3ccc(N(c4ccc5c(c4)C4(c6ccccc6-c6ccccc64)c4ccccc4-5)c4ccccc4-c4cccc5sc6ccccc6c45)cc3)cc2)cc1 ; c1ccc(-c2cc(-c3ccccc3)cc(N(c3ccc4c(c3)C3(c5ccccc5-c5ccccc53)c3ccccc3-4)c3ccccc3-c3cccc4sc5ccccc5c34)c2)cc1 ; c1ccc(-c2ccccc2-c2cccc(N(c3ccc4c(c3)C3(c5ccccc5-c5ccccc53)c3ccccc3-4)c3ccccc3-c3cccc4sc5ccccc5c34)c2)cc1]

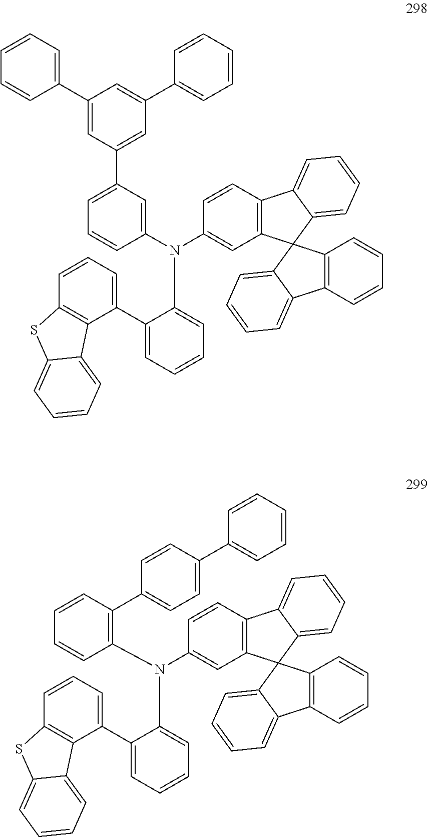 [SMILES: c1ccc(-c2cc(-c3ccccc3)cc(-c3cccc(N(c4ccc5c(c4)C4(c6ccccc6-c6ccccc64)c4ccccc4-5)c4ccccc4-c4cccc5sc6ccccc6c45)c3)c2)cc1 ; c1ccc(-c2ccc(-c3ccccc3N(c3ccc4c(c3)C3(c5ccccc5-c5ccccc53)c3ccccc3-4)c3ccccc3-c3cccc4sc5ccccc5c34)cc2)cc1]

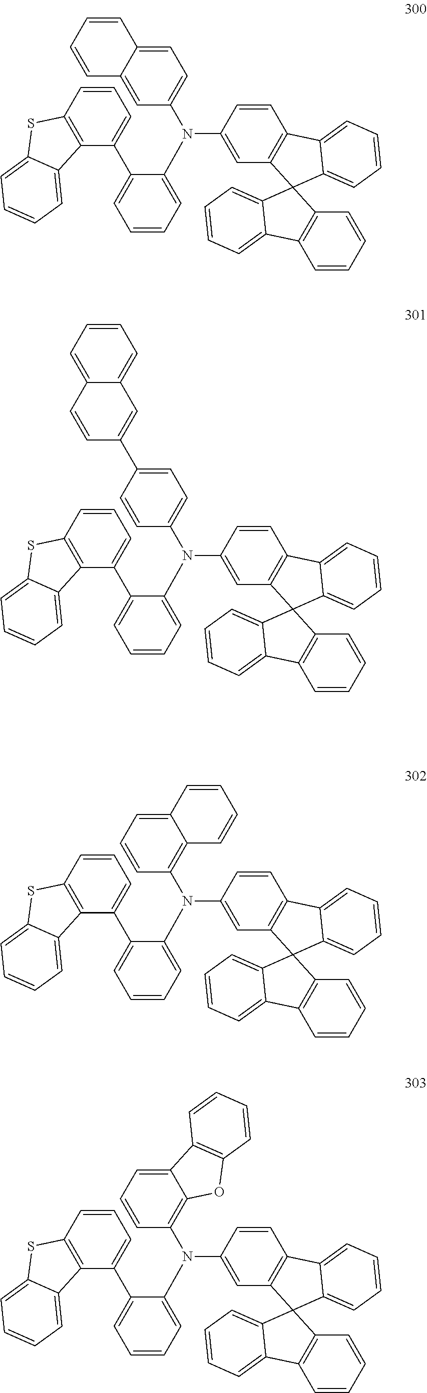 [SMILES: c1ccc(N(c2ccc3c(c2)C2(c4ccccc4-c4ccccc42)c2ccccc2-3)c2ccc3ccccc3c2)c(-c2cccc3sc4ccccc4c23)c1 ; c1ccc(N(c2ccc(-c3ccc4ccccc4c3)cc2)c2ccc3c(c2)C2(c4ccccc4-c4ccccc42)c2ccccc2-3)c(-c2cccc3sc4ccccc4c23)c1 ; c1ccc(N(c2ccc3c(c2)C2(c4ccccc4-c4ccccc42)c2ccccc2-3)c2cccc3ccccc23)c(-c2cccc3sc4ccccc4c23)c1 ; c1ccc(N(c2ccc3c(c2)C2(c4ccccc4-c4ccccc42)c2ccccc2-3)c2cccc3c2oc2ccccc23)c(-c2cccc3sc4ccccc4c23)c1]

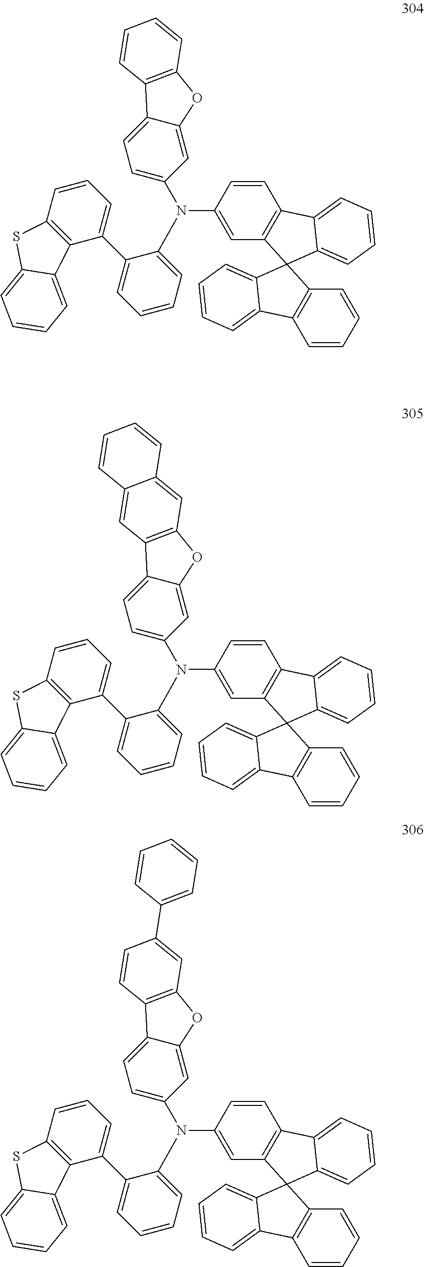 [SMILES: c1ccc(N(c2ccc3c(c2)C2(c4ccccc4-c4ccccc42)c2ccccc2-3)c2ccc3c(c2)oc2ccccc23)c(-c2cccc3sc4ccccc4c23)c1 ; c1ccc(N(c2ccc3c(c2)C2(c4ccccc4-c4ccccc42)c2ccccc2-3)c2ccc3c(c2)oc2cc4ccccc4cc23)c(-c2cccc3sc4ccccc4c23)c1 ; c1ccc(-c2ccc3c(c2)oc2cc(N(c4ccc5c(c4)C4(c6ccccc6-c6ccccc64)c4ccccc4-5)c4ccccc4-c4cccc5sc6ccccc6c45)ccc23)cc1]

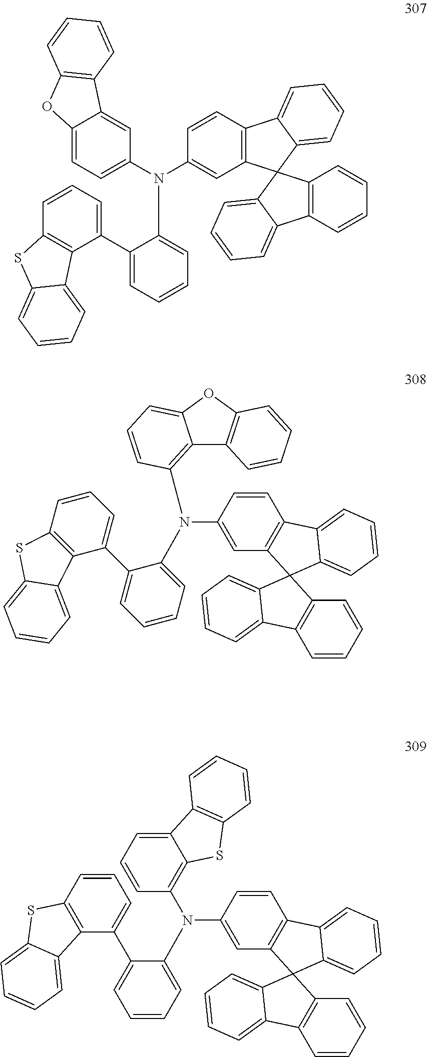 [SMILES: c1ccc(N(c2ccc3c(c2)C2(c4ccccc4-c4ccccc42)c2ccccc2-3)c2ccc3oc4ccccc4c3c2)c(-c2cccc3sc4ccccc4c23)c1 ; c1ccc(N(c2ccc3c(c2)C2(c4ccccc4-c4ccccc42)c2ccccc2-3)c2cccc3oc4ccccc4c23)c(-c2cccc3sc4ccccc4c23)c1 ; c1ccc(N(c2ccc3c(c2)C2(c4ccccc4-c4ccccc42)c2ccccc2-3)c2cccc3c2sc2ccccc23)c(-c2cccc3sc4ccccc4c23)c1]

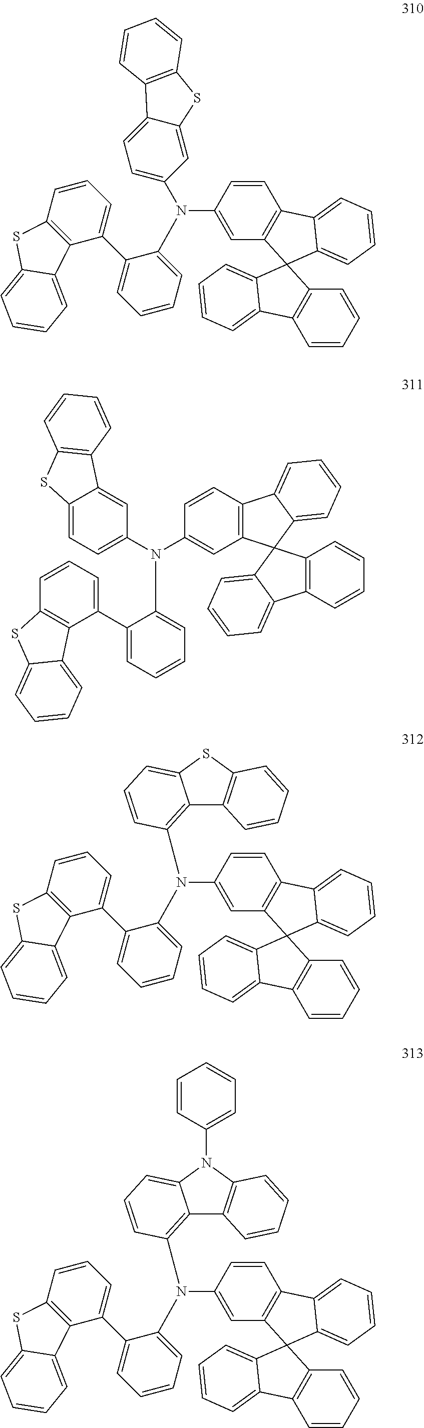 [SMILES: c1ccc(N(c2ccc3c(c2)C2(c4ccccc4-c4ccccc42)c2ccccc2-3)c2ccc3c(c2)sc2ccccc23)c(-c2cccc3sc4ccccc4c23)c1 ; c1ccc(N(c2ccc3c(c2)C2(c4ccccc4-c4ccccc42)c2ccccc2-3)c2ccc3sc4ccccc4c3c2)c(-c2cccc3sc4ccccc4c23)c1 ; c1ccc(N(c2ccc3c(c2)C2(c4ccccc4-c4ccccc42)c2ccccc2-3)c2cccc3sc4ccccc4c23)c(-c2cccc3sc4ccccc4c23)c1 ; c1ccc(-n2c3ccccc3c3c(N(c4ccc5c(c4)C4(c6ccccc6-c6ccccc64)c4ccccc4-5)c4ccccc4-c4cccc5sc6ccccc6c45)cccc32)cc1]

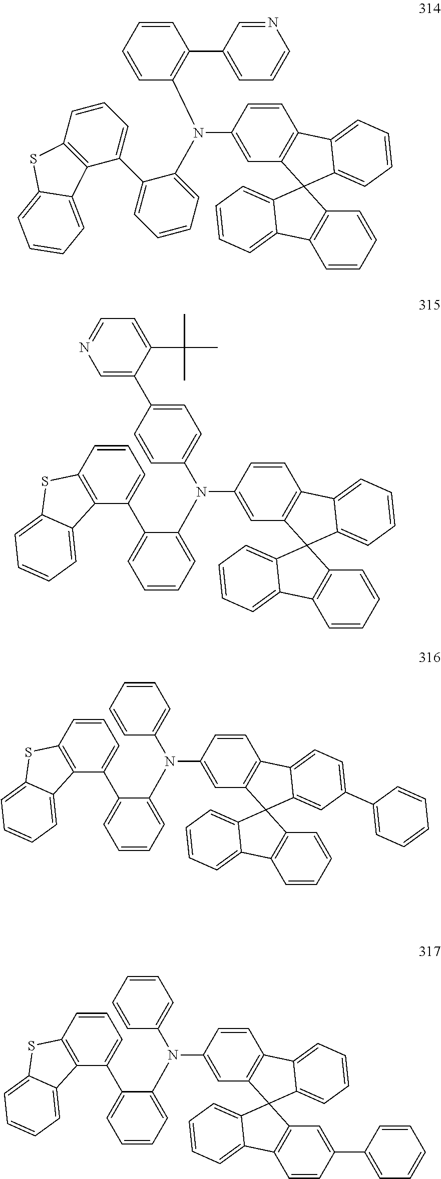 [SMILES: c1cncc(-c2ccccc2N(c2ccc3c(c2)C2(c4ccccc4-c4ccccc42)c2ccccc2-3)c2ccccc2-c2cccc3sc4ccccc4c23)c1 ; CC(C)(C)c1ccncc1-c1ccc(N(c2ccc3c(c2)C2(c4ccccc4-c4ccccc42)c2ccccc2-3)c2ccccc2-c2cccc3sc4ccccc4c23)cc1 ; c1ccc(-c2ccc3c(c2)C2(c4ccccc4-c4ccccc42)c2cc(N(c4ccccc4)c4ccccc4-c4cccc5sc6ccccc6c45)ccc2-3)cc1 ; c1ccc(-c2ccc3c(c2)C2(c4ccccc4-3)c3ccccc3-c3ccc(N(c4ccccc4)c4ccccc4-c4cccc5sc6ccccc6c45)cc32)cc1]

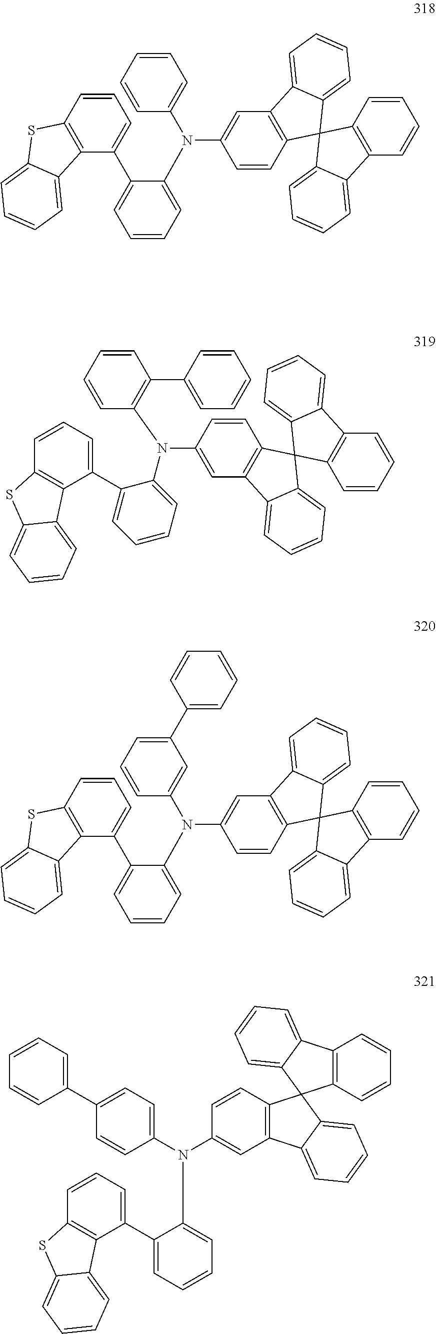 [SMILES: c1ccc(N(c2ccc3c(c2)-c2ccccc2C32c3ccccc3-c3ccccc32)c2ccccc2-c2cccc3sc4ccccc4c23)cc1 ; c1ccc(-c2ccccc2N(c2ccc3c(c2)-c2ccccc2C32c3ccccc3-c3ccccc32)c2ccccc2-c2cccc3sc4ccccc4c23)cc1 ; c1ccc(-c2cccc(N(c3ccc4c(c3)-c3ccccc3C43c4ccccc4-c4ccccc43)c3ccccc3-c3cccc4sc5ccccc5c34)c2)cc1 ; c1ccc(-c2ccc(N(c3ccc4c(c3)-c3ccccc3C43c4ccccc4-c4ccccc43)c3ccccc3-c3cccc4sc5ccccc5c34)cc2)cc1]

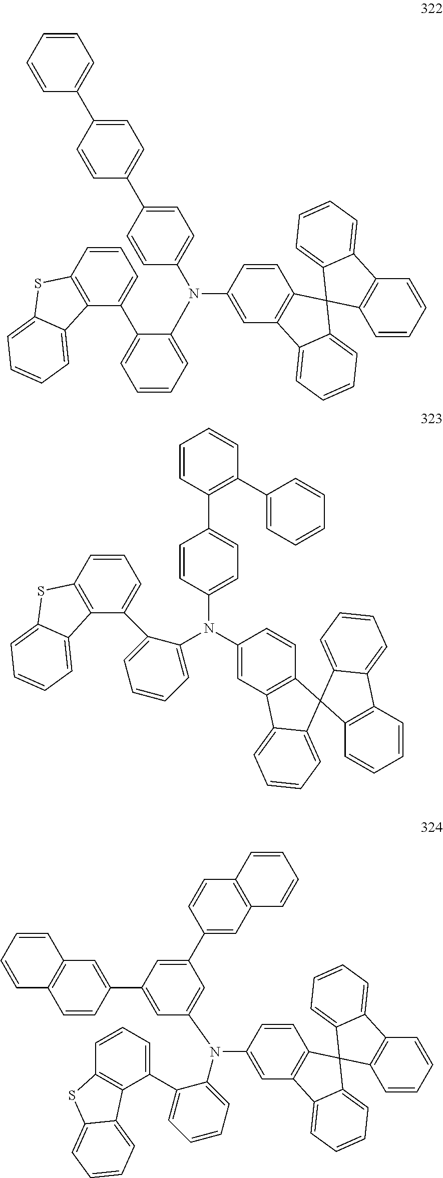 [SMILES: c1ccc(-c2ccc(-c3ccc(N(c4ccc5c(c4)-c4ccccc4C54c5ccccc5-c5ccccc54)c4ccccc4-c4cccc5sc6ccccc6c45)cc3)cc2)cc1 ; c1ccc(-c2ccccc2-c2ccc(N(c3ccc4c(c3)-c3ccccc3C43c4ccccc4-c4ccccc43)c3ccccc3-c3cccc4sc5ccccc5c34)cc2)cc1 ; c1ccc(N(c2cc(-c3ccc4ccccc4c3)cc(-c3ccc4ccccc4c3)c2)c2ccc3c(c2)-c2ccccc2C32c3ccccc3-c3ccccc32)c(-c2cccc3sc4ccccc4c23)c1]

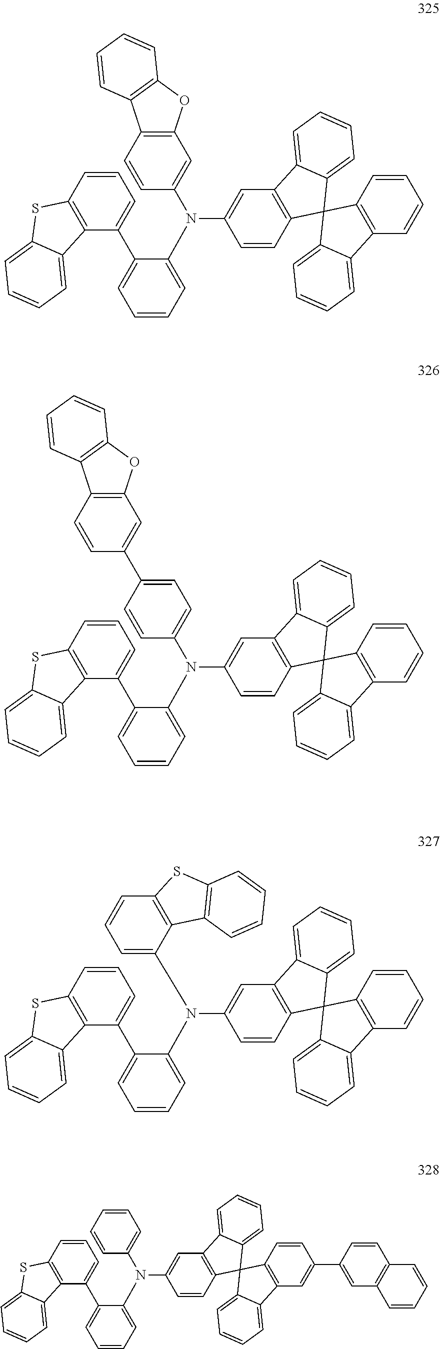

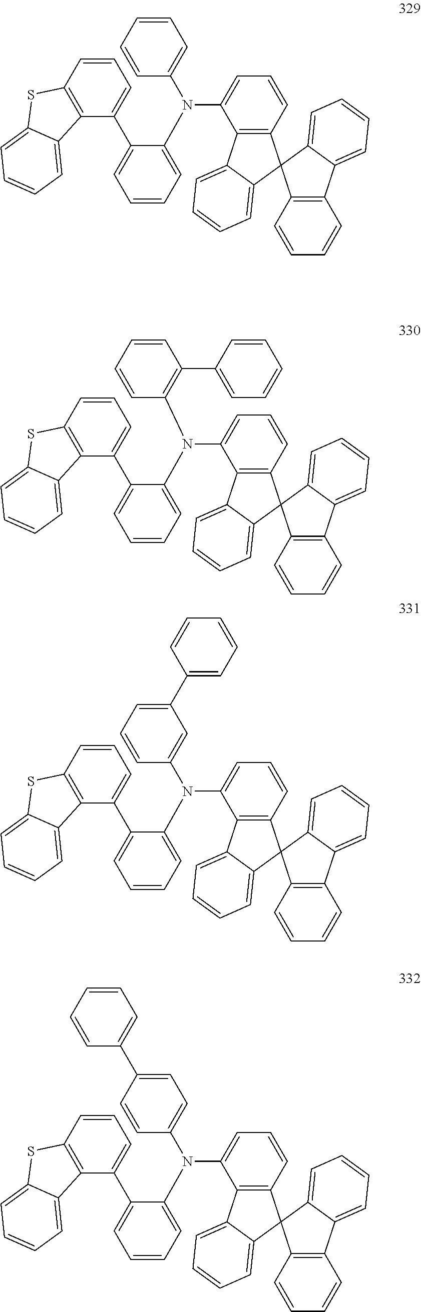 [SMILES: c1ccc(N(c2ccccc2-c2cccc3sc4ccccc4c23)c2cccc3c2-c2ccccc2C32c3ccccc3-c3ccccc32)cc1 ; c1ccc(-c2ccccc2N(c2ccccc2-c2cccc3sc4ccccc4c23)c2cccc3c2-c2ccccc2C32c3ccccc3-c3ccccc32)cc1 ; c1ccc(-c2cccc(N(c3ccccc3-c3cccc4sc5ccccc5c34)c3cccc4c3-c3ccccc3C43c4ccccc4-c4ccccc43)c2)cc1 ; c1ccc(-c2ccc(N(c3ccccc3-c3cccc4sc5ccccc5c34)c3cccc4c3-c3ccccc3C43c4ccccc4-c4ccccc43)cc2)cc1]

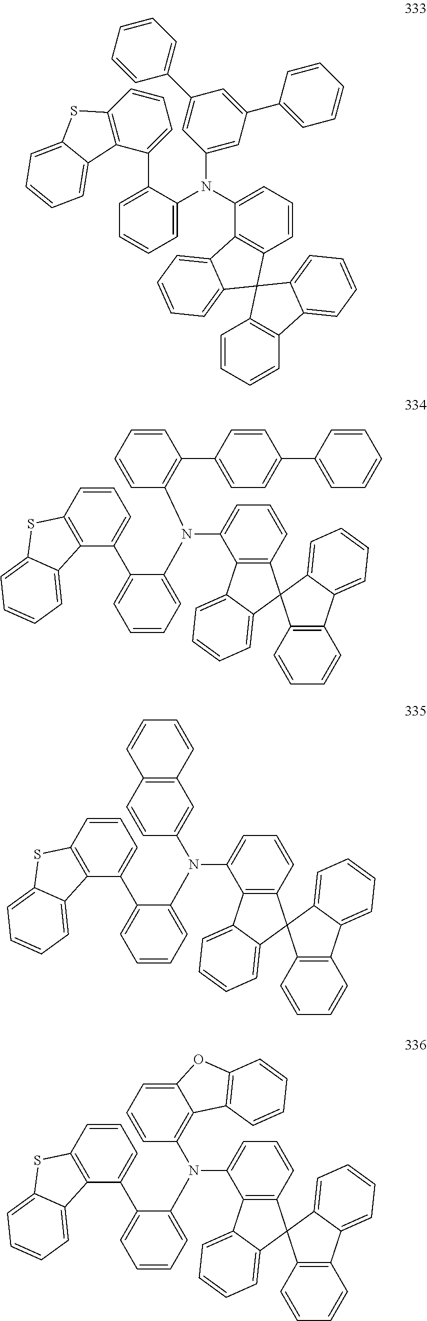 [SMILES: c1ccc(-c2cc(-c3ccccc3)cc(N(c3ccccc3-c3cccc4sc5ccccc5c34)c3cccc4c3-c3ccccc3C43c4ccccc4-c4ccccc43)c2)cc1 ; c1ccc(-c2ccc(-c3ccccc3N(c3ccccc3-c3cccc4sc5ccccc5c34)c3cccc4c3-c3ccccc3C43c4ccccc4-c4ccccc43)cc2)cc1 ; c1ccc(N(c2ccc3ccccc3c2)c2cccc3c2-c2ccccc2C32c3ccccc3-c3ccccc32)c(-c2cccc3sc4ccccc4c23)c1 ; c1ccc(N(c2cccc3c2-c2ccccc2C32c3ccccc3-c3ccccc32)c2cccc3oc4ccccc4c23)c(-c2cccc3sc4ccccc4c23)c1]

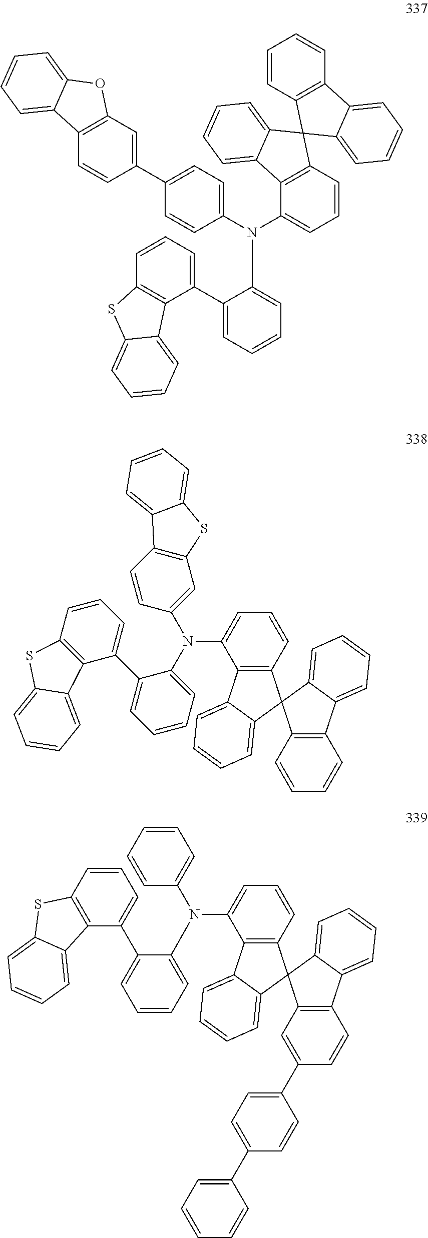 [SMILES: c1ccc(N(c2ccc(-c3ccc4c(c3)oc3ccccc34)cc2)c2cccc3c2-c2ccccc2C32c3ccccc3-c3ccccc32)c(-c2cccc3sc4ccccc4c23)c1 ; c1ccc(N(c2ccc3c(c2)sc2ccccc23)c2cccc3c2-c2ccccc2C32c3ccccc3-c3ccccc32)c(-c2cccc3sc4ccccc4c23)c1 ; c1ccc(-c2ccc(-c3ccc4c(c3)C3(c5ccccc5-4)c4ccccc4-c4c(N(c5ccccc5)c5ccccc5-c5cccc6sc7ccccc7c56)cccc43)cc2)cc1]

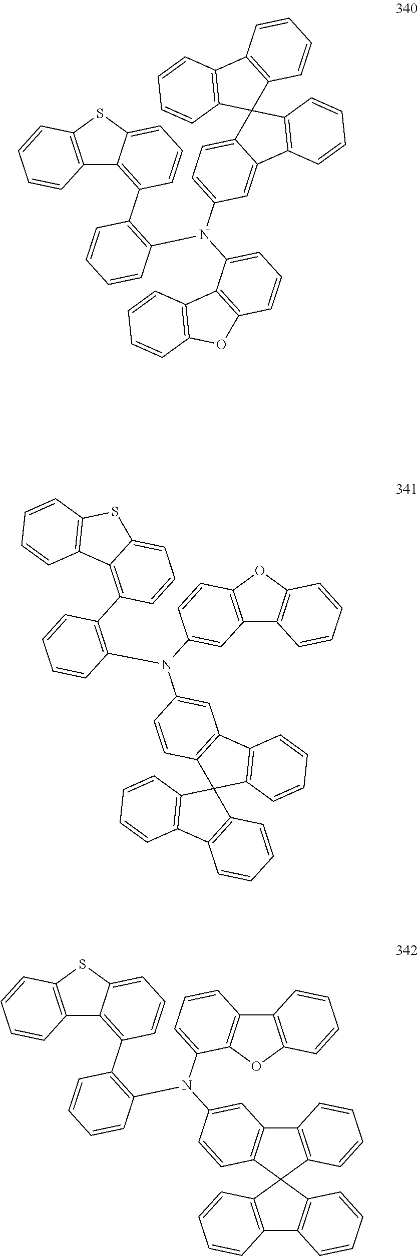 [SMILES: c1ccc(N(c2ccc3c(c2)-c2ccccc2C32c3ccccc3-c3ccccc32)c2cccc3oc4ccccc4c23)c(-c2cccc3sc4ccccc4c23)c1 ; c1ccc(N(c2ccc3c(c2)-c2ccccc2C32c3ccccc3-c3ccccc32)c2ccc3oc4ccccc4c3c2)c(-c2cccc3sc4ccccc4c23)c1 ; c1ccc(N(c2ccc3c(c2)-c2ccccc2C32c3ccccc3-c3ccccc32)c2cccc3c2oc2ccccc23)c(-c2cccc3sc4ccccc4c23)c1]

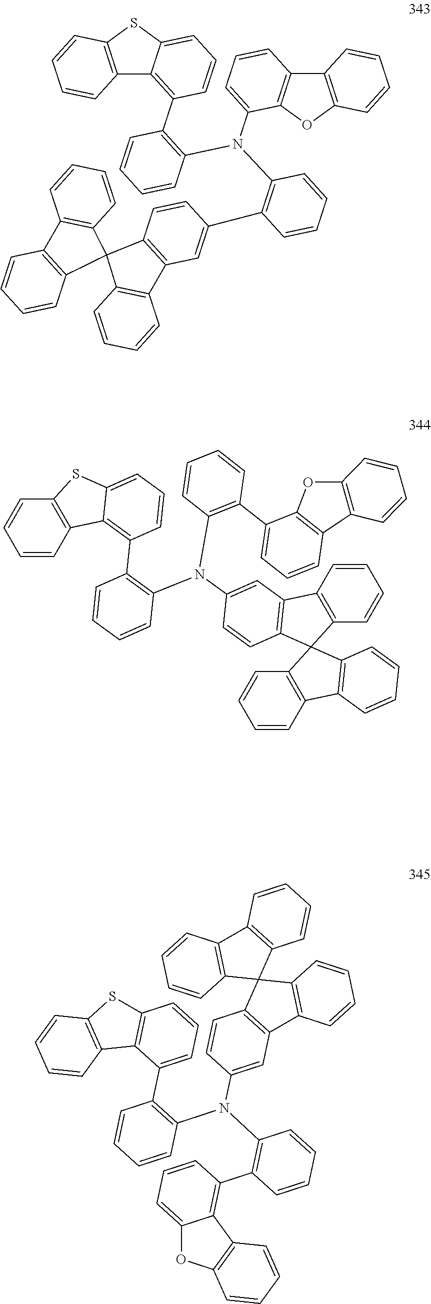 [SMILES: c1ccc(N(c2ccccc2-c2cccc3sc4ccccc4c23)c2cccc3c2oc2ccccc23)c(-c2ccc3c(c2)-c2ccccc2C32c3ccccc3-c3ccccc32)c1 ; c1ccc(N(c2ccc3c(c2)-c2ccccc2C32c3ccccc3-c3ccccc32)c2ccccc2-c2cccc3sc4ccccc4c23)c(-c2cccc3c2oc2ccccc23)c1 ; c1ccc(N(c2ccc3c(c2)-c2ccccc2C32c3ccccc3-c3ccccc32)c2ccccc2-c2cccc3sc4ccccc4c23)c(-c2cccc3oc4ccccc4c23)c1]

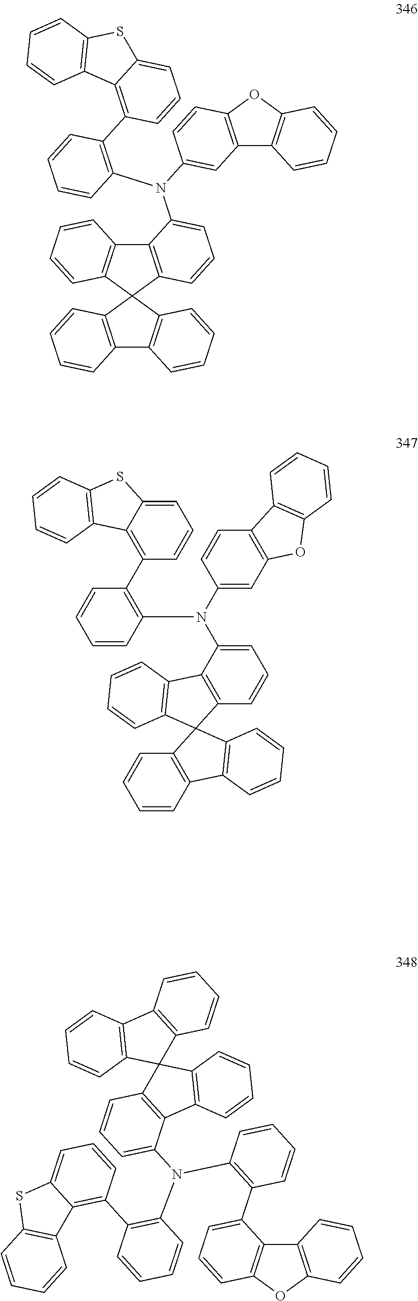 [SMILES: c1ccc(N(c2ccc3oc4ccccc4c3c2)c2cccc3c2-c2ccccc2C32c3ccccc3-c3ccccc32)c(-c2cccc3sc4ccccc4c23)c1 ; c1ccc(N(c2ccc3c(c2)oc2ccccc23)c2cccc3c2-c2ccccc2C32c3ccccc3-c3ccccc32)c(-c2cccc3sc4ccccc4c23)c1 ; c1ccc(N(c2ccccc2-c2cccc3sc4ccccc4c23)c2cccc3c2-c2ccccc2C32c3ccccc3-c3ccccc32)c(-c2cccc3oc4ccccc4c23)c1]

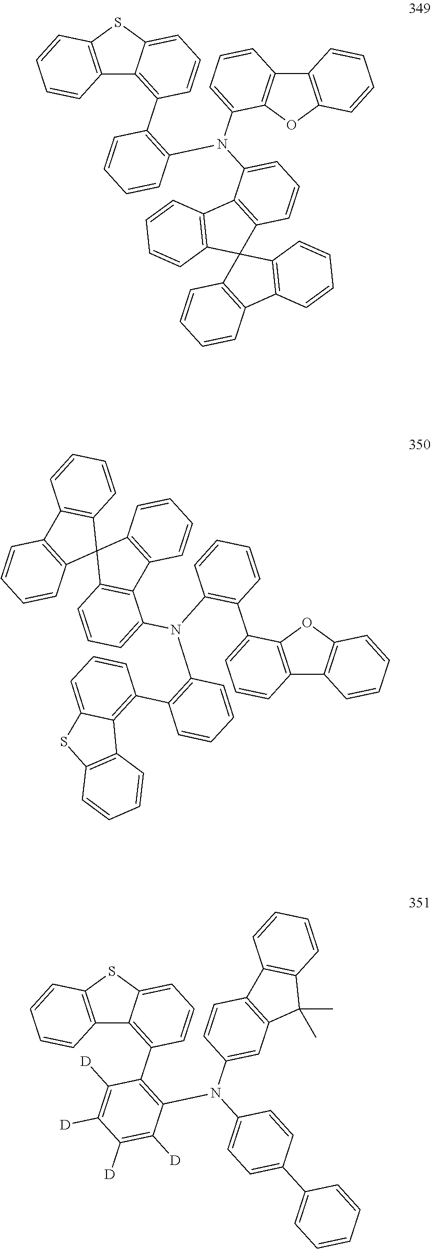 [SMILES: c1ccc(N(c2cccc3c2-c2ccccc2C32c3ccccc3-c3ccccc32)c2cccc3c2oc2ccccc23)c(-c2cccc3sc4ccccc4c23)c1 ; c1ccc(N(c2ccccc2-c2cccc3sc4ccccc4c23)c2cccc3c2-c2ccccc2C32c3ccccc3-c3ccccc32)c(-c2cccc3c2oc2ccccc23)c1 ; [2H]c1c([2H])c([2H])c(N(c2ccc(-c3ccccc3)cc2)c2ccc3c(c2)C(C)(C)c2ccccc2-3)c(-c2cccc3sc4ccccc4c23)c1[2H]]

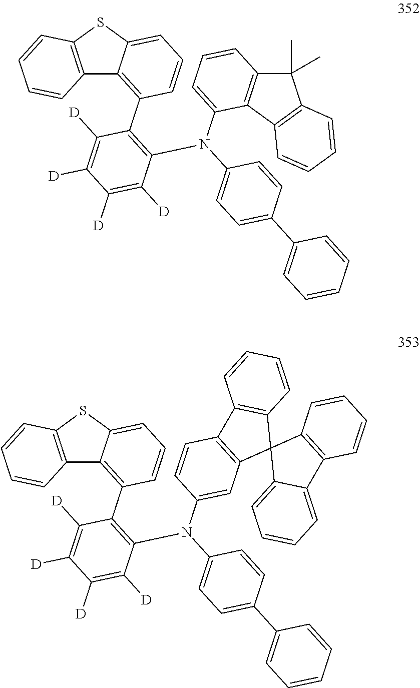 [SMILES: [2H]c1c([2H])c([2H])c(N(c2ccc(-c3ccccc3)cc2)c2cccc3c2-c2ccccc2C3(C)C)c(-c2cccc3sc4ccccc4c23)c1[2H] ; [2H]c1c([2H])c([2H])c(N(c2ccc(-c3ccccc3)cc2)c2ccc3c(c2)C2(c4ccccc4-c4ccccc42)c2ccccc2-3)c(-c2cccc3sc4ccccc4c23)c1[2H]]

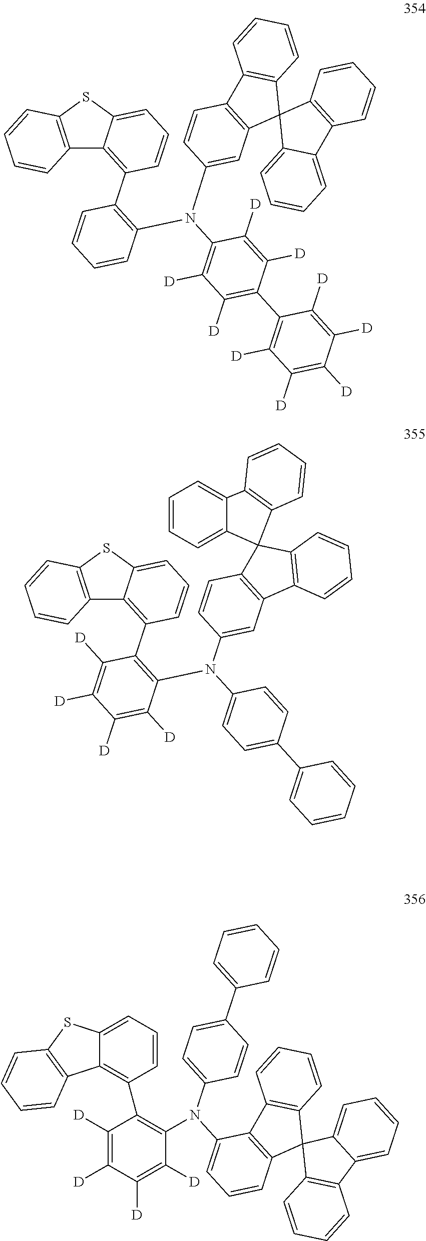

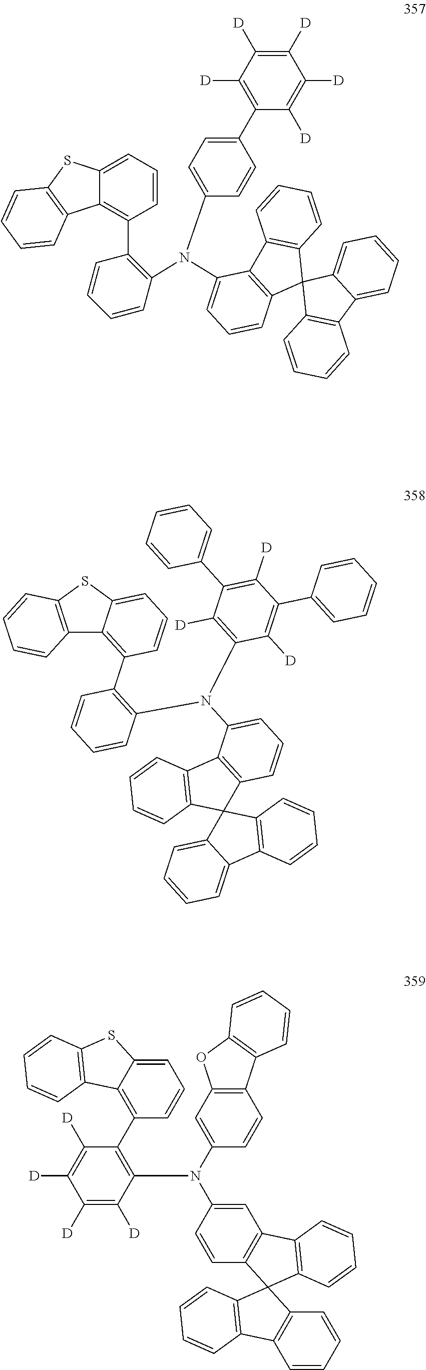 [SMILES: [2H]c1c([2H])c([2H])c(-c2ccc(N(c3ccccc3-c3cccc4sc5ccccc5c34)c3cccc4c3-c3ccccc3C43c4ccccc4-c4ccccc43)cc2)c([2H])c1[2H] ; [2H]c1c(-c2ccccc2)c([2H])c(N(c2ccccc2-c2cccc3sc4ccccc4c23)c2cccc3c2-c2ccccc2C32c3ccccc3-c3ccccc32)c([2H])c1-c1ccccc1 ; [2H]c1c([2H])c([2H])c(N(c2ccc3c(c2)-c2ccccc2C32c3ccccc3-c3ccccc32)c2ccc3c(c2)oc2ccccc23)c(-c2cccc3sc4ccccc4c23)c1[2H]]

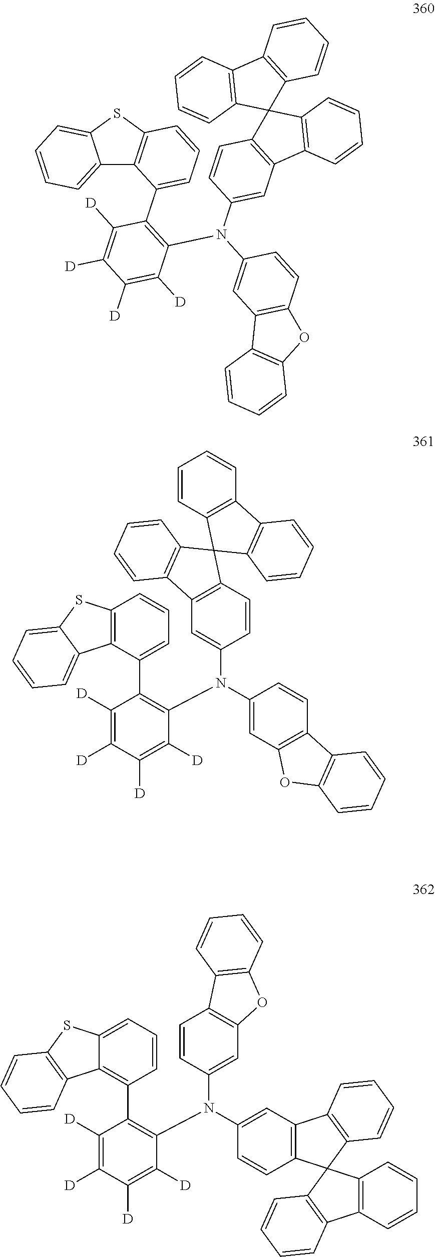 [SMILES: [2H]c1c([2H])c([2H])c(N(c2ccc3c(c2)-c2ccccc2C32c3ccccc3-c3ccccc32)c2ccc3oc4ccccc4c3c2)c(-c2cccc3sc4ccccc4c23)c1[2H] ; [2H]c1c([2H])c([2H])c(N(c2ccc3c(c2)-c2ccccc2C32c3ccccc3-c3ccccc32)c2ccc3c(c2)oc2ccccc23)c(-c2cccc3sc4ccccc4c23)c1[2H] ; [2H]c1c([2H])c([2H])c(N(c2ccc3c(c2)-c2ccccc2C32c3ccccc3-c3ccccc32)c2ccc3c(c2)oc2ccccc23)c(-c2cccc3sc4ccccc4c23)c1[2H]]

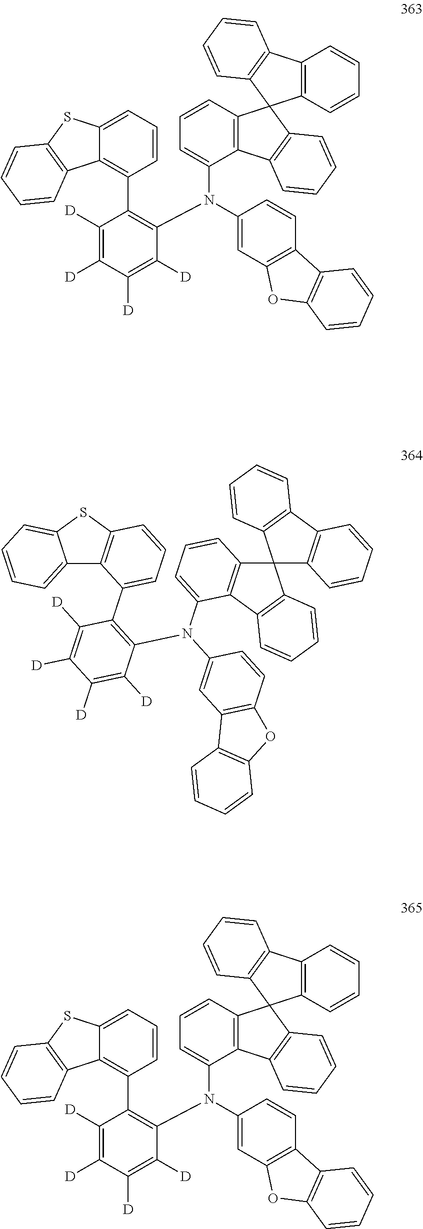 [SMILES: [2H]c1c([2H])c([2H])c(N(c2ccc3c(c2)oc2ccccc23)c2cccc3c2-c2ccccc2C32c3ccccc3-c3ccccc32)c(-c2cccc3sc4ccccc4c23)c1[2H] ; [2H]c1c([2H])c([2H])c(N(c2ccc3oc4ccccc4c3c2)c2cccc3c2-c2ccccc2C32c3ccccc3-c3ccccc32)c(-c2cccc3sc4ccccc4c23)c1[2H] ; [2H]c1c([2H])c([2H])c(N(c2ccc3c(c2)oc2ccccc23)c2cccc3c2-c2ccccc2C32c3ccccc3-c3ccccc32)c(-c2cccc3sc4ccccc4c23)c1[2H]]

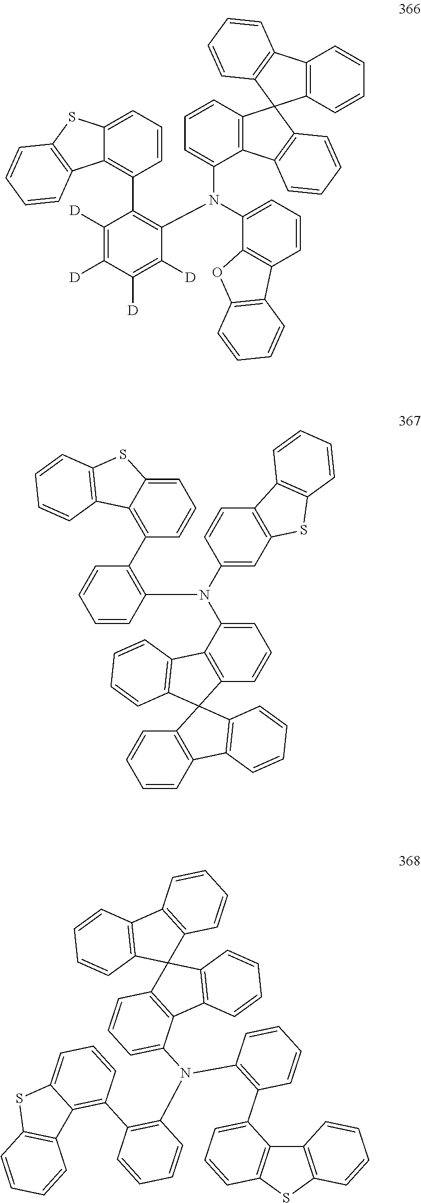 [SMILES: [2H]c1c([2H])c([2H])c(N(c2cccc3c2-c2ccccc2C32c3ccccc3-c3ccccc32)c2cccc3c2oc2ccccc23)c(-c2cccc3sc4ccccc4c23)c1[2H] ; c1ccc(N(c2ccc3c(c2)sc2ccccc23)c2cccc3c2-c2ccccc2C32c3ccccc3-c3ccccc32)c(-c2cccc3sc4ccccc4c23)c1 ; c1ccc(N(c2ccccc2-c2cccc3sc4ccccc4c23)c2cccc3c2-c2ccccc2C32c3ccccc3-c3ccccc32)c(-c2cccc3sc4ccccc4c23)c1]

As such, the organic compound according to the present invention may be synthesized with various characteristics using a characteristic skeleton that exhibits inherent characteristics and moieties with inherent characteristics introduced thereto. As a result, the organic compound according to the present invention may be applied as a material for various organic layers of a light-emitting layer, a hole transport layer, an electron transport layer, an electron blocking layer, and a hole blocking layer, and is used as preferably a material for the hole transport layer to the electron blocking layer to further improve luminescent properties such as luminous efficiency, etc. of the device.

Further, the compound of the present invention may be applied to the device according to a general method for manufacturing an organic light-emitting device. An organic light-emitting device according to an embodiment of the present invention may have a structure including a first electrode, a second electrode, and organic layers arranged therebetween. The organic light-emitting device may be manufactured using a general device manufacturing method and material, except for using the organic compound according to the present invention for the organic layer of the device.

The organic layer of the organic light-emitting device according to an embodiment of the present invention may have a monolayer structure or a multilayer structure in which two or more organic layers are stacked. For example, the structure of the organic layers may include a hole injecting layer, a hole transport layer, a light-emitting layer, an electron transport layer, an electron injecting layer, an electron blocking layer, a hole blocking layer, and a light efficiency improving layer (capping layer). The number of the organic layers is not limited and may be increased or decreased.

Preferred structures of the organic layers of the organic light-emitting according to an embodiment of the present invention will be explained in more detail in the examples to be described later.

In addition, the organic light-emitting device of an embodiment of the present invention may be manufactured by depositing a metal, a conductive metal oxide or an alloy thereof on a substrate by a physical vapor deposition (PVD) method such as sputtering or e-beam evaporation to form an anode, forming organic layers including a hole injecting layer, a hole transport layer, a light-emitting layer, and an electron transport layer thereon, and depositing a cathode material thereon.

In addition to the above methods, the organic light-emitting device may be fabricated by depositing a cathode material, organic layer materials, and an anode material in this order on a substrate. The organic layers may have a multilayer structure including a hole injecting layer, a hole transport layer, a light-emitting layer, and an electron transport layer, but is not limited thereto and may have a monolayer structure. In addition, the organic layers may be manufactured in a smaller number of layers by a solvent process using various polymer materials rather than by a deposition process, such as spin coating, dip coating, doctor blading, screen printing, inkjet printing or thermal transfer.

As the anode, a material having a high work function is generally preferred for easy injection of holes into the organic layers. Specific examples of anode materials suitable for use in an embodiment of the present invention include, but are not limited to: metals such as vanadium, chromium, copper, zinc, and gold and alloys thereof; metal oxides such as zinc oxide, indium oxide, indium thin oxide (ITO), and indium zinc oxide (IZO); combinations of metals and oxides such as ZnO:Al and SnO₂:Sb; and conductive polymers such as poly(3-methylthiophene), poly[3,4-(ethylene-1,2-dioxy)thiophene] (PEDT), polypyrrole, and polyaniline.

As the cathode, a material having a low work function is generally preferred for easy injection of electrons into the organic layers. Specific examples of suitable cathode materials include, but are not limited to: metals such as magnesium, calcium, sodium, potassium, titanium, indium, yttrium, lithium, gadolinium, aluminum, silver, tin, and lead and alloys thereof; and multilayer structure materials such as LiF/Al and LiO₂/Al.

The hole injecting layer is preferably a material that may receive holes injected from the anode at low voltage. The highest occupied molecular orbital (HOMO) of the hole injecting material is preferably between the work function of the anode material and the HOMO of the adjacent organic layer. Specific examples of hole injecting materials include, but are not limited to, metal porphyrin, oligothiophene, arylamine-based organic materials, hexanitrile hexaazatriphenylene, quinacridone-based organic materials, perylene-based organic materials, anthraquinone, polyaniline, and polythiophene-based conductive polymers.

The hole transport layer is a material that may receive holes transported from the anode or the hole injecting layer and may transfer the holes to the light-emitting layer. A material with high hole mobility is suitable. Specific examples thereof include arylamine-based organic materials, conductive polymers, and block copolymers consisting of conjugated and non-conjugated segments. The use of the organic compound according to an embodiment of the present invention ensures further improved low-voltage driving characteristics, high luminous efficiency, and life characteristics of the device.

The electron blocking layer is a layer that blocks the movement of electrons and may be formed on the hole transport layer, and may be used to block the movement of electrons without affecting the transport of holes. In addition, on the electron blocking layer, the light-emitting layer may be formed, and the hole blocking layer, the electron transport layer, and the electron injecting layer may be formed.

The hole blocking layer may be used to block the movement of holes without affecting the transport of electrons, and examples of the hole blocking layer are 1,3,5-tri(1-phenyl-1H-benzo[d]imidazol-2-yl)phenyl (TPBi), 2,9-dimethyl4,7-diphenyl-1,10-phenanthroline (BCP), 4,4-bis(N-carbazolyl)-1,1′-biphenyl (CBP), 2-(4-biphenyl)-5-(4-t-butylphenyl)-1,3,4-oxadiazole (PBD), bisbenzimidazo[2,1-a:1′,2-b′]anthra[2,1,9-def:6,5,10-d′e′f′]diisoguinoline-10,21-dione (PTCBI), 4,7-diphenyl-1,10-phenanthroline (BPhen), or the like, but are not limited thereto.

The light-emitting layer is a material that may receive and recombine holes from the hole transport layer and electrons from the electron transport layer to emit light in the visible ray area. A material with high quantum efficiency for fluorescence and phosphorescence is preferred. Specific examples thereof include, but are not limited to, 8-hydroxyquinoline aluminum complex (Alq₃), carbazole-based compounds, dimerized styryl compounds, BAlq, 10-hydroxybenzoquinoline-metal compounds, benzoxazole-based compounds, benzthiazole-based compounds, and benzimidazole-based compounds, poly(p-phenylenevinylene) (PPV)-based polymers, spiro compounds, polyfluorene, and rubrene.

The electron injecting layer may be used to have high injection efficiency of electrons transferred from the cathode. Examples of such an electron injecting layer include lithium quinolate (Liq), etc., but are not limited thereto.

The electron transport layer is a material that may receive electrons injected from the cathode and may transfer the electrons to the light-emitting layer. A material with high electron mobility is suitable. Specific examples thereof include, but are not limited to, 8-hydroxyquinoline Al complex, Alq₃ complexes, organic radical compounds, hydroxyflavone-metal complexes.

The organic light-emitting device according to an embodiment of the present invention may be of a top emission, bottom emission or dual emission type according to the materials used.

In addition, the organic compound according to an embodiment of the present invention may perform its function even in organic electronic devices, including organic solar cells, organic photoconductors, and organic transistors, based on a similar principle to that applied to the organic light-emitting device.

### Mode for Carrying Out Invention

Hereinafter, the present invention will be explained in more detail with reference to the preferred examples. However, these examples are provided for illustrative purposes and do not serve to limit the scope of the invention. It will be obvious to those skilled in the art that various modifications and changes are possible without departing from the scope and technical spirit of the present invention.

### Synthesis Example 1: Synthesis of Compound 2

### (1) Preparation Example 1: Synthesis of Intermediate 2-1

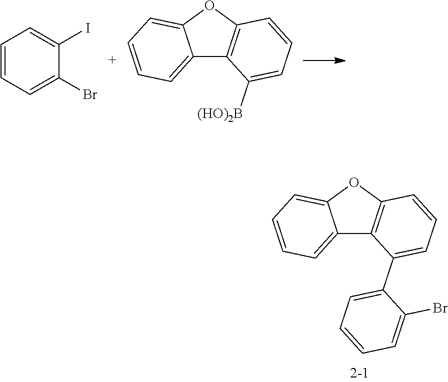

1-Bromo-2-iodobenzene (10.0 g, 0.035 mol), 1-Dibenzofuranylboronic acid (9.0 g, 0.042 mol), K₂CO₃ (14.7 g, 0.105 mol), and Pd(PPh₃)₄ (0.8 g, 0.7 mmol) were added with Toluene 200 mL, EtOH 50 mL, and H₂O 50 mL and reacted while stirring at 100° C. for 6 hours. After completion of the reaction, the reaction mixture was extracted, concentrated, and then columned to obtain 9.0 g of Intermediate 2-1 (yield 78.8%).

### (2) Preparation Example 2: Synthesis of Intermediate 2-2

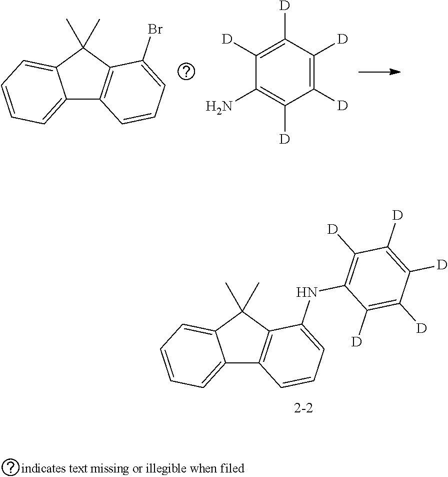 [SMILES: CC1(C)c2ccccc2-c2cccc(Br)c21 ; [2H]c1c([2H])c([2H])c(N)c([2H])c1[2H] ; [2H]c1c([2H])c([2H])c(Nc2cccc3c2C(C)(C)c2ccccc2-3)c([2H])c1[2H]]

1-Bromo-9,9-dimethyl-9H-fluorene (10.0 g, 0.037 mol), Aniline-d5 (5.4 g, 0.056 mol), NaOtBu (10.6 g, 0.112 mol), Pd (dba) 2 (0.8 g, 1.5 mmol), and t-Bu₃P (0.6 g, 3.0 mmol) were added with Toluene 150 mL and reacted while stirring at 70° C. for 4 hours. After completion of the reaction, the reaction mixture was extracted, concentrated, and then column and recrystallized to obtain 6.7 g of Intermediate 2-2 (yield 63.0%).

### (3) Preparation Example 3: Synthesis of Compound 2

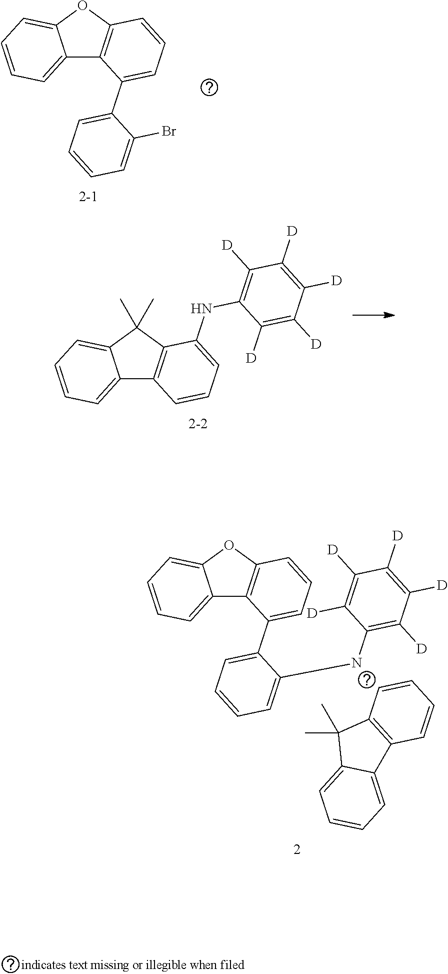

Intermediate 2-1 (10.0 g, 0.031 mol), Intermediate 2-2 (13.5 g, 0.047 mol), NaOtBu (8.9 g, 0.094 mol), Pd (dba) 2 (0.7 g, 1.2 mmol), and t-Bu₃P (0.5 g, 2.4 mmol) were added with Toluene 150 mL and reacted while stirring at 70° C. for 4 hours. After completion of the reaction, the reaction mixture was extracted, concentrated, and then column and recrystallized to obtain 12.5 g of Compound 2 (yield 75.8%).

LC/MS: m/z=532 [(M)⁺]

### Synthesis Example 2: Synthesis of Compound 18

### (1) Preparation Example 1: Synthesis of Intermediate 18-1

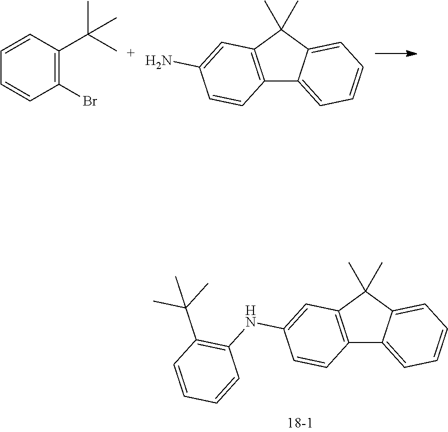 [SMILES: CC(C)(C)c1ccccc1Br ; CC1(C)c2ccccc2-c2ccc(N)cc21 ; CC(C)(C)c1ccccc1Nc1ccc2c(c1)C(C)(C)c1ccccc1-2]

1-Bromo-2-tert-butylbenzene (10.0 g, 0.047 mol), 2-Amino-9,9-dimethylfluorene (14.7 g, 0.071 mol), NaOtBu (13.5 g, 0.142 mol), Pd (dba) 2 (1.1 g, 1.9 mmol), and t-Bu₃P (0.8 g, 3.8 mmol) were added with Toluene 150 mL and reacted while stirring at 70° C. for 4 hours. After completion of the reaction, the reaction mixture was extracted, concentrated, and then column and recrystallized to obtain 10.8 g of Intermediate 18-1 (yield 67.4%).

### (2) Preparation Example 2: Synthesis of Compound 18

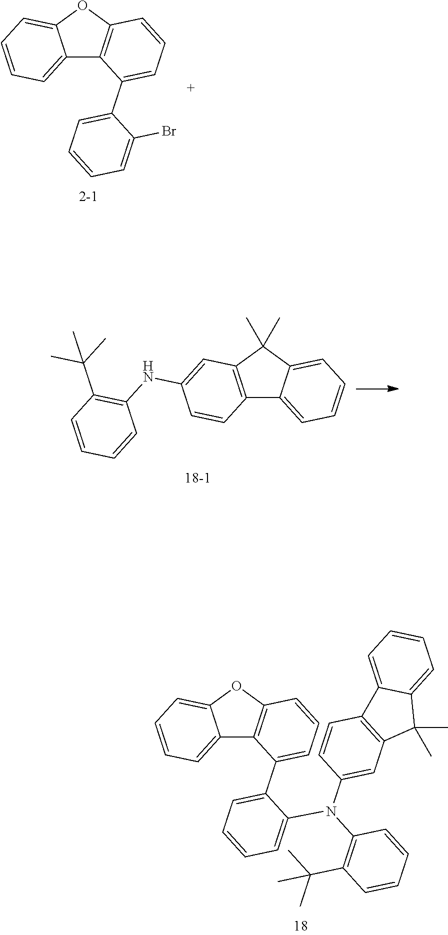 [SMILES: Brc1ccccc1-c1cccc2oc3ccccc3c12 ; CC(C)(C)c1ccccc1Nc1ccc2c(c1)C(C)(C)c1ccccc1-2 ; CC(C)(C)c1ccccc1N(c1ccc2c(c1)C(C)(C)c1ccccc1-2)c1ccccc1-c1cccc2oc3ccccc3c12]

Intermediate 2-1 (10.0 g, 0.031 mol), Intermediate 18-1 (15.9 g, 0.047 mol), NaOtBu (8.9 g, 0.094 mol), Pd (dba) 2 (0.7 g, 1.2 mmol), and t-Bu₃P (0.5 g, 2.4 mmol) were added with Toluene 150 mL and reacted while stirring at 70° C. for 4 hours. After completion of the reaction, the reaction mixture was extracted, concentrated, and then column and recrystallized to obtain 14.1 g of Compound 18 (yield 78.1%).

LC/MS: m/z=583 [(M)⁺]

### Synthesis Example 3: Synthesis of Compound 23

### (1) Preparation Example 1: Synthesis of Intermediate 23-1

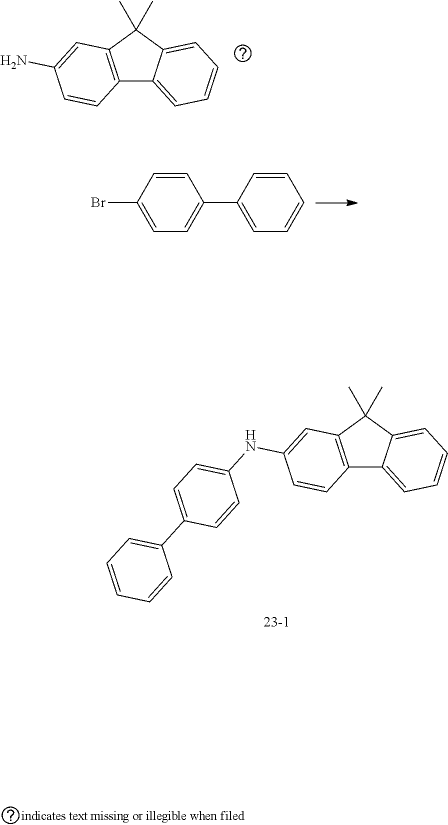 [SMILES: CC1(C)c2ccccc2-c2ccc(N)cc21 ; Brc1ccc(-c2ccccc2)cc1 ; CC1(C)c2ccccc2-c2ccc(Nc3ccc(-c4ccccc4)cc3)cc21]

9,9-Dimethyl-9H-fluoren-2-amine (10.0 g, 0.048 mol), 4-Bromobiphenyl (16.7 g, 0.072 mol), NaOtBu (13.8 g, 0.143 mol), Pd (dba) 2 (1.1 g, 1.9 mmol), and t-Bu₃P (0.8 g, 3.8 mmol) were added with Toluene 150 mL and reacted while stirring at 70° C. for 4 hours. After completion of the reaction, the reaction mixture was extracted, concentrated, and then column and recrystallized to obtain 9.2 g of Intermediate 23-1 (yield 53.3%).

### (2) Preparation Example 2: Synthesis of Compound 23

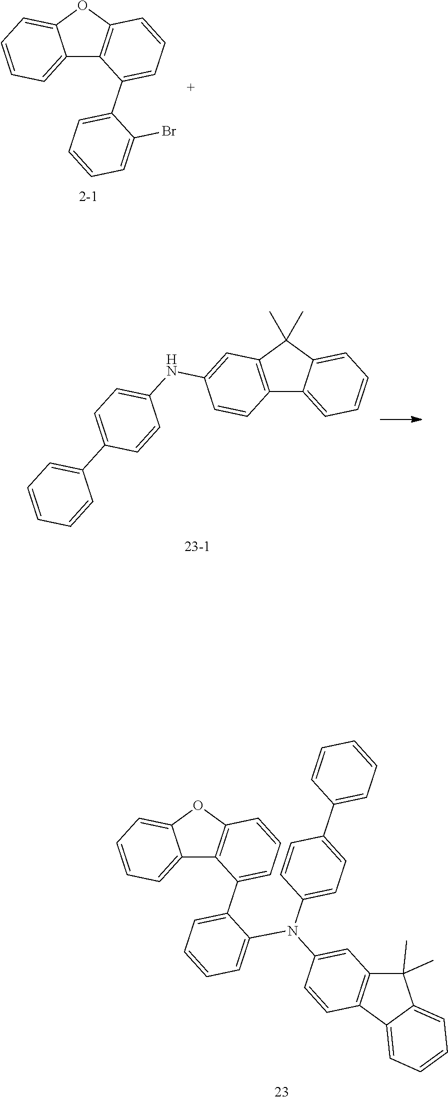 [SMILES: Brc1ccccc1-c1cccc2oc3ccccc3c12 ; CC1(C)c2ccccc2-c2ccc(Nc3ccc(-c4ccccc4)cc3)cc21 ; CC1(C)c2ccccc2-c2ccc(N(c3ccc(-c4ccccc4)cc3)c3ccccc3-c3cccc4oc5ccccc5c34)cc21]

Intermediate 2-1 (10.0 g, 0.031 mol), Intermediate 23-1 (16.8 g, 0.046 mol), NaOtBu (8.9 g, 0.093 mol), Pd (dba) 2 (0.7 g, 1.2 mmol), and t-Bu₃P (0.5 g, 2.5 mmol) were added with Toluene 150 mL and reacted while stirring at 70° C. for 4 hours. After completion of the reaction, the reaction mixture was extracted, concentrated, and then column and recrystallized to obtain 14.1 g of Compound 23 (yield 78.1%).

### Synthesis Example 4: Synthesis of Compound 27

### (1) Preparation Example 1: Synthesis of Intermediate 27-1

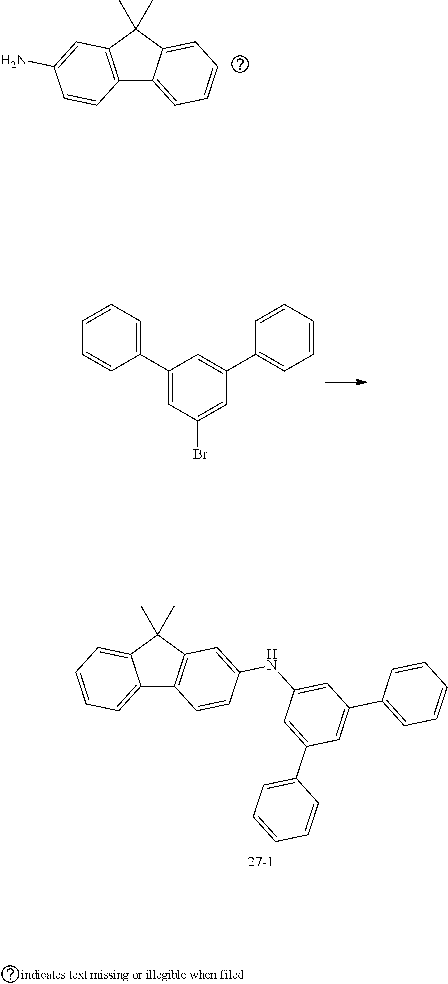 [SMILES: CC1(C)c2ccccc2-c2ccc(N)cc21 ; Brc1cc(-c2ccccc2)cc(-c2ccccc2)c1 ; CC1(C)c2ccccc2-c2ccc(Nc3cc(-c4ccccc4)cc(-c4ccccc4)c3)cc21]

2-Amino-9,9-dimethylfluorene (10.0 g, 0.048 mol), 1-Bromo-3,5-diphenylbenzene (22.2 g, 0.072 mol), NaOtBu (13.8 g, 0.143 mol), Pd (dba) 2 (1.1 g, 1.9 mmol), and t-Bu₃P (0.8 g, 3.8 mmol) were added with Toluene 150 mL and reacted while stirring at 70° C. for 4 hours. After completion of the reaction, the reaction mixture was extracted, concentrated, and then column and recrystallized to obtain 11.5 g of Intermediate 27-1 (yield 55.0%).

### (2) Preparation Example 2: Synthesis of Compound 27

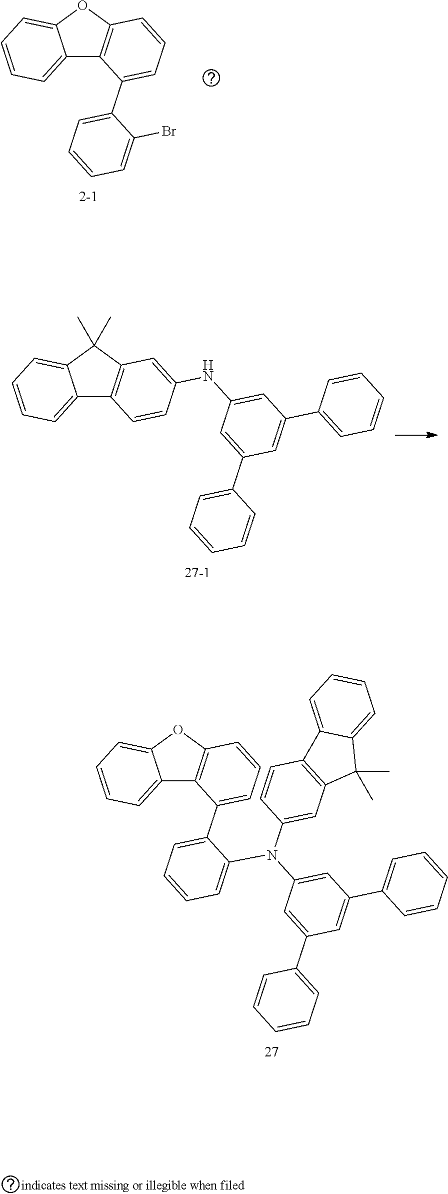 [SMILES: Brc1ccccc1-c1cccc2oc3ccccc3c12 ; CC1(C)c2ccccc2-c2ccc(Nc3cc(-c4ccccc4)cc(-c4ccccc4)c3)cc21 ; CC1(C)c2ccccc2-c2ccc(N(c3cc(-c4ccccc4)cc(-c4ccccc4)c3)c3ccccc3-c3cccc4oc5ccccc5c34)cc21]

Intermediate 2-1 (10.0 g, 0.031 mol), Intermediate 27-1 (20.3 g, 0.047 mol), NaOtBu (8.9 g, 0.094 mol), Pd (dba) 2 (0.7 g, 1.2 mmol), and t-Bu₃P (0.5 g, 2.4 mmol) were added with Toluene 150 mL and reacted while stirring at 70° C. for 4 hours. After completion of the reaction, the reaction mixture was extracted, concentrated, and then column and recrystallized to obtain 15.5 g of Compound 27 (yield 73.7%).

LC/MS: m/z=679 [(M)⁺]

### Synthesis Example 5: Synthesis of Compound 48

### (1) Preparation Example 1: Synthesis of Intermediate 48-1

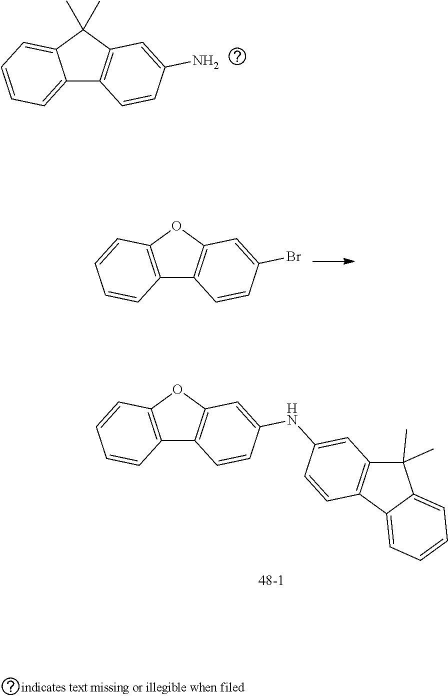 [SMILES: CC1(C)c2ccccc2-c2ccc(N)cc21 ; Brc1ccc2c(c1)oc1ccccc12 ; CC1(C)c2ccccc2-c2ccc(Nc3ccc4c(c3)oc3ccccc34)cc21]

2-Amino-9,9-dimethylfluorene (10.0 g, 0.048 mol), 3-Bromodibenzofuran (17.7 g, 0.072 mol), NaOtBu (13.8 g, 0.143 mol), Pd (dba) 2 (1.1 g, 1.9 mmol), and t-Bu₃P (0.8 g, 3.8 mmol) were added with Toluene 150 mL and reacted while stirring at 70° C. for 4 hours. After completion of the reaction, the reaction mixture was extracted, concentrated, and then column and recrystallized to obtain 10.8 g of Intermediate 48-1 (yield 60.2%).

### (2) Preparation Example 2: Synthesis of Compound 48

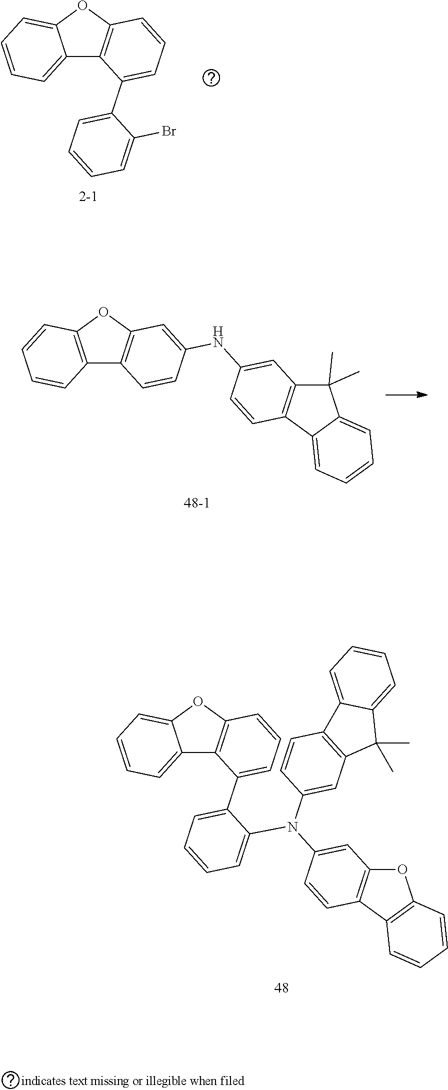 [SMILES: Brc1ccccc1-c1cccc2oc3ccccc3c12 ; CC1(C)c2ccccc2-c2ccc(Nc3ccc4c(c3)oc3ccccc34)cc21 ; CC1(C)c2ccccc2-c2ccc(N(c3ccc4c(c3)oc3ccccc34)c3ccccc3-c3cccc4oc5ccccc5c34)cc21]

Intermediate 2-1 (10.0 g, 0.031 mol), Intermediate 48-1 (17.4 g, 0.047 mol), NaOtBu (8.9 g, 0.094 mol), Pd (dba) 2 (0.7 g, 1.2 mmol), and t-Bu₃P (0.5 g, 2.4 mmol) were added with Toluene 150 mL and reacted while stirring at 70° C. for 4 hours. After completion of the reaction, the reaction mixture was extracted, concentrated, and then column and recrystallized to obtain 13.8 g of Compound 48 (yield 72.2%).

LC/MS: m/z=617 [(M)⁺]

### Synthesis Example 6: Synthesis of Compound 73

### (1) Preparation Example 1: Synthesis of Intermediate 73-1

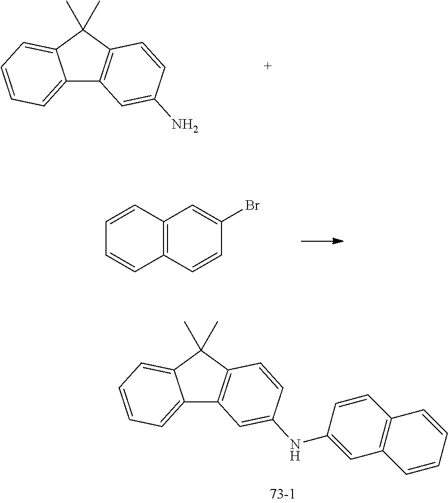 [SMILES: CC1(C)c2ccccc2-c2cc(N)ccc21 ; Brc1ccc2ccccc2c1 ; CC1(C)c2ccccc2-c2cc(Nc3ccc4ccccc4c3)ccc21]

3-Amino-9,9-dimethylfluorene (10.0 g, 0.048 mol), 2-Bromonaphthalene (14.8 g, 0.072 mol), NaOtBu (13.8 g, 0.143 mol), Pd (dba) 2 (1.1 g, 1.9 mmol), and t-Bu₃P (0.8 g, 3.8 mmol) were added with Toluene 150 mL and reacted while stirring at 70° C. for 4 hours. After completion of the reaction, the reaction mixture was extracted, concentrated, and then column and recrystallized to obtain 8.7 g of Intermediate 73-1 (yield 54.3%).

### (2) Preparation Example 2: Synthesis of Compound 73

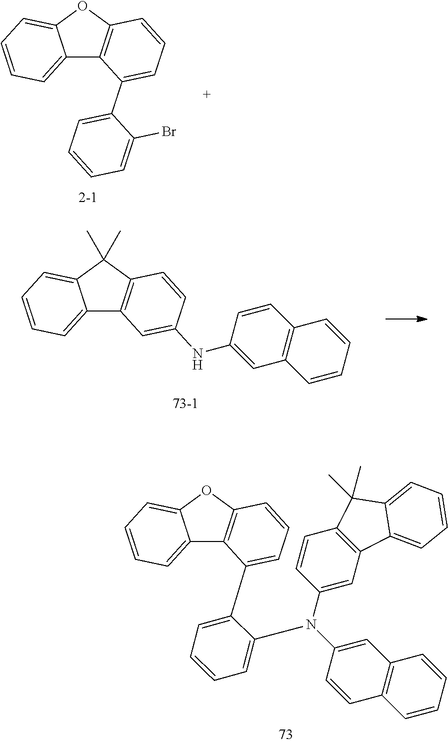 [SMILES: Brc1ccccc1-c1cccc2oc3ccccc3c12 ; CC1(C)c2ccccc2-c2cc(Nc3ccc4ccccc4c3)ccc21 ; CC1(C)c2ccccc2-c2cc(N(c3ccc4ccccc4c3)c3ccccc3-c3cccc4oc5ccccc5c34)ccc21]

Intermediate 2-1 (10.0 g, 0.031 mol), Intermediate 73-1 (15.6 g, 0.047 mol), NaOtBu (8.9 g, 0.094 mol), Pd (dba) 2 (0.7 g, 1.2 mmol), and t-Bu₃P (0.5 g, 2.4 mmol) were added with Toluene 150 mL and reacted while stirring at 70° C. for 4 hours. After completion of the reaction, the reaction mixture was extracted, concentrated, and then column and recrystallized to obtain 12.9 g of Compound 73 (yield 72.2%).

LC/MS: m/z=577 [(M)⁺]

### Synthesis Example 7: Synthesis of Compound 89

### (1) Preparation Example 1: Synthesis of Intermediate 89-1

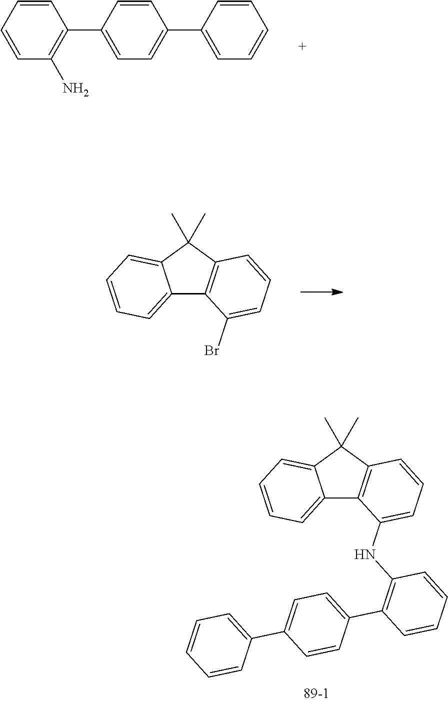 [SMILES: Nc1ccccc1-c1ccc(-c2ccccc2)cc1 ; CC1(C)c2ccccc2-c2c(Br)cccc21 ; CC1(C)c2ccccc2-c2c(Nc3ccccc3-c3ccc(-c4ccccc4)cc3)cccc21]

2-(4-Phenylphenyl) aniline (10.0 g, 0.041 mol), 4-Bromo-9,9-dimethylfluorene (16.7 g, 0.061 mol), NaOtBu (11.8 g, 0.122 mol), Pd (dba) 2 (0.9 g, 1.6 mmol), and t-Bu₃P (0.7 g, 3.3 mmol) were added with Toluene 150 mL and reacted while stirring at 70° C. for 4 hours. After completion of the reaction, the reaction mixture was extracted, concentrated, and then column and recrystallized to obtain 9.1 g of Intermediate 89-1 (yield 51.0%).

### (2) Preparation Example 2: Synthesis of Compound 89

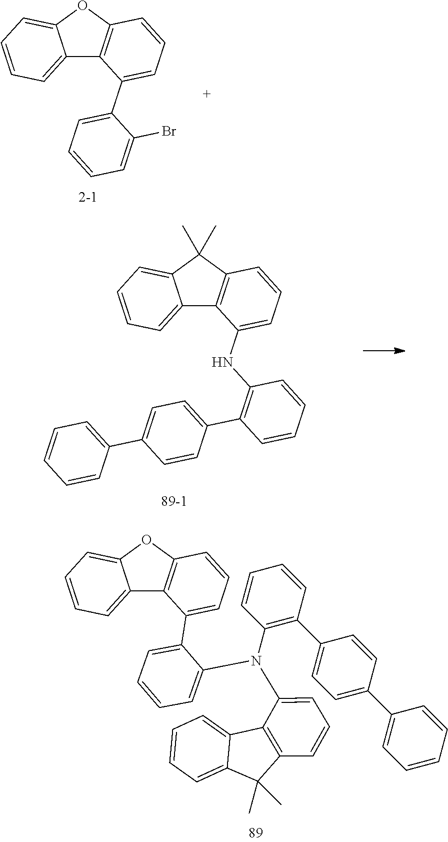 [SMILES: Brc1ccccc1-c1cccc2oc3ccccc3c12 ; CC1(C)c2ccccc2-c2c(Nc3ccccc3-c3ccc(-c4ccccc4)cc3)cccc21 ; CC1(C)c2ccccc2-c2c(N(c3ccccc3-c3ccc(-c4ccccc4)cc3)c3ccccc3-c3cccc4oc5ccccc5c34)cccc21]

Intermediate 2-1 (10.0 g, 0.031 mol), Intermediate 89-1 (20.3 g, 0.047 mol), NaOtBu (8.9 g, 0.094 mol), Pd (dba) 2 (0.7 g, 1.2 mmol), and t-Bu₃P (0.5 g, 2.4 mmol) were added with Toluene 150 mL and reacted while stirring at 70° C. for 4 hours. After completion of the reaction, the reaction mixture was extracted, concentrated, and then column and recrystallized to obtain 16.5 g of Compound 89 (yield 78.4%).

LC/MS: m/z=679 [(M)⁺]

### Synthesis Example 8: Synthesis of Compound 102

### (1) Preparation Example 1: Synthesis of Intermediate 102-1

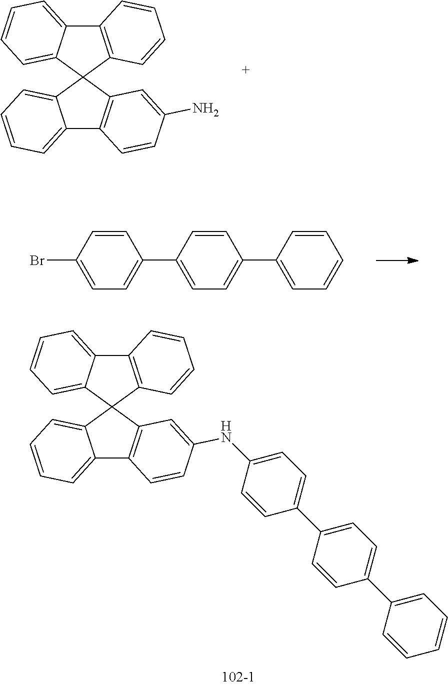

9,9′-Spirobi[9H-fluoren]-2-amine (10.0 g, 0.030 mol), 4-Bromo-p-terphenyl (14.0 g, 0.045 mol), NaOtBu (8.7 g, 0.091 mol), Pd (dba) 2 (0.7 g, 1.2 mmol), and t-Bu₃P (0.5 g, 2.4 mmol) were added with Toluene 150 mL and reacted while stirring at 70° C. for 4 hours. After completion of the reaction, the reaction mixture was extracted, concentrated, and then column and recrystallized to obtain 8.2 g of Intermediate 102-1 (yield 48.6%).

### (2) Preparation Example 2: Synthesis of Compound 102

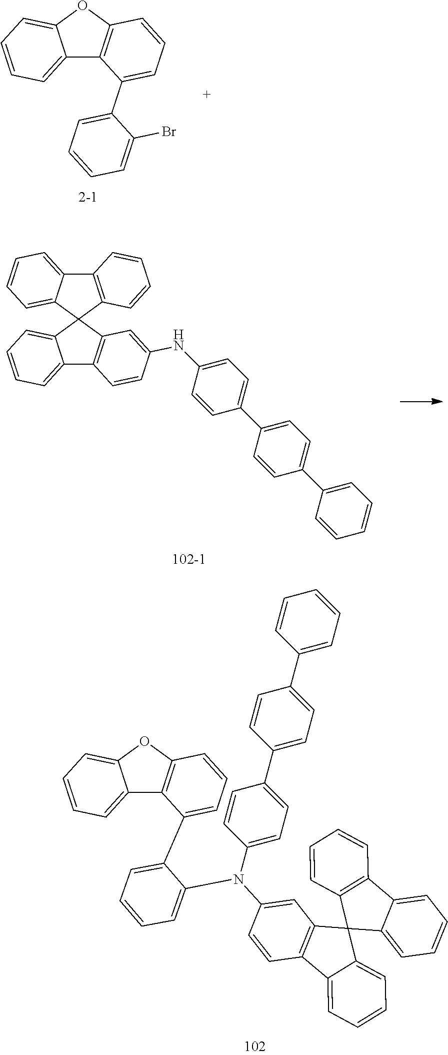 [SMILES: Brc1ccccc1-c1cccc2oc3ccccc3c12 ; c1ccc(-c2ccc(-c3ccc(Nc4ccc5c(c4)C4(c6ccccc6-c6ccccc64)c4ccccc4-5)cc3)cc2)cc1 ; c1ccc(-c2ccc(-c3ccc(N(c4ccc5c(c4)C4(c6ccccc6-c6ccccc64)c4ccccc4-5)c4ccccc4-c4cccc5oc6ccccc6c45)cc3)cc2)cc1]

Intermediate 2-1 (10.0 g, 0.031 mol), Intermediate 102-1 (26.0 g, 0.047 mol), NaOtBu (8.9 g, 0.094 mol), Pd (dba) 2 (0.7 g, 1.2 mmol), and t-Bu₃P (0.5 g, 2.4 mmol) were added with Toluene 150 mL and reacted while stirring at 70° C. for 4 hours. After completion of the reaction, the reaction mixture was extracted, concentrated, and then column and recrystallized to obtain 20.3 g of Compound 102 (yield 81.8%).

LC/MS: m/z=801 [(M)⁺]

### Synthesis Example 9: Synthesis of Compound 112

### (1) Preparation Example 1: Synthesis of Intermediate 112-1

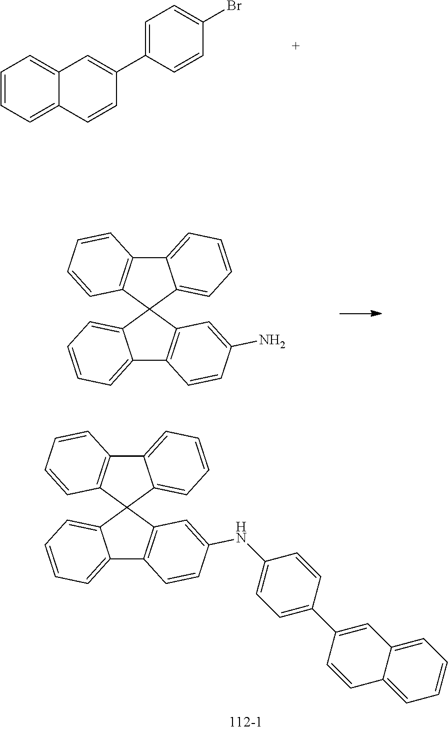 [SMILES: Brc1ccc(-c2ccc3ccccc3c2)cc1 ; Nc1ccc2c(c1)C1(c3ccccc3-c3ccccc31)c1ccccc1-2 ; c1ccc2c(c1)-c1ccccc1C21c2ccccc2-c2ccc(Nc3ccc(-c4ccc5ccccc5c4)cc3)cc21]

2-(4-Bromophenyl) naphthalene (10.0 g, 0.030 mol), 9,9′-Spirobi[9H-fluoren]-2-amine (12.8 g, 0.045 mol), NaOtBu (8.7 g, 0.091 mol), Pd (dba) 2 (0.7 g, 1.2 mmol), and t-Bu₃P (0.5 g, 2.4 mmol) were added with Toluene 150 mL and reacted while stirring at 70° C. for 4 hours. After completion of the reaction, the reaction mixture was extracted, concentrated, and then column and recrystallized to obtain 8.4 g of Intermediate 112-1 (yield 52.2%).

### (2) Preparation Example 2: Synthesis of Compound 112

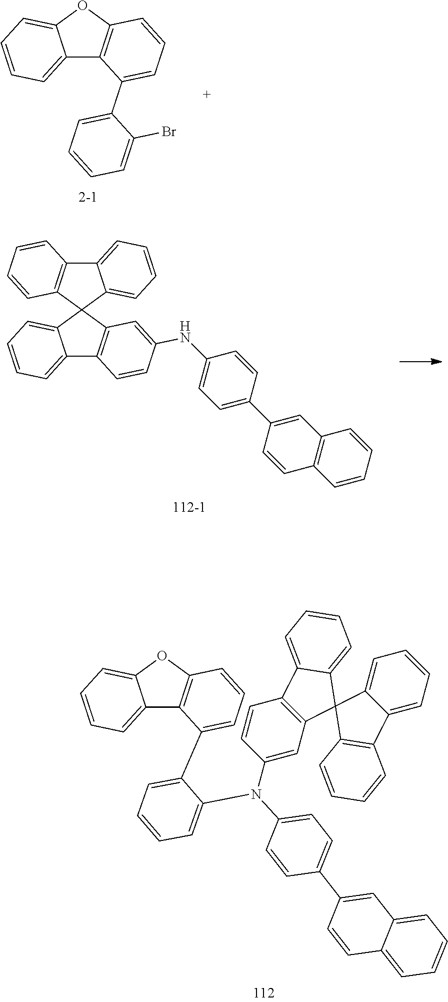 [SMILES: Brc1ccccc1-c1cccc2oc3ccccc3c12 ; c1ccc2c(c1)-c1ccccc1C21c2ccccc2-c2ccc(Nc3ccc(-c4ccc5ccccc5c4)cc3)cc21 ; c1ccc(N(c2ccc(-c3ccc4ccccc4c3)cc2)c2ccc3c(c2)C2(c4ccccc4-c4ccccc42)c2ccccc2-3)c(-c2cccc3oc4ccccc4c23)c1]

Intermediate 2-1 (10.0 g, 0.031 mol), Intermediate 112-1 (27.8 g, 0.047 mol), NaOtBu (8.9 g, 0.094 mol), Pd (dba) 2 (0.7 g, 1.2 mmol), and t-Bu₃P (0.5 g, 2.4 mmol) were added with Toluene 150 mL and reacted while stirring at 70° C. for 4 hours. After completion of the reaction, the reaction mixture was extracted, concentrated, and then column and recrystallized to obtain 18.4 g of Compound 112 (yield 76.6%).

LC/MS: m/z=775 [(M)⁺]

### Synthesis Example 10: Synthesis of Compound 135

### (1) Preparation Example 1: Synthesis of Intermediate 135-1

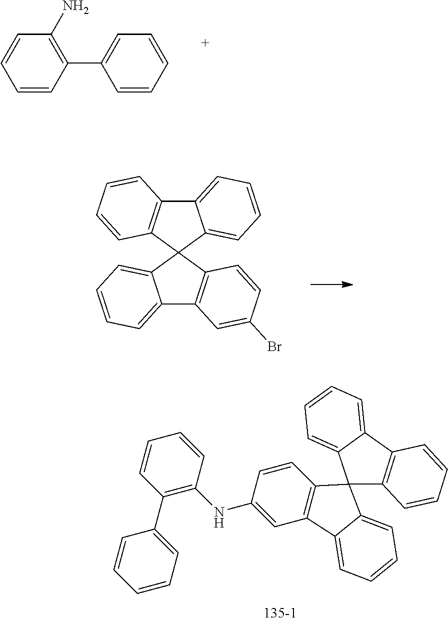 [SMILES: Nc1ccccc1-c1ccccc1 ; Brc1ccc2c(c1)-c1ccccc1C21c2ccccc2-c2ccccc21 ; c1ccc(-c2ccccc2Nc2ccc3c(c2)-c2ccccc2C32c3ccccc3-c3ccccc32)cc1]

2-Aminobiphenyl (10.0 g, 0.059 mol), 3-Bromo-9,9′-spirobi[9H-fluorene] (35.0 g, 0.089 mol), NaOtBu (17.0 g, 0.178 mol), Pd (dba) 2 (1.4 g, 2.4 mmol), and t-Bu₃P (1.0 g, 4.7 mmol) were added with Toluene 150 mL and reacted while stirring at 70° C. for 4 hours. After completion of the reaction, the reaction mixture was extracted, concentrated, and then column and recrystallized to obtain 13.2 g of Intermediate 135-1 (yield 46.2%).

### (2) Preparation Example 2: Synthesis of Compound 135

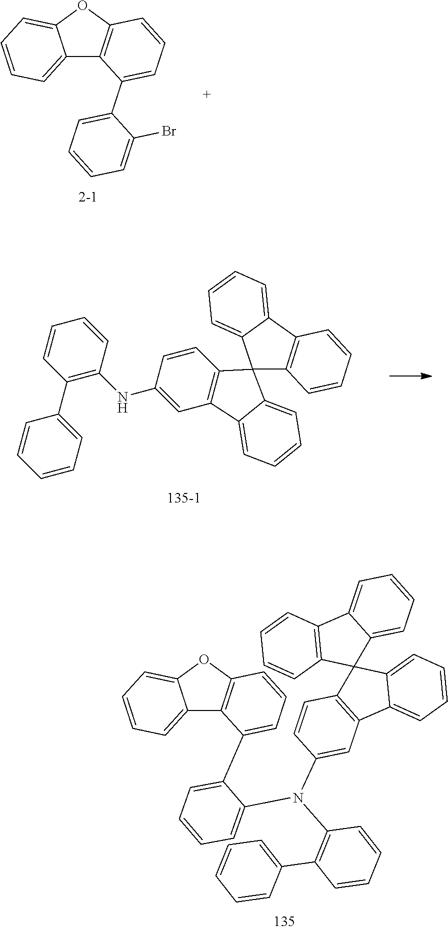 [SMILES: Brc1ccccc1-c1cccc2oc3ccccc3c12 ; c1ccc(-c2ccccc2Nc2ccc3c(c2)-c2ccccc2C32c3ccccc3-c3ccccc32)cc1 ; c1ccc(-c2ccccc2N(c2ccc3c(c2)-c2ccccc2C32c3ccccc3-c3ccccc32)c2ccccc2-c2cccc3oc4ccccc4c23)cc1]

Intermediate 2-1 (10.0 g, 0.031 mol), Intermediate 135-1 (22.5 g, 0.047 mol), NaOtBu (8.9 g, 0.094 mol), Pd (dba) 2 (0.7 g, 1.2 mmol), and t-Bu₃P (0.5 g, 2.4 mmol) were added with Toluene 150 mL and reacted while stirring at 70° C. for 4 hours. After completion of the reaction, the reaction mixture was extracted, concentrated, and then column and recrystallized to obtain 15.5 g of Compound 135 (yield 69.0%).

LC/MS: m/z=725 [(M)⁺]

### Synthesis Example 11: Synthesis of Compound 153

### (1) Preparation Example 1: Synthesis of Intermediate 153-1

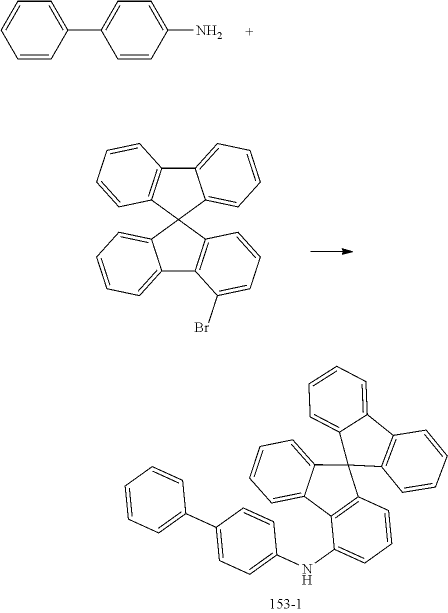 [SMILES: Nc1ccc(-c2ccccc2)cc1 ; Brc1cccc2c1-c1ccccc1C21c2ccccc2-c2ccccc21 ; c1ccc(-c2ccc(Nc3cccc4c3-c3ccccc3C43c4ccccc4-c4ccccc43)cc2)cc1]

4-Aminobiphenyl (10.0 g, 0.059 mol), 4-Bromo-9,9′-spirobi[9H-fluorene] (35.0 g, 0.089 mol), NaOtBu (17.0 g, 0.178 mol), Pd (dba) 2 (1.4 g, 2.4 mmol), and t-Bu₃P (1.4 g, 2.4 mmol) were added with Toluene 150 mL and reacted while stirring at 70° C. for 4 hours. After completion of the reaction, the reaction mixture was extracted, concentrated, and then column and recrystallized to obtain 15.1 g of Intermediate 153-1 (yield 52.8%).

### (2) Preparation Example 2: Synthesis of Compound 153

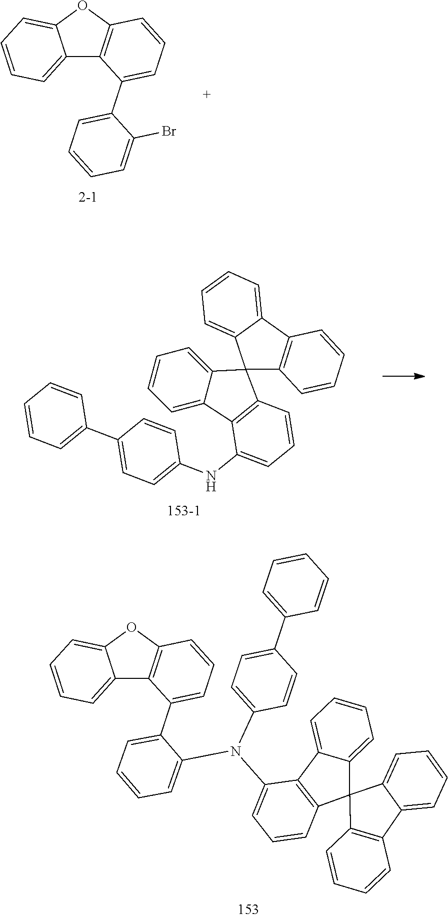 [SMILES: Brc1ccccc1-c1cccc2oc3ccccc3c12 ; c1ccc(-c2ccc(Nc3cccc4c3-c3ccccc3C43c4ccccc4-c4ccccc43)cc2)cc1 ; c1ccc(-c2ccc(N(c3ccccc3-c3cccc4oc5ccccc5c34)c3cccc4c3-c3ccccc3C43c4ccccc4-c4ccccc43)cc2)cc1]

Intermediate 2-1 (10.0 g, 0.031 mol), Intermediate 153-1 (22.5 g, 0.046 mol), NaOtBu (8.9 g, 0.093 mol), Pd (dba) 2 (0.7 g, 1.2 mmol), and t-Bu₃P (0.5 g, 2.5 mmol) were added with Toluene 150 mL and reacted while stirring at 70° C. for 4 hours. After completion of the reaction, the reaction mixture was extracted, concentrated, and then column and recrystallized to obtain 16.2 g of Compound 153 (yield 72.1%).

LC/MS: m/z=725 [(M)⁺]

### Synthesis Example 12: Synthesis of Compound 185

### (1) Preparation Example 1: Synthesis of Intermediate 185-1

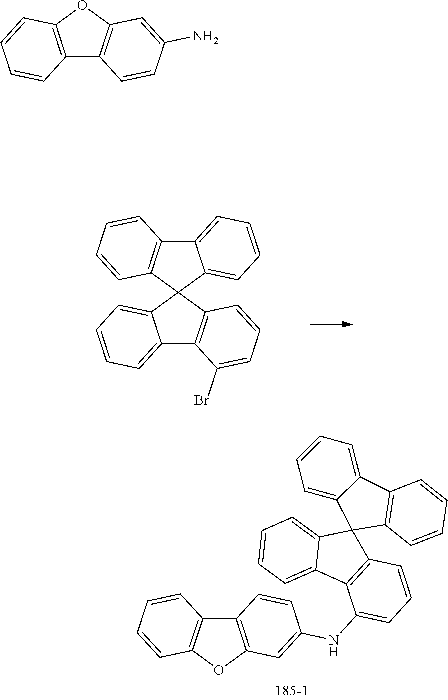 [SMILES: Nc1ccc2c(c1)oc1ccccc12 ; Brc1cccc2c1-c1ccccc1C21c2ccccc2-c2ccccc21 ; c1ccc2c(c1)-c1ccccc1C21c2ccccc2-c2c(Nc3ccc4c(c3)oc3ccccc34)cccc21]

Dibenzo[b,d]furan-3-amine (10.0 g, 0.055 mol), 4-Bromo-9,9′-spirobi[9H-fluorene] (32.4 g, 0.082 mol), NaOtBu (15.7 g, 0.164 mol), Pd (dba) 2 (1.3 g, 2.2 mmol), and t-Bu₃P (0.9 g, 4.4 mmol) were added with Toluene 150 mL and reacted while stirring at 70° C. for 4 hours. After completion of the reaction, the reaction mixture was extracted, concentrated, and then column and recrystallized to obtain 15.2 g of Intermediate 185-1 (yield 56.0%).

### (2) Preparation Example 2: Synthesis of Compound 185

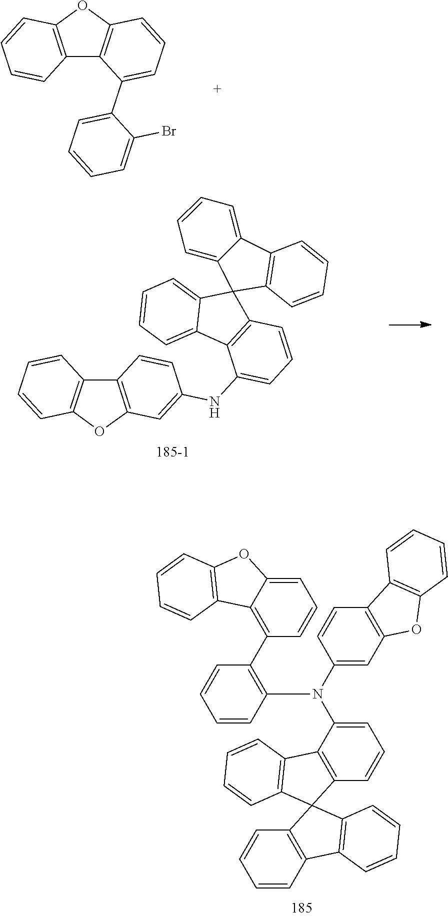 [SMILES: Brc1ccccc1-c1cccc2oc3ccccc3c12 ; c1ccc2c(c1)-c1ccccc1C21c2ccccc2-c2c(Nc3ccc4c(c3)oc3ccccc34)cccc21 ; c1ccc(N(c2ccc3c(c2)oc2ccccc23)c2cccc3c2-c2ccccc2C32c3ccccc3-c3ccccc32)c(-c2cccc3oc4ccccc4c23)c1]

Intermediate 2-1 (10.0 g, 0.031 mol), Intermediate 185-1 (23.1 g, 0.046 mol), NaOtBu (8.9 g, 0.093 mol), Pd (dba) 2 (0.7 g, 1.2 mmol), and t-Bu₃P (0.5 g, 2.5 mmol) were added with Toluene 150 mL and reacted while stirring at 70° C. for 4 hours. After completion of the reaction, the reaction mixture was extracted, concentrated, and then column and recrystallized to obtain 14.8 g of Compound 185 (yield 64.7%).

LC/MS: m/z=739 [(M)⁺]

### Synthesis Example 13: Synthesis of Compound 189

### (1) Preparation Example 1: Synthesis of Intermediate 189-1

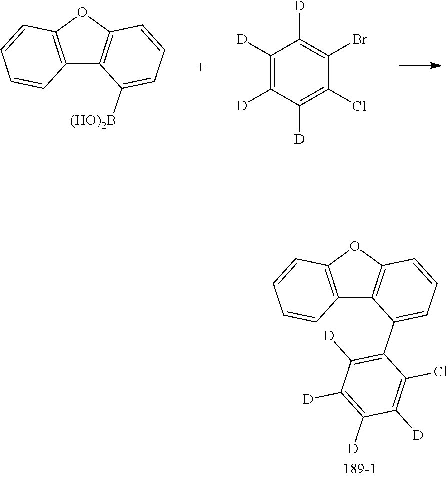 [SMILES: OB(O)c1cccc2oc3ccccc3c12 ; [2H]c1c([2H])c([2H])c(Br)c(Cl)c1[2H] ; [2H]c1c([2H])c([2H])c(-c2cccc3oc4ccccc4c23)c(Cl)c1[2H]]

1-dibenzofuranylboronic acid (10.0 g, 0.047 mol), 5-Bromo-6-chlorobenzene-1,2,3,4-d4 (11.1 g, 0.057 mol), K₂CO₃ (19.6 g, 0.142 mol), and Pd(PPh₃)₄ (1.0 g, 0.9 mmol) were added with Toluene 200 mL, EtOH 50 mL, and H₂O 50 mL and reacted while stirring at 100° C. for 6 hours. After completion of the reaction, the reaction mixture was extracted, concentrated, and then column and recrystallized to obtain 9.1 g of Intermediate 189-1 (yield 68.2%).

### (2) Preparation Example 2: Synthesis of Compound 189

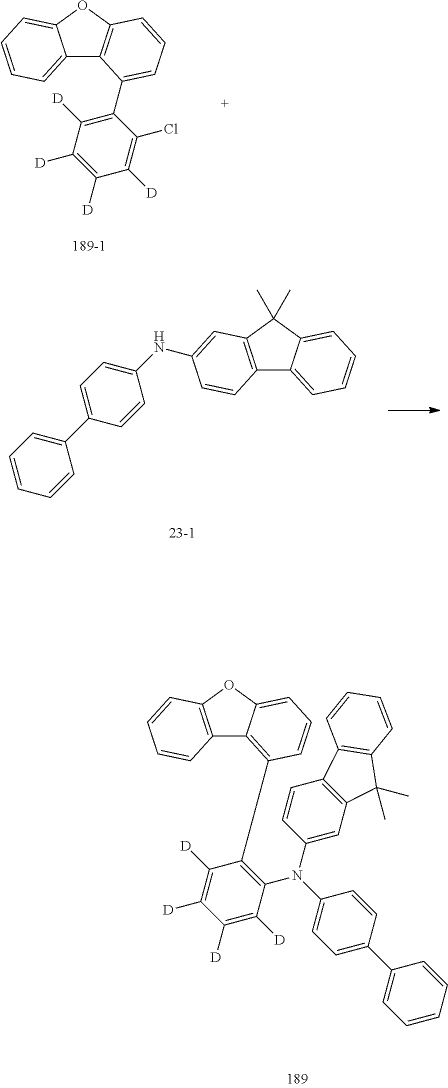 [SMILES: [2H]c1c([2H])c([2H])c(-c2cccc3oc4ccccc4c23)c(Cl)c1[2H] ; CC1(C)c2ccccc2-c2ccc(Nc3ccc(-c4ccccc4)cc3)cc21 ; [2H]c1c([2H])c([2H])c(N(c2ccc(-c3ccccc3)cc2)c2ccc3c(c2)C(C)(C)c2ccccc2-3)c(-c2cccc3oc4ccccc4c23)c1[2H]]

Intermediate 189-1 (10.0 g, 0.035 mol), Intermediate 23-1 (19.2 g, 0.053 mol), NaOtBu (10.2 g, 0.106 mol), Pd (dba) 2 (0.8 g, 1.4 mmol), and t-Bu₃P (0.6 g, 2.8 mmol) were added with Toluene 150 mL and reacted while stirring at 70° C. for 4 hours. After completion of the reaction, the reaction mixture was extracted, concentrated, and then column and recrystallized to obtain 13.3 g of Compound 189 (yield 61.9%).

LC/MS: m/z=607 [(M)⁺]

### Synthesis Example 14: Synthesis of Compound 202

### (1) Preparation Example 1: Synthesis of Compound 202

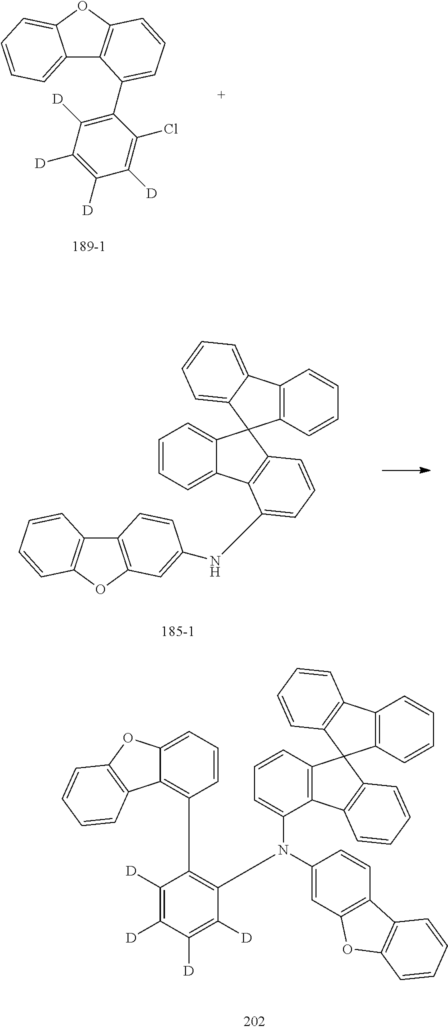 [SMILES: [2H]c1c([2H])c([2H])c(-c2cccc3oc4ccccc4c23)c(Cl)c1[2H] ; c1ccc2c(c1)-c1ccccc1C21c2ccccc2-c2c(Nc3ccc4c(c3)oc3ccccc34)cccc21 ; [2H]c1c([2H])c([2H])c(N(c2ccc3c(c2)oc2ccccc23)c2cccc3c2-c2ccccc2C32c3ccccc3-c3ccccc32)c(-c2cccc3oc4ccccc4c23)c1[2H]]

Intermediate 189-1 (10.0 g, 0.035 mol), Intermediate 185-1 (26.4 g, 0.053 mol), NaOtBu (10.2 g, 0.106 mol), Pd (dba) 2 (0.8 g, 1.4 mmol), and t-Bu₃P (0.6 g, 2.8 mmol) were added with Toluene 150 mL and reacted while stirring at 70° C. for 4 hours. After completion of the reaction, the reaction mixture was extracted, concentrated, and then column and recrystallized to obtain 14.8 g of Compound 202 (yield 56.3%).

LC/MS: m/z=743 [(M)⁺]

### Synthesis Example 15: Synthesis of Compound 233

### (1) Preparation Example 1: Synthesis of Intermediate 233-1

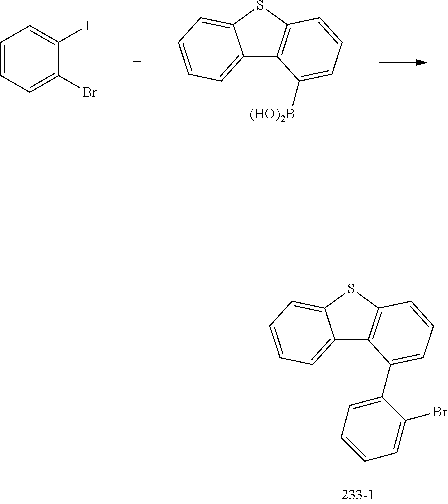 [SMILES: Brc1ccccc1I ; OB(O)c1cccc2sc3ccccc3c12 ; Brc1ccccc1-c1cccc2sc3ccccc3c12]

1-Bromo-2-iodobenzene (10.0 g, 0.035 mol), dibenzothiophene-1-boronic acid (9.7 g, 0.042 mol), K₂CO₃ (14.7 g, 0.105 mol), and Pd(PPh₃)₄ (0.8 g, 0.7 mmol) were added with Toluene 200 mL, EtOH 50 mL, and H₂O 50 mL and reacted while stirring at 100° C. for 6 hours. After completion of the reaction, the reaction mixture was extracted, concentrated, and then columned to obtain 9.7 g of Intermediate 233-1 (yield 80.9%).

### (2) Preparation Example 2: Synthesis of Intermediate 233-2

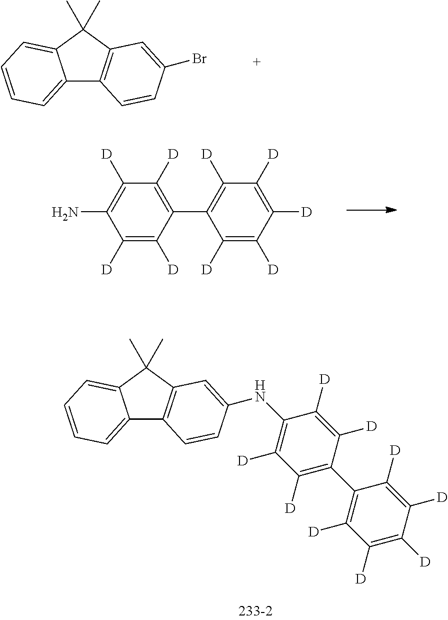 [SMILES: CC1(C)c2ccccc2-c2ccc(Br)cc21 ; [2H]c1c([2H])c([2H])c(-c2c([2H])c([2H])c(N)c([2H])c2[2H])c([2H])c1[2H] ; [2H]c1c([2H])c([2H])c(-c2c([2H])c([2H])c(Nc3ccc4c(c3)C(C)(C)c3ccccc3-4)c([2H])c2[2H])c([2H])c1[2H]]

2-Bromo-9,9-dimethylfluorene (10.0 g, 0.037 mol), 4-Aminobiphenyl-d9 (9.8 g, 0.056 mol), NaOtBu (10.6 g, 0.112 mol), Pd (dba) 2 (0.8 g, 1.5 mmol), and t-Bu₃P (0.6 g, 3.0 mmol) were added with Toluene 150 mL and reacted while stirring at 70° C. for 4 hours. After completion of the reaction, the reaction mixture was extracted, concentrated, and then column and recrystallized to obtain 9.2 g of Intermediate 233-2 (yield 67.8%).

### (3) Preparation Example 3: Synthesis of Compound 233

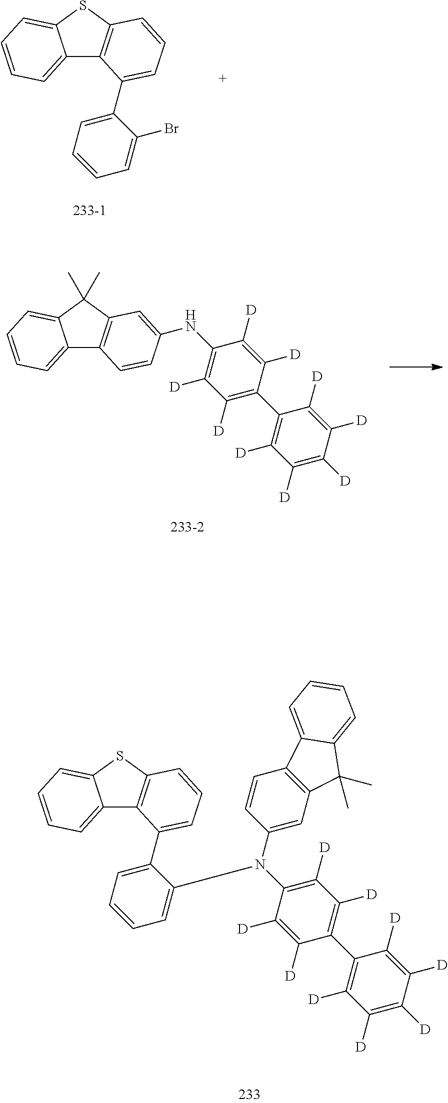 [SMILES: Brc1ccccc1-c1cccc2sc3ccccc3c12 ; [2H]c1c([2H])c([2H])c(-c2c([2H])c([2H])c(Nc3ccc4c(c3)C(C)(C)c3ccccc3-4)c([2H])c2[2H])c([2H])c1[2H] ; [2H]c1c([2H])c([2H])c(-c2c([2H])c([2H])c(N(c3ccc4c(c3)C(C)(C)c3ccccc3-4)c3ccccc3-c3cccc4sc5ccccc5c34)c([2H])c2[2H])c([2H])c1[2H]]

Intermediate 233-1 (10.0 g, 0.030 mol), Intermediate 233-2 (16.4 g, 0.045 mol), NaOtBu (8.5 g, 0.090 mol), Pd (dba) 2 (0.7 g, 1.2 mmol), and t-Bu₃P (0.5 g, 2.4 mmol) were added with Toluene 150 mL and reacted while stirring at 70° C. for 4 hours. After completion of the reaction, the reaction mixture was extracted, concentrated, and then column and recrystallized to obtain 13.7 g of Compound 233 (yield 73.9%).

LC/MS: m/z=628 [(M)⁺]

### Synthesis Example 16: Synthesis of Compound 284

### (1) Preparation Example 1: Synthesis of Intermediate 284-1

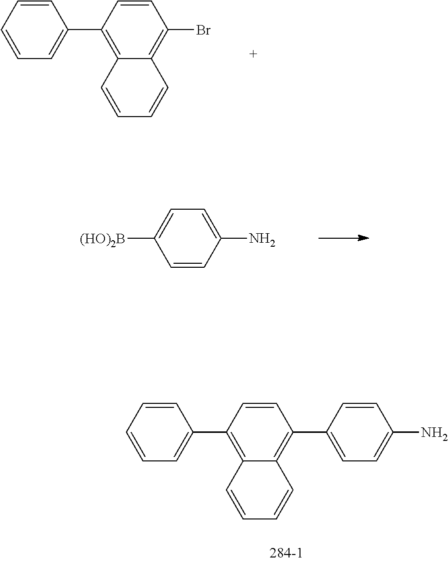 [SMILES: Brc1ccc(-c2ccccc2)c2ccccc12 ; Nc1ccc(B(O)O)cc1 ; Nc1ccc(-c2ccc(-c3ccccc3)c3ccccc23)cc1]

1-Bromo-4-phenylnaphthalene (10.0 g, 0.035 mol), 4-Aminophenylboronic acid (5.8 g, 0.042 mol), K₂CO₃ (14.6 g, 0.106 mol), and Pd(PPh₃)₄ (0.8 g, 0.7 mmol) were added with Toluene 200 mL, EtOH 50 mL, and H₂O 50 mL and reacted while stirring at 100° C. for 6 hours. After completion of the reaction, the reaction mixture was extracted, concentrated, and then columned to obtain 7.9 g of Intermediate 284-1 (yield 75.7%).

### (2) Preparation Example 2: Synthesis of Intermediate 284-2

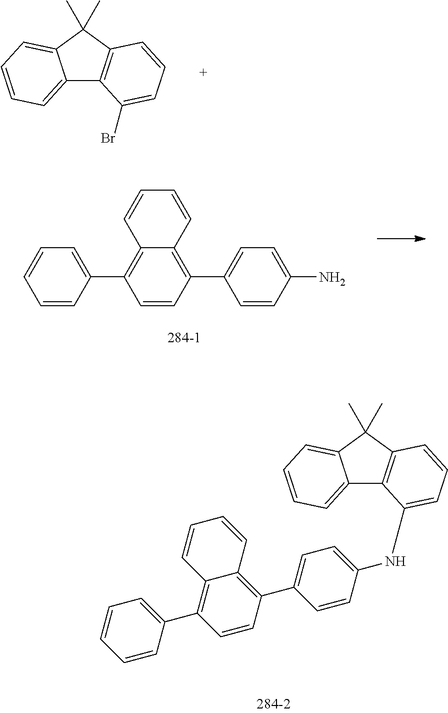 [SMILES: CC1(C)c2ccccc2-c2c(Br)cccc21 ; Nc1ccc(-c2ccc(-c3ccccc3)c3ccccc23)cc1 ; CC1(C)c2ccccc2-c2c(Nc3ccc(-c4ccc(-c5ccccc5)c5ccccc45)cc3)cccc21]

4-Bromo-9,9-dimethylfluorene (10.0 g, 0.037 mol), Intermediate 284-1 (16.2 g, 0.056 mol), NaOtBu (10.6 g, 0.112 mol), Pd (dba) 2 (0.8 g, 1.5 mmol), and t-Bu₃P (0.6 g, 3.0 mmol) were added with Toluene 150 mL and reacted while stirring at 70° C. for 4 hours. After completion of the reaction, the reaction mixture was extracted, concentrated, and then column and recrystallized to obtain 11.8 g of Intermediate 284-2 (yield 66.1%).

### (3) Preparation Example 3: Synthesis of Compound 284

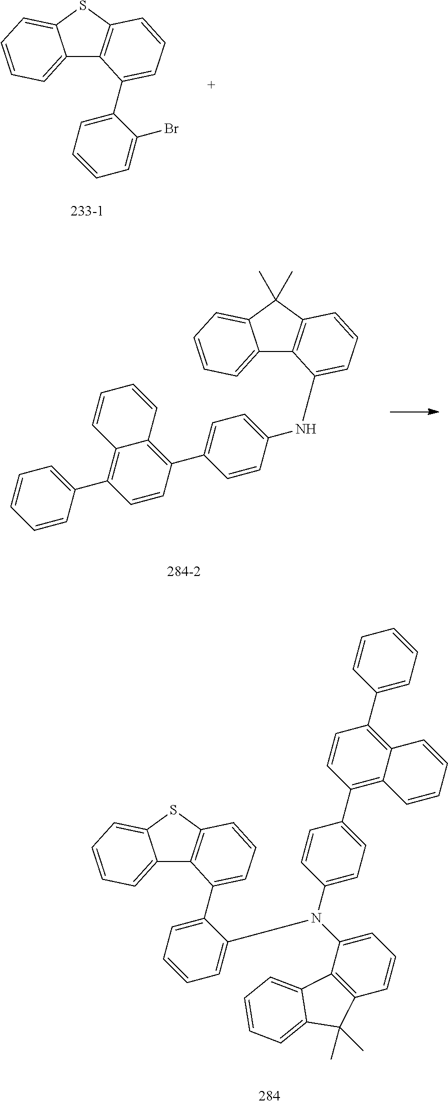 [SMILES: Brc1ccccc1-c1cccc2sc3ccccc3c12 ; CC1(C)c2ccccc2-c2c(Nc3ccc(-c4ccc(-c5ccccc5)c5ccccc45)cc3)cccc21 ; CC1(C)c2ccccc2-c2c(N(c3ccc(-c4ccc(-c5ccccc5)c5ccccc45)cc3)c3ccccc3-c3cccc4sc5ccccc5c34)cccc21]

Intermediate 233-1 (10.0 g, 0.030 mol), Intermediate 284-2 (21.6 g, 0.045 mol), NaOtBu (8.5 g, 0.090 mol), Pd (dba) 2 (0.7 g, 1.2 mmol), and t-Bu₃P (0.5 g, 2.4 mmol) were added with Toluene 150 mL and reacted while stirring at 70° C. for 4 hours. After completion of the reaction, the reaction mixture was extracted, concentrated, and then column and recrystallized to obtain 16.5 g of Compound 284 (yield 75.0%). LC/MS: m/z=745 [(M)⁺]

### Synthesis Example 17: Synthesis of Compound 323

### (1) Preparation Example 1: Synthesis of Intermediate 323-1

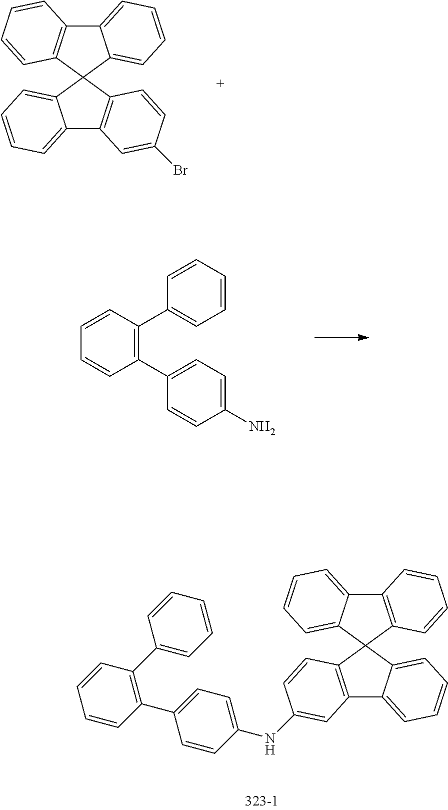 [SMILES: Brc1ccc2c(c1)-c1ccccc1C21c2ccccc2-c2ccccc21 ; Nc1ccc(-c2ccccc2-c2ccccc2)cc1 ; c1ccc(-c2ccccc2-c2ccc(Nc3ccc4c(c3)-c3ccccc3C43c4ccccc4-c4ccccc43)cc2)cc1]

3-Bromo-9,9′-spirobi[9H-fluorene] (10.0 g, 0.034 mol), 4-(2-phenylphenyl) aniline (16.7 g, 0.051 mol), NaOtBu (9.7 g, 0.102 mol), Pd (dba) 2 (0.8 g, 1.4 mmol), and t-Bu₃P (0.6 g, 2.8 mmol) were added with Toluene 150 mL and reacted while stirring at 70° C. for 4 hours. After completion of the reaction, the reaction mixture was extracted, concentrated, and then column and recrystallized to obtain 11.1 g of Intermediate 323-1 (yield 60.2%).

### (2) Preparation Example 2: Synthesis of Compound 323

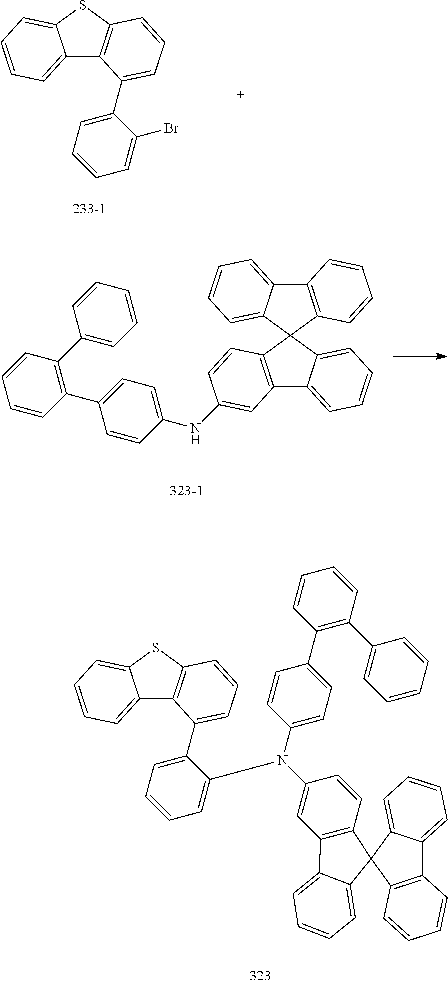 [SMILES: Brc1ccccc1-c1cccc2sc3ccccc3c12 ; c1ccc(-c2ccccc2-c2ccc(Nc3ccc4c(c3)-c3ccccc3C43c4ccccc4-c4ccccc43)cc2)cc1 ; c1ccc(-c2ccccc2-c2ccc(N(c3ccc4c(c3)-c3ccccc3C43c4ccccc4-c4ccccc43)c3ccccc3-c3cccc4sc5ccccc5c34)cc2)cc1]

Intermediate 233-1 (10.0 g, 0.030 mol), Intermediate 323-1 (24.2 g, 0.045 mol), NaOtBu (8.5 g, 0.090 mol), Pd (dba) 2 (0.7 g, 1.2 mol), and t-Bu₃P (0.5 g, 2.4 mmol) were added with Toluene 150 mL and reacted while stirring at 70° C. for 4 hours. After completion of the reaction, the reaction mixture was extracted, concentrated, and then column and recrystallized to obtain 16.8 g of Compound 323 (yield 70.7%).

LC/MS: m/z=805 [(M)⁺]

### Synthesis Example 18: Synthesis of Compound 342

### (1) Preparation Example 1: Synthesis of Intermediate 342-1

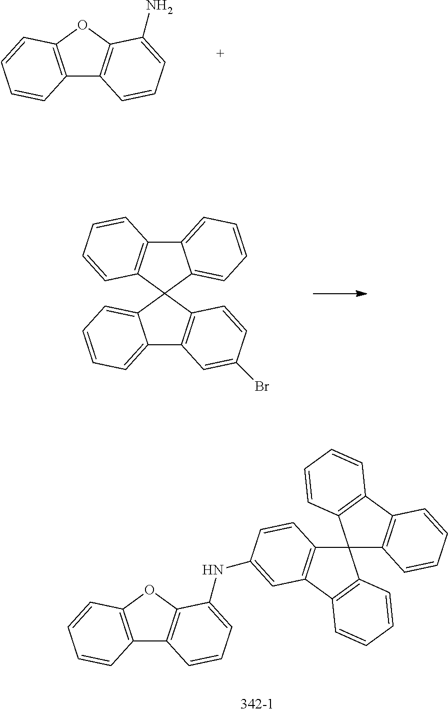 [SMILES: Nc1cccc2c1oc1ccccc12 ; Brc1ccc2c(c1)-c1ccccc1C21c2ccccc2-c2ccccc21 ; c1ccc2c(c1)-c1ccccc1C21c2ccccc2-c2cc(Nc3cccc4c3oc3ccccc34)ccc21]

Dibenzo[b,d]furan-4-amine (10.0 g, 0.055 mol), 3-Bromo-9,9′-spirobi[9H-fluorene] (32.4 g, 0.082 mol), NaOtBu (15.7 g, 0.164 mol), Pd (dba) 2 (1.3 g, 2.2 mol), and t-Bu₃P (0.9 g, 4.4 mmol) were added with Toluene 150 mL and reacted while stirring at 70° C. for 4 hours. After completion of the reaction, the reaction mixture was extracted, concentrated, and then column and recrystallized to obtain 14.1 g of Intermediate 342-1 (yield 51.9%).

### (2) Preparation Example 2: Synthesis of Compound 342

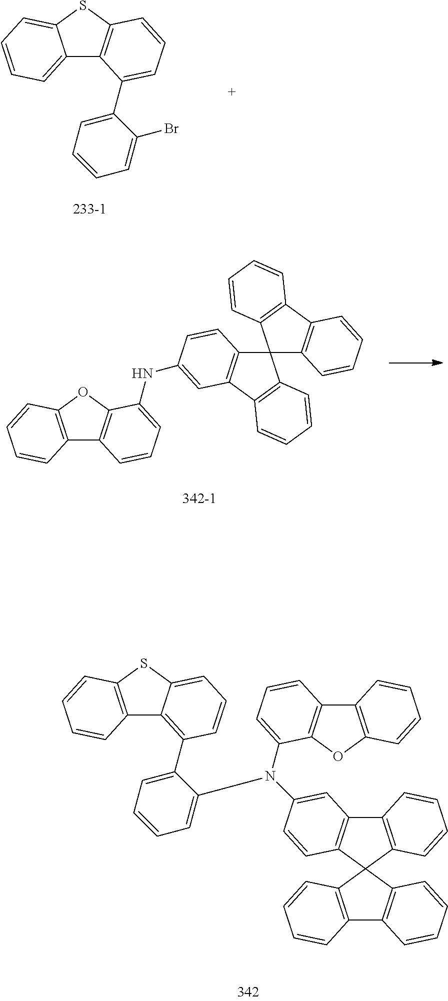 [SMILES: Brc1ccccc1-c1cccc2sc3ccccc3c12 ; c1ccc2c(c1)-c1ccccc1C21c2ccccc2-c2cc(Nc3cccc4c3oc3ccccc34)ccc21 ; c1ccc(N(c2ccc3c(c2)-c2ccccc2C32c3ccccc3-c3ccccc32)c2cccc3c2oc2ccccc23)c(-c2cccc3sc4ccccc4c23)c1]

Intermediate 233-1 (10.0 g, 0.030 mol), Intermediate 342-1 (22.0 g, 0.044 mol), NaOtBu (8.5 g, 0.088 mol), Pd (dba) 2 (0.7 g, 1.2 mol), and t-Bu₃P (0.5 g, 2.4 mmol) were added with Toluene 150 mL and reacted while stirring at 70° C. for 4 hours. After completion of the reaction, the reaction mixture was extracted, concentrated, and then column and recrystallized to obtain 14.8 g of Compound 342 (yield 66.4%). LC/MS: m/z=755 [(M)⁺]

### Synthesis Example 19: Synthesis of Compound 353

### (1) Preparation Example 1: Synthesis of Intermediate 353-1

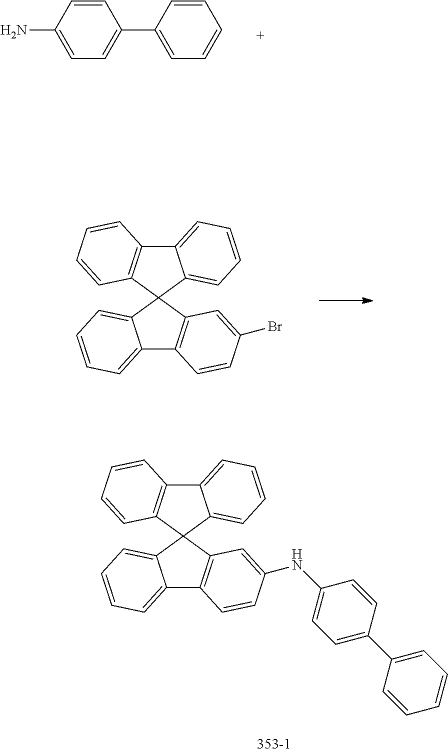 [SMILES: Nc1ccc(-c2ccccc2)cc1 ; Brc1ccc2c(c1)C1(c3ccccc3-c3ccccc31)c1ccccc1-2 ; c1ccc(-c2ccc(Nc3ccc4c(c3)C3(c5ccccc5-c5ccccc53)c3ccccc3-4)cc2)cc1]

4-Aminobiphenyl (10.0 g, 0.059 mol), 2-Bromo-9,9′-spirobi[9H-fluorene] (35.0 g, 0.089 mol), NaOtBu (17.0 g, 0.177 mol), Pd (dba) 2 (1.4 g, 2.4 mol), and t-Bu₃P (1.0 g, 4.7 mmol) were added with Toluene 150 mL and reacted while stirring at 70° C. for 4 hours. After completion of the reaction, the reaction mixture was extracted, concentrated, and then columned to obtain 14.2 g of Intermediate 353-1 (yield 49.7%).

### (2) Preparation Example 2: Synthesis of Intermediate 353-2

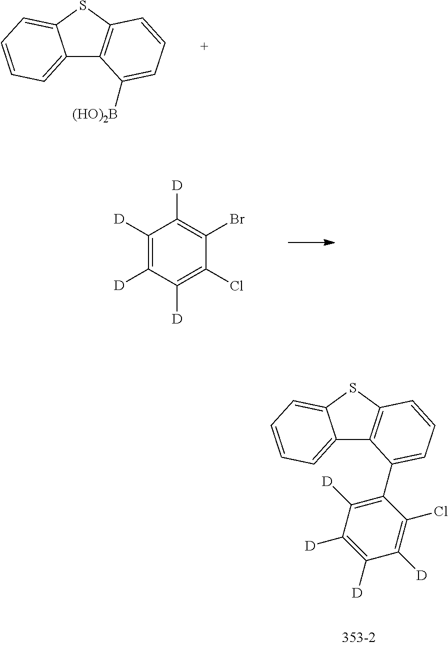 [SMILES: OB(O)c1cccc2sc3ccccc3c12 ; [2H]c1c([2H])c([2H])c(Br)c(Cl)c1[2H] ; [2H]c1c([2H])c([2H])c(-c2cccc3sc4ccccc4c23)c(Cl)c1[2H]]

Dibenzothiophene-1-boronic acid (10.0 g, 0.044 mol), 5-Bromo-6-chlorobenzene-1,2,3,4-d4 (10.3 g, 0.053 mol), K₂CO₃ (18.2 g, 0.132 mol), and Pd(PPh₃)₄ (1.0 g, 0.9 mmol) were added with Toluene 200 mL, EtOH 50 mL, and H₂O 50 mL and reacted while stirring at 100° C. for 6 hours. After completion of the reaction, the reaction mixture was extracted, concentrated, and then column and recrystallized to obtain 8.8 g of Intermediate 353-2 (yield 67.2%).

### (3) Preparation Example 3: Synthesis of Compound 353

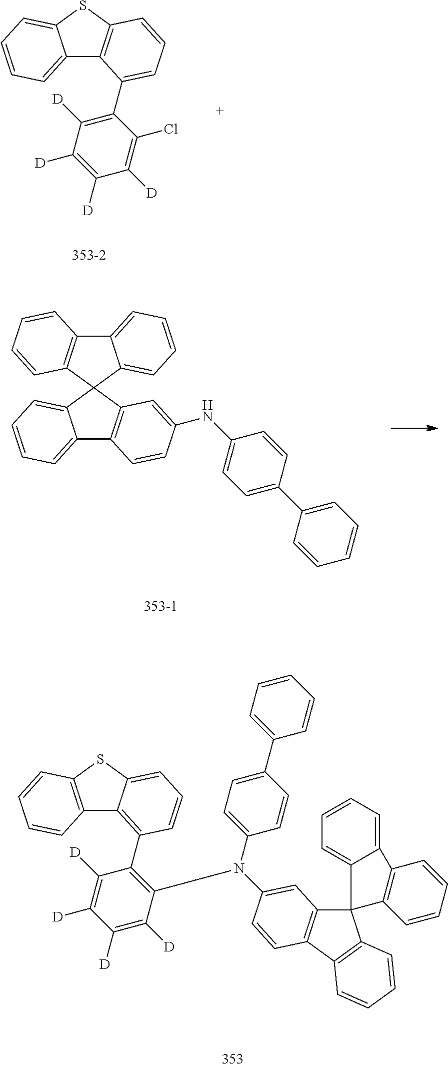 [SMILES: [2H]c1c([2H])c([2H])c(-c2cccc3sc4ccccc4c23)c(Cl)c1[2H] ; c1ccc(-c2ccc(Nc3ccc4c(c3)C3(c5ccccc5-c5ccccc53)c3ccccc3-4)cc2)cc1 ; [2H]c1c([2H])c([2H])c(N(c2ccc(-c3ccccc3)cc2)c2ccc3c(c2)C2(c4ccccc4-c4ccccc42)c2ccccc2-3)c(-c2cccc3sc4ccccc4c23)c1[2H]]

Intermediate 353-1 (10.0 g, 0.034 mol), Intermediate 353-1 (24.3 g, 0.050 mol), NaOtBu (9.7 g, 0.100 mol), Pd (dba) 2 (0.8 g, 1.3 mmol), and t-Bu₃P (0.5 g, 2.7 mmol) were added with Toluene 150 mL and reacted while stirring at 70° C. for 4 hours. After completion of the reaction, the reaction mixture was extracted, concentrated, and then column and recrystallized to obtain 15.5 g of Compound 353 (yield 62.1%).

LC/MS: m/z=745 [(M)⁺]

### Device Example (HTL)

In Example according to the present invention, an ITO transparent electrode was patterned on a glass substrate having dimensions of 25 mm×25 mm×0.7 mm using an ITO glass substrate attached with the ITO transparent electrode to have a light-emitting area of 2 mm×2 mm, followed by cleaning. The substrate was mounted in a vacuum chamber and then organic materials and metals were deposited on the ITO in the following structure after a base pressure was set to 1×10⁻⁶ torr.

### Device Examples 1 to 30

The compound implemented according to the present invention was employed to the hole transport layer to fabricate an organic light-emitting device having the following device structure, and then the luminescent and driving properties of the compound implemented according to the present invention were measured.

ITO/hole injecting layer (HAT-CN, 5 nm)/hole transport layer (100 nm)/electron blocking layer (EB1, 10 nm)/light-emitting layer (20 nm)/electron transport layer (ET1:Liq, 30 nm)/LiF (1 nm)/Al (100 nm)

On the ITO transparent electrode, HAT-CN was formed into a 5 nm-thick film to form a hole injecting layer and then the compounds according to the present invention shown in Table 1 below was formed into a 100 nm-thick film to form a hole transport layer. Thereafter, EB1 was formed into a 10 nm-thick film to form an electron blocking layer and a light-emitting layer was formed by co-depositing BH1 as a host compound and BD1 as a dopant compound to a thickness of 20 nm. Thereafter, a 30 nm-thick electron transport layer (doped with Liq 50% of the following Compound ET1) was deposited, and then LiF was formed into a 1 nm-thick film to form an electron injecting layer. Thereafter, AI was formed into a 100 nm-thick film to fabricate an organic light-emitting device.

### Device Comparative Example 1

An organic light-emitting device for Device Comparative Example 1 was fabricated in the same method, except for using α-NPB instead of the compound according to the present invention in the hole transport layers in the device structures of Examples 1 to 30 above.

### Device Comparative Example 2

An organic light-emitting device for Device Comparative Example 2 was fabricated in the same method, except for using HT1 instead of the compound according to the present invention in the hole transport layers in the device structures of Examples 1 to 30 above.

### Experiment Example 1: Luminescent Properties of Device Examples 1 to 30

The driving voltages, current efficiencies, and color coordinates of the organic light-emitting devices fabricated in Examples and Comparative Examples above were measured using a source meter (Model 237, Keithley) and a spectroradiometer (PR-650, Photo Research). The result values at 1,000 nits were shown in Table 1 below.

TABLE 1

| | Hole transport | | | | |
|---|---|---|---|---|---|
| Example | layer | V | cd/A | CIEx | CIEy |
| 1 | Formula 2 | 3.74 | 7.94 | 0.1317 | 0.1296 |
| 2 | Formula 3 | 3.58 | 7.38 | 0.1363 | 0.1294 |
| 3 | Formula 6 | 3.85 | 7.94 | 0.1303 | 0.1348 |
| 4 | Formula 9 | 3.05 | 7.91 | 0.1309 | 0.1404 |
| 5 | Formula 16 | 3.66 | 8.18 | 0.1333 | 0.1397 |
| 6 | Formula 21 | 3.62 | 7.56 | 0.1341 | 0.1400 |
| 7 | Formula 22 | 4.05 | 7.62 | 0.1315 | 0.1375 |
| 8 | Formula 31 | 3.67 | 7.56 | 0.1341 | 0.1329 |
| 9 | Formula 33 | 3.73 | 8.18 | 0.1285 | 0.1360 |
| 10 | Formula 40 | 4.07 | 7.56 | 0.1311 | 0.1335 |
| 11 | Formula 58 | 4.04 | 7.79 | 0.1386 | 0.1296 |
| 12 | Formula 66 | 3.61 | 7.80 | 0.1341 | 0.1363 |
| 13 | Formula 69 | 3.85 | 7.78 | 0.1307 | 0.1323 |
| 14 | Formula 73 | 3.49 | 8.12 | 0.1330 | 0.1341 |
| 15 | Formula 83 | 3.69 | 7.52 | 0.1316 | 0.1382 |
| 16 | Formula 86 | 3.75 | 7.48 | 0.1320 | 0.1400 |
| 17 | Formula 91 | 4.04 | 7.81 | 0.1330 | 0.1341 |
| 18 | Formula 102 | 3.67 | 7.64 | 0.1325 | 0.1311 |
| 19 | Formula 105 | 3.79 | 7.93 | 0.1296 | 0.1396 |
| 20 | Formula 108 | 3.77 | 8.18 | 0.1307 | 0.1311 |
| 21 | Formula 112 | 4.05 | 7.76 | 0.1315 | 0.1382 |
| 22 | Formula 113 | 3.98 | 7.56 | 0.1325 | 0.1369 |
| 23 | Formula 133 | 3.58 | 7.78 | 0.1320 | 0.1294 |
| 24 | Formula 135 | 3.96 | 7.30 | 0.1303 | 0.1341 |
| 25 | Formula 139 | 3.72 | 7.61 | 0.1341 | 0.1360 |
| 26 | Formula 141 | 3.48 | 8.10 | 0.1315 | 0.1307 |
| 27 | Formula 211 | 3.44 | 7.67 | 0.1315 | 0.1360 |
| 28 | Formula 243 | 3.89 | 7.76 | 0.1333 | 0.1323 |
| 29 | Formula 274 | 4.08 | 7.94 | 0.1288 | 0.1325 |
| 30 | Formula 288 | 3.63 | 7.62 | 0.1294 | 0.1376 |
| Comparative | α-NPB | 4.67 | 6.65 | 0.1353 | 0.1517 |
| Example 1 | | | | | |
| Comparative | HT1 | 5.09 | 7.02 | 0.1312 | 0.1422 |
| Example 2 | | | | | |

As described in the results in Table 1, it was confirmed that the organic light-emitting device in which the compound according to the present invention was employed to the hole transport layer in the device had reduced driving voltage and improved current efficiency compared to a conventional device using a compound used as a hole transport material (Comparative Example 1) and a conventional device using a compound contrasted with the characteristic structure of the compound according to the present invention (Comparative Example 2).

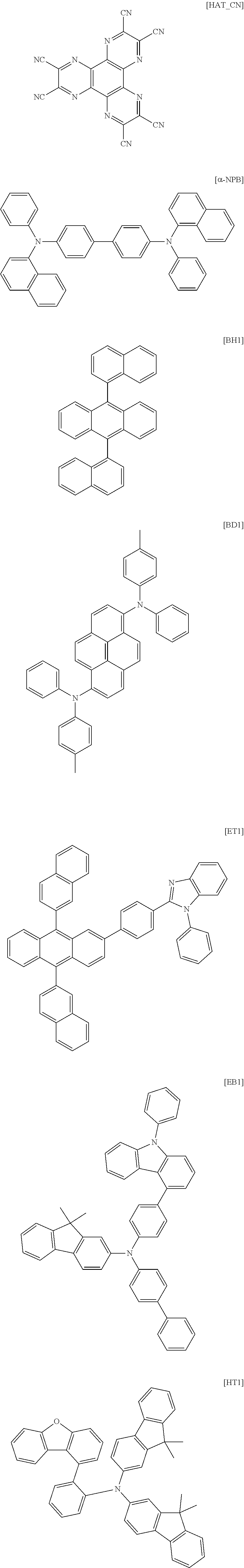

### Device Example (EBL)

In Example according to the present invention, an ITO transparent electrode was patterned on a glass substrate having dimensions of 25 mm×25 mm×0.7 mm using an ITO glass substrate attached with the ITO transparent electrode to have a light-emitting area of 2 mm×2 mm, followed by cleaning. The substrate was mounted in a vacuum chamber and then organic materials and metals were deposited on the ITO in the following structure after a base pressure was set to 1×10⁻⁶ torr.

### Device Examples 31 to 65

The compound implemented according to the present invention was employed to the electron blocking layer to fabricate an organic light-emitting device having the following device structure, and then the luminescent and driving properties of the compound implemented according to the present invention were measured.

ITO/hole injecting layer (HAT-CN, 5 nm)/hole transport layer (α-NPB, 100 nm)/electron blocking layer (10 nm)/light-emitting layer (20 nm)/electron transport layer (ET1:Liq, 30 nm)/LiF (1 nm)/Al (100 nm)

On the ITO transparent electrode, HAT-CN was formed into a 5 nm-thick film to form a hole injecting layer and then α-NPB was formed into a 100 nm-thick film to form a hole transport layer. Thereafter, the compound according to the present invention shown in Table 1 below was formed into a 10 nm-thick film to form an electron blocking layer and a light-emitting layer was formed by co-depositing BH1 as a host compound and BD1 as a dopant compound to a thickness of 20 nm. Thereafter, a 30 nm-thick electron transport layer (doped with Liq 50% of the following Compound ET1) was deposited, and then LiF was formed into a 1 nm-thick film to form an electron injection layer. Thereafter, AI was formed into a 100 nm-thick film to fabricate an organic light-emitting device.

### Device Comparative Example 3

An organic light-emitting device for Device Comparative Example 3 was fabricated in the same manner, except for using EB1 instead of the compound according to the present invention in the electron blocking layers in the device structures of Examples 31 to 65 above.

### Device Comparative Example 4

An organic light-emitting device for Device Comparative Example 4 was fabricated in the same method, except for using EB2 instead of the compound according to the present invention in the electron blocking layers in the device structures of Examples 31 to 65 above.

### Device Comparative Example 5

An organic light-emitting device for Device Comparative Example 5 was fabricated in the same method, except for using EB3 instead of the compound according to the present invention in the electron blocking layers in the device structures of Examples 31 to 65 above.

### Device Comparative Example 6

An organic light-emitting device for Device Comparative Example 6 was fabricated in the same method, except for using EB4 instead of the compound according to the present invention in the electron blocking layers in the device structures of Examples 31 to 65 above.

### Experiment Example 2: Luminescent Properties of Device Examples 31 to 65

The driving voltages, current efficiencies, and color coordinates of the organic light-emitting devices fabricated in Examples and Comparative Examples above were measured using a source meter (Model 237, Keithley) and a spectroradiometer (PR-650, Photo Research). The result values at 1,000 nits were shown in Table 2 below.

TABLE 2

| | Electron | | | | |
|---|---|---|---|---|---|
| Example | blocking layer | V | cd/A | CIEx | CIEy |
| 31 | Formula 4 | 4.05 | 7.87 | 0.1320 | 0.1335 |
| 32 | Formula 5 | 3.63 | 7.56 | 0.1298 | 0.1375 |
| 33 | Formula 17 | 3.63 | 7.60 | 0.1294 | 0.1339 |
| 34 | Formula 18 | 3.61 | 7.30 | 0.1334 | 0.1320 |
| 35 | Formula 20 | 3.49 | 7.63 | 0.1315 | 0.1341 |
| 36 | Formula 23 | 3.63 | 7.32 | 0.1317 | 0.1313 |
| 37 | Formula 27 | 3.02 | 7.61 | 0.1298 | 0.1348 |
| 38 | Formula 28 | 3.62 | 7.87 | 0.1320 | 0.1399 |
| 39 | Formula 35 | 3.48 | 7.60 | 0.1353 | 0.1323 |
| 40 | Formula 36 | 3.73 | 7.30 | 0.1341 | 0.1404 |
| 41 | Formula 48 | 3.96 | 7.43 | 0.1286 | 0.1369 |
| 42 | Formula 55 | 3.54 | 7.38 | 0.1317 | 0.1375 |
| 43 | Formula 67 | 4.28 | 7.30 | 0.1334 | 0.1311 |
| 44 | Formula 68 | 3.49 | 7.41 | 0.1285 | 0.1320 |
| 45 | Formula 76 | 3.63 | 7.48 | 0.1285 | 0.1379 |
| 46 | Formula 81 | 3.67 | 7.34 | 0.1331 | 0.1360 |
| 47 | Formula 82 | 3.87 | 7.60 | 0.1314 | 0.1335 |
| 48 | Formula 87 | 3.49 | 8.14 | 0.1294 | 0.1382 |
| 49 | Formula 89 | 3.52 | 7.63 | 0.1313 | 0.1408 |
| 50 | Formula 99 | 3.62 | 7.78 | 0.1296 | 0.1360 |
| 51 | Formula 100 | 3.51 | 7.67 | 0.1311 | 0.1279 |
| 52 | Formula 101 | 3.59 | 7.31 | 0.1285 | 0.1404 |
| 53 | Formula 118 | 3.74 | 7.94 | 0.1295 | 0.1341 |
| 54 | Formula 125 | 3.83 | 8.11 | 0.1341 | 0.1339 |
| 55 | Formula 153 | 3.95 | 7.79 | 0.1285 | 0.1408 |
| 56 | Formula 185 | 4.02 | 7.30 | 0.1334 | 0.1341 |
| 57 | Formula 189 | 3.99 | 7.61 | 0.1301 | 0.1353 |
| 58 | Formula 202 | 3.75 | 8.08 | 0.1298 | 0.1321 |
| 59 | Formula 222 | 3.66 | 8.12 | 0.1298 | 0.1320 |
| 60 | Formula 233 | 3.74 | 7.91 | 0.1385 | 0.1307 |
| 61 | Formula 256 | 3.61 | 7.41 | 0.1307 | 0.1296 |
| 62 | Formula 257 | 3.34 | 7.30 | 0.1341 | 0.1400 |
| 63 | Formula 342 | 3.95 | 7.92 | 0.1322 | 0.1302 |
| 64 | Formula 353 | 3.61 | 8.18 | 0.1295 | 0.1310 |
| 65 | Formula 356 | 3.75 | 8.01 | 0.1303 | 0.1321 |
| Comparative | EB1 | 4.67 | 6.65 | 0.1353 | 0.1517 |
| Example 3 | | | | | |
| Comparative | EB2 | 4.82 | 6.68 | 0.1366 | 0.1344 |
| Example 4 | | | | | |
| Comparative | EB3 | 4.65 | 6.54 | 0.1357 | 0.1359 |
| Example 5 | | | | | |
| Comparative | EB4 | 4.71 | 6.52 | 0.1361 | 0.1337 |
| Example 6 | | | | | |

As described in the results shown in Table 2 above, it was confirmed that in the case of the organic light-emitting device in which the compound of the present invention was employed to the electron blocking layer in the device, low-voltage driving properties and luminescent properties such as luminous efficiency, quantum efficiency, etc. were significantly excellent compared to conventional devices (Comparative Examples 3 to 6) employing compounds contrasted with the characteristic structure of the compound according to the present invention as the compound used as the electron blocking layer material.

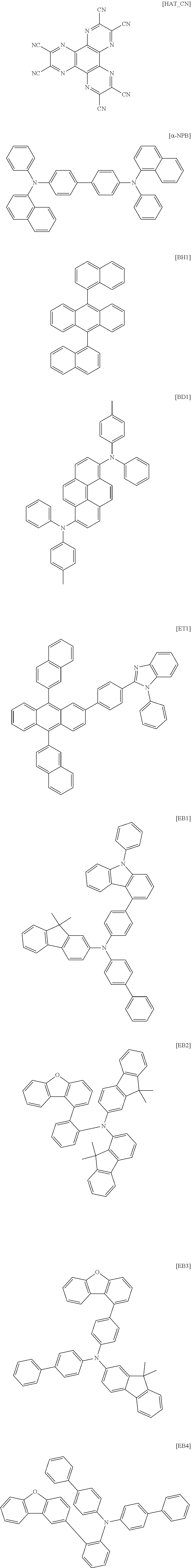

### INDUSTRIAL APPLICABILITY

The present invention can implement an organic compound characterized to be employed as an organic layer material in an organic light-emitting device and an organic light-emitting device with low-voltage driving of the device and significantly improved luminescent properties such as excellent luminous efficiency, etc. by employing the organic compound, and thus the present invention can be effectively used industrially in various displays and lighting devices.

1. A compound represented by Formula I below: 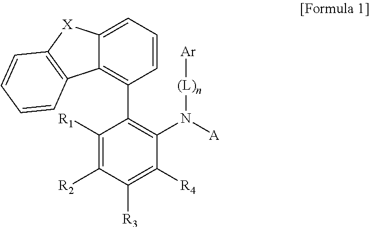
In Formula I above,
X is O or S,
R₁ to R₄ are the same as or different from each other, and are each independently hydrogen or deuterium,
L is a single bond or any one selected from a substituted or unsubstituted arylene group (however, excluding fluorenylene group) having 6 to 30 carbon atoms and a substituted or unsubstituted heteroarylene group having 3 to 30 carbon atoms,
n is an integer of 0 to 2, and when the n is 2, a plurality of L is the same as or different from each other,
Ar is any one selected from a substituted or unsubstituted alkyl group having 1 to 20 carbon atoms, a substituted or unsubstituted cycloalkyl group having 3 to 20 carbon atoms, a substituted or unsubstituted aryl group (however, excluding a fluorenyl group) having 6 to 30 carbon atoms, and a substituted or unsubstituted heteroaryl group having 3 to 30 carbon atoms,
A is represented by Structural Formula 1 or 2 below, 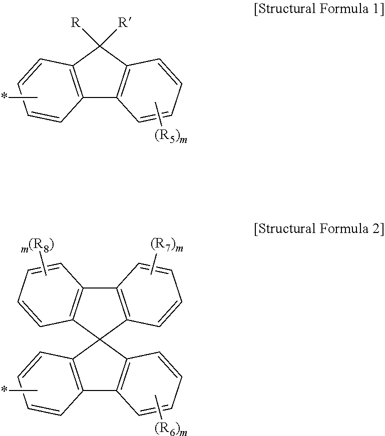
In Structural Formula 1 or Structural Formula 2 above,
R and R′ are each independently alkyl groups having 1 to 7 carbon atoms,
R₅ to R₈ are the same as or different from each other, and are each independently any one selected from hydrogen, deuterium, a cyano group, halogen group, a substituted or unsubstituted alkyl group having 1 to 20 carbon atoms, a substituted or unsubstituted cycloalkyl group having 3 to 20 carbon atoms, a substituted or unsubstituted aryl group having 6 to 30 carbon atoms and a substituted or unsubstituted heteroaryl group having 3 to 30 carbon atoms, and
m is each an integer of 0 to 4, and when each m is 2 or more, a plurality of R₅ to R₈ are the same as or different from each other.

2. The compound of claim 1, wherein in the definitions of L, Ar and R₅ to R₈ above, the ‘substituted or unsubstituted’ means substitution of L, Ar and R₅ to R₈ above with one or at least two substituents selected from the group consisting of deuterium, a halogen group, a cyano group, a nitro group, a hydroxy group, an alkyl group, a halogenated alkyl group, a deuterated alkyl group, a cycloalkyl group, a heterocycloalkyl group, an alkoxy group, a halogenated alkoxy group, a deuterated alkoxy group, an amine group, an aryl group, a heteroaryl group, an alkylsilyl group and an arylsilyl group, substitution with a substituent to which two or more of the substituents are linked, or having no substituent.

3. The compound of claim 1, wherein Formula 1 above is any one selected from Compounds 1 to 368 below:

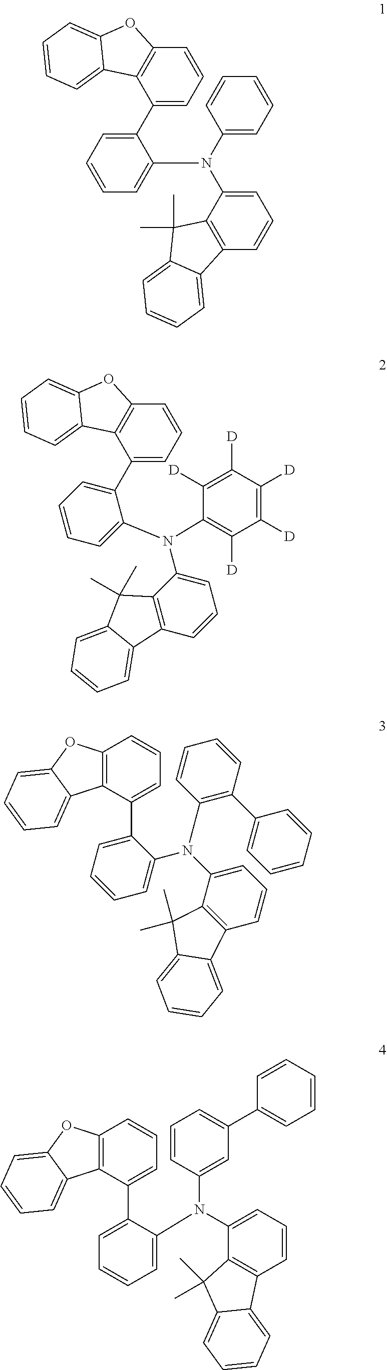 [SMILES: CC1(C)c2ccccc2-c2cccc(N(c3ccccc3)c3ccccc3-c3cccc4oc5ccccc5c34)c21 ; [2H]c1c([2H])c([2H])c(N(c2ccccc2-c2cccc3oc4ccccc4c23)c2cccc3c2C(C)(C)c2ccccc2-3)c([2H])c1[2H] ; CC1(C)c2ccccc2-c2cccc(N(c3ccccc3-c3ccccc3)c3ccccc3-c3cccc4oc5ccccc5c34)c21 ; CC1(C)c2ccccc2-c2cccc(N(c3cccc(-c4ccccc4)c3)c3ccccc3-c3cccc4oc5ccccc5c34)c21]

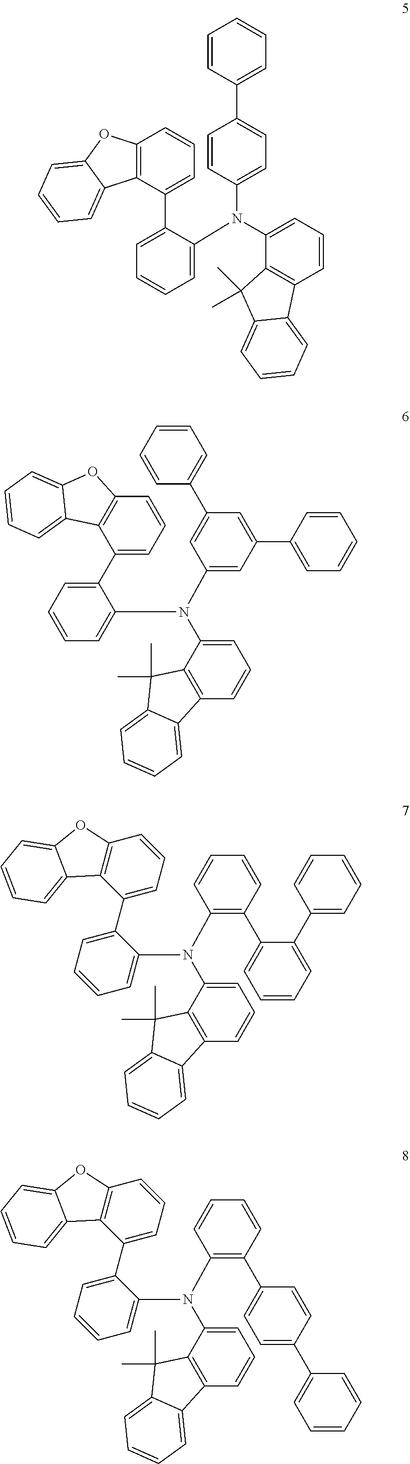 [SMILES: CC1(C)c2ccccc2-c2cccc(N(c3ccc(-c4ccccc4)cc3)c3ccccc3-c3cccc4oc5ccccc5c34)c21 ; CC1(C)c2ccccc2-c2cccc(N(c3cc(-c4ccccc4)cc(-c4ccccc4)c3)c3ccccc3-c3cccc4oc5ccccc5c34)c21 ; CC1(C)c2ccccc2-c2cccc(N(c3ccccc3-c3ccccc3-c3ccccc3)c3ccccc3-c3cccc4oc5ccccc5c34)c21 ; CC1(C)c2ccccc2-c2cccc(N(c3ccccc3-c3ccc(-c4ccccc4)cc3)c3ccccc3-c3cccc4oc5ccccc5c34)c21]

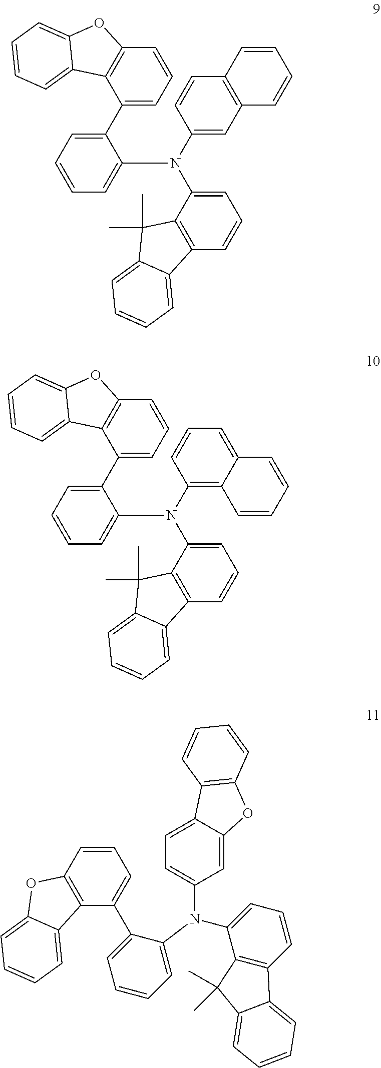 [SMILES: CC1(C)c2ccccc2-c2cccc(N(c3ccc4ccccc4c3)c3ccccc3-c3cccc4oc5ccccc5c34)c21 ; CC1(C)c2ccccc2-c2cccc(N(c3ccccc3-c3cccc4oc5ccccc5c34)c3cccc4ccccc34)c21 ; CC1(C)c2ccccc2-c2cccc(N(c3ccc4c(c3)oc3ccccc34)c3ccccc3-c3cccc4oc5ccccc5c34)c21]

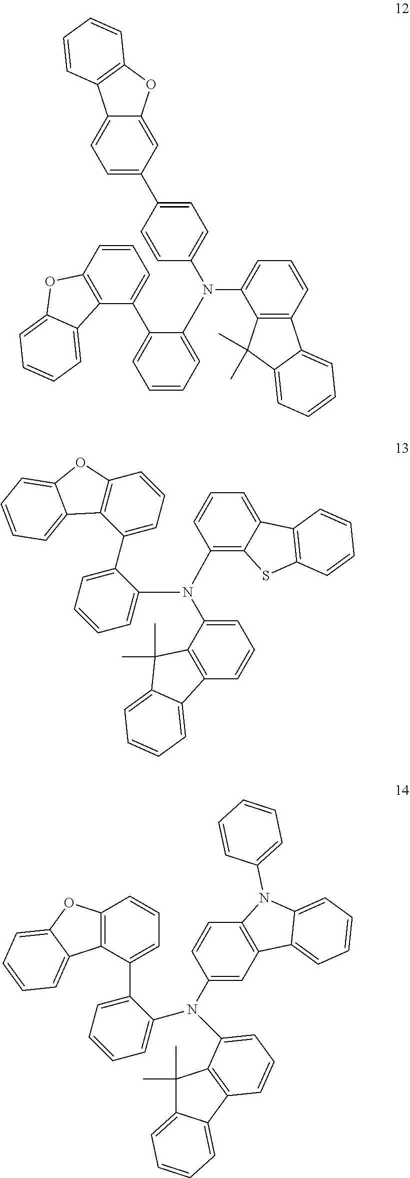 [SMILES: CC1(C)c2ccccc2-c2cccc(N(c3ccc(-c4ccc5c(c4)oc4ccccc45)cc3)c3ccccc3-c3cccc4oc5ccccc5c34)c21 ; CC1(C)c2ccccc2-c2cccc(N(c3ccccc3-c3cccc4oc5ccccc5c34)c3cccc4c3sc3ccccc34)c21 ; CC1(C)c2ccccc2-c2cccc(N(c3ccc4c(c3)c3ccccc3n4-c3ccccc3)c3ccccc3-c3cccc4oc5ccccc5c34)c21]

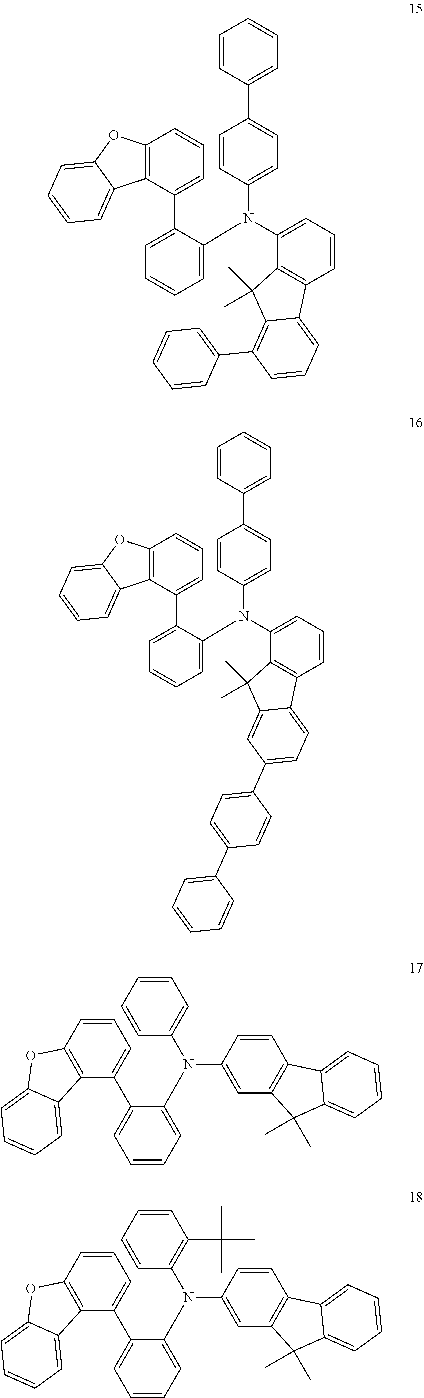 [SMILES: CC1(C)c2c(-c3ccccc3)cccc2-c2cccc(N(c3ccc(-c4ccccc4)cc3)c3ccccc3-c3cccc4oc5ccccc5c34)c21 ; CC1(C)c2cc(-c3ccc(-c4ccccc4)cc3)ccc2-c2cccc(N(c3ccc(-c4ccccc4)cc3)c3ccccc3-c3cccc4oc5ccccc5c34)c21 ; CC1(C)c2ccccc2-c2ccc(N(c3ccccc3)c3ccccc3-c3cccc4oc5ccccc5c34)cc21 ; CC(C)(C)c1ccccc1N(c1ccc2c(c1)C(C)(C)c1ccccc1-2)c1ccccc1-c1cccc2oc3ccccc3c12]

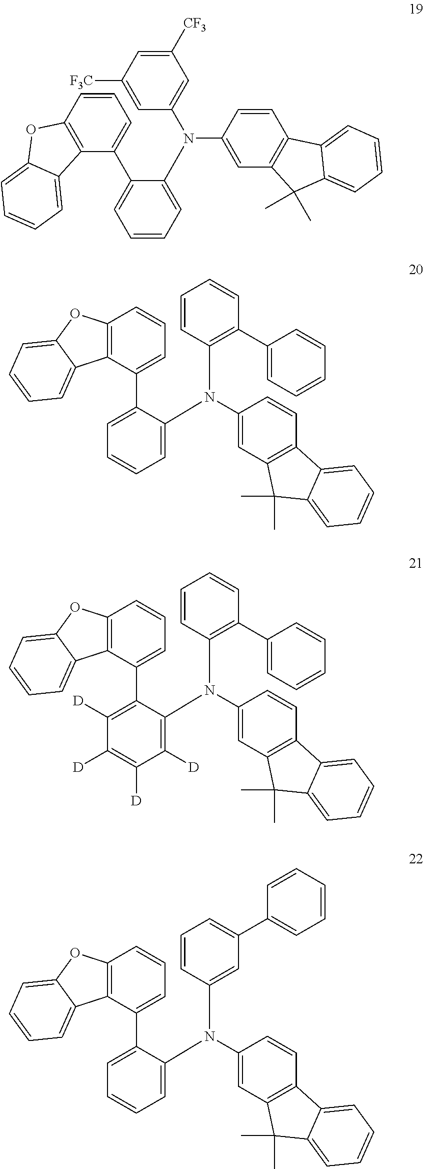 [SMILES: CC1(C)c2ccccc2-c2ccc(N(c3cc(C(F)(F)F)cc(C(F)(F)F)c3)c3ccccc3-c3cccc4oc5ccccc5c34)cc21 ; CC1(C)c2ccccc2-c2ccc(N(c3ccccc3-c3ccccc3)c3ccccc3-c3cccc4oc5ccccc5c34)cc21 ; [2H]c1c([2H])c([2H])c(N(c2ccc3c(c2)C(C)(C)c2ccccc2-3)c2ccccc2-c2ccccc2)c(-c2cccc3oc4ccccc4c23)c1[2H] ; CC1(C)c2ccccc2-c2ccc(N(c3cccc(-c4ccccc4)c3)c3ccccc3-c3cccc4oc5ccccc5c34)cc21]

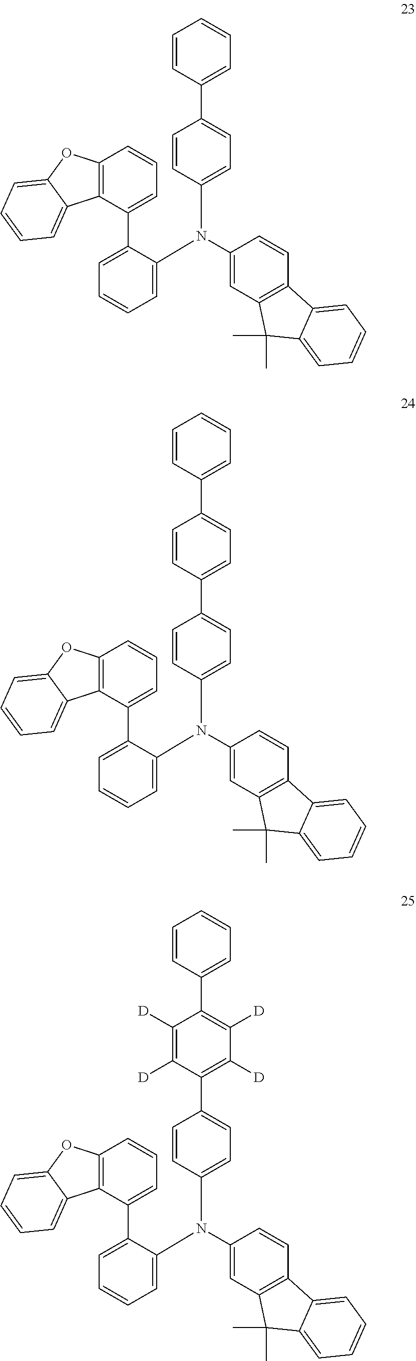 [SMILES: CC1(C)c2ccccc2-c2ccc(N(c3ccc(-c4ccccc4)cc3)c3ccccc3-c3cccc4oc5ccccc5c34)cc21 ; CC1(C)c2ccccc2-c2ccc(N(c3ccc(-c4ccc(-c5ccccc5)cc4)cc3)c3ccccc3-c3cccc4oc5ccccc5c34)cc21 ; [2H]c1c([2H])c(-c2ccc(N(c3ccc4c(c3)C(C)(C)c3ccccc3-4)c3ccccc3-c3cccc4oc5ccccc5c34)cc2)c([2H])c([2H])c1-c1ccccc1]

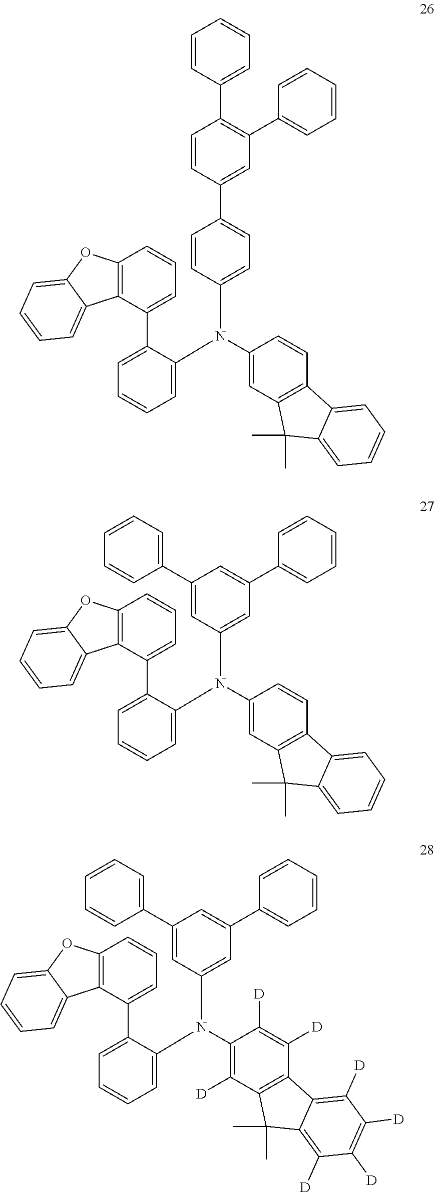

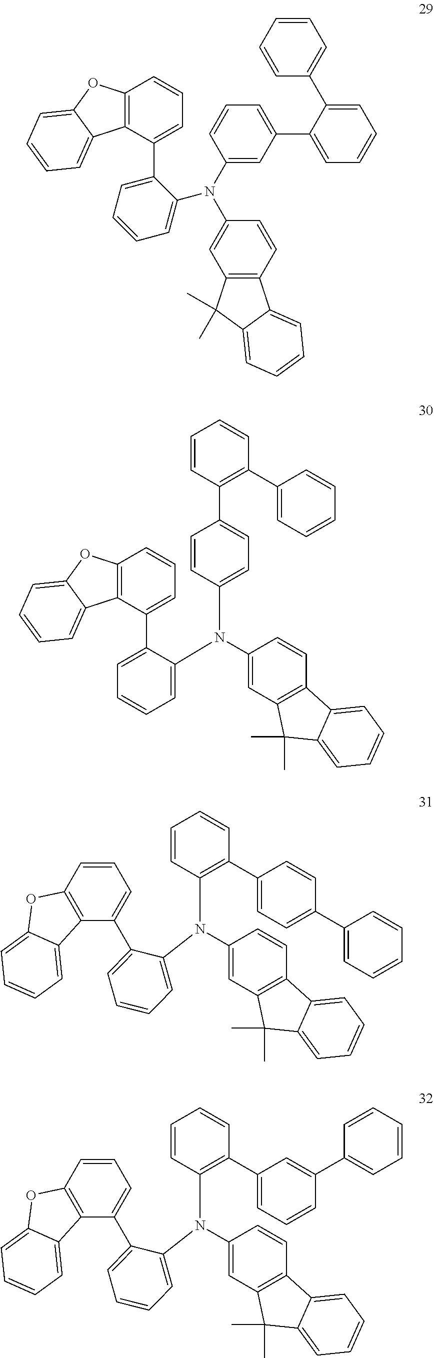 [SMILES: CC1(C)c2ccccc2-c2ccc(N(c3cccc(-c4ccccc4-c4ccccc4)c3)c3ccccc3-c3cccc4oc5ccccc5c34)cc21 ; CC1(C)c2ccccc2-c2ccc(N(c3ccc(-c4ccccc4-c4ccccc4)cc3)c3ccccc3-c3cccc4oc5ccccc5c34)cc21 ; CC1(C)c2ccccc2-c2ccc(N(c3ccccc3-c3ccc(-c4ccccc4)cc3)c3ccccc3-c3cccc4oc5ccccc5c34)cc21 ; CC1(C)c2ccccc2-c2ccc(N(c3ccccc3-c3cccc(-c4ccccc4)c3)c3ccccc3-c3cccc4oc5ccccc5c34)cc21]

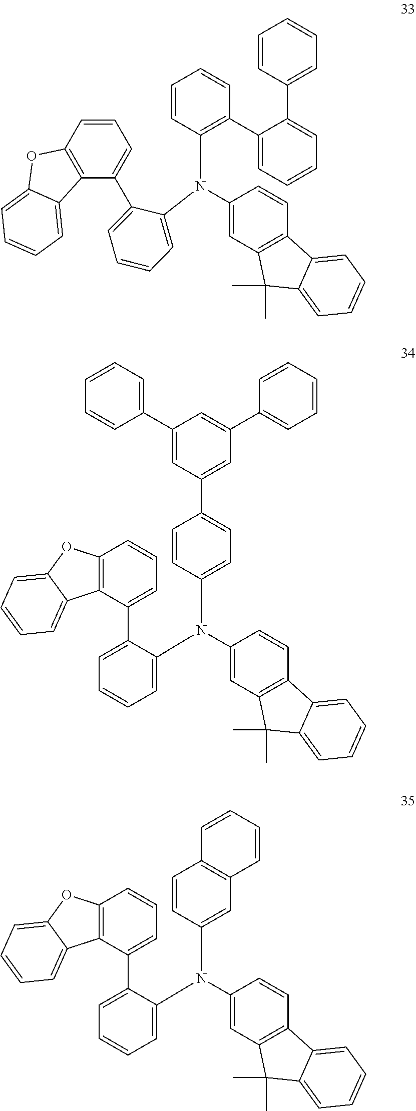 [SMILES: CC1(C)c2ccccc2-c2ccc(N(c3ccccc3-c3ccccc3-c3ccccc3)c3ccccc3-c3cccc4oc5ccccc5c34)cc21 ; CC1(C)c2ccccc2-c2ccc(N(c3ccc(-c4cc(-c5ccccc5)cc(-c5ccccc5)c4)cc3)c3ccccc3-c3cccc4oc5ccccc5c34)cc21 ; CC1(C)c2ccccc2-c2ccc(N(c3ccc4ccccc4c3)c3ccccc3-c3cccc4oc5ccccc5c34)cc21]

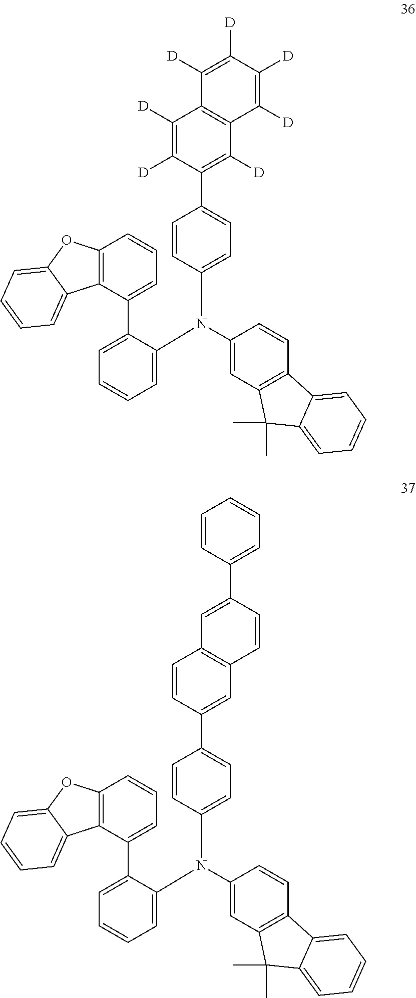 [SMILES: [2H]c1c([2H])c([2H])c2c([2H])c(-c3ccc(N(c4ccc5c(c4)C(C)(C)c4ccccc4-5)c4ccccc4-c4cccc5oc6ccccc6c45)cc3)c([2H])c([2H])c2c1[2H] ; CC1(C)c2ccccc2-c2ccc(N(c3ccc(-c4ccc5cc(-c6ccccc6)ccc5c4)cc3)c3ccccc3-c3cccc4oc5ccccc5c34)cc21]

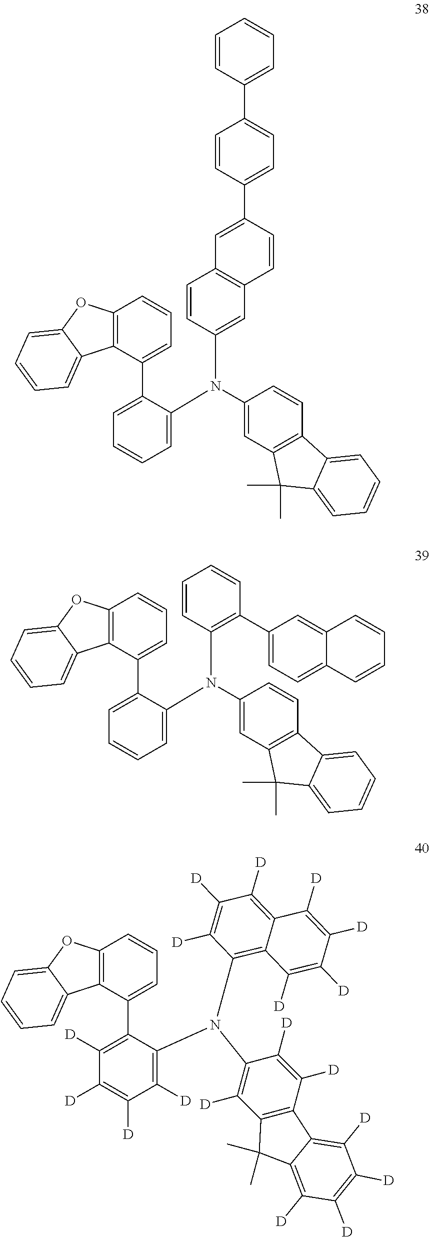 [SMILES: CC1(C)c2ccccc2-c2ccc(N(c3ccc4cc(-c5ccc(-c6ccccc6)cc5)ccc4c3)c3ccccc3-c3cccc4oc5ccccc5c34)cc21 ; CC1(C)c2ccccc2-c2ccc(N(c3ccccc3-c3ccc4ccccc4c3)c3ccccc3-c3cccc4oc5ccccc5c34)cc21 ; [2H]c1c([2H])c([2H])c(N(c2c([2H])c([2H])c3c(c2[2H])C(C)(C)c2c([2H])c([2H])c([2H])c([2H])c2-3)c2c([2H])c([2H])c([2H])c3c([2H])c([2H])c([2H])c([2H])c23)c(-c2cccc3oc4ccccc4c23)c1[2H]]

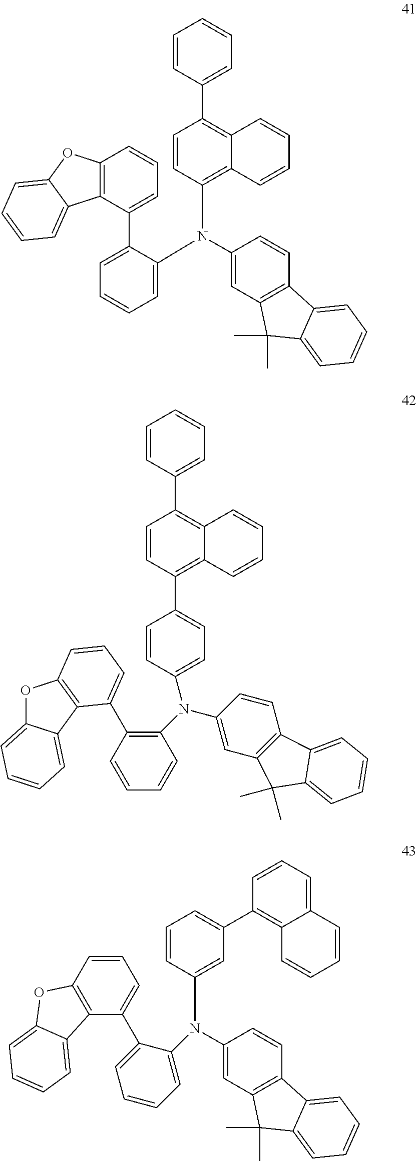

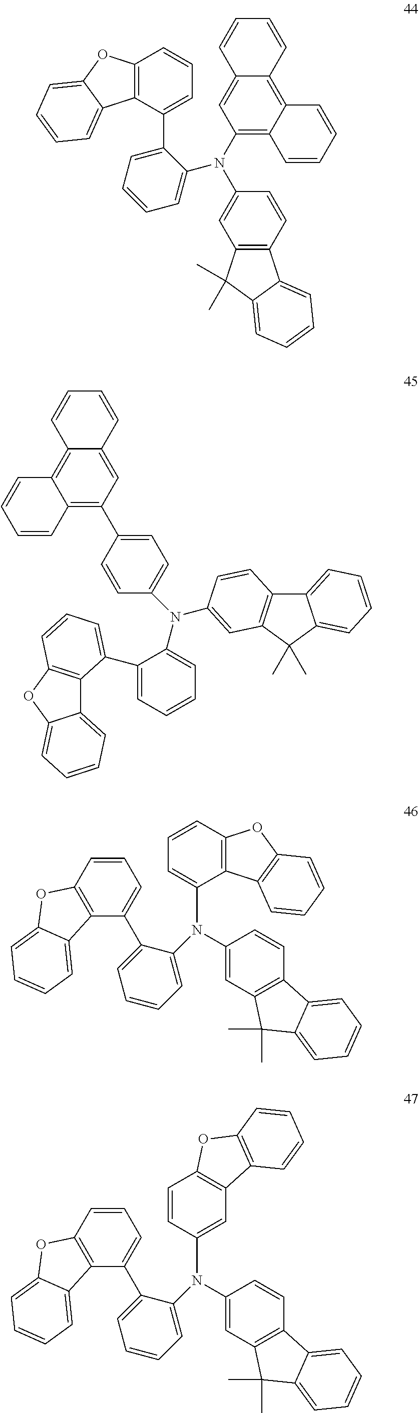

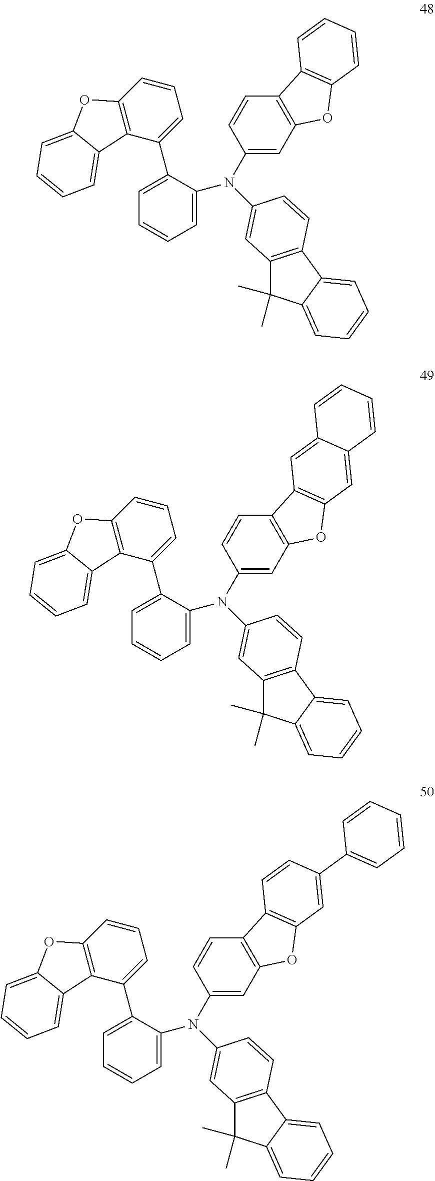 [SMILES: CC1(C)c2ccccc2-c2ccc(N(c3ccc4c(c3)oc3ccccc34)c3ccccc3-c3cccc4oc5ccccc5c34)cc21 ; CC1(C)c2ccccc2-c2ccc(N(c3ccc4c(c3)oc3cc5ccccc5cc34)c3ccccc3-c3cccc4oc5ccccc5c34)cc21 ; CC1(C)c2ccccc2-c2ccc(N(c3ccc4c(c3)oc3cc(-c5ccccc5)ccc34)c3ccccc3-c3cccc4oc5ccccc5c34)cc21]

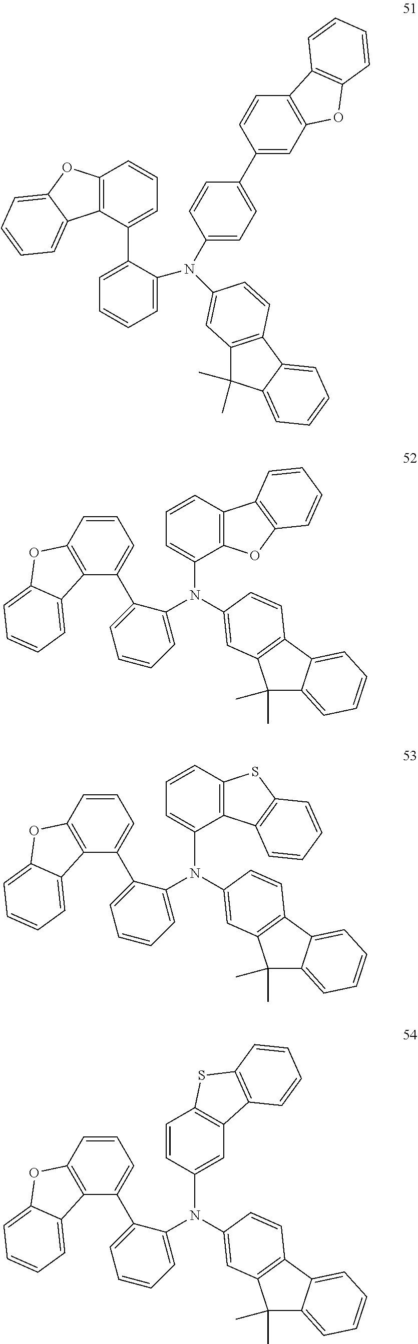

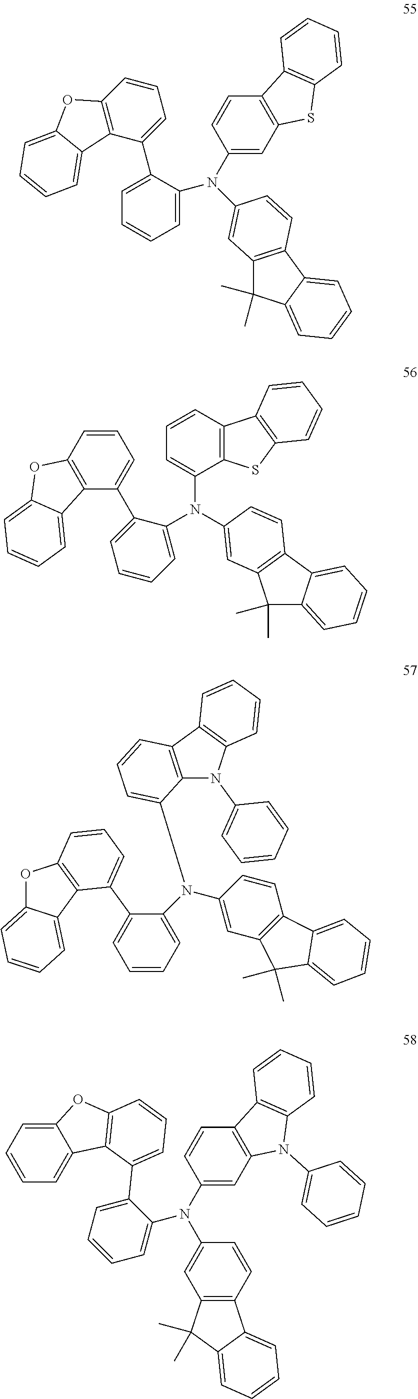 [SMILES: CC1(C)c2ccccc2-c2ccc(N(c3ccc4c(c3)sc3ccccc34)c3ccccc3-c3cccc4oc5ccccc5c34)cc21 ; CC1(C)c2ccccc2-c2ccc(N(c3ccccc3-c3cccc4oc5ccccc5c34)c3cccc4c3sc3ccccc34)cc21 ; CC1(C)c2ccccc2-c2ccc(N(c3ccccc3-c3cccc4oc5ccccc5c34)c3cccc4c5ccccc5n(-c5ccccc5)c34)cc21 ; CC1(C)c2ccccc2-c2ccc(N(c3ccc4c5ccccc5n(-c5ccccc5)c4c3)c3ccccc3-c3cccc4oc5ccccc5c34)cc21]

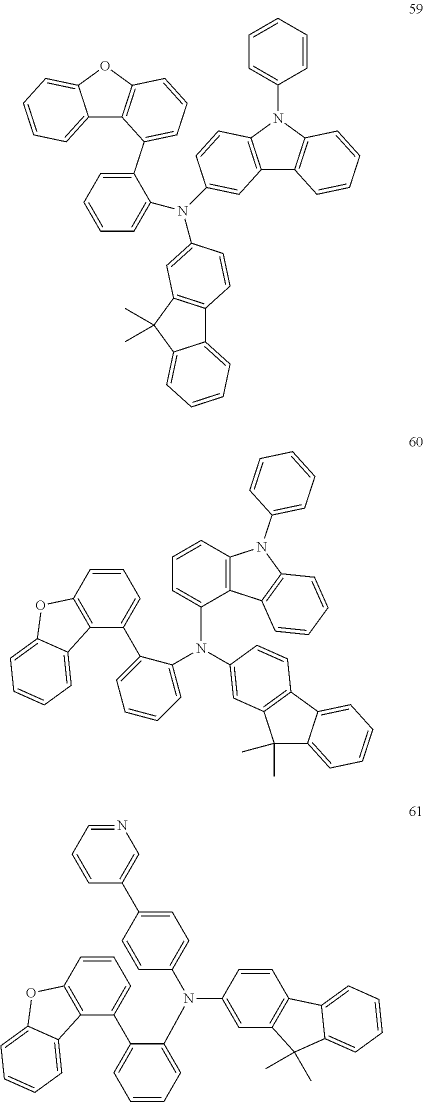 [SMILES: CC1(C)c2ccccc2-c2ccc(N(c3ccc4c(c3)c3ccccc3n4-c3ccccc3)c3ccccc3-c3cccc4oc5ccccc5c34)cc21 ; CC1(C)c2ccccc2-c2ccc(N(c3ccccc3-c3cccc4oc5ccccc5c34)c3cccc4c3c3ccccc3n4-c3ccccc3)cc21 ; CC1(C)c2ccccc2-c2ccc(N(c3ccc(-c4cccnc4)cc3)c3ccccc3-c3cccc4oc5ccccc5c34)cc21]

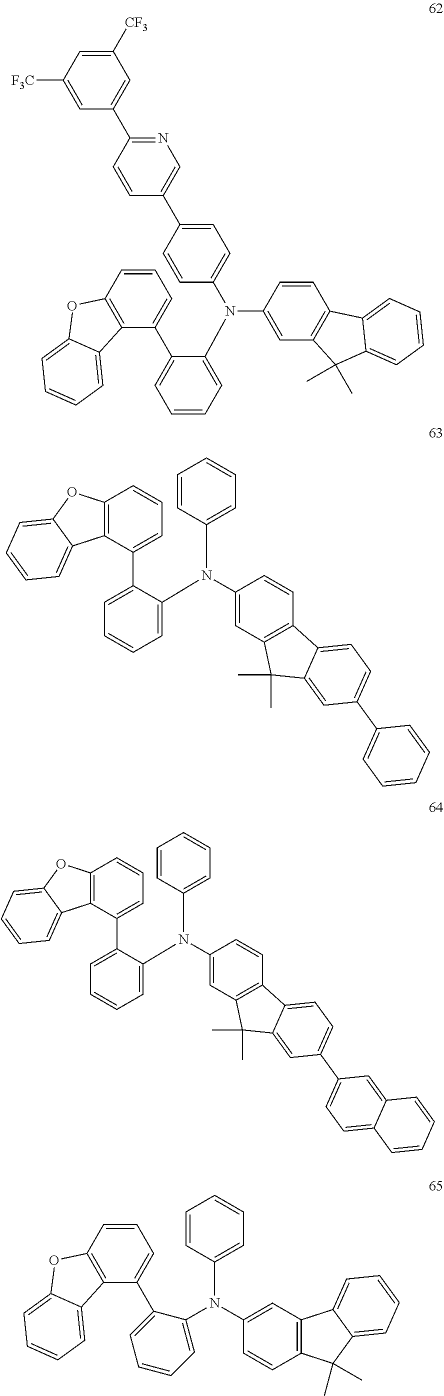

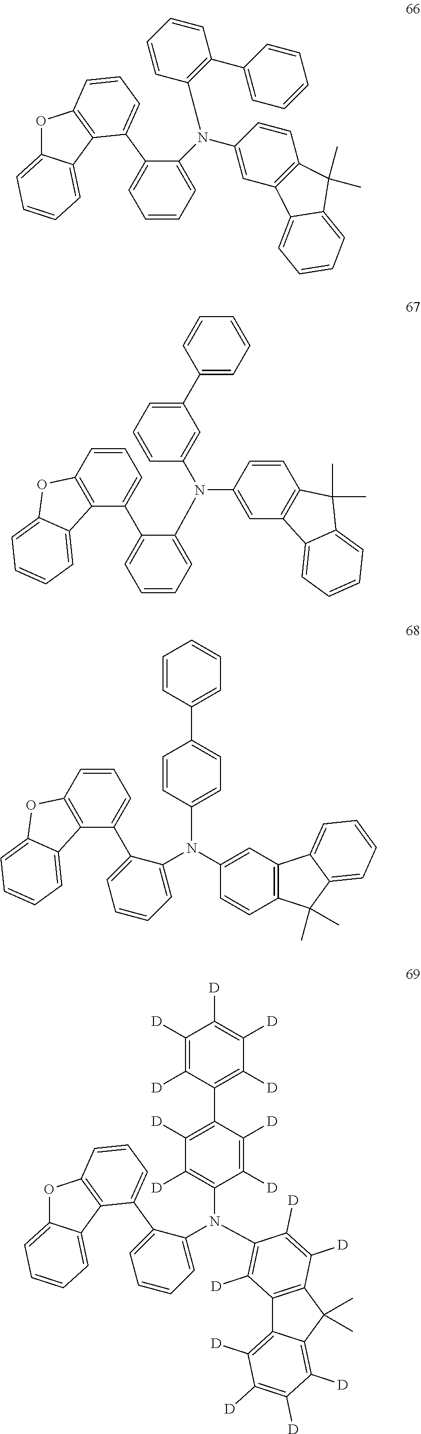 [SMILES: CC1(C)c2ccccc2-c2cc(N(c3ccccc3-c3ccccc3)c3ccccc3-c3cccc4oc5ccccc5c34)ccc21 ; CC1(C)c2ccccc2-c2cc(N(c3cccc(-c4ccccc4)c3)c3ccccc3-c3cccc4oc5ccccc5c34)ccc21 ; CC1(C)c2ccccc2-c2cc(N(c3ccc(-c4ccccc4)cc3)c3ccccc3-c3cccc4oc5ccccc5c34)ccc21 ; [2H]c1c([2H])c([2H])c(-c2c([2H])c([2H])c(N(c3ccccc3-c3cccc4oc5ccccc5c34)c3c([2H])c([2H])c4c(c3[2H])-c3c([2H])c([2H])c([2H])c([2H])c3C4(C)C)c([2H])c2[2H])c([2H])c1[2H]]

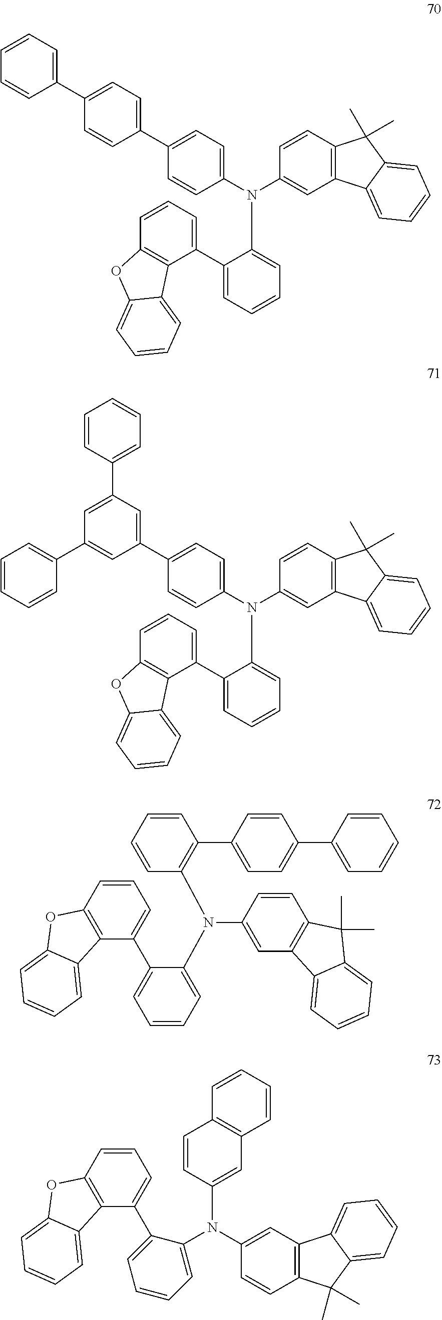 [SMILES: CC1(C)c2ccccc2-c2cc(N(c3ccc(-c4ccc(-c5ccccc5)cc4)cc3)c3ccccc3-c3cccc4oc5ccccc5c34)ccc21 ; CC1(C)c2ccccc2-c2cc(N(c3ccc(-c4cc(-c5ccccc5)cc(-c5ccccc5)c4)cc3)c3ccccc3-c3cccc4oc5ccccc5c34)ccc21 ; CC1(C)c2ccccc2-c2cc(N(c3ccccc3-c3ccc(-c4ccccc4)cc3)c3ccccc3-c3cccc4oc5ccccc5c34)ccc21 ; CC1(C)c2ccccc2-c2cc(N(c3ccc4ccccc4c3)c3ccccc3-c3cccc4oc5ccccc5c34)ccc21]

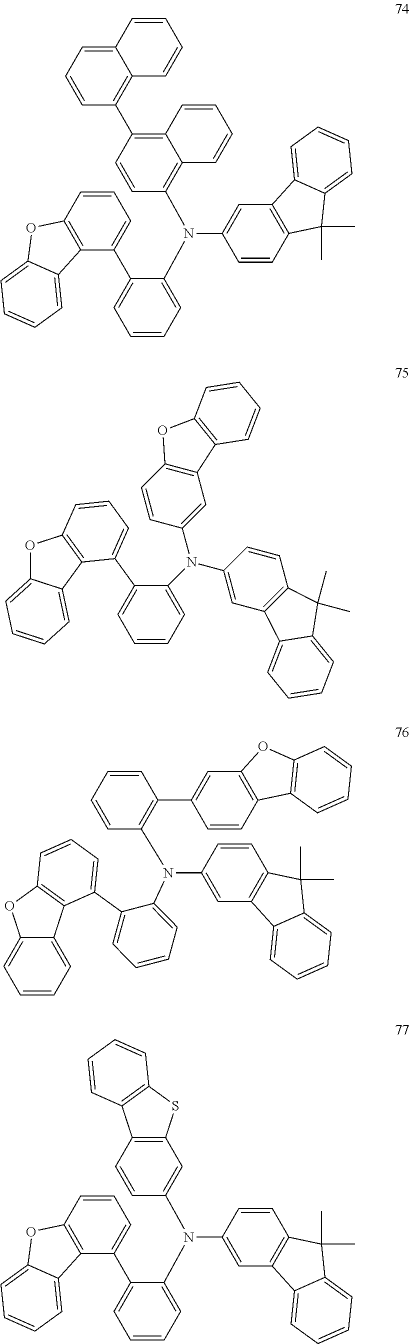 [SMILES: CC1(C)c2ccccc2-c2cc(N(c3ccccc3-c3cccc4oc5ccccc5c34)c3ccc(-c4cccc5ccccc45)c4ccccc34)ccc21 ; CC1(C)c2ccccc2-c2cc(N(c3ccc4oc5ccccc5c4c3)c3ccccc3-c3cccc4oc5ccccc5c34)ccc21 ; CC1(C)c2ccccc2-c2cc(N(c3ccccc3-c3ccc4c(c3)oc3ccccc34)c3ccccc3-c3cccc4oc5ccccc5c34)ccc21 ; CC1(C)c2ccccc2-c2cc(N(c3ccc4c(c3)sc3ccccc34)c3ccccc3-c3cccc4oc5ccccc5c34)ccc21]

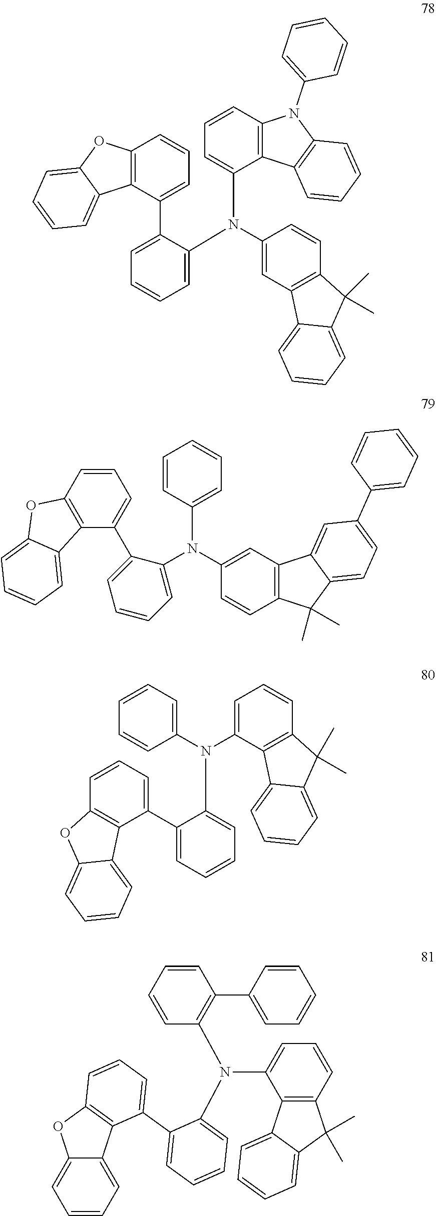 [SMILES: CC1(C)c2ccccc2-c2cc(N(c3ccccc3-c3cccc4oc5ccccc5c34)c3cccc4c3c3ccccc3n4-c3ccccc3)ccc21 ; CC1(C)c2ccc(-c3ccccc3)cc2-c2cc(N(c3ccccc3)c3ccccc3-c3cccc4oc5ccccc5c34)ccc21 ; CC1(C)c2ccccc2-c2c(N(c3ccccc3)c3ccccc3-c3cccc4oc5ccccc5c34)cccc21 ; CC1(C)c2ccccc2-c2c(N(c3ccccc3-c3ccccc3)c3ccccc3-c3cccc4oc5ccccc5c34)cccc21]

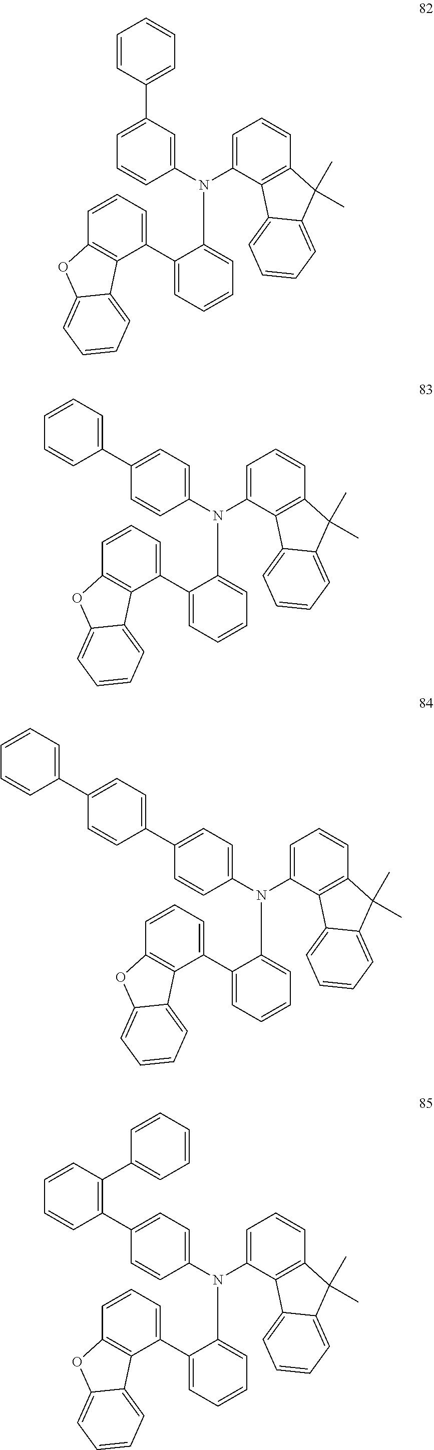

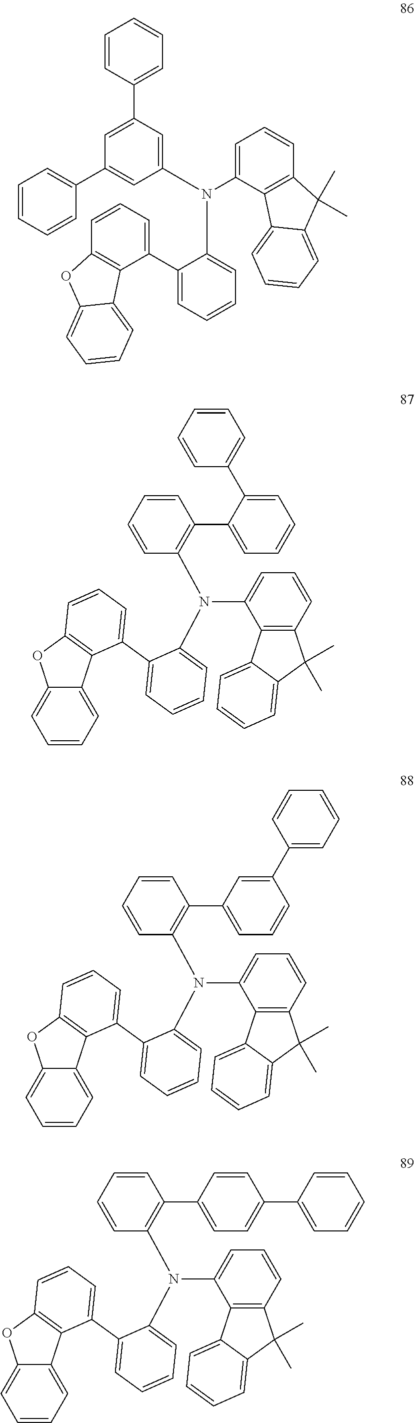 [SMILES: CC1(C)c2ccccc2-c2c(N(c3cc(-c4ccccc4)cc(-c4ccccc4)c3)c3ccccc3-c3cccc4oc5ccccc5c34)cccc21 ; CC1(C)c2ccccc2-c2c(N(c3ccccc3-c3ccccc3-c3ccccc3)c3ccccc3-c3cccc4oc5ccccc5c34)cccc21 ; CC1(C)c2ccccc2-c2c(N(c3ccccc3-c3cccc(-c4ccccc4)c3)c3ccccc3-c3cccc4oc5ccccc5c34)cccc21 ; CC1(C)c2ccccc2-c2c(N(c3ccccc3-c3ccc(-c4ccccc4)cc3)c3ccccc3-c3cccc4oc5ccccc5c34)cccc21]

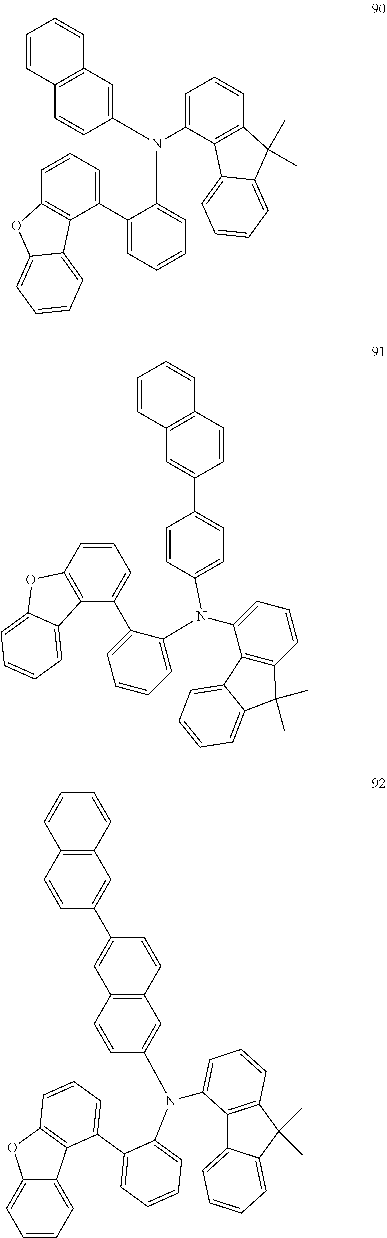 [SMILES: CC1(C)c2ccccc2-c2c(N(c3ccc4ccccc4c3)c3ccccc3-c3cccc4oc5ccccc5c34)cccc21 ; CC1(C)c2ccccc2-c2c(N(c3ccc(-c4ccc5ccccc5c4)cc3)c3ccccc3-c3cccc4oc5ccccc5c34)cccc21 ; CC1(C)c2ccccc2-c2c(N(c3ccc4cc(-c5ccc6ccccc6c5)ccc4c3)c3ccccc3-c3cccc4oc5ccccc5c34)cccc21]

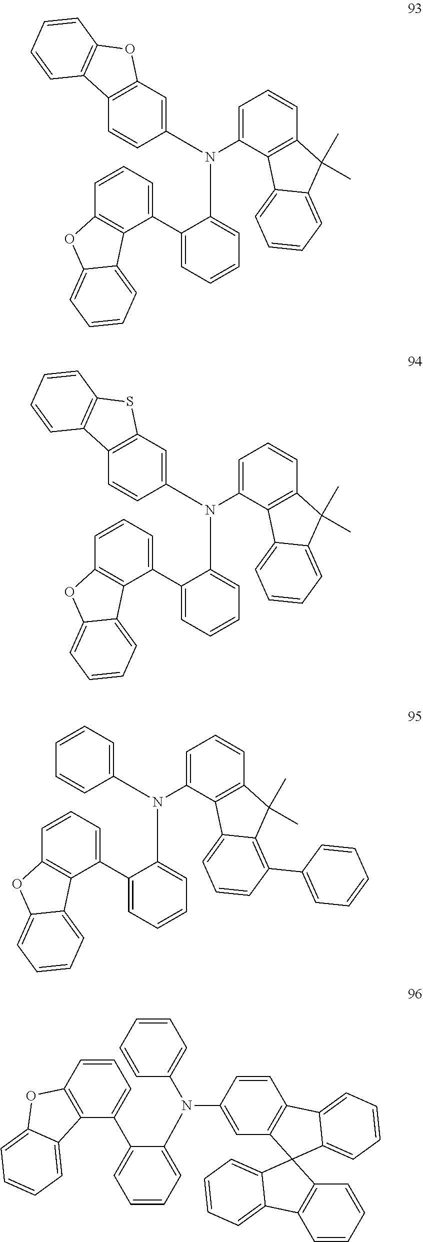

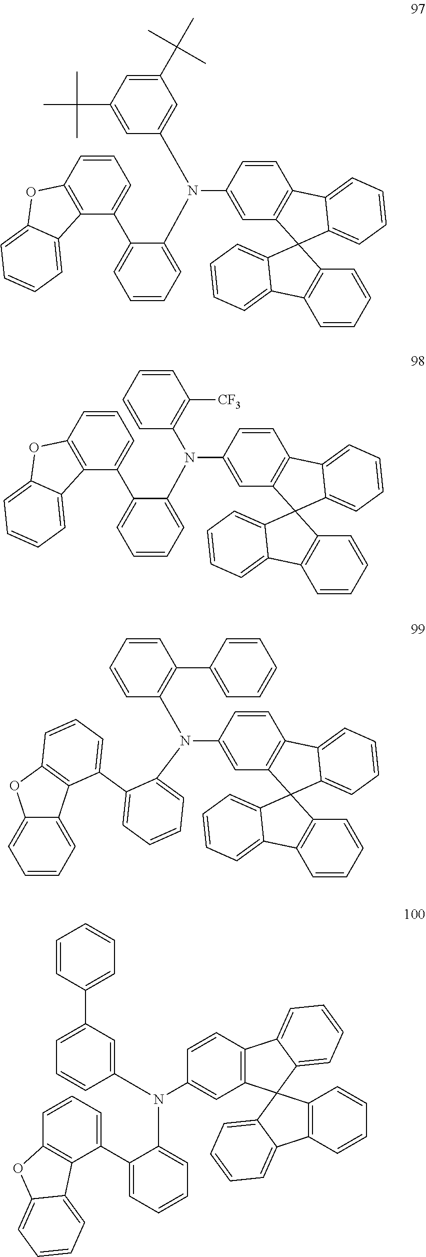 [SMILES: CC(C)(C)c1cc(N(c2ccc3c(c2)C2(c4ccccc4-c4ccccc42)c2ccccc2-3)c2ccccc2-c2cccc3oc4ccccc4c23)cc(C(C)(C)C)c1 ; FC(F)(F)c1ccccc1N(c1ccc2c(c1)C1(c3ccccc3-c3ccccc31)c1ccccc1-2)c1ccccc1-c1cccc2oc3ccccc3c12 ; c1ccc(-c2ccccc2N(c2ccc3c(c2)C2(c4ccccc4-c4ccccc42)c2ccccc2-3)c2ccccc2-c2cccc3oc4ccccc4c23)cc1 ; c1ccc(-c2cccc(N(c3ccc4c(c3)C3(c5ccccc5-c5ccccc53)c3ccccc3-4)c3ccccc3-c3cccc4oc5ccccc5c34)c2)cc1]

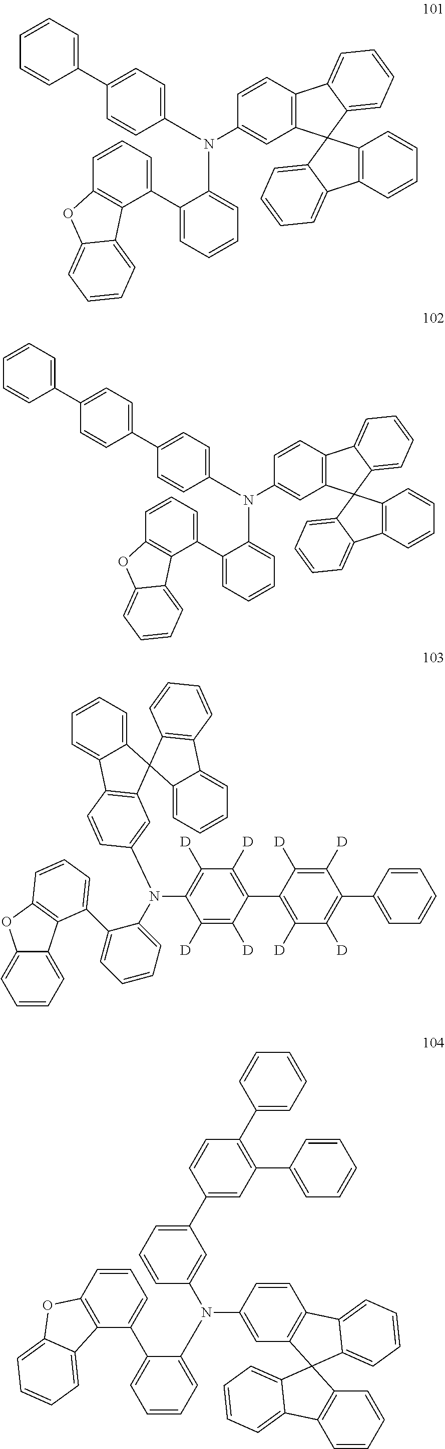 [SMILES: c1ccc(-c2ccc(N(c3ccc4c(c3)C3(c5ccccc5-c5ccccc53)c3ccccc3-4)c3ccccc3-c3cccc4oc5ccccc5c34)cc2)cc1 ; c1ccc(-c2ccc(-c3ccc(N(c4ccc5c(c4)C4(c6ccccc6-c6ccccc64)c4ccccc4-5)c4ccccc4-c4cccc5oc6ccccc6c45)cc3)cc2)cc1 ; [2H]c1c([2H])c(-c2c([2H])c([2H])c(N(c3ccc4c(c3)C3(c5ccccc5-c5ccccc53)c3ccccc3-4)c3ccccc3-c3cccc4oc5ccccc5c34)c([2H])c2[2H])c([2H])c([2H])c1-c1ccccc1 ; c1ccc(-c2ccc(-c3cccc(N(c4ccc5c(c4)C4(c6ccccc6-c6ccccc64)c4ccccc4-5)c4ccccc4-c4cccc5oc6ccccc6c45)c3)cc2-c2ccccc2)cc1]

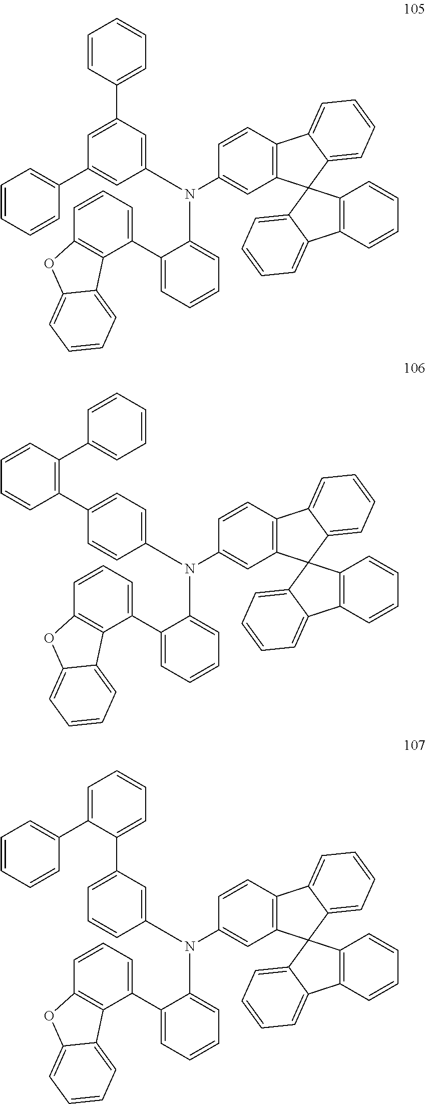 [SMILES: c1ccc(-c2cc(-c3ccccc3)cc(N(c3ccc4c(c3)C3(c5ccccc5-c5ccccc53)c3ccccc3-4)c3ccccc3-c3cccc4oc5ccccc5c34)c2)cc1 ; c1ccc(-c2ccccc2-c2ccc(N(c3ccc4c(c3)C3(c5ccccc5-c5ccccc53)c3ccccc3-4)c3ccccc3-c3cccc4oc5ccccc5c34)cc2)cc1 ; c1ccc(-c2ccccc2-c2cccc(N(c3ccc4c(c3)C3(c5ccccc5-c5ccccc53)c3ccccc3-4)c3ccccc3-c3cccc4oc5ccccc5c34)c2)cc1]

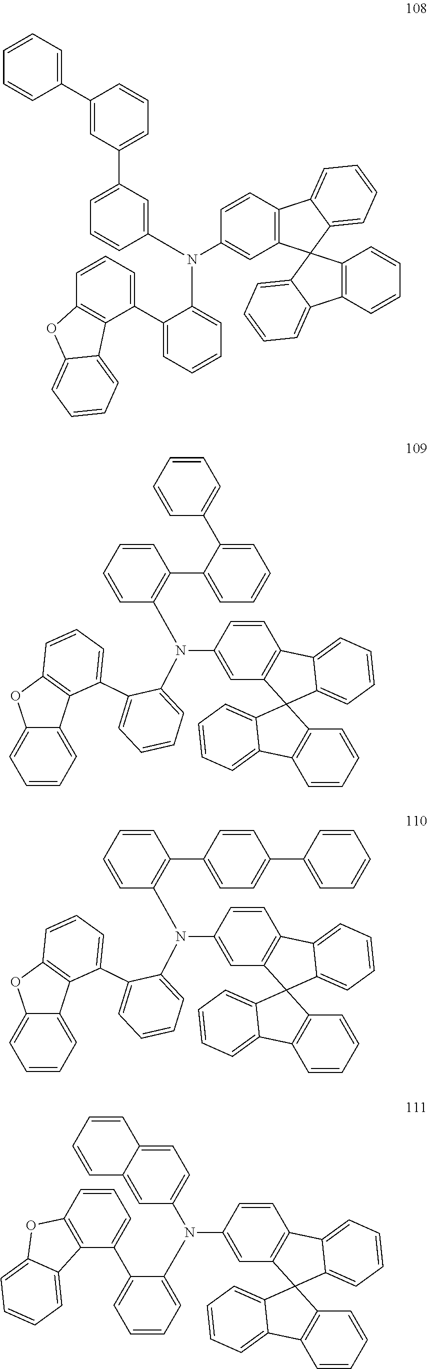

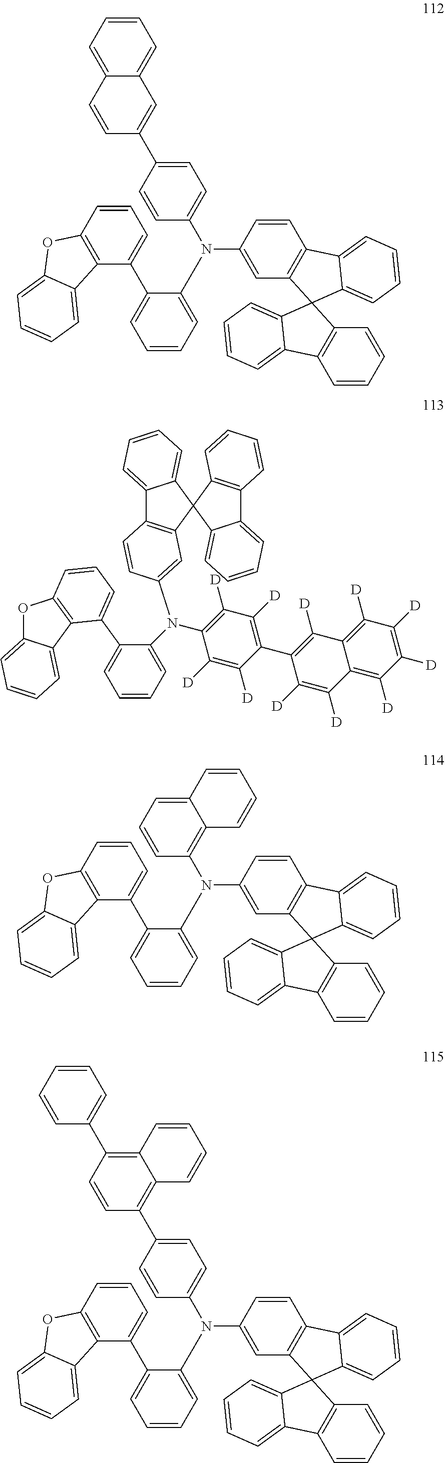 [SMILES: c1ccc(N(c2ccc(-c3ccc4ccccc4c3)cc2)c2ccc3c(c2)C2(c4ccccc4-c4ccccc42)c2ccccc2-3)c(-c2cccc3oc4ccccc4c23)c1 ; [2H]c1c([2H])c(N(c2ccc3c(c2)C2(c4ccccc4-c4ccccc42)c2ccccc2-3)c2ccccc2-c2cccc3oc4ccccc4c23)c([2H])c([2H])c1-c1c([2H])c([2H])c2c([2H])c([2H])c([2H])c([2H])c2c1[2H] ; c1ccc(N(c2ccc3c(c2)C2(c4ccccc4-c4ccccc42)c2ccccc2-3)c2cccc3ccccc23)c(-c2cccc3oc4ccccc4c23)c1 ; c1ccc(-c2ccc(-c3ccc(N(c4ccc5c(c4)C4(c6ccccc6-c6ccccc64)c4ccccc4-5)c4ccccc4-c4cccc5oc6ccccc6c45)cc3)c3ccccc23)cc1]

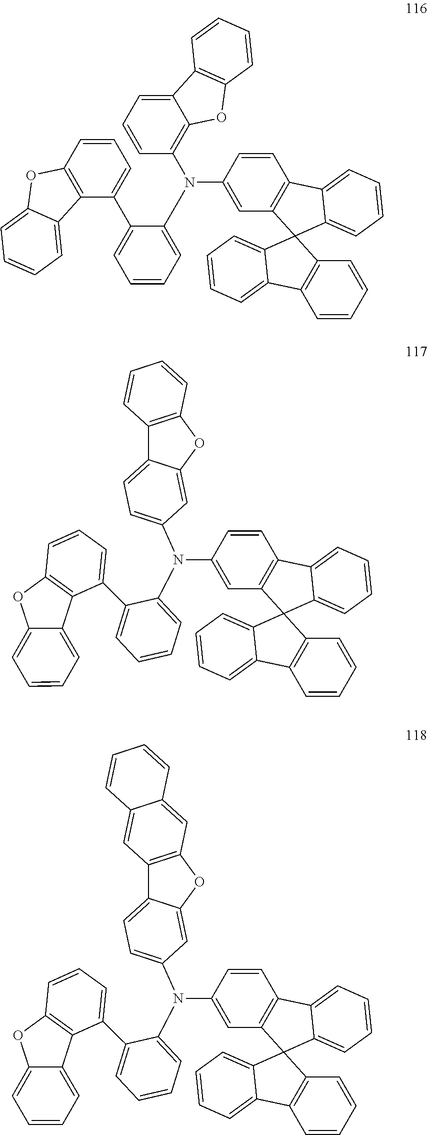 [SMILES: c1ccc(N(c2ccc3c(c2)C2(c4ccccc4-c4ccccc42)c2ccccc2-3)c2cccc3c2oc2ccccc23)c(-c2cccc3oc4ccccc4c23)c1 ; c1ccc(N(c2ccc3c(c2)C2(c4ccccc4-c4ccccc42)c2ccccc2-3)c2ccc3c(c2)oc2ccccc23)c(-c2cccc3oc4ccccc4c23)c1 ; c1ccc(N(c2ccc3c(c2)C2(c4ccccc4-c4ccccc42)c2ccccc2-3)c2ccc3c(c2)oc2cc4ccccc4cc23)c(-c2cccc3oc4ccccc4c23)c1]

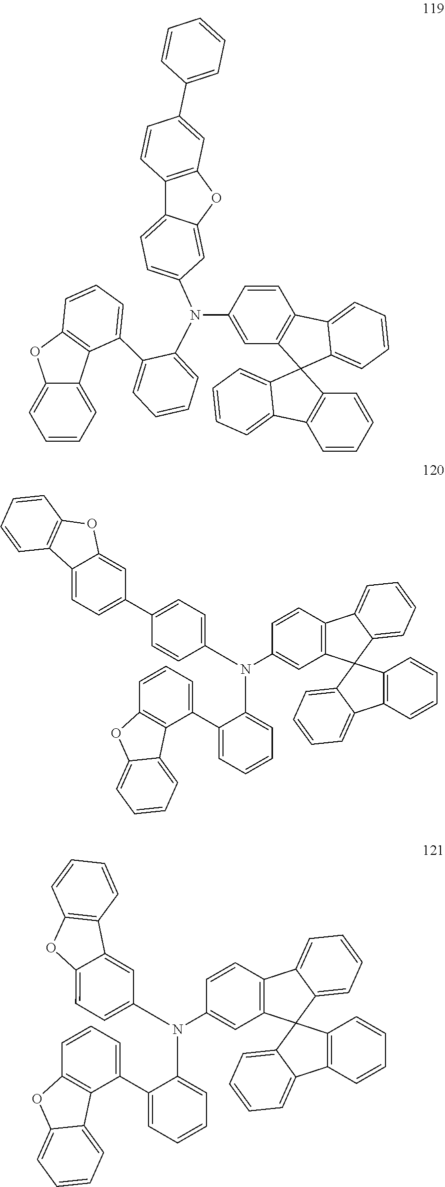 [SMILES: c1ccc(-c2ccc3c(c2)oc2cc(N(c4ccc5c(c4)C4(c6ccccc6-c6ccccc64)c4ccccc4-5)c4ccccc4-c4cccc5oc6ccccc6c45)ccc23)cc1 ; c1ccc(N(c2ccc(-c3ccc4c(c3)oc3ccccc34)cc2)c2ccc3c(c2)C2(c4ccccc4-c4ccccc42)c2ccccc2-3)c(-c2cccc3oc4ccccc4c23)c1 ; c1ccc(N(c2ccc3c(c2)C2(c4ccccc4-c4ccccc42)c2ccccc2-3)c2ccc3oc4ccccc4c3c2)c(-c2cccc3oc4ccccc4c23)c1]

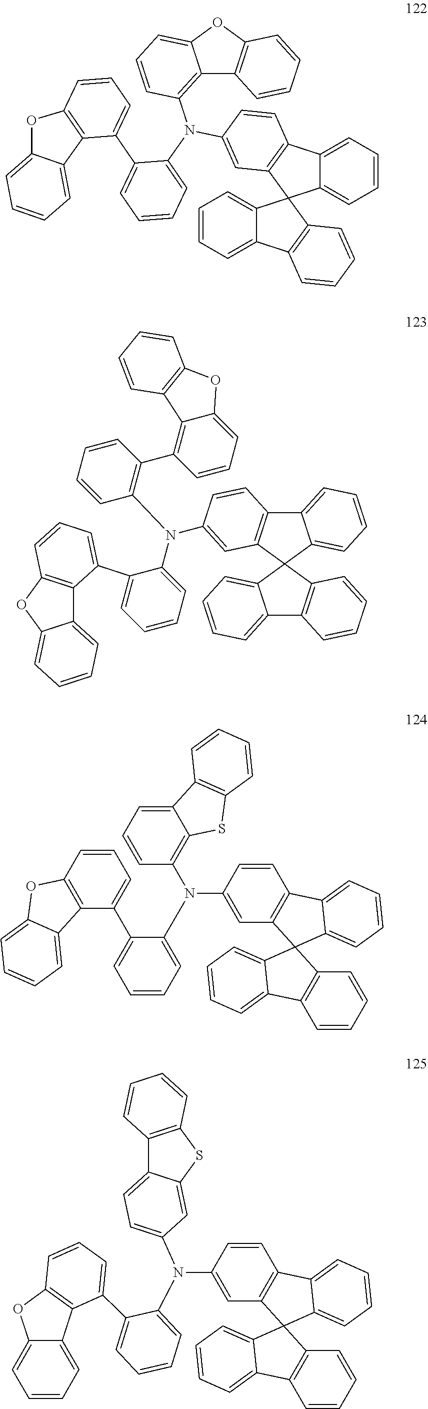 [SMILES: c1ccc(N(c2ccc3c(c2)C2(c4ccccc4-c4ccccc42)c2ccccc2-3)c2cccc3oc4ccccc4c23)c(-c2cccc3oc4ccccc4c23)c1 ; c1ccc(N(c2ccc3c(c2)C2(c4ccccc4-c4ccccc42)c2ccccc2-3)c2ccccc2-c2cccc3oc4ccccc4c23)c(-c2cccc3oc4ccccc4c23)c1 ; c1ccc(N(c2ccc3c(c2)C2(c4ccccc4-c4ccccc42)c2ccccc2-3)c2cccc3c2sc2ccccc23)c(-c2cccc3oc4ccccc4c23)c1 ; c1ccc(N(c2ccc3c(c2)C2(c4ccccc4-c4ccccc42)c2ccccc2-3)c2ccc3c(c2)sc2ccccc23)c(-c2cccc3oc4ccccc4c23)c1]

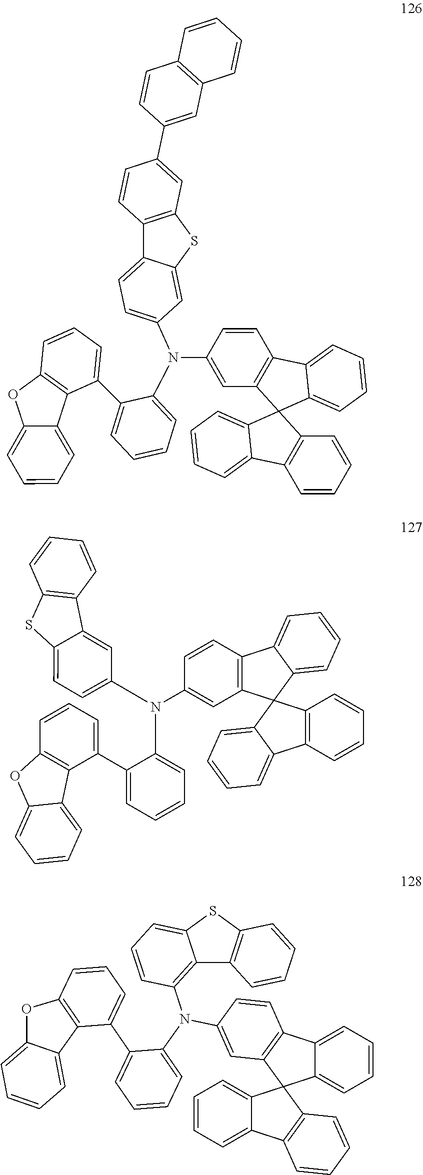 [SMILES: c1ccc(N(c2ccc3c(c2)C2(c4ccccc4-c4ccccc42)c2ccccc2-3)c2ccc3c(c2)sc2cc(-c4ccc5ccccc5c4)ccc23)c(-c2cccc3oc4ccccc4c23)c1 ; c1ccc(N(c2ccc3c(c2)C2(c4ccccc4-c4ccccc42)c2ccccc2-3)c2ccc3sc4ccccc4c3c2)c(-c2cccc3oc4ccccc4c23)c1 ; c1ccc(N(c2ccc3c(c2)C2(c4ccccc4-c4ccccc42)c2ccccc2-3)c2cccc3sc4ccccc4c23)c(-c2cccc3oc4ccccc4c23)c1]

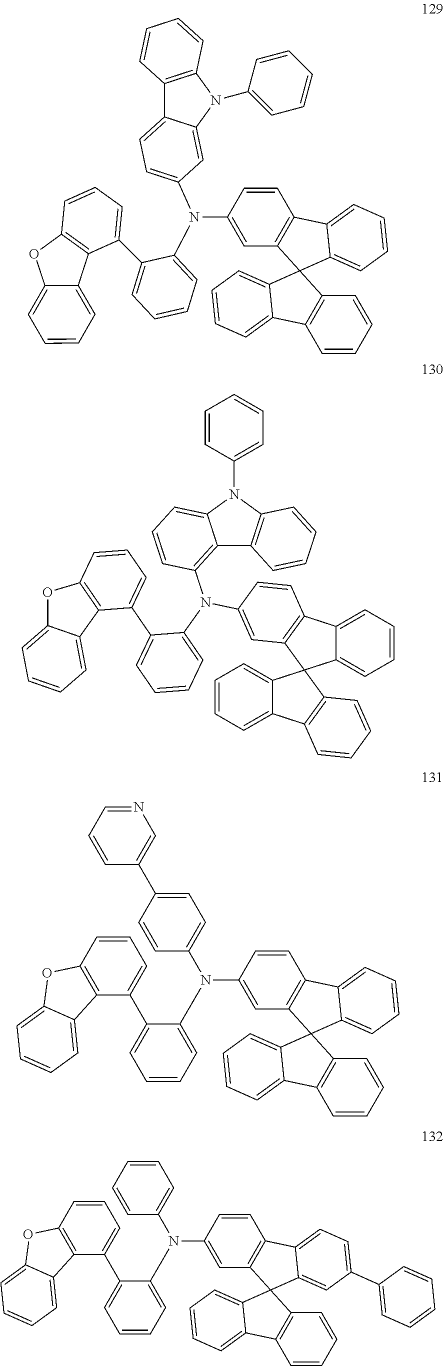 [SMILES: c1ccc(-n2c3ccccc3c3ccc(N(c4ccc5c(c4)C4(c6ccccc6-c6ccccc64)c4ccccc4-5)c4ccccc4-c4cccc5oc6ccccc6c45)cc32)cc1 ; c1ccc(-n2c3ccccc3c3c(N(c4ccc5c(c4)C4(c6ccccc6-c6ccccc64)c4ccccc4-5)c4ccccc4-c4cccc5oc6ccccc6c45)cccc32)cc1 ; c1cncc(-c2ccc(N(c3ccc4c(c3)C3(c5ccccc5-c5ccccc53)c3ccccc3-4)c3ccccc3-c3cccc4oc5ccccc5c34)cc2)c1 ; c1ccc(-c2ccc3c(c2)C2(c4ccccc4-c4ccccc42)c2cc(N(c4ccccc4)c4ccccc4-c4cccc5oc6ccccc6c45)ccc2-3)cc1]

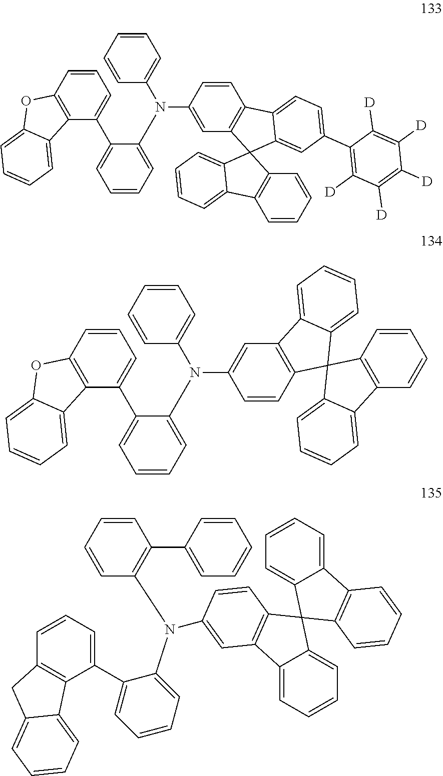

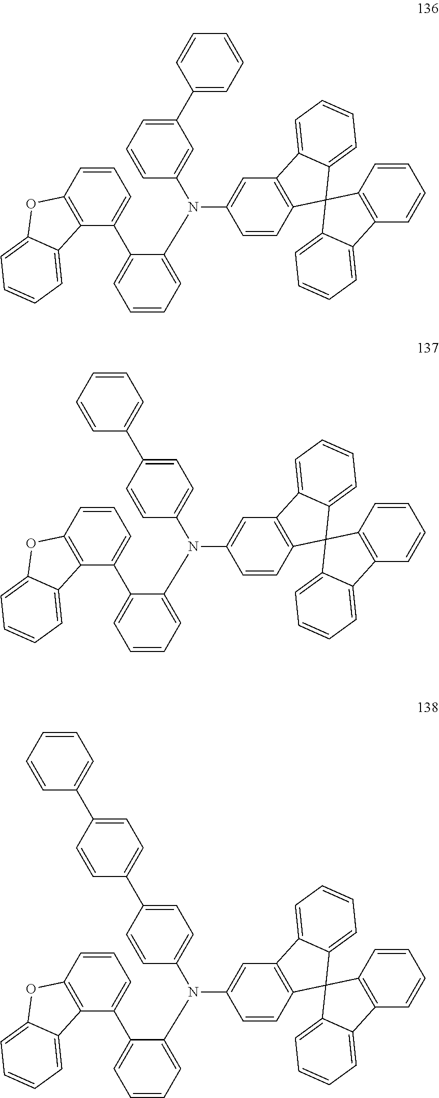 [SMILES: c1ccc(-c2cccc(N(c3ccc4c(c3)-c3ccccc3C43c4ccccc4-c4ccccc43)c3ccccc3-c3cccc4oc5ccccc5c34)c2)cc1 ; c1ccc(-c2ccc(N(c3ccc4c(c3)-c3ccccc3C43c4ccccc4-c4ccccc43)c3ccccc3-c3cccc4oc5ccccc5c34)cc2)cc1 ; c1ccc(-c2ccc(-c3ccc(N(c4ccc5c(c4)-c4ccccc4C54c5ccccc5-c5ccccc54)c4ccccc4-c4cccc5oc6ccccc6c45)cc3)cc2)cc1]

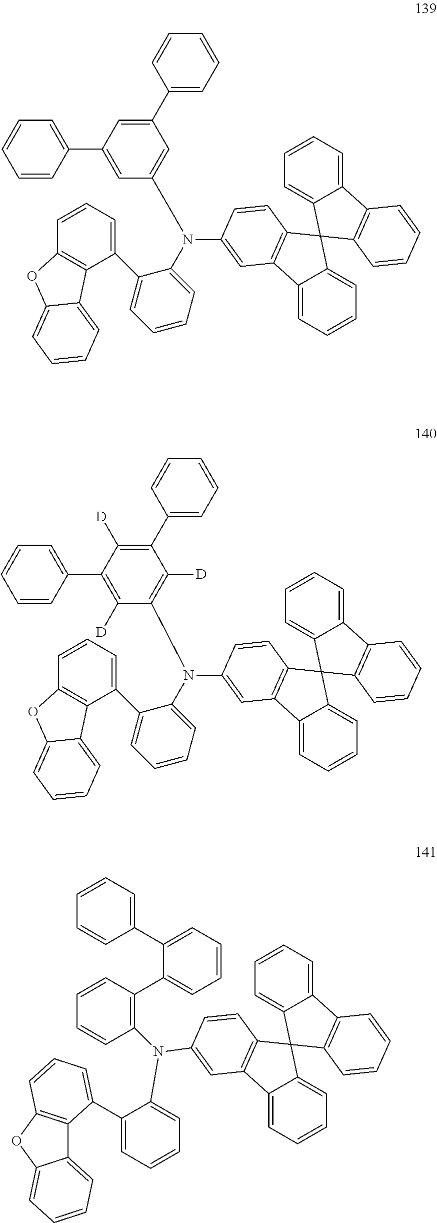 [SMILES: c1ccc(-c2cc(-c3ccccc3)cc(N(c3ccc4c(c3)-c3ccccc3C43c4ccccc4-c4ccccc43)c3ccccc3-c3cccc4oc5ccccc5c34)c2)cc1 ; [2H]c1c(-c2ccccc2)c([2H])c(N(c2ccc3c(c2)-c2ccccc2C32c3ccccc3-c3ccccc32)c2ccccc2-c2cccc3oc4ccccc4c23)c([2H])c1-c1ccccc1 ; c1ccc(-c2ccccc2-c2ccccc2N(c2ccc3c(c2)-c2ccccc2C32c3ccccc3-c3ccccc32)c2ccccc2-c2cccc3oc4ccccc4c23)cc1]

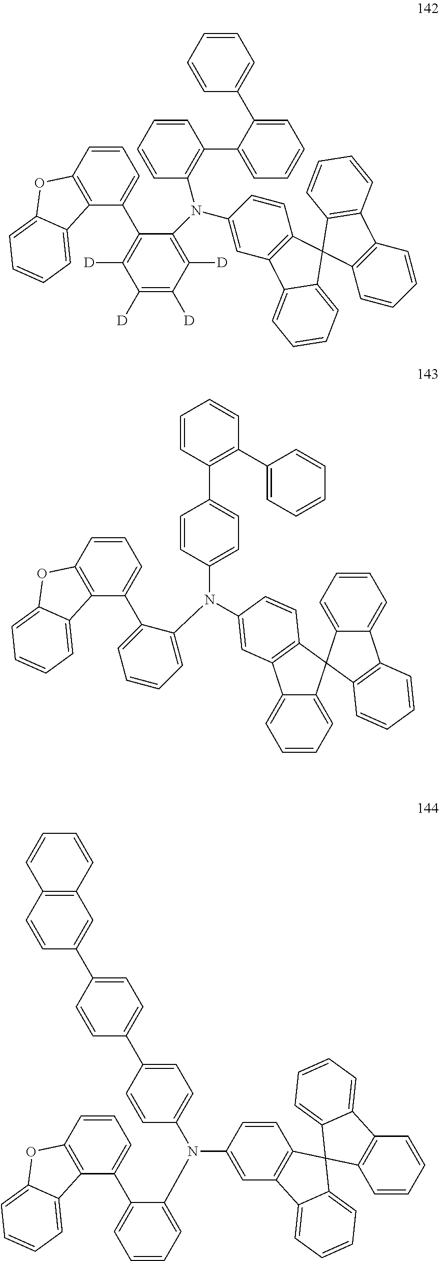 [SMILES: [2H]c1c([2H])c([2H])c(N(c2ccc3c(c2)-c2ccccc2C32c3ccccc3-c3ccccc32)c2ccccc2-c2ccccc2-c2ccccc2)c(-c2cccc3oc4ccccc4c23)c1[2H] ; c1ccc(-c2ccccc2-c2ccc(N(c3ccc4c(c3)-c3ccccc3C43c4ccccc4-c4ccccc43)c3ccccc3-c3cccc4oc5ccccc5c34)cc2)cc1 ; c1ccc(N(c2ccc(-c3ccc(-c4ccc5ccccc5c4)cc3)cc2)c2ccc3c(c2)-c2ccccc2C32c3ccccc3-c3ccccc32)c(-c2cccc3oc4ccccc4c23)c1]

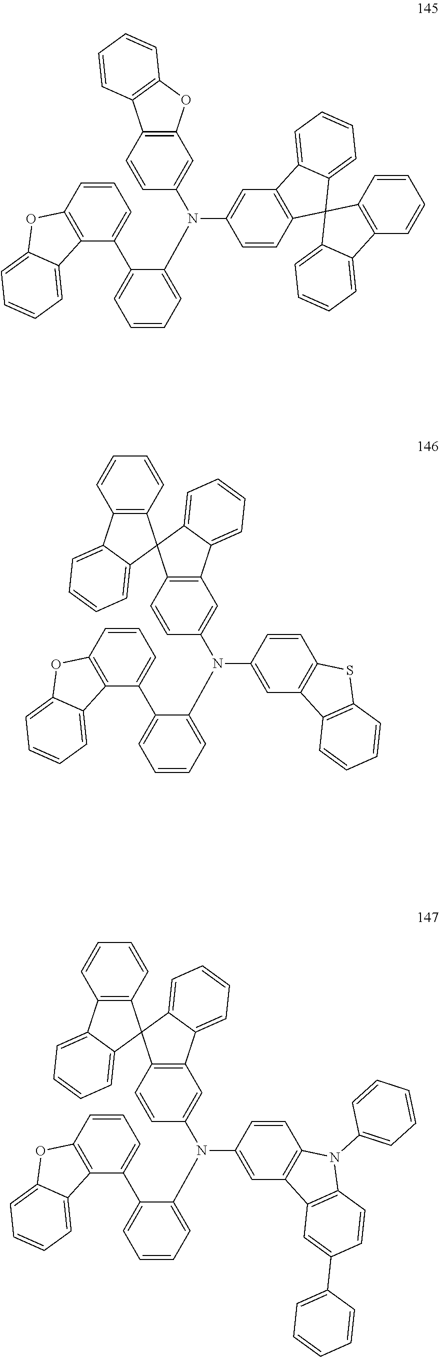 [SMILES: c1ccc(N(c2ccc3c(c2)-c2ccccc2C32c3ccccc3-c3ccccc32)c2ccc3c(c2)oc2ccccc23)c(-c2cccc3oc4ccccc4c23)c1 ; c1ccc(N(c2ccc3c(c2)-c2ccccc2C32c3ccccc3-c3ccccc32)c2ccc3sc4ccccc4c3c2)c(-c2cccc3oc4ccccc4c23)c1 ; c1ccc(-c2ccc3c(c2)c2cc(N(c4ccc5c(c4)-c4ccccc4C54c5ccccc5-c5ccccc54)c4ccccc4-c4cccc5oc6ccccc6c45)ccc2n3-c2ccccc2)cc1]

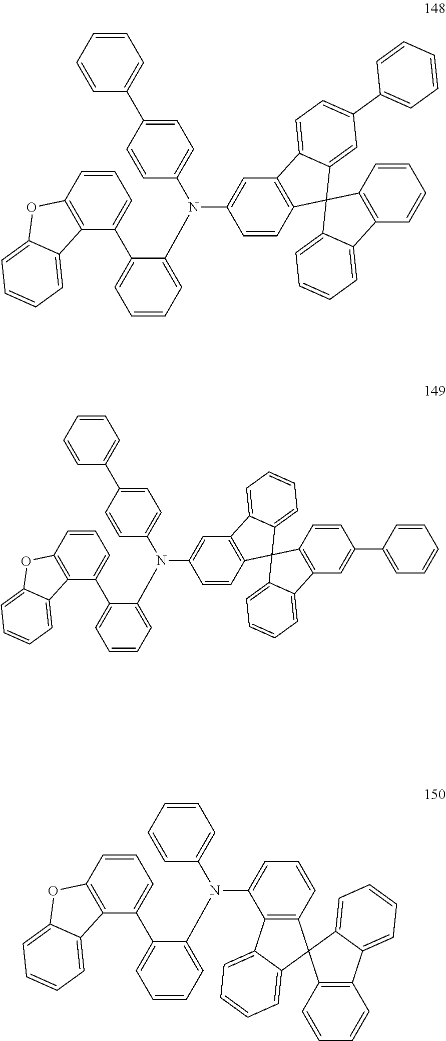 [SMILES: c1ccc(-c2ccc(N(c3ccc4c(c3)-c3ccc(-c5ccccc5)cc3C43c4ccccc4-c4ccccc43)c3ccccc3-c3cccc4oc5ccccc5c34)cc2)cc1 ; c1ccc(-c2ccc(N(c3ccc4c(c3)-c3ccccc3C43c4ccccc4-c4cc(-c5ccccc5)ccc43)c3ccccc3-c3cccc4oc5ccccc5c34)cc2)cc1 ; c1ccc(N(c2ccccc2-c2cccc3oc4ccccc4c23)c2cccc3c2-c2ccccc2C32c3ccccc3-c3ccccc32)cc1]

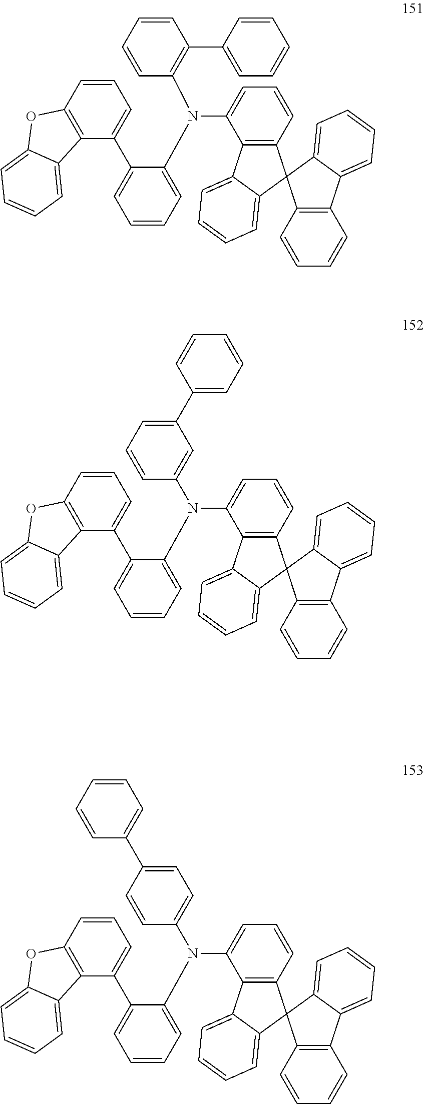 [SMILES: c1ccc(-c2ccccc2N(c2ccccc2-c2cccc3oc4ccccc4c23)c2cccc3c2-c2ccccc2C32c3ccccc3-c3ccccc32)cc1 ; c1ccc(-c2cccc(N(c3ccccc3-c3cccc4oc5ccccc5c34)c3cccc4c3-c3ccccc3C43c4ccccc4-c4ccccc43)c2)cc1 ; c1ccc(-c2ccc(N(c3ccccc3-c3cccc4oc5ccccc5c34)c3cccc4c3-c3ccccc3C43c4ccccc4-c4ccccc43)cc2)cc1]

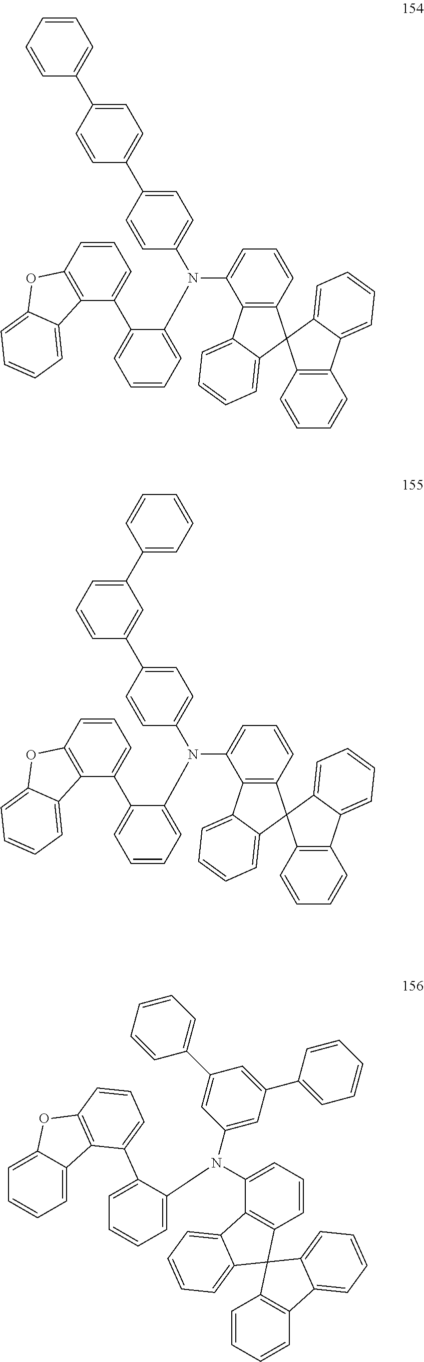 [SMILES: c1ccc(-c2ccc(-c3ccc(N(c4ccccc4-c4cccc5oc6ccccc6c45)c4cccc5c4-c4ccccc4C54c5ccccc5-c5ccccc54)cc3)cc2)cc1 ; c1ccc(-c2cccc(-c3ccc(N(c4ccccc4-c4cccc5oc6ccccc6c45)c4cccc5c4-c4ccccc4C54c5ccccc5-c5ccccc54)cc3)c2)cc1 ; c1ccc(-c2cc(-c3ccccc3)cc(N(c3ccccc3-c3cccc4oc5ccccc5c34)c3cccc4c3-c3ccccc3C43c4ccccc4-c4ccccc43)c2)cc1]

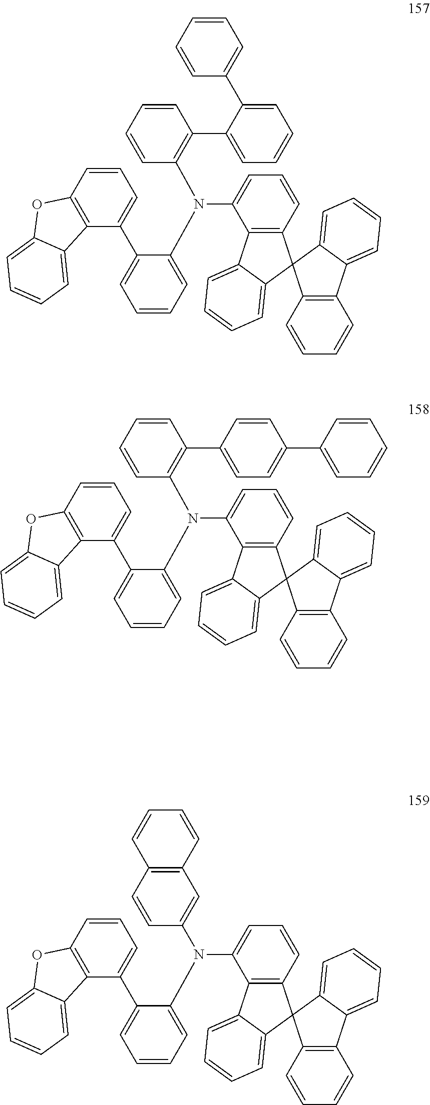 [SMILES: c1ccc(-c2ccccc2-c2ccccc2N(c2ccccc2-c2cccc3oc4ccccc4c23)c2cccc3c2-c2ccccc2C32c3ccccc3-c3ccccc32)cc1 ; c1ccc(-c2ccc(-c3ccccc3N(c3ccccc3-c3cccc4oc5ccccc5c34)c3cccc4c3-c3ccccc3C43c4ccccc4-c4ccccc43)cc2)cc1 ; c1ccc(N(c2ccc3ccccc3c2)c2cccc3c2-c2ccccc2C32c3ccccc3-c3ccccc32)c(-c2cccc3oc4ccccc4c23)c1]

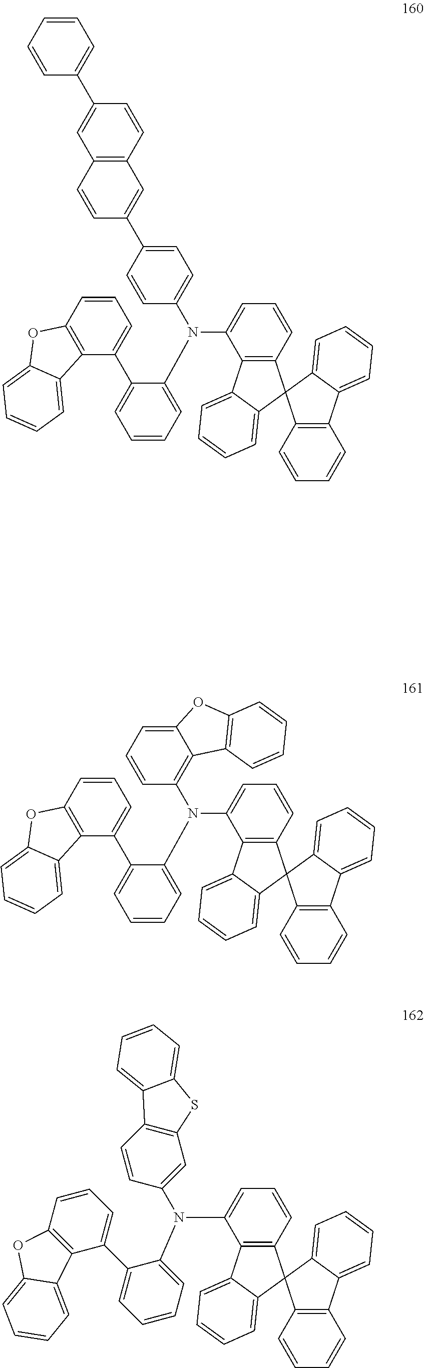 [SMILES: c1ccc(-c2ccc3cc(-c4ccc(N(c5ccccc5-c5cccc6oc7ccccc7c56)c5cccc6c5-c5ccccc5C65c6ccccc6-c6ccccc65)cc4)ccc3c2)cc1 ; c1ccc(N(c2cccc3c2-c2ccccc2C32c3ccccc3-c3ccccc32)c2cccc3oc4ccccc4c23)c(-c2cccc3oc4ccccc4c23)c1 ; c1ccc(N(c2ccc3c(c2)sc2ccccc23)c2cccc3c2-c2ccccc2C32c3ccccc3-c3ccccc32)c(-c2cccc3oc4ccccc4c23)c1]

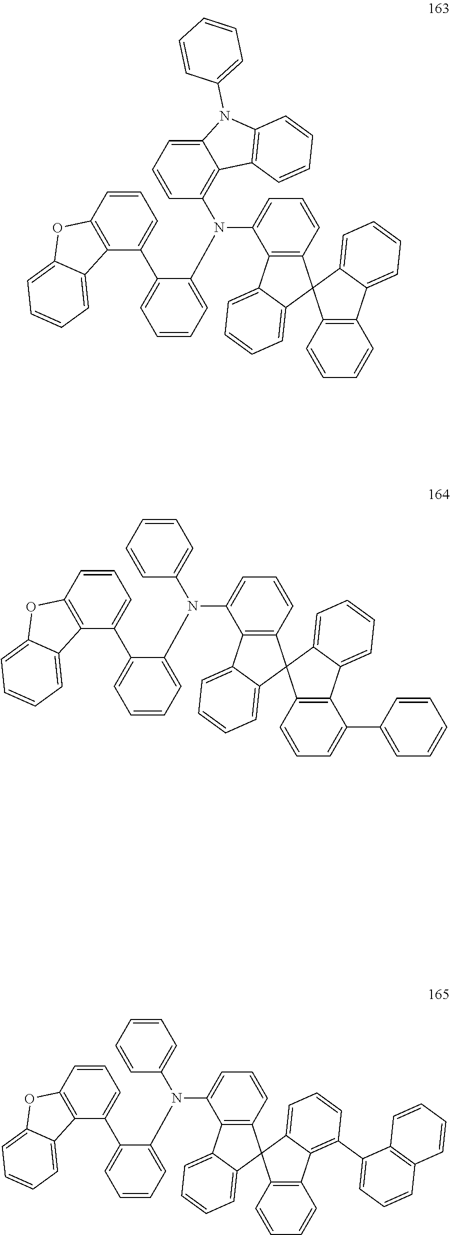 [SMILES: c1ccc(-n2c3ccccc3c3c(N(c4ccccc4-c4cccc5oc6ccccc6c45)c4cccc5c4-c4ccccc4C54c5ccccc5-c5ccccc54)cccc32)cc1 ; c1ccc(-c2cccc3c2-c2ccccc2C32c3ccccc3-c3c(N(c4ccccc4)c4ccccc4-c4cccc5oc6ccccc6c45)cccc32)cc1 ; c1ccc(N(c2ccccc2-c2cccc3oc4ccccc4c23)c2cccc3c2-c2ccccc2C32c3ccccc3-c3c(-c4cccc5ccccc45)cccc32)cc1]

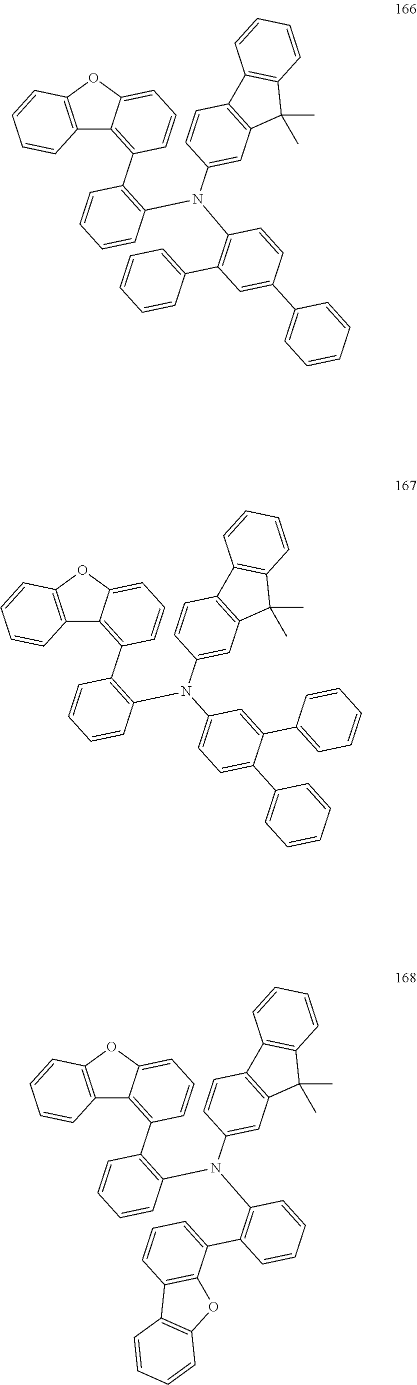 [SMILES: CC1(C)c2ccccc2-c2ccc(N(c3ccc(-c4ccccc4)cc3-c3ccccc3)c3ccccc3-c3cccc4oc5ccccc5c34)cc21 ; CC1(C)c2ccccc2-c2ccc(N(c3ccc(-c4ccccc4)c(-c4ccccc4)c3)c3ccccc3-c3cccc4oc5ccccc5c34)cc21 ; CC1(C)c2ccccc2-c2ccc(N(c3ccccc3-c3cccc4c3oc3ccccc34)c3ccccc3-c3cccc4oc5ccccc5c34)cc21]

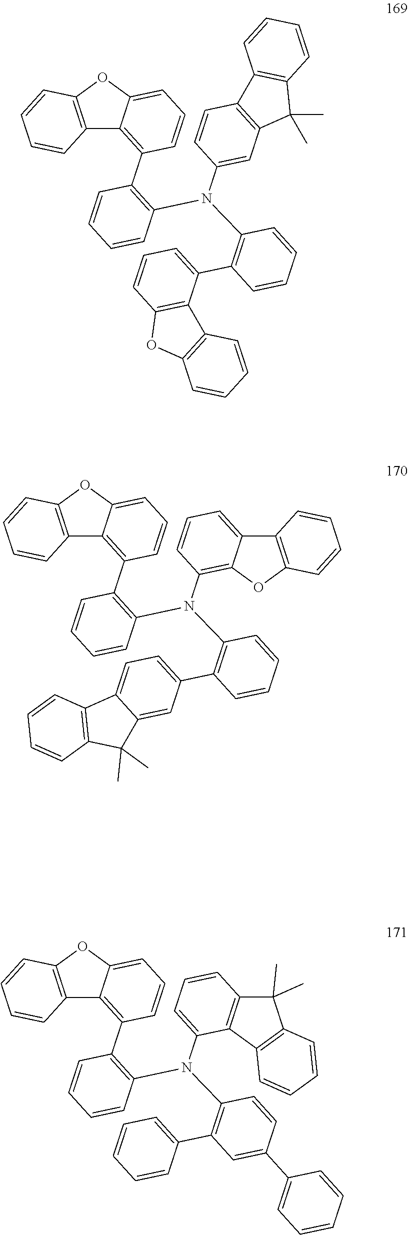 [SMILES: CC1(C)c2ccccc2-c2ccc(N(c3ccccc3-c3cccc4oc5ccccc5c34)c3ccccc3-c3cccc4oc5ccccc5c34)cc21 ; CC1(C)c2ccccc2-c2ccc(-c3ccccc3N(c3ccccc3-c3cccc4oc5ccccc5c34)c3cccc4c3oc3ccccc34)cc21 ; CC1(C)c2ccccc2-c2c(N(c3ccc(-c4ccccc4)cc3-c3ccccc3)c3ccccc3-c3cccc4oc5ccccc5c34)cccc21]

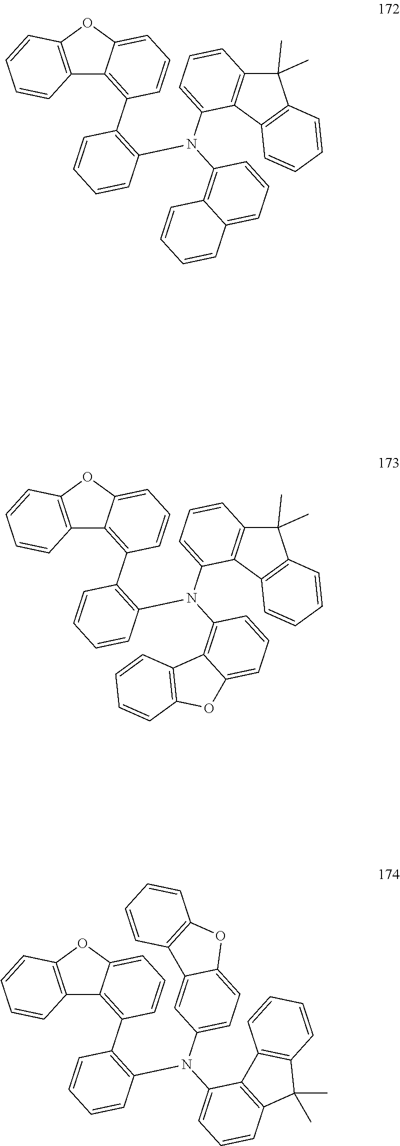 [SMILES: CC1(C)c2ccccc2-c2c(N(c3ccccc3-c3cccc4oc5ccccc5c34)c3cccc4ccccc34)cccc21 ; CC1(C)c2ccccc2-c2c(N(c3ccccc3-c3cccc4oc5ccccc5c34)c3cccc4oc5ccccc5c34)cccc21 ; CC1(C)c2ccccc2-c2c(N(c3ccc4oc5ccccc5c4c3)c3ccccc3-c3cccc4oc5ccccc5c34)cccc21]

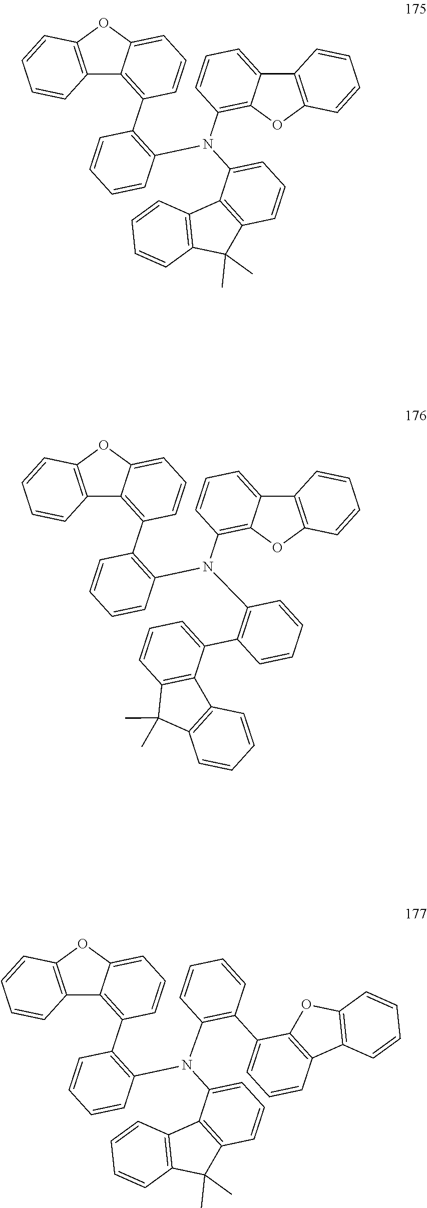 [SMILES: CC1(C)c2ccccc2-c2c(N(c3ccccc3-c3cccc4oc5ccccc5c34)c3cccc4c3oc3ccccc34)cccc21 ; CC1(C)c2ccccc2-c2c(-c3ccccc3N(c3ccccc3-c3cccc4oc5ccccc5c34)c3cccc4c3oc3ccccc34)cccc21 ; CC1(C)c2ccccc2-c2c(N(c3ccccc3-c3cccc4c3oc3ccccc34)c3ccccc3-c3cccc4oc5ccccc5c34)cccc21]

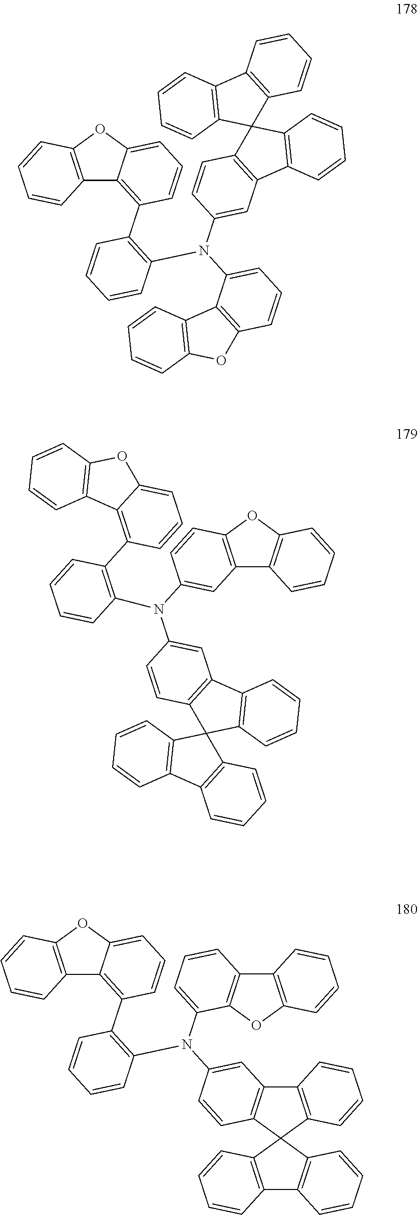 [SMILES: c1ccc(N(c2ccc3c(c2)-c2ccccc2C32c3ccccc3-c3ccccc32)c2cccc3oc4ccccc4c23)c(-c2cccc3oc4ccccc4c23)c1 ; c1ccc(N(c2ccc3c(c2)-c2ccccc2C32c3ccccc3-c3ccccc32)c2ccc3oc4ccccc4c3c2)c(-c2cccc3oc4ccccc4c23)c1 ; c1ccc(N(c2ccc3c(c2)-c2ccccc2C32c3ccccc3-c3ccccc32)c2cccc3c2oc2ccccc23)c(-c2cccc3oc4ccccc4c23)c1]

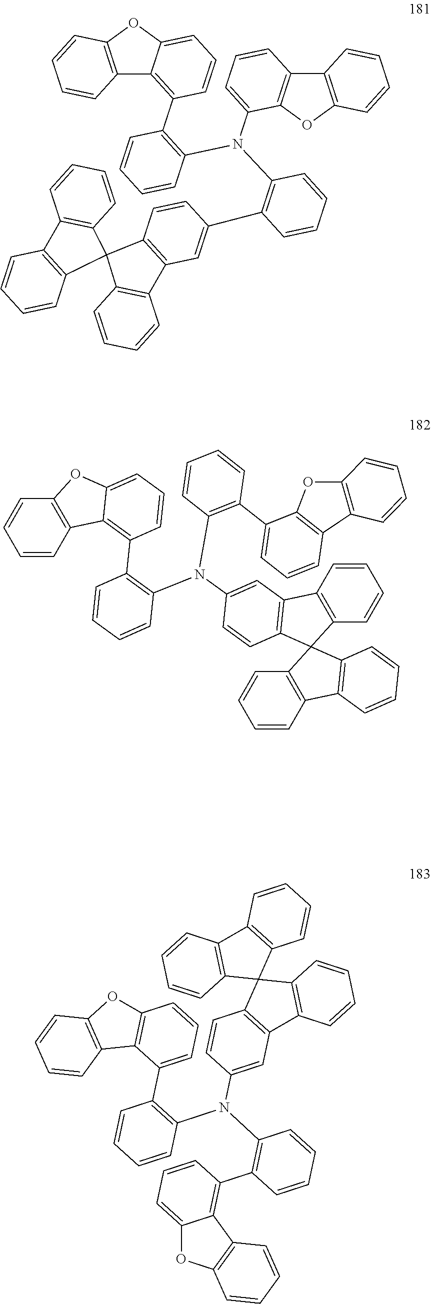 [SMILES: c1ccc(N(c2ccccc2-c2cccc3oc4ccccc4c23)c2cccc3c2oc2ccccc23)c(-c2ccc3c(c2)-c2ccccc2C32c3ccccc3-c3ccccc32)c1 ; c1ccc(N(c2ccc3c(c2)-c2ccccc2C32c3ccccc3-c3ccccc32)c2ccccc2-c2cccc3oc4ccccc4c23)c(-c2cccc3c2oc2ccccc23)c1 ; c1ccc(N(c2ccc3c(c2)-c2ccccc2C32c3ccccc3-c3ccccc32)c2ccccc2-c2cccc3oc4ccccc4c23)c(-c2cccc3oc4ccccc4c23)c1]

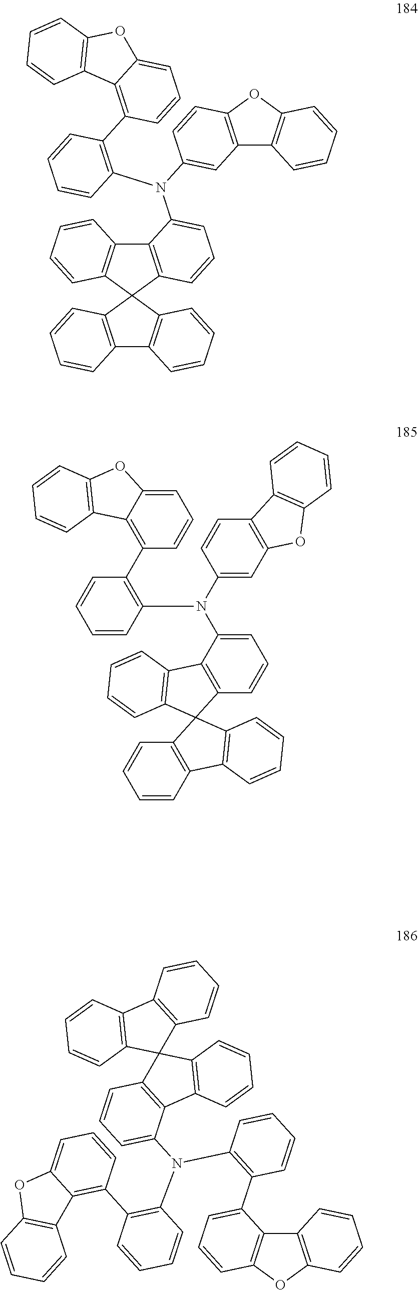 [SMILES: c1ccc(N(c2ccc3oc4ccccc4c3c2)c2cccc3c2-c2ccccc2C32c3ccccc3-c3ccccc32)c(-c2cccc3oc4ccccc4c23)c1 ; c1ccc(N(c2ccc3c(c2)oc2ccccc23)c2cccc3c2-c2ccccc2C32c3ccccc3-c3ccccc32)c(-c2cccc3oc4ccccc4c23)c1 ; c1ccc(N(c2ccccc2-c2cccc3oc4ccccc4c23)c2cccc3c2-c2ccccc2C32c3ccccc3-c3ccccc32)c(-c2cccc3oc4ccccc4c23)c1]

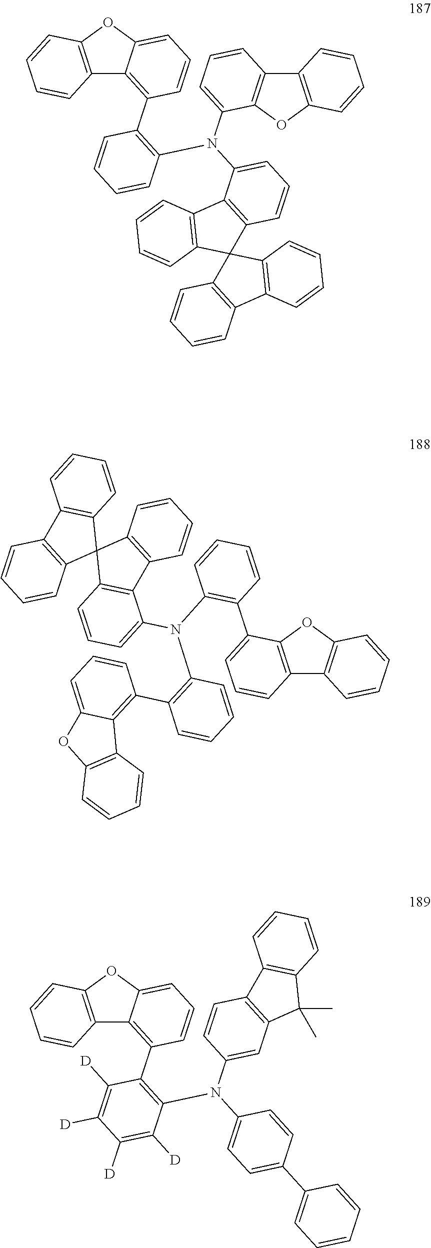 [SMILES: c1ccc(N(c2cccc3c2-c2ccccc2C32c3ccccc3-c3ccccc32)c2cccc3c2oc2ccccc23)c(-c2cccc3oc4ccccc4c23)c1 ; c1ccc(N(c2ccccc2-c2cccc3oc4ccccc4c23)c2cccc3c2-c2ccccc2C32c3ccccc3-c3ccccc32)c(-c2cccc3c2oc2ccccc23)c1 ; [2H]c1c([2H])c([2H])c(N(c2ccc(-c3ccccc3)cc2)c2ccc3c(c2)C(C)(C)c2ccccc2-3)c(-c2cccc3oc4ccccc4c23)c1[2H]]

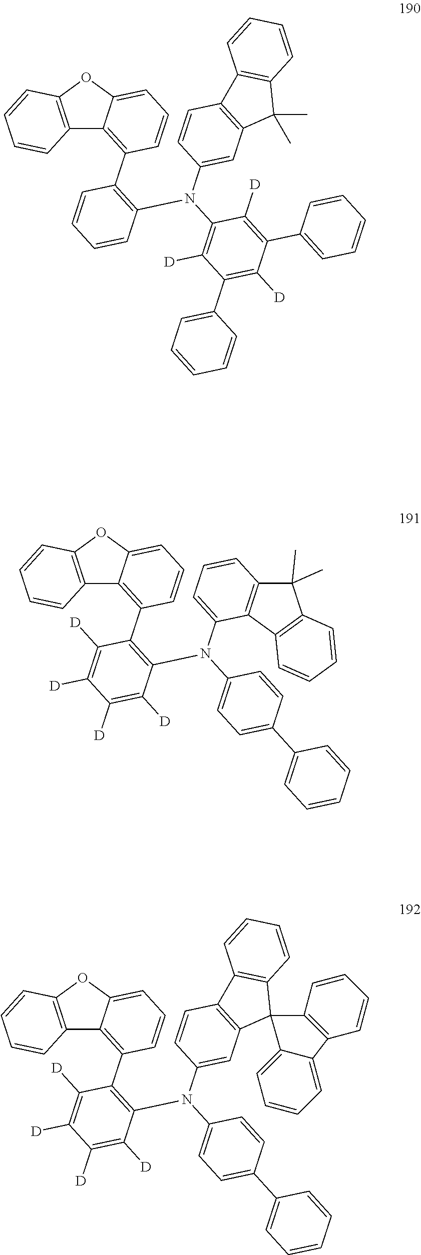 [SMILES: [2H]c1c(-c2ccccc2)c([2H])c(N(c2ccc3c(c2)C(C)(C)c2ccccc2-3)c2ccccc2-c2cccc3oc4ccccc4c23)c([2H])c1-c1ccccc1 ; [2H]c1c([2H])c([2H])c(N(c2ccc(-c3ccccc3)cc2)c2cccc3c2-c2ccccc2C3(C)C)c(-c2cccc3oc4ccccc4c23)c1[2H] ; [2H]c1c([2H])c([2H])c(N(c2ccc(-c3ccccc3)cc2)c2ccc3c(c2)C2(c4ccccc4-c4ccccc42)c2ccccc2-3)c(-c2cccc3oc4ccccc4c23)c1[2H]]

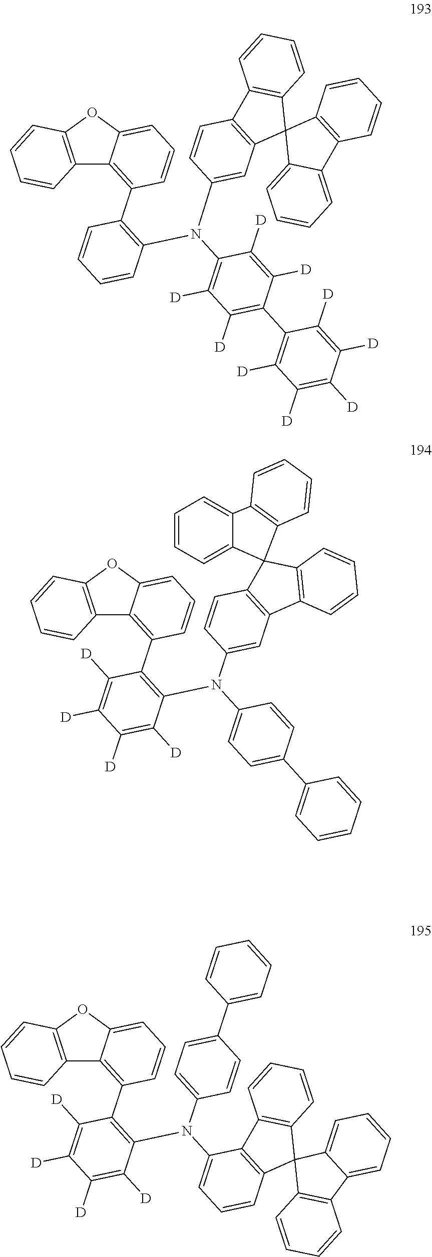

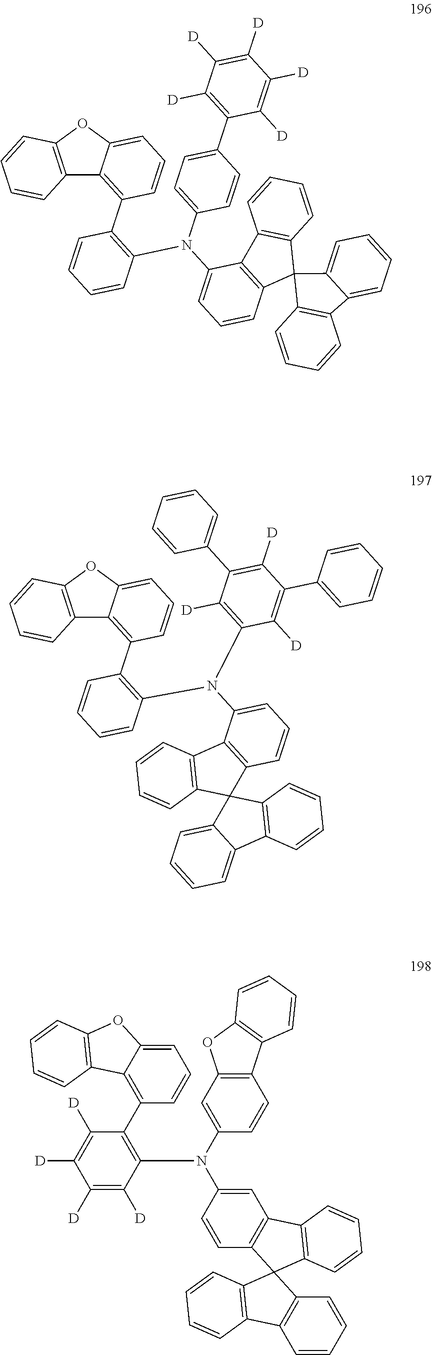 [SMILES: [2H]c1c([2H])c([2H])c(-c2ccc(N(c3ccccc3-c3cccc4oc5ccccc5c34)c3cccc4c3-c3ccccc3C43c4ccccc4-c4ccccc43)cc2)c([2H])c1[2H] ; [2H]c1c(-c2ccccc2)c([2H])c(N(c2ccccc2-c2cccc3oc4ccccc4c23)c2cccc3c2-c2ccccc2C32c3ccccc3-c3ccccc32)c([2H])c1-c1ccccc1 ; [2H]c1c([2H])c([2H])c(N(c2ccc3c(c2)-c2ccccc2C32c3ccccc3-c3ccccc32)c2ccc3c(c2)oc2ccccc23)c(-c2cccc3oc4ccccc4c23)c1[2H]]

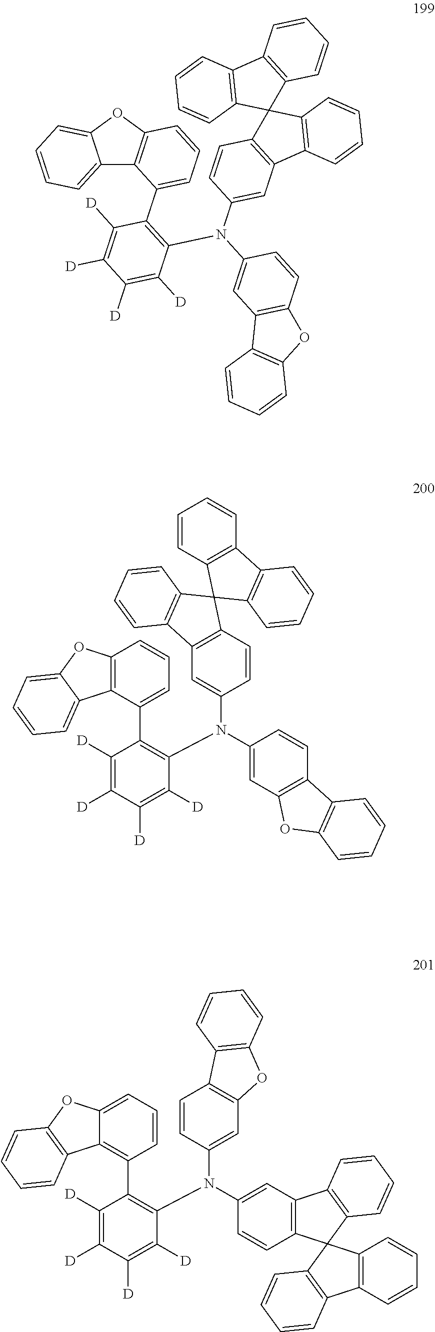 [SMILES: [2H]c1c([2H])c([2H])c(N(c2ccc3c(c2)-c2ccccc2C32c3ccccc3-c3ccccc32)c2ccc3oc4ccccc4c3c2)c(-c2cccc3oc4ccccc4c23)c1[2H] ; [2H]c1c([2H])c([2H])c(N(c2ccc3c(c2)-c2ccccc2C32c3ccccc3-c3ccccc32)c2ccc3c(c2)oc2ccccc23)c(-c2cccc3oc4ccccc4c23)c1[2H] ; [2H]c1c([2H])c([2H])c(N(c2ccc3c(c2)-c2ccccc2C32c3ccccc3-c3ccccc32)c2ccc3c(c2)oc2ccccc23)c(-c2cccc3oc4ccccc4c23)c1[2H]]

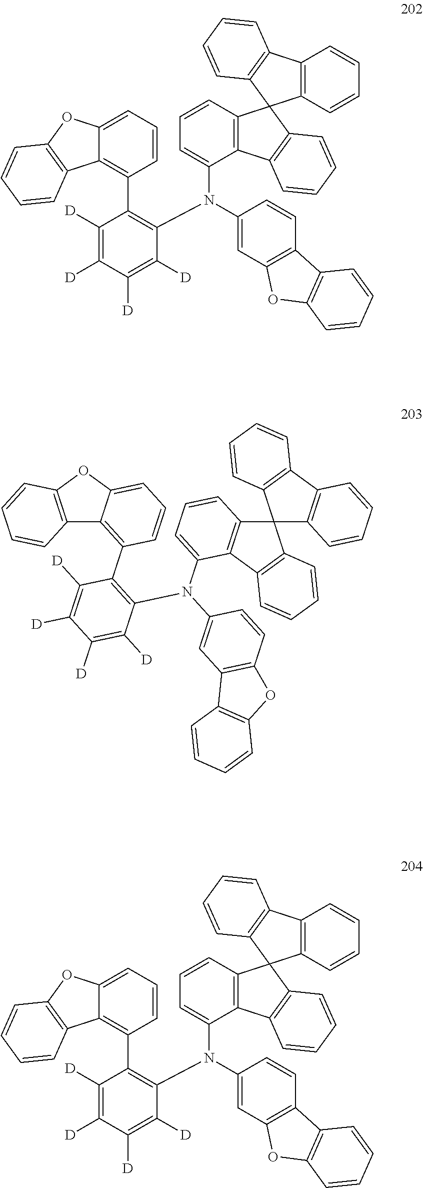 [SMILES: [2H]c1c([2H])c([2H])c(N(c2ccc3c(c2)oc2ccccc23)c2cccc3c2-c2ccccc2C32c3ccccc3-c3ccccc32)c(-c2cccc3oc4ccccc4c23)c1[2H] ; [2H]c1c([2H])c([2H])c(N(c2ccc3oc4ccccc4c3c2)c2cccc3c2-c2ccccc2C32c3ccccc3-c3ccccc32)c(-c2cccc3oc4ccccc4c23)c1[2H] ; [2H]c1c([2H])c([2H])c(N(c2ccc3c(c2)oc2ccccc23)c2cccc3c2-c2ccccc2C32c3ccccc3-c3ccccc32)c(-c2cccc3oc4ccccc4c23)c1[2H]]

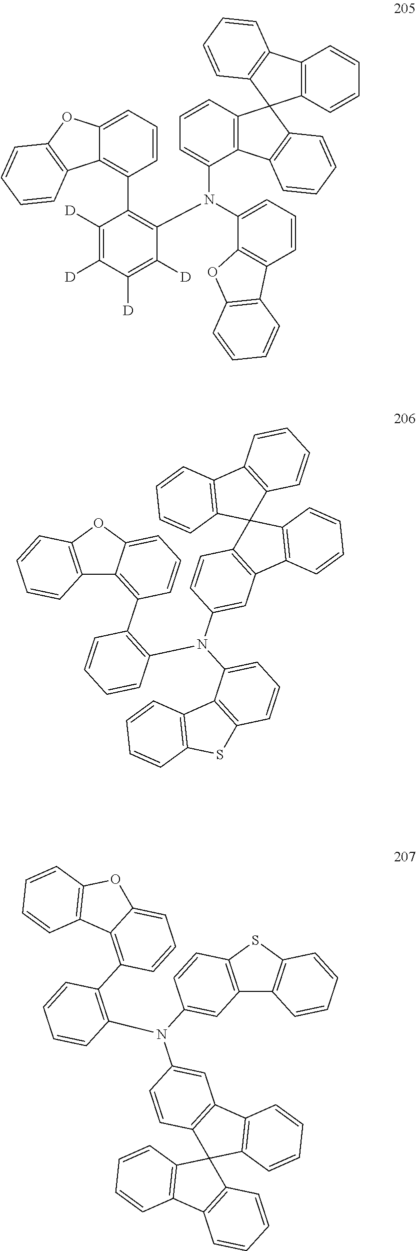 [SMILES: [2H]c1c([2H])c([2H])c(N(c2cccc3c2-c2ccccc2C32c3ccccc3-c3ccccc32)c2cccc3c2oc2ccccc23)c(-c2cccc3oc4ccccc4c23)c1[2H] ; c1ccc(N(c2ccc3c(c2)-c2ccccc2C32c3ccccc3-c3ccccc32)c2cccc3sc4ccccc4c23)c(-c2cccc3oc4ccccc4c23)c1 ; c1ccc(N(c2ccc3c(c2)-c2ccccc2C32c3ccccc3-c3ccccc32)c2ccc3sc4ccccc4c3c2)c(-c2cccc3oc4ccccc4c23)c1]

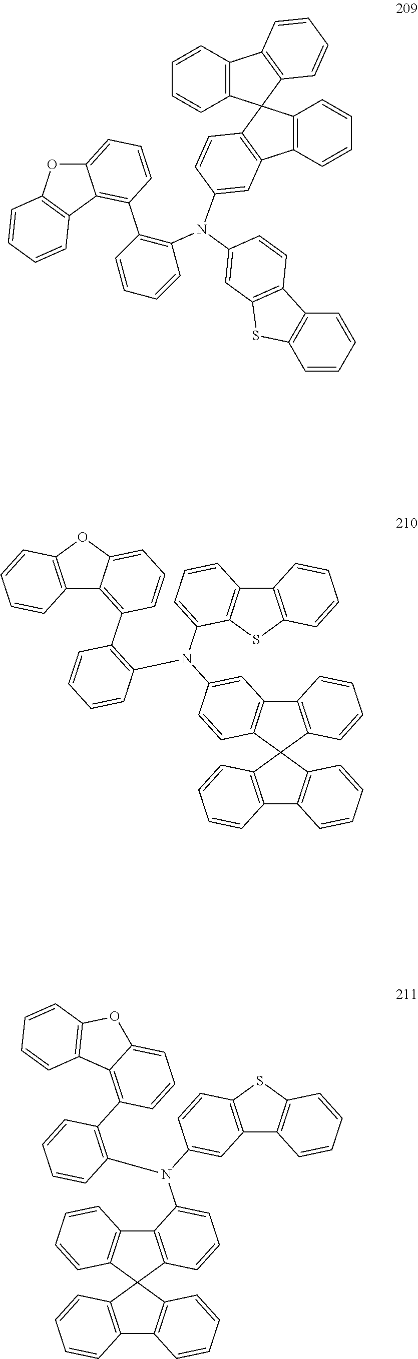 [SMILES: c1ccc(N(c2ccc3c(c2)-c2ccccc2C32c3ccccc3-c3ccccc32)c2ccc3c(c2)sc2ccccc23)c(-c2cccc3oc4ccccc4c23)c1 ; c1ccc(N(c2ccc3c(c2)-c2ccccc2C32c3ccccc3-c3ccccc32)c2cccc3c2sc2ccccc23)c(-c2cccc3oc4ccccc4c23)c1 ; c1ccc(N(c2ccc3sc4ccccc4c3c2)c2cccc3c2-c2ccccc2C32c3ccccc3-c3ccccc32)c(-c2cccc3oc4ccccc4c23)c1]

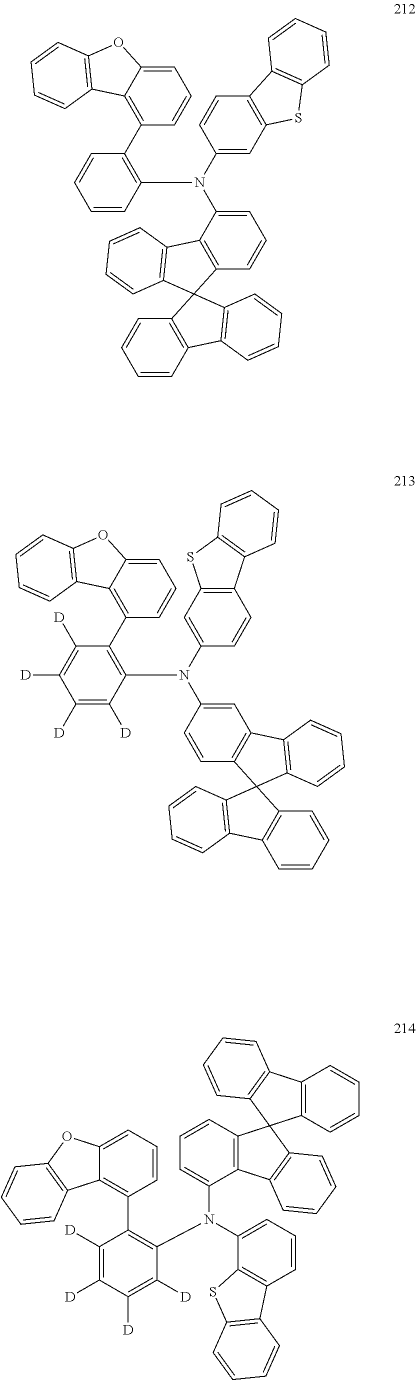 [SMILES: c1ccc(N(c2ccc3c(c2)sc2ccccc23)c2cccc3c2-c2ccccc2C32c3ccccc3-c3ccccc32)c(-c2cccc3oc4ccccc4c23)c1 ; [2H]c1c([2H])c([2H])c(N(c2ccc3c(c2)-c2ccccc2C32c3ccccc3-c3ccccc32)c2ccc3c(c2)sc2ccccc23)c(-c2cccc3oc4ccccc4c23)c1[2H] ; [2H]c1c([2H])c([2H])c(N(c2cccc3c2-c2ccccc2C32c3ccccc3-c3ccccc32)c2cccc3c2sc2ccccc23)c(-c2cccc3oc4ccccc4c23)c1[2H]]

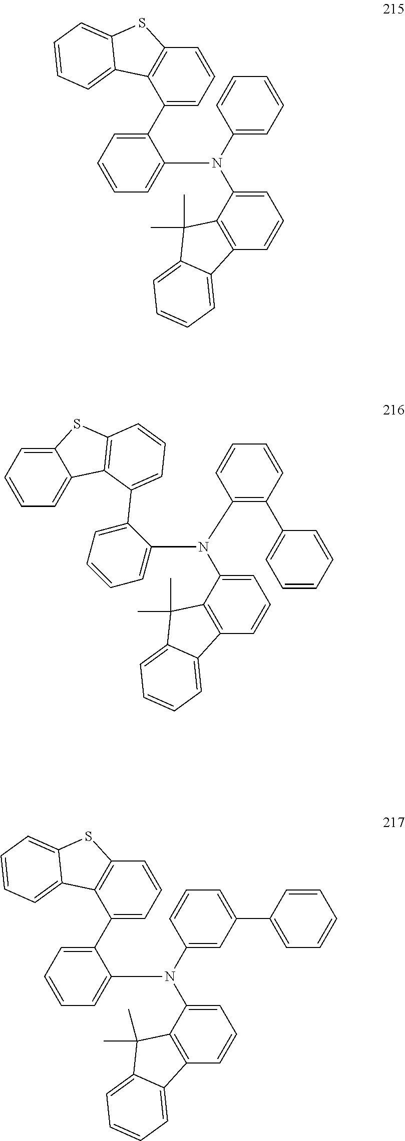 [SMILES: CC1(C)c2ccccc2-c2cccc(N(c3ccccc3)c3ccccc3-c3cccc4sc5ccccc5c34)c21 ; CC1(C)c2ccccc2-c2cccc(N(c3ccccc3-c3ccccc3)c3ccccc3-c3cccc4sc5ccccc5c34)c21 ; CC1(C)c2ccccc2-c2cccc(N(c3cccc(-c4ccccc4)c3)c3ccccc3-c3cccc4sc5ccccc5c34)c21]

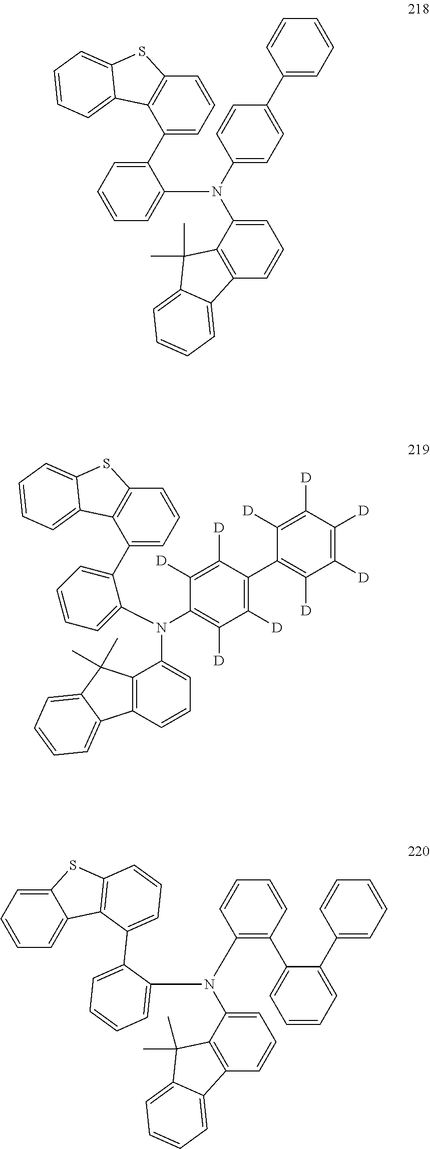 [SMILES: CC1(C)c2ccccc2-c2cccc(N(c3ccc(-c4ccccc4)cc3)c3ccccc3-c3cccc4sc5ccccc5c34)c21 ; [2H]c1c([2H])c([2H])c(-c2c([2H])c([2H])c(N(c3ccccc3-c3cccc4sc5ccccc5c34)c3cccc4c3C(C)(C)c3ccccc3-4)c([2H])c2[2H])c([2H])c1[2H] ; CC1(C)c2ccccc2-c2cccc(N(c3ccccc3-c3ccccc3-c3ccccc3)c3ccccc3-c3cccc4sc5ccccc5c34)c21]

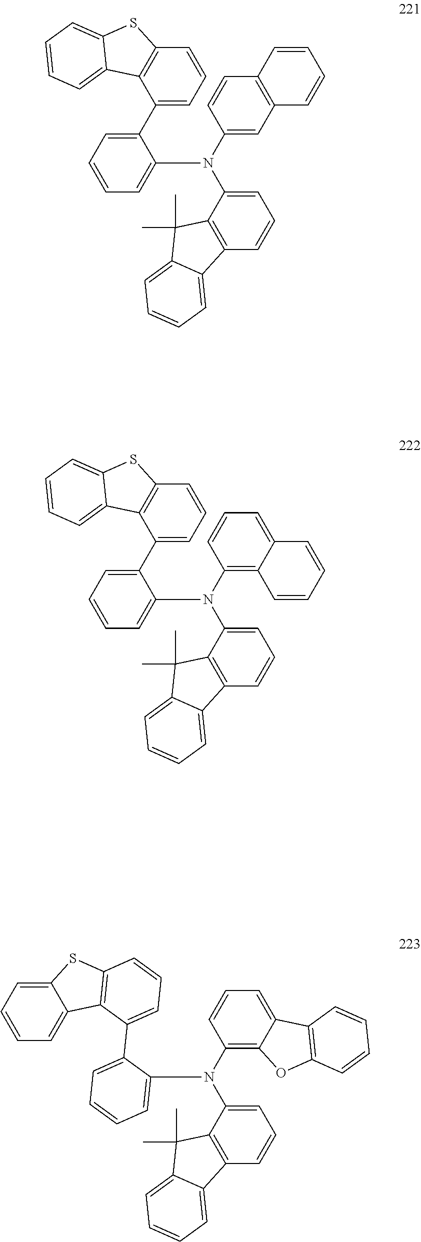 [SMILES: CC1(C)c2ccccc2-c2cccc(N(c3ccc4ccccc4c3)c3ccccc3-c3cccc4sc5ccccc5c34)c21 ; CC1(C)c2ccccc2-c2cccc(N(c3ccccc3-c3cccc4sc5ccccc5c34)c3cccc4ccccc34)c21 ; CC1(C)c2ccccc2-c2cccc(N(c3ccccc3-c3cccc4sc5ccccc5c34)c3cccc4c3oc3ccccc34)c21]

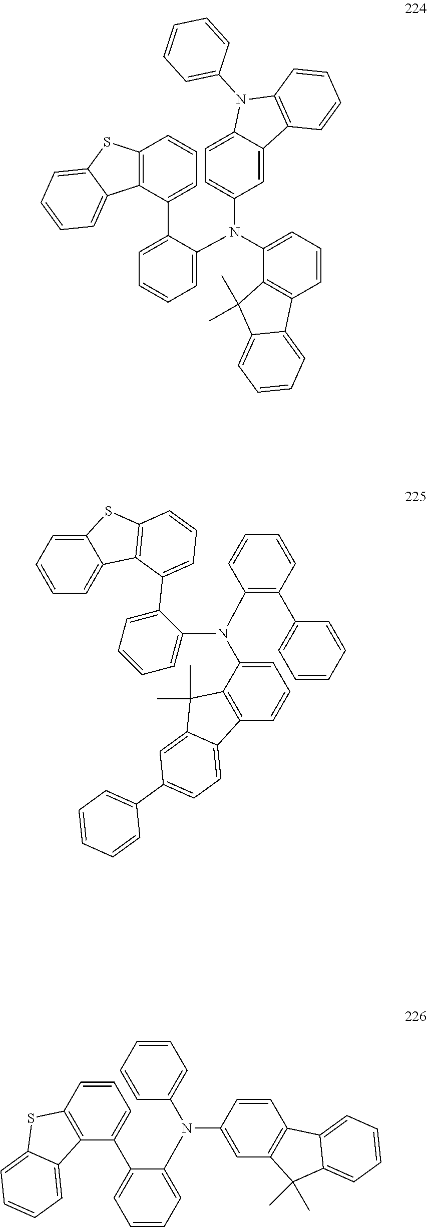 [SMILES: CC1(C)c2ccccc2-c2cccc(N(c3ccc4c(c3)c3ccccc3n4-c3ccccc3)c3ccccc3-c3cccc4sc5ccccc5c34)c21 ; CC1(C)c2cc(-c3ccccc3)ccc2-c2cccc(N(c3ccccc3-c3ccccc3)c3ccccc3-c3cccc4sc5ccccc5c34)c21 ; CC1(C)c2ccccc2-c2ccc(N(c3ccccc3)c3ccccc3-c3cccc4sc5ccccc5c34)cc21]

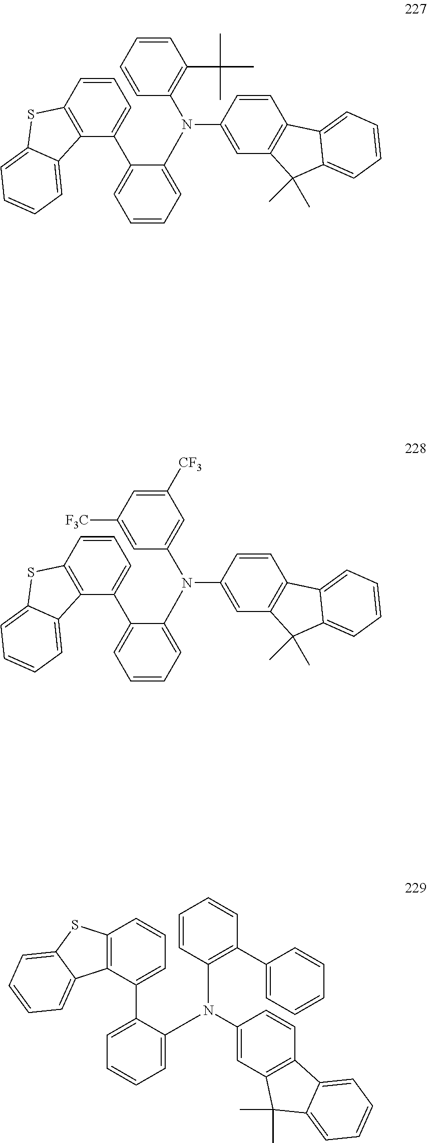 [SMILES: CC(C)(C)c1ccccc1N(c1ccc2c(c1)C(C)(C)c1ccccc1-2)c1ccccc1-c1cccc2sc3ccccc3c12 ; CC1(C)c2ccccc2-c2ccc(N(c3cc(C(F)(F)F)cc(C(F)(F)F)c3)c3ccccc3-c3cccc4sc5ccccc5c34)cc21 ; CC1(C)c2ccccc2-c2ccc(N(c3ccccc3-c3ccccc3)c3ccccc3-c3cccc4sc5ccccc5c34)cc21]

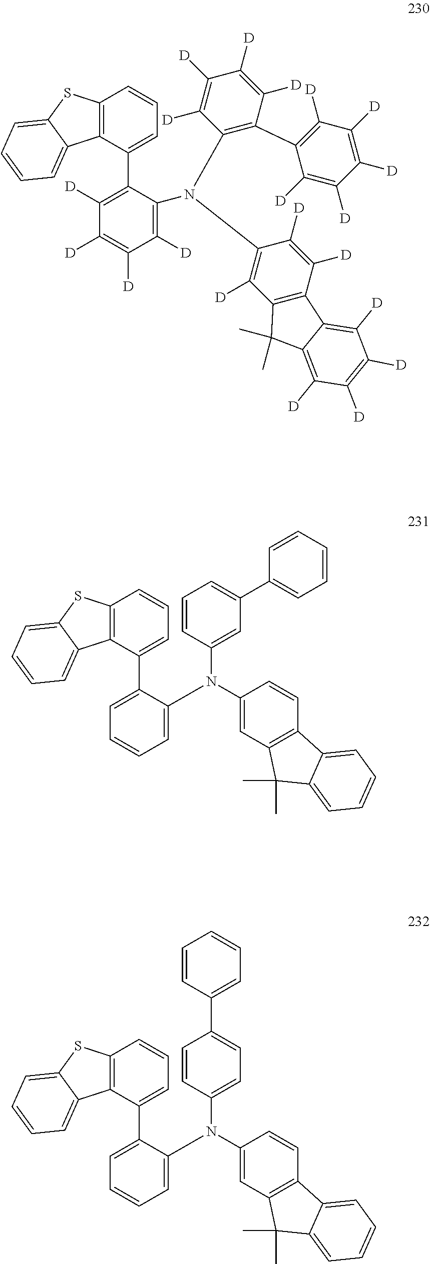 [SMILES: [2H]c1c([2H])c([2H])c(-c2c([2H])c([2H])c([2H])c([2H])c2N(c2c([2H])c([2H])c3c(c2[2H])C(C)(C)c2c([2H])c([2H])c([2H])c([2H])c2-3)c2c([2H])c([2H])c([2H])c([2H])c2-c2cccc3sc4ccccc4c23)c([2H])c1[2H] ; CC1(C)c2ccccc2-c2ccc(N(c3cccc(-c4ccccc4)c3)c3ccccc3-c3cccc4sc5ccccc5c34)cc21 ; CC1(C)c2ccccc2-c2ccc(N(c3ccc(-c4ccccc4)cc3)c3ccccc3-c3cccc4sc5ccccc5c34)cc21]

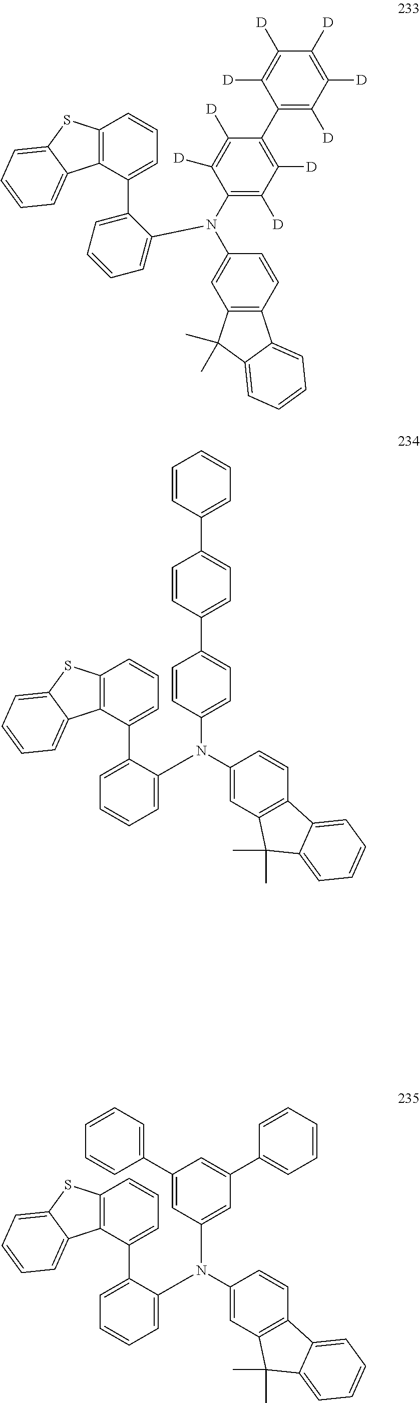 [SMILES: [2H]c1c([2H])c([2H])c(-c2c([2H])c([2H])c(N(c3ccc4c(c3)C(C)(C)c3ccccc3-4)c3ccccc3-c3cccc4sc5ccccc5c34)c([2H])c2[2H])c([2H])c1[2H] ; CC1(C)c2ccccc2-c2ccc(N(c3ccc(-c4ccc(-c5ccccc5)cc4)cc3)c3ccccc3-c3cccc4sc5ccccc5c34)cc21 ; CC1(C)c2ccccc2-c2ccc(N(c3cc(-c4ccccc4)cc(-c4ccccc4)c3)c3ccccc3-c3cccc4sc5ccccc5c34)cc21]

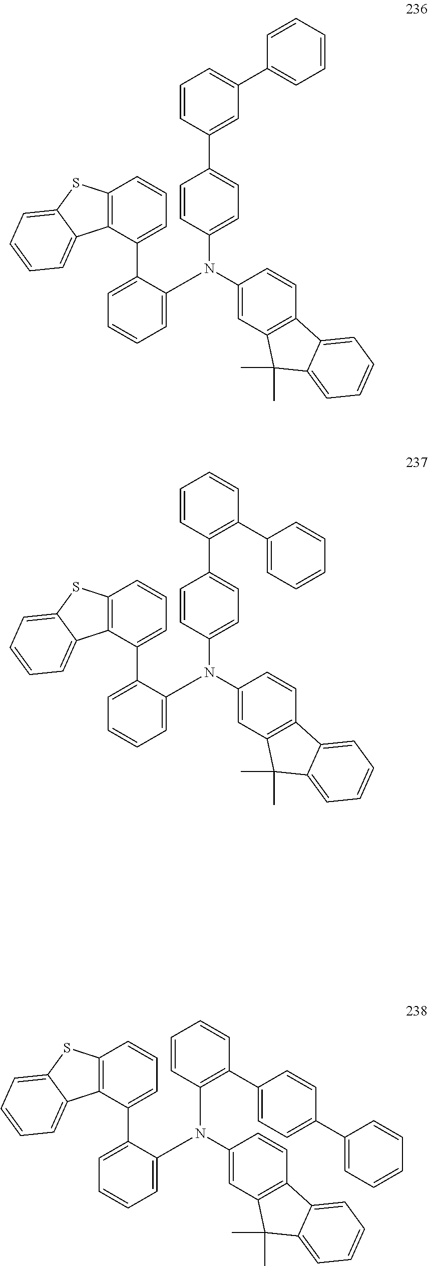 [SMILES: CC1(C)c2ccccc2-c2ccc(N(c3ccc(-c4cccc(-c5ccccc5)c4)cc3)c3ccccc3-c3cccc4sc5ccccc5c34)cc21 ; CC1(C)c2ccccc2-c2ccc(N(c3ccc(-c4ccccc4-c4ccccc4)cc3)c3ccccc3-c3cccc4sc5ccccc5c34)cc21 ; CC1(C)c2ccccc2-c2ccc(N(c3ccccc3-c3ccc(-c4ccccc4)cc3)c3ccccc3-c3cccc4sc5ccccc5c34)cc21]

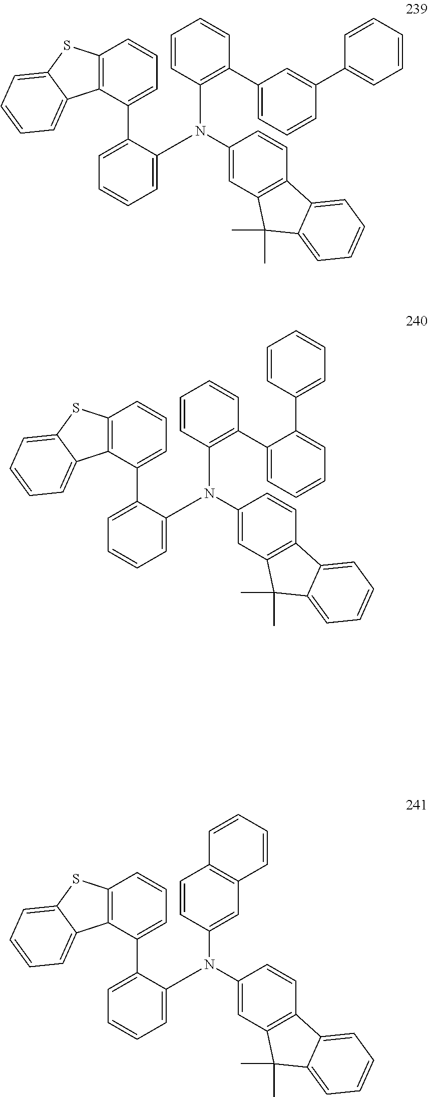 [SMILES: CC1(C)c2ccccc2-c2ccc(N(c3ccccc3-c3cccc(-c4ccccc4)c3)c3ccccc3-c3cccc4sc5ccccc5c34)cc21 ; CC1(C)c2ccccc2-c2ccc(N(c3ccccc3-c3ccccc3-c3ccccc3)c3ccccc3-c3cccc4sc5ccccc5c34)cc21 ; CC1(C)c2ccccc2-c2ccc(N(c3ccc4ccccc4c3)c3ccccc3-c3cccc4sc5ccccc5c34)cc21]

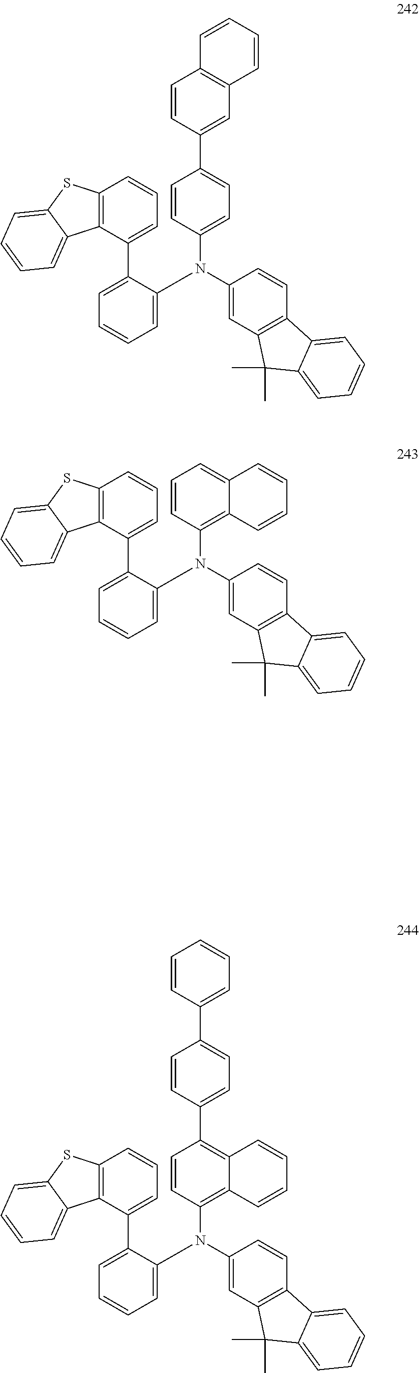 [SMILES: CC1(C)c2ccccc2-c2ccc(N(c3ccc(-c4ccc5ccccc5c4)cc3)c3ccccc3-c3cccc4sc5ccccc5c34)cc21 ; CC1(C)c2ccccc2-c2ccc(N(c3ccccc3-c3cccc4sc5ccccc5c34)c3cccc4ccccc34)cc21 ; CC1(C)c2ccccc2-c2ccc(N(c3ccccc3-c3cccc4sc5ccccc5c34)c3ccc(-c4ccc(-c5ccccc5)cc4)c4ccccc34)cc21]

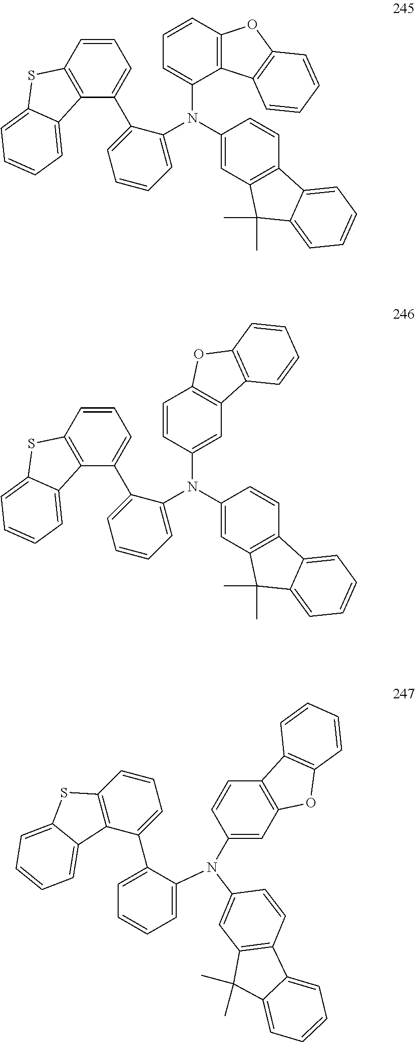 [SMILES: CC1(C)c2ccccc2-c2ccc(N(c3ccccc3-c3cccc4sc5ccccc5c34)c3cccc4oc5ccccc5c34)cc21 ; CC1(C)c2ccccc2-c2ccc(N(c3ccc4oc5ccccc5c4c3)c3ccccc3-c3cccc4sc5ccccc5c34)cc21 ; CC1(C)c2ccccc2-c2ccc(N(c3ccc4c(c3)oc3ccccc34)c3ccccc3-c3cccc4sc5ccccc5c34)cc21]

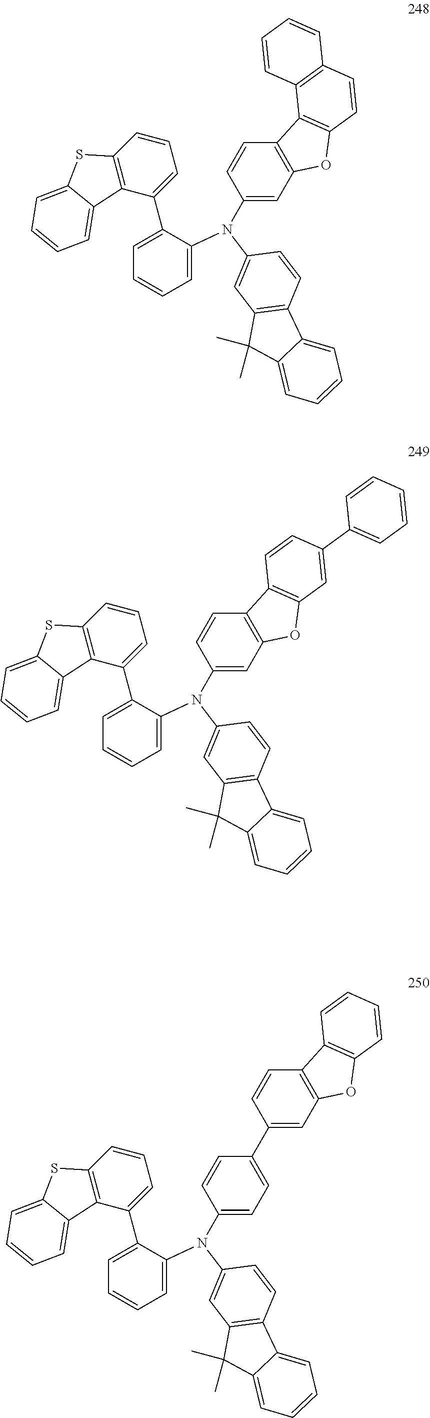 [SMILES: CC1(C)c2ccccc2-c2ccc(N(c3ccc4c(c3)oc3ccc5ccccc5c34)c3ccccc3-c3cccc4sc5ccccc5c34)cc21 ; CC1(C)c2ccccc2-c2ccc(N(c3ccc4c(c3)oc3cc(-c5ccccc5)ccc34)c3ccccc3-c3cccc4sc5ccccc5c34)cc21 ; CC1(C)c2ccccc2-c2ccc(N(c3ccc(-c4ccc5c(c4)oc4ccccc45)cc3)c3ccccc3-c3cccc4sc5ccccc5c34)cc21]

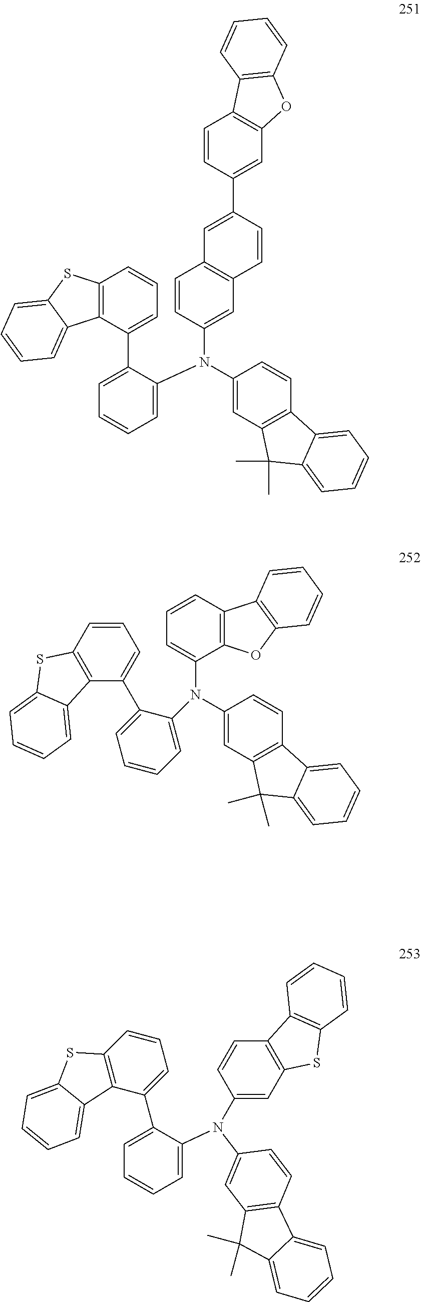 [SMILES: CC1(C)c2ccccc2-c2ccc(N(c3ccc4cc(-c5ccc6c(c5)oc5ccccc56)ccc4c3)c3ccccc3-c3cccc4sc5ccccc5c34)cc21 ; CC1(C)c2ccccc2-c2ccc(N(c3ccccc3-c3cccc4sc5ccccc5c34)c3cccc4c3oc3ccccc34)cc21 ; CC1(C)c2ccccc2-c2ccc(N(c3ccc4c(c3)sc3ccccc34)c3ccccc3-c3cccc4sc5ccccc5c34)cc21]

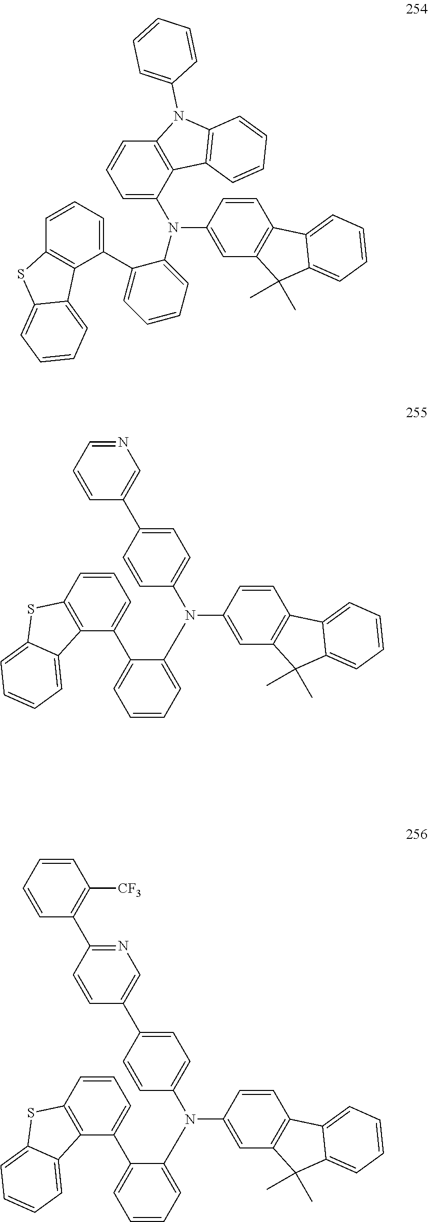 [SMILES: CC1(C)c2ccccc2-c2ccc(N(c3ccccc3-c3cccc4sc5ccccc5c34)c3cccc4c3c3ccccc3n4-c3ccccc3)cc21 ; CC1(C)c2ccccc2-c2ccc(N(c3ccc(-c4cccnc4)cc3)c3ccccc3-c3cccc4sc5ccccc5c34)cc21 ; CC1(C)c2ccccc2-c2ccc(N(c3ccc(-c4ccc(-c5ccccc5C(F)(F)F)nc4)cc3)c3ccccc3-c3cccc4sc5ccccc5c34)cc21]

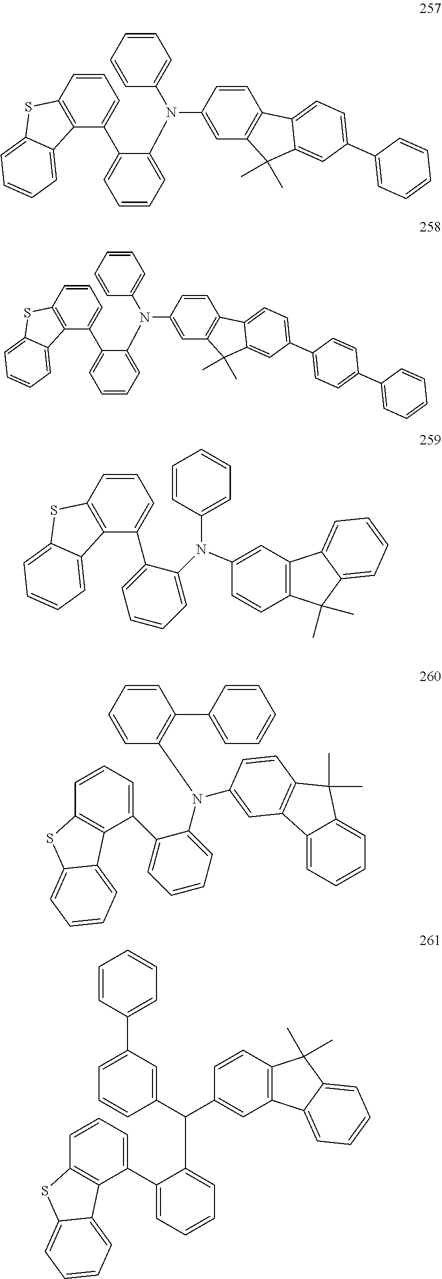

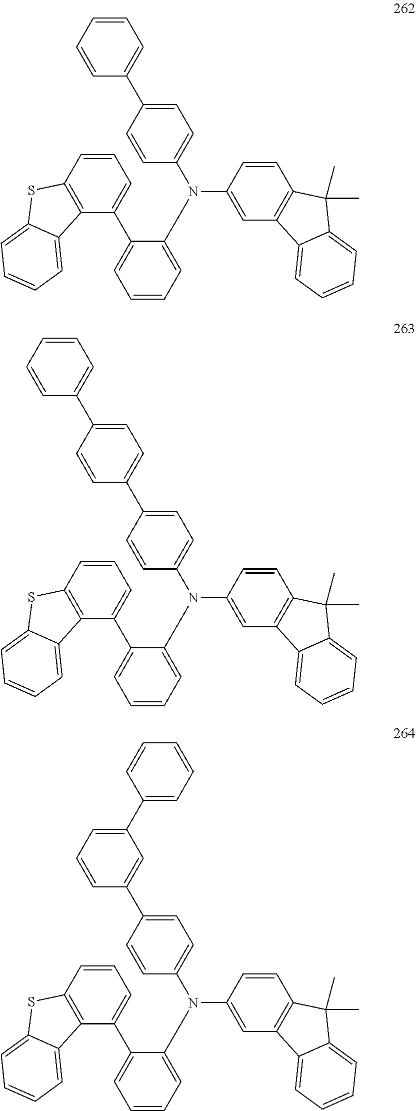

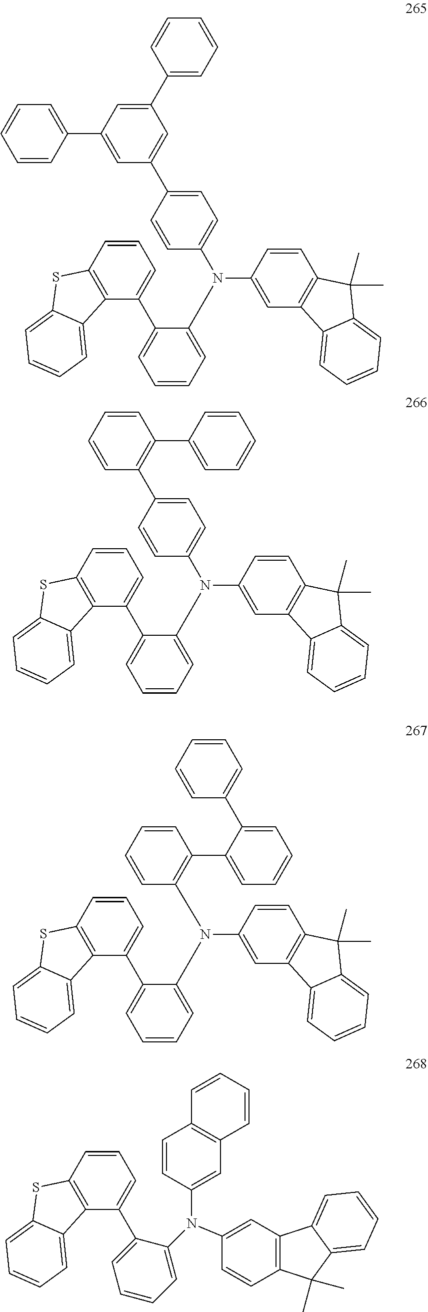

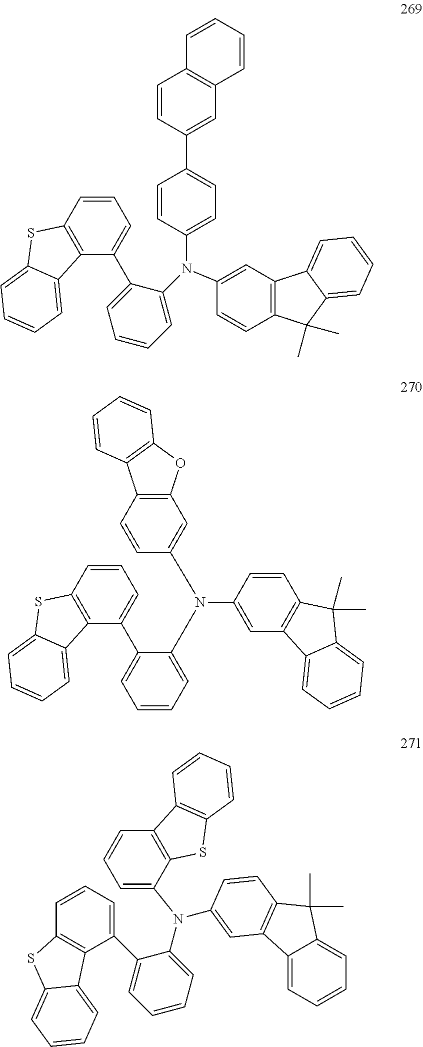 [SMILES: CC1(C)c2ccccc2-c2cc(N(c3ccc(-c4ccc5ccccc5c4)cc3)c3ccccc3-c3cccc4sc5ccccc5c34)ccc21 ; CC1(C)c2ccccc2-c2cc(N(c3ccc4c(c3)oc3ccccc34)c3ccccc3-c3cccc4sc5ccccc5c34)ccc21 ; CC1(C)c2ccccc2-c2cc(N(c3ccccc3-c3cccc4sc5ccccc5c34)c3cccc4c3sc3ccccc34)ccc21]

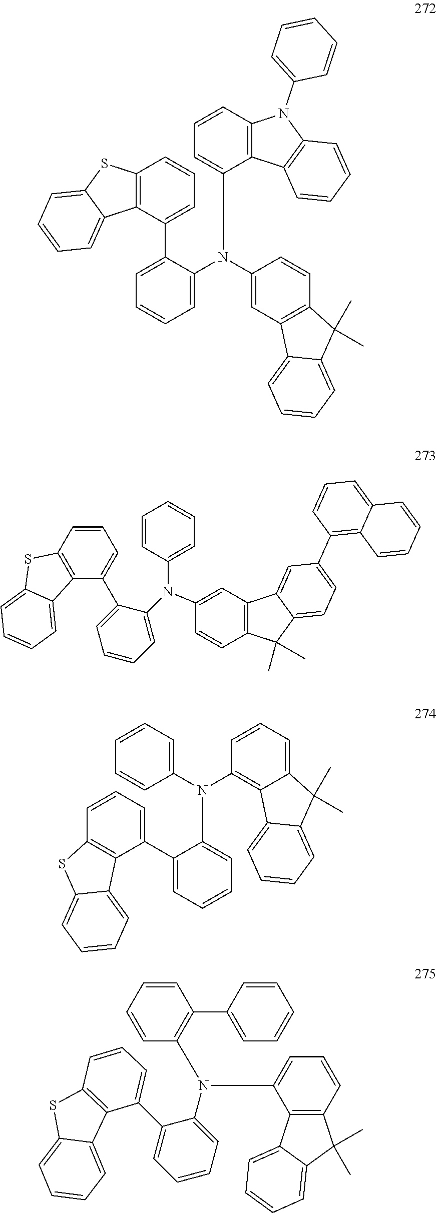

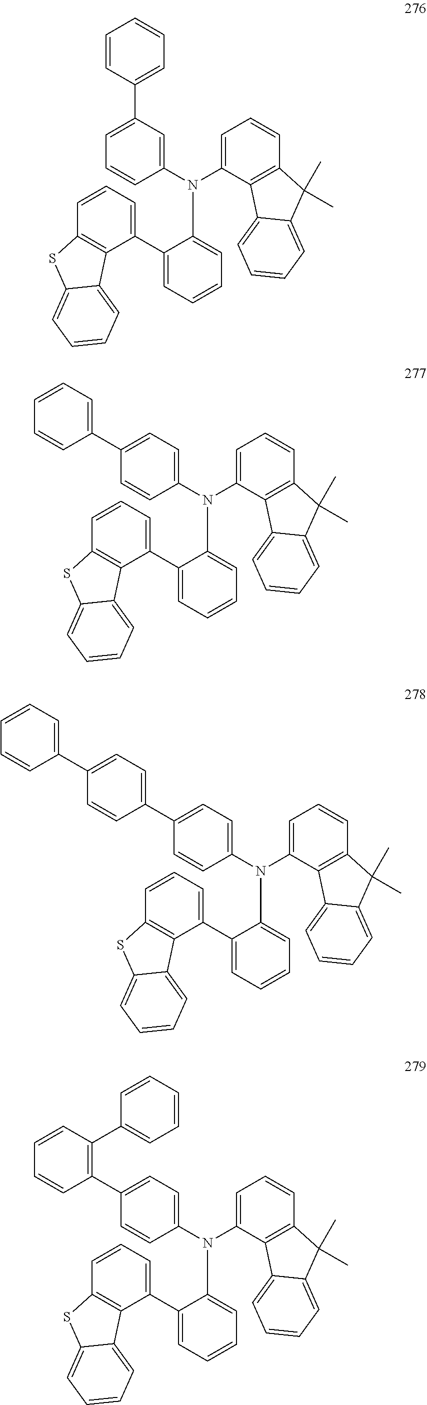

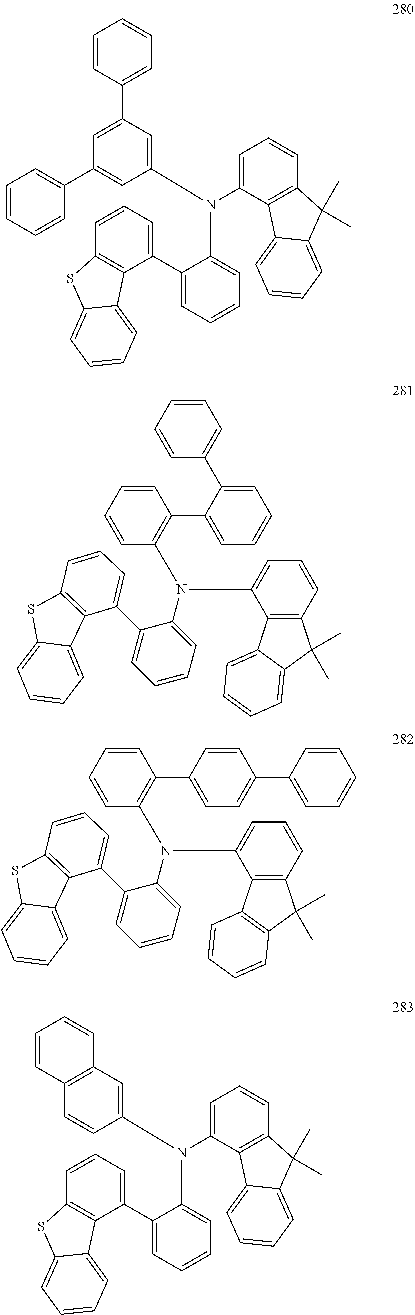

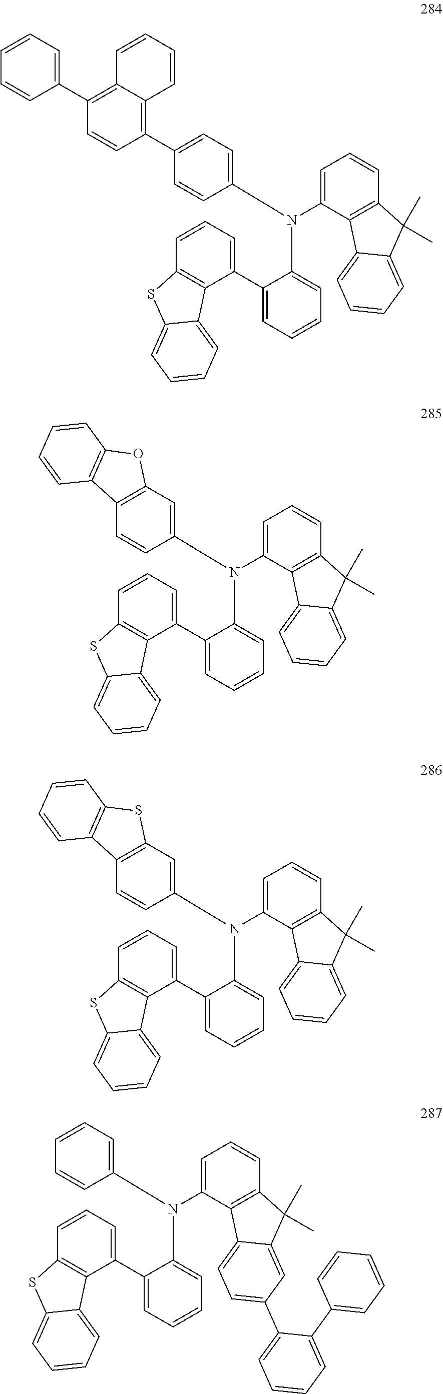

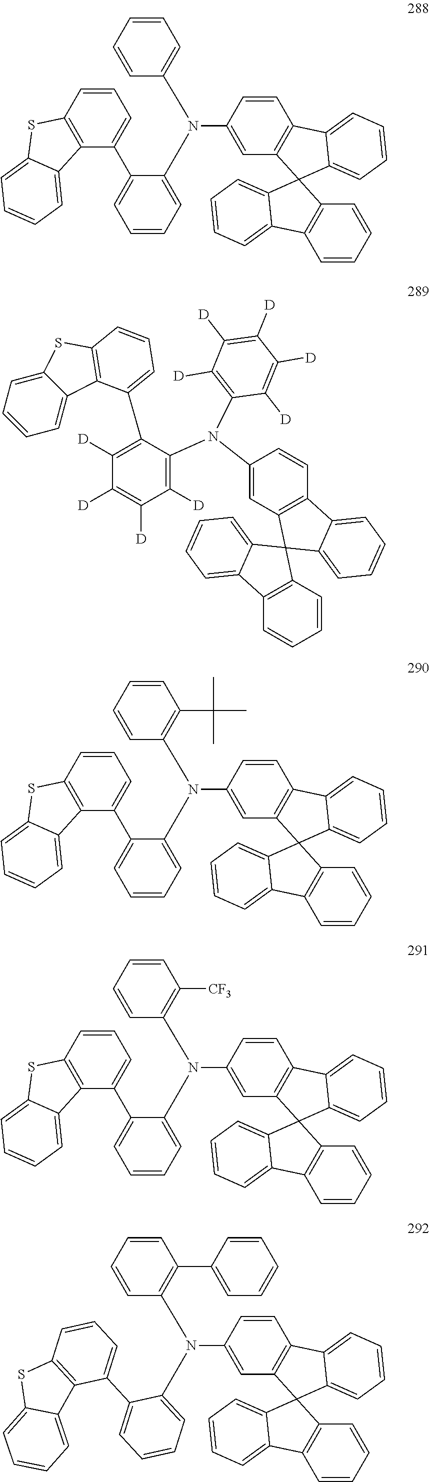

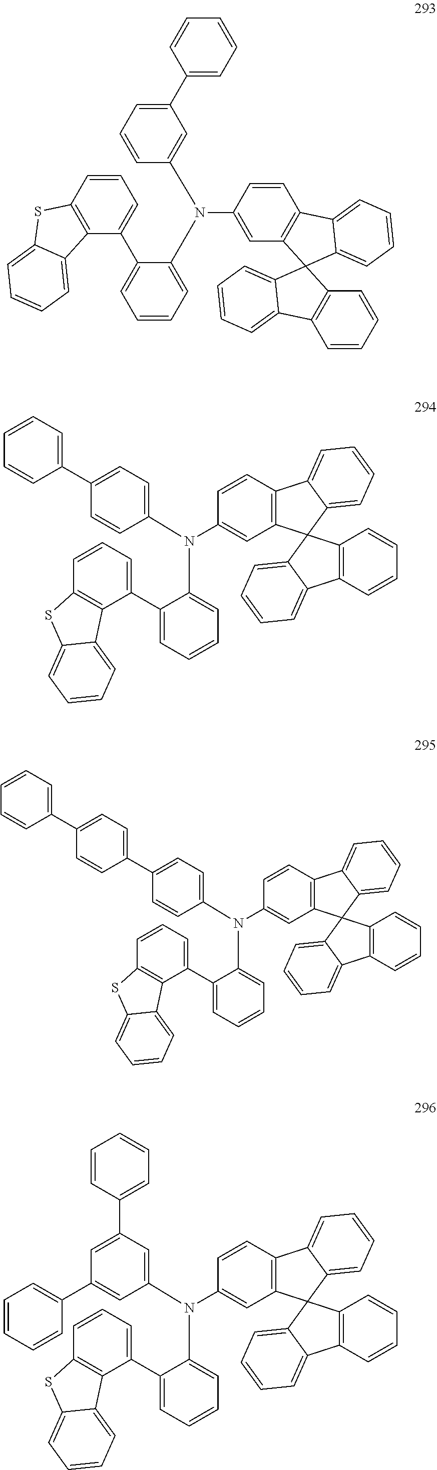 [SMILES: c1ccc(-c2cccc(N(c3ccc4c(c3)C3(c5ccccc5-c5ccccc53)c3ccccc3-4)c3ccccc3-c3cccc4sc5ccccc5c34)c2)cc1 ; c1ccc(-c2ccc(N(c3ccc4c(c3)C3(c5ccccc5-c5ccccc53)c3ccccc3-4)c3ccccc3-c3cccc4sc5ccccc5c34)cc2)cc1 ; c1ccc(-c2ccc(-c3ccc(N(c4ccc5c(c4)C4(c6ccccc6-c6ccccc64)c4ccccc4-5)c4ccccc4-c4cccc5sc6ccccc6c45)cc3)cc2)cc1 ; c1ccc(-c2cc(-c3ccccc3)cc(N(c3ccc4c(c3)C3(c5ccccc5-c5ccccc53)c3ccccc3-4)c3ccccc3-c3cccc4sc5ccccc5c34)c2)cc1]

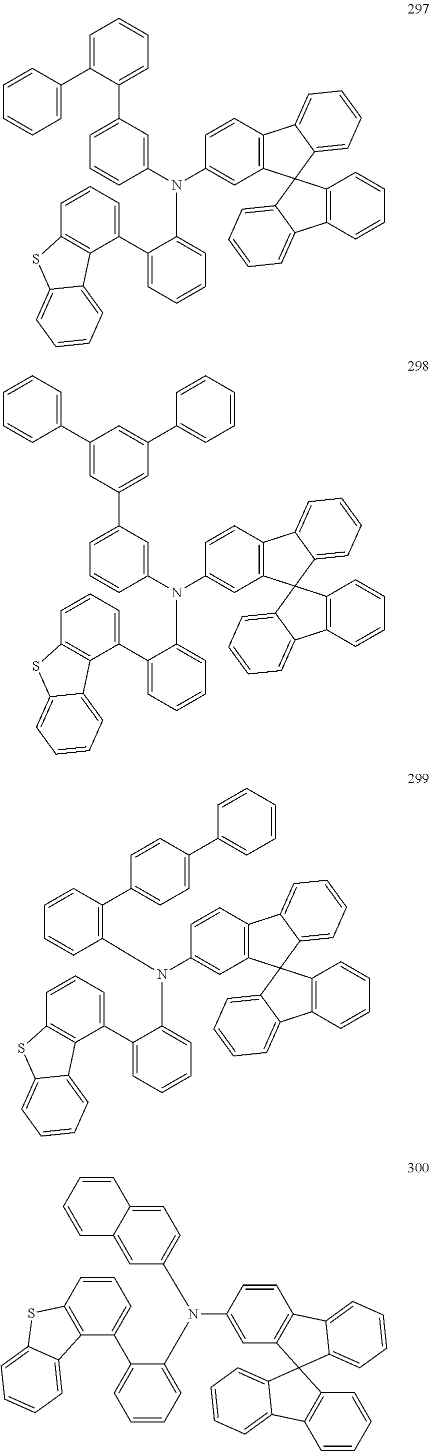

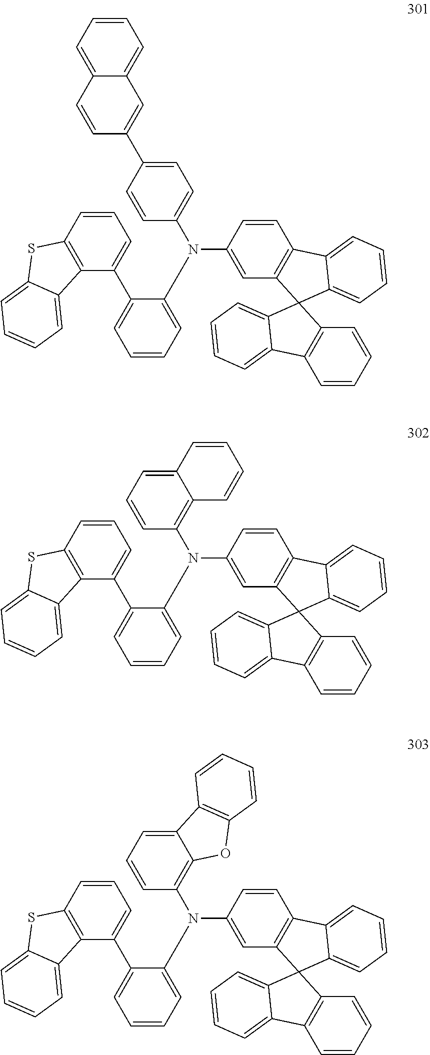 [SMILES: c1ccc(N(c2ccc(-c3ccc4ccccc4c3)cc2)c2ccc3c(c2)C2(c4ccccc4-c4ccccc42)c2ccccc2-3)c(-c2cccc3sc4ccccc4c23)c1 ; c1ccc(N(c2ccc3c(c2)C2(c4ccccc4-c4ccccc42)c2ccccc2-3)c2cccc3ccccc23)c(-c2cccc3sc4ccccc4c23)c1 ; c1ccc(N(c2ccc3c(c2)C2(c4ccccc4-c4ccccc42)c2ccccc2-3)c2cccc3c2oc2ccccc23)c(-c2cccc3sc4ccccc4c23)c1]

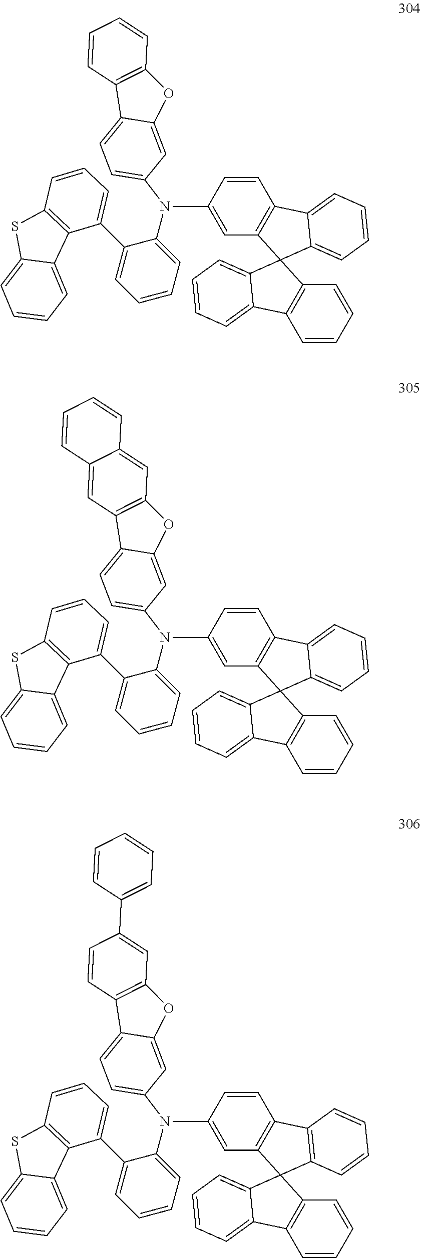 [SMILES: c1ccc(N(c2ccc3c(c2)C2(c4ccccc4-c4ccccc42)c2ccccc2-3)c2ccc3c(c2)oc2ccccc23)c(-c2cccc3sc4ccccc4c23)c1 ; c1ccc(N(c2ccc3c(c2)C2(c4ccccc4-c4ccccc42)c2ccccc2-3)c2ccc3c(c2)oc2cc4ccccc4cc23)c(-c2cccc3sc4ccccc4c23)c1 ; c1ccc(-c2ccc3c(c2)oc2cc(N(c4ccc5c(c4)C4(c6ccccc6-c6ccccc64)c4ccccc4-5)c4ccccc4-c4cccc5sc6ccccc6c45)ccc23)cc1]

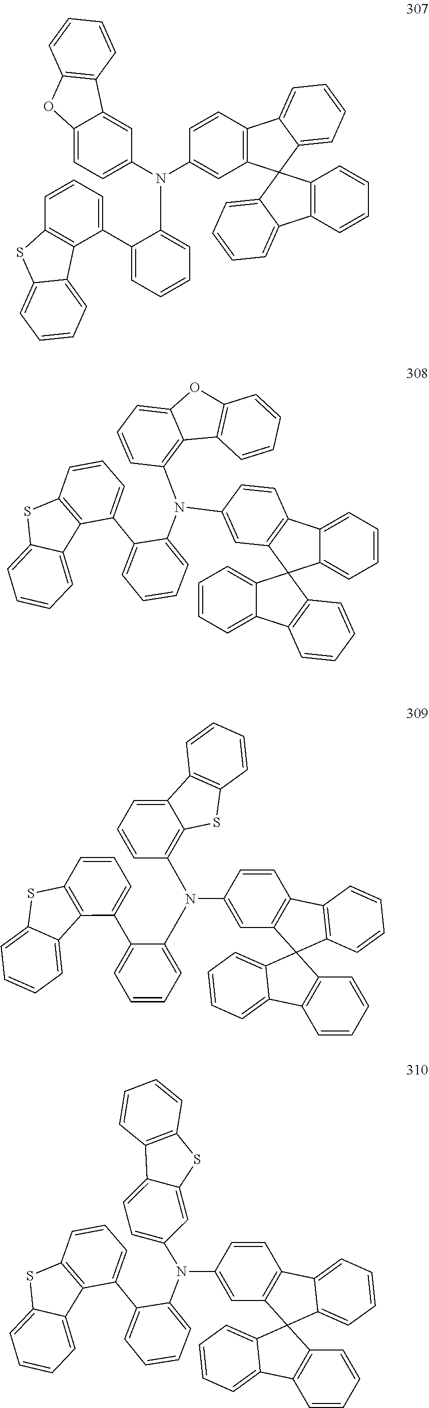 [SMILES: c1ccc(N(c2ccc3c(c2)C2(c4ccccc4-c4ccccc42)c2ccccc2-3)c2ccc3oc4ccccc4c3c2)c(-c2cccc3sc4ccccc4c23)c1 ; c1ccc(N(c2ccc3c(c2)C2(c4ccccc4-c4ccccc42)c2ccccc2-3)c2cccc3oc4ccccc4c23)c(-c2cccc3sc4ccccc4c23)c1 ; c1ccc(N(c2ccc3c(c2)C2(c4ccccc4-c4ccccc42)c2ccccc2-3)c2cccc3c2sc2ccccc23)c(-c2cccc3sc4ccccc4c23)c1 ; c1ccc(N(c2ccc3c(c2)C2(c4ccccc4-c4ccccc42)c2ccccc2-3)c2ccc3c(c2)sc2ccccc23)c(-c2cccc3sc4ccccc4c23)c1]

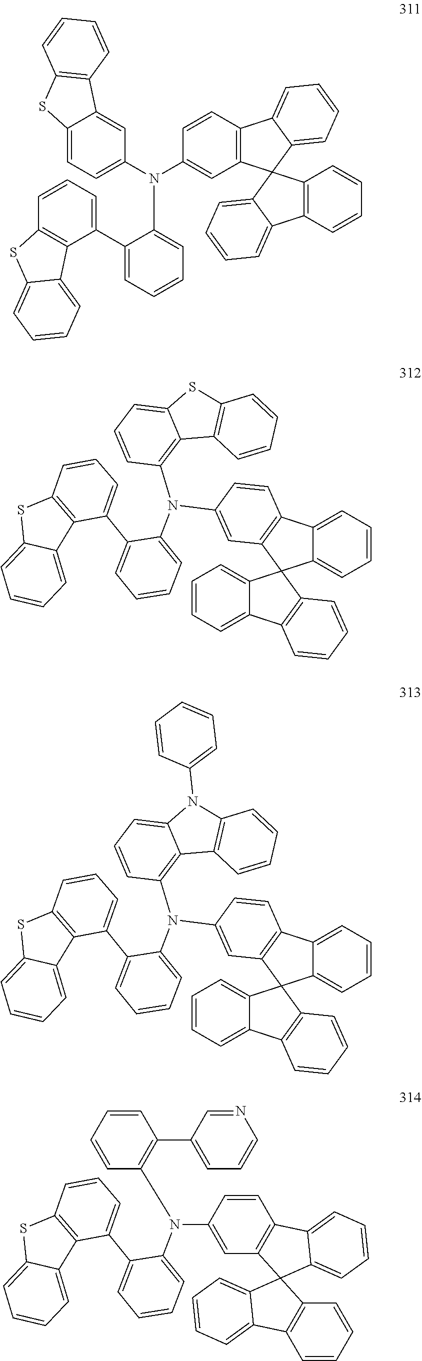 [SMILES: c1ccc(N(c2ccc3c(c2)C2(c4ccccc4-c4ccccc42)c2ccccc2-3)c2ccc3sc4ccccc4c3c2)c(-c2cccc3sc4ccccc4c23)c1 ; c1ccc(N(c2ccc3c(c2)C2(c4ccccc4-c4ccccc42)c2ccccc2-3)c2cccc3sc4ccccc4c23)c(-c2cccc3sc4ccccc4c23)c1 ; c1ccc(-n2c3ccccc3c3c(N(c4ccc5c(c4)C4(c6ccccc6-c6ccccc64)c4ccccc4-5)c4ccccc4-c4cccc5sc6ccccc6c45)cccc32)cc1 ; c1cncc(-c2ccccc2N(c2ccc3c(c2)C2(c4ccccc4-c4ccccc42)c2ccccc2-3)c2ccccc2-c2cccc3sc4ccccc4c23)c1]

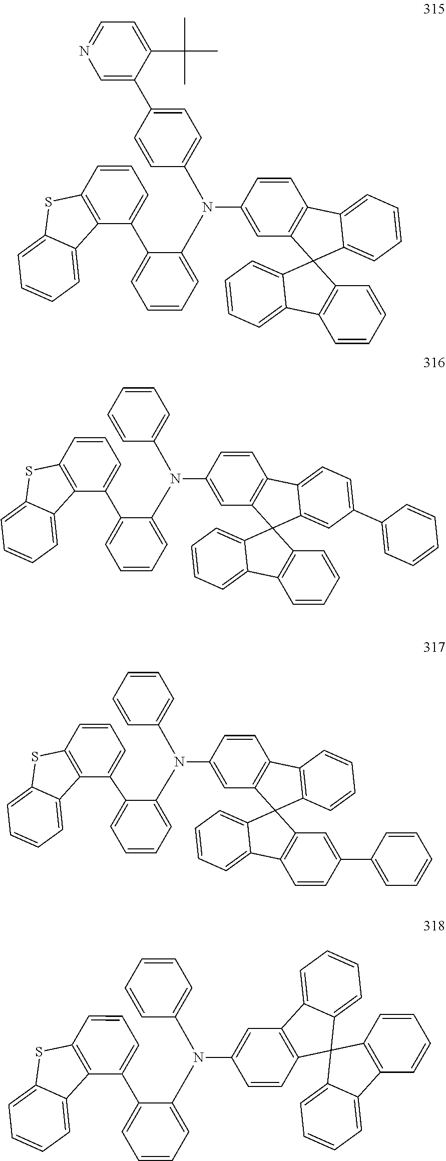

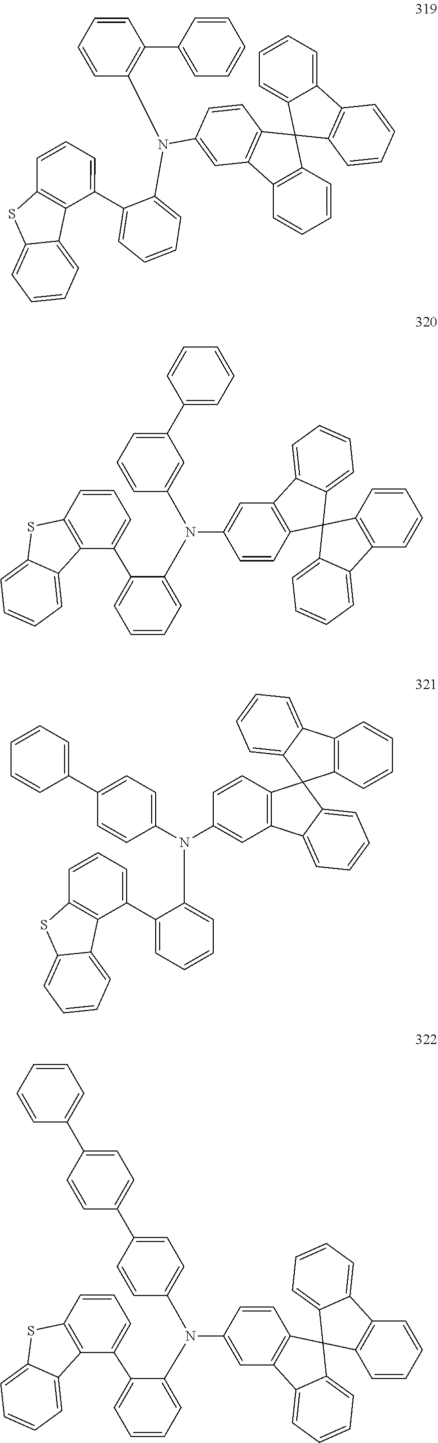 [SMILES: c1ccc(-c2ccccc2N(c2ccc3c(c2)-c2ccccc2C32c3ccccc3-c3ccccc32)c2ccccc2-c2cccc3sc4ccccc4c23)cc1 ; c1ccc(-c2cccc(N(c3ccc4c(c3)-c3ccccc3C43c4ccccc4-c4ccccc43)c3ccccc3-c3cccc4sc5ccccc5c34)c2)cc1 ; c1ccc(-c2ccc(N(c3ccc4c(c3)-c3ccccc3C43c4ccccc4-c4ccccc43)c3ccccc3-c3cccc4sc5ccccc5c34)cc2)cc1 ; c1ccc(-c2ccc(-c3ccc(N(c4ccc5c(c4)-c4ccccc4C54c5ccccc5-c5ccccc54)c4ccccc4-c4cccc5sc6ccccc6c45)cc3)cc2)cc1]

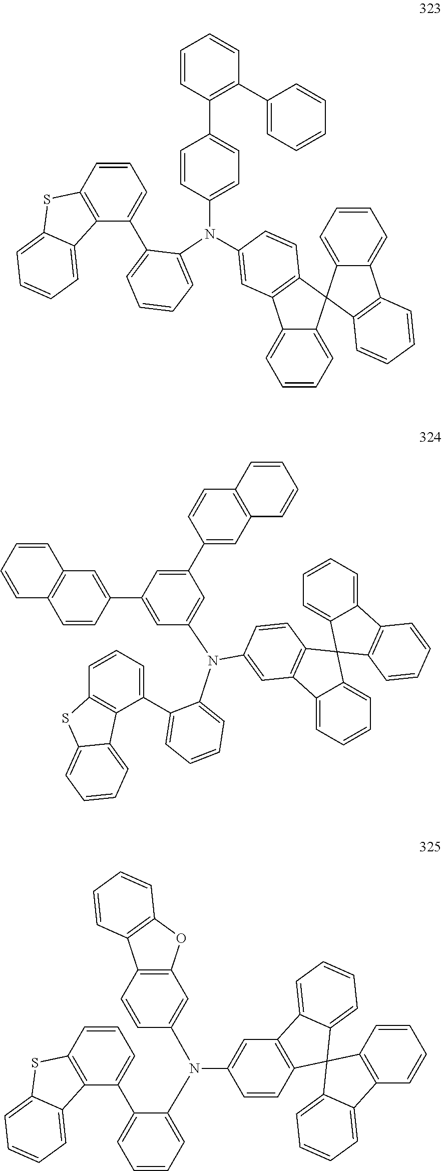 [SMILES: c1ccc(-c2ccccc2-c2ccc(N(c3ccc4c(c3)-c3ccccc3C43c4ccccc4-c4ccccc43)c3ccccc3-c3cccc4sc5ccccc5c34)cc2)cc1 ; c1ccc(N(c2cc(-c3ccc4ccccc4c3)cc(-c3ccc4ccccc4c3)c2)c2ccc3c(c2)-c2ccccc2C32c3ccccc3-c3ccccc32)c(-c2cccc3sc4ccccc4c23)c1 ; c1ccc(N(c2ccc3c(c2)-c2ccccc2C32c3ccccc3-c3ccccc32)c2ccc3c(c2)oc2ccccc23)c(-c2cccc3sc4ccccc4c23)c1]

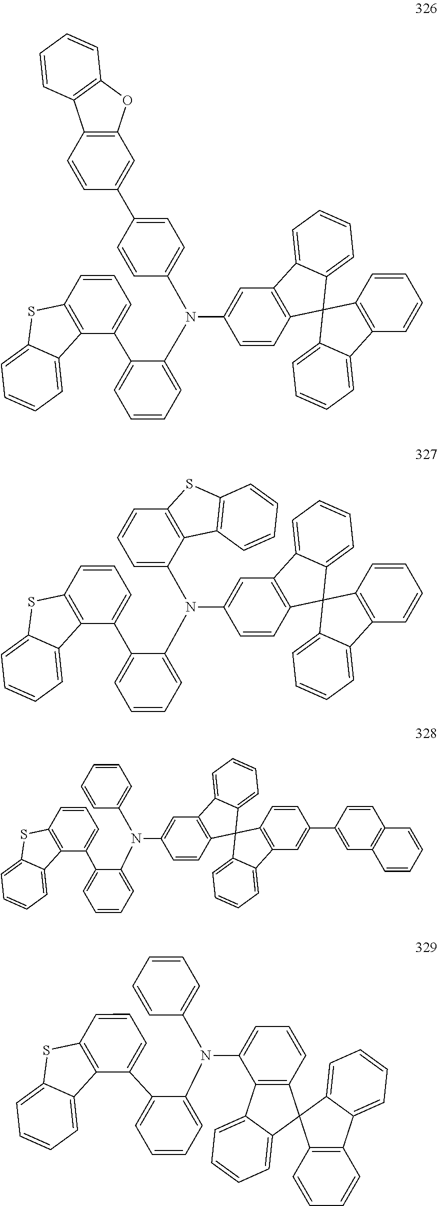 [SMILES: c1ccc(N(c2ccc(-c3ccc4c(c3)oc3ccccc34)cc2)c2ccc3c(c2)-c2ccccc2C32c3ccccc3-c3ccccc32)c(-c2cccc3sc4ccccc4c23)c1 ; c1ccc(N(c2ccc3c(c2)-c2ccccc2C32c3ccccc3-c3ccccc32)c2cccc3sc4ccccc4c23)c(-c2cccc3sc4ccccc4c23)c1 ; c1ccc(N(c2ccc3c(c2)-c2ccccc2C32c3ccccc3-c3cc(-c4ccc5ccccc5c4)ccc32)c2ccccc2-c2cccc3sc4ccccc4c23)cc1 ; c1ccc(N(c2ccccc2-c2cccc3sc4ccccc4c23)c2cccc3c2-c2ccccc2C32c3ccccc3-c3ccccc32)cc1]

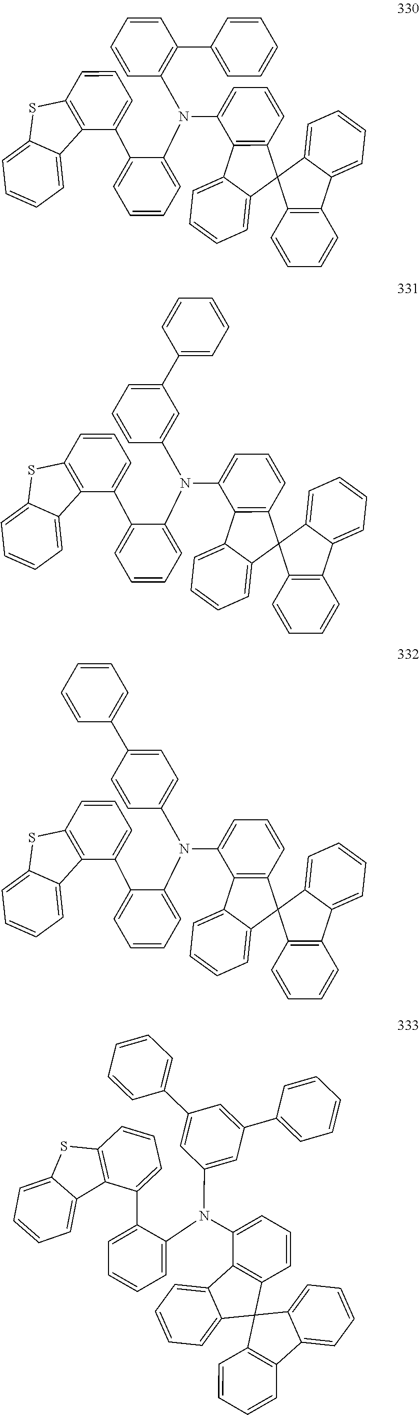 [SMILES: c1ccc(-c2ccccc2N(c2ccccc2-c2cccc3sc4ccccc4c23)c2cccc3c2-c2ccccc2C32c3ccccc3-c3ccccc32)cc1 ; c1ccc(-c2cccc(N(c3ccccc3-c3cccc4sc5ccccc5c34)c3cccc4c3-c3ccccc3C43c4ccccc4-c4ccccc43)c2)cc1 ; c1ccc(-c2ccc(N(c3ccccc3-c3cccc4sc5ccccc5c34)c3cccc4c3-c3ccccc3C43c4ccccc4-c4ccccc43)cc2)cc1 ; c1ccc(-c2cc(-c3ccccc3)cc(N(c3ccccc3-c3cccc4sc5ccccc5c34)c3cccc4c3-c3ccccc3C43c4ccccc4-c4ccccc43)c2)cc1]

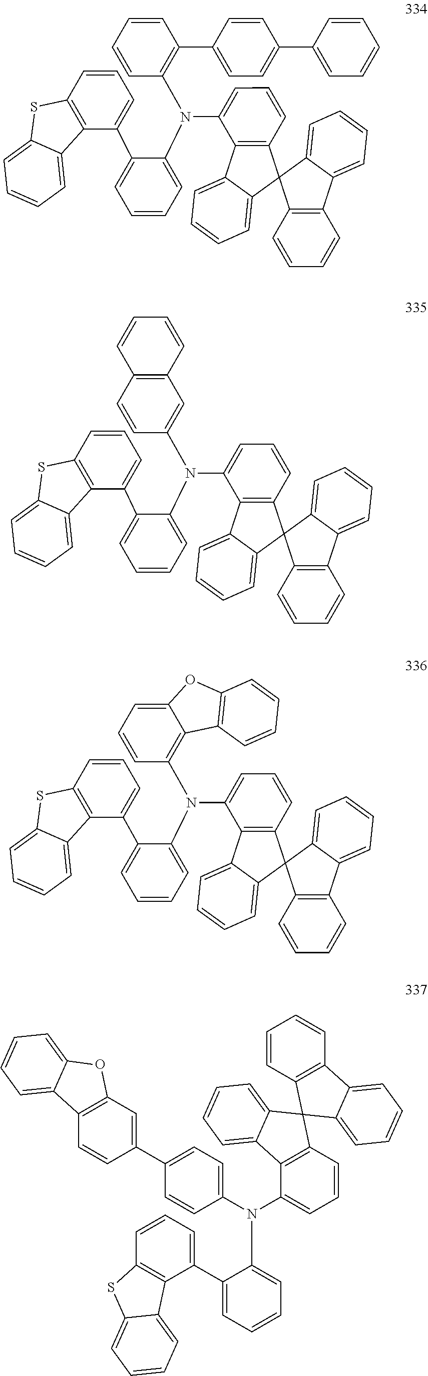 [SMILES: c1ccc(-c2ccc(-c3ccccc3N(c3ccccc3-c3cccc4sc5ccccc5c34)c3cccc4c3-c3ccccc3C43c4ccccc4-c4ccccc43)cc2)cc1 ; c1ccc(N(c2ccc3ccccc3c2)c2cccc3c2-c2ccccc2C32c3ccccc3-c3ccccc32)c(-c2cccc3sc4ccccc4c23)c1 ; c1ccc(N(c2cccc3c2-c2ccccc2C32c3ccccc3-c3ccccc32)c2cccc3oc4ccccc4c23)c(-c2cccc3sc4ccccc4c23)c1 ; c1ccc(N(c2ccc(-c3ccc4c(c3)oc3ccccc34)cc2)c2cccc3c2-c2ccccc2C32c3ccccc3-c3ccccc32)c(-c2cccc3sc4ccccc4c23)c1]

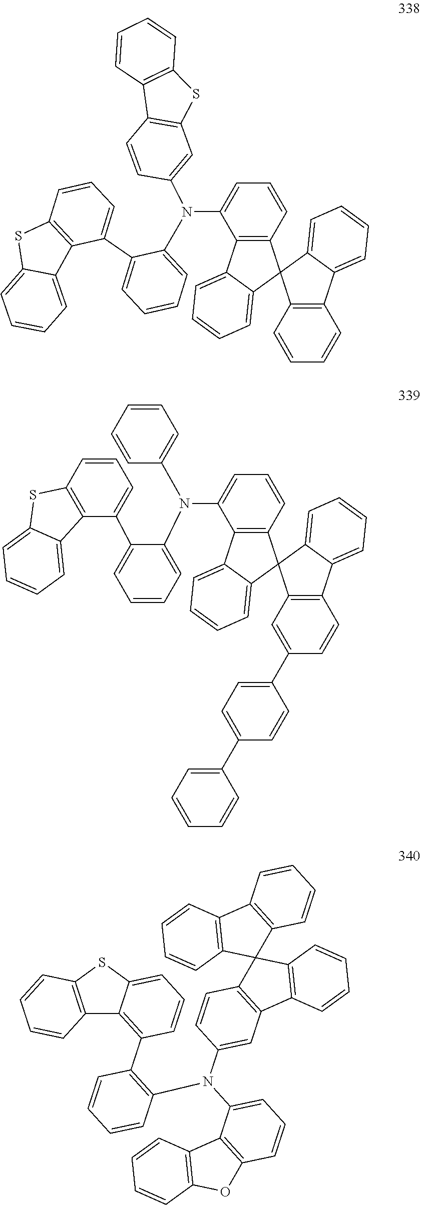 [SMILES: c1ccc(N(c2ccc3c(c2)sc2ccccc23)c2cccc3c2-c2ccccc2C32c3ccccc3-c3ccccc32)c(-c2cccc3sc4ccccc4c23)c1 ; c1ccc(-c2ccc(-c3ccc4c(c3)C3(c5ccccc5-4)c4ccccc4-c4c(N(c5ccccc5)c5ccccc5-c5cccc6sc7ccccc7c56)cccc43)cc2)cc1 ; c1ccc(N(c2ccc3c(c2)-c2ccccc2C32c3ccccc3-c3ccccc32)c2cccc3oc4ccccc4c23)c(-c2cccc3sc4ccccc4c23)c1]

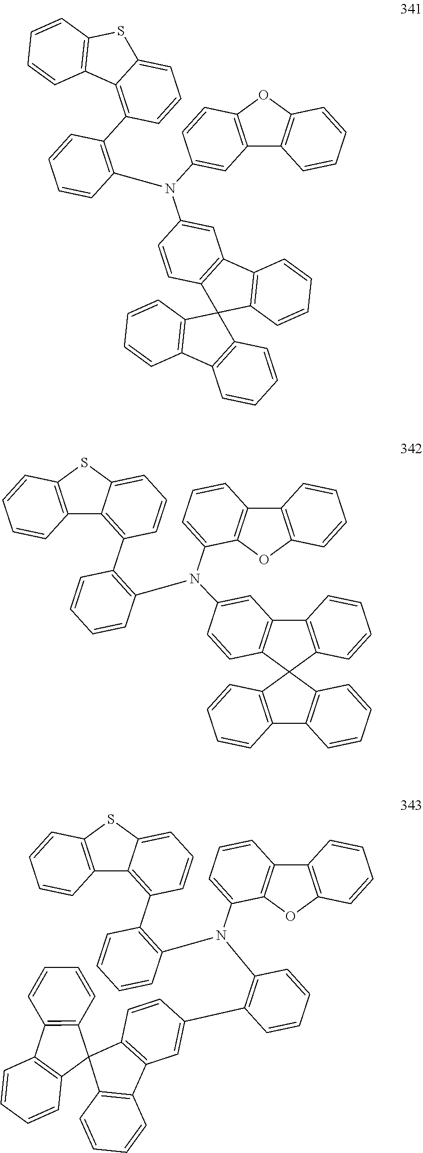 [SMILES: c1ccc(N(c2ccc3c(c2)-c2ccccc2C32c3ccccc3-c3ccccc32)c2ccc3oc4ccccc4c3c2)c(-c2cccc3sc4ccccc4c23)c1 ; c1ccc(N(c2ccc3c(c2)-c2ccccc2C32c3ccccc3-c3ccccc32)c2cccc3c2oc2ccccc23)c(-c2cccc3sc4ccccc4c23)c1 ; c1ccc(N(c2ccccc2-c2cccc3sc4ccccc4c23)c2cccc3c2oc2ccccc23)c(-c2ccc3c(c2)-c2ccccc2C32c3ccccc3-c3ccccc32)c1]

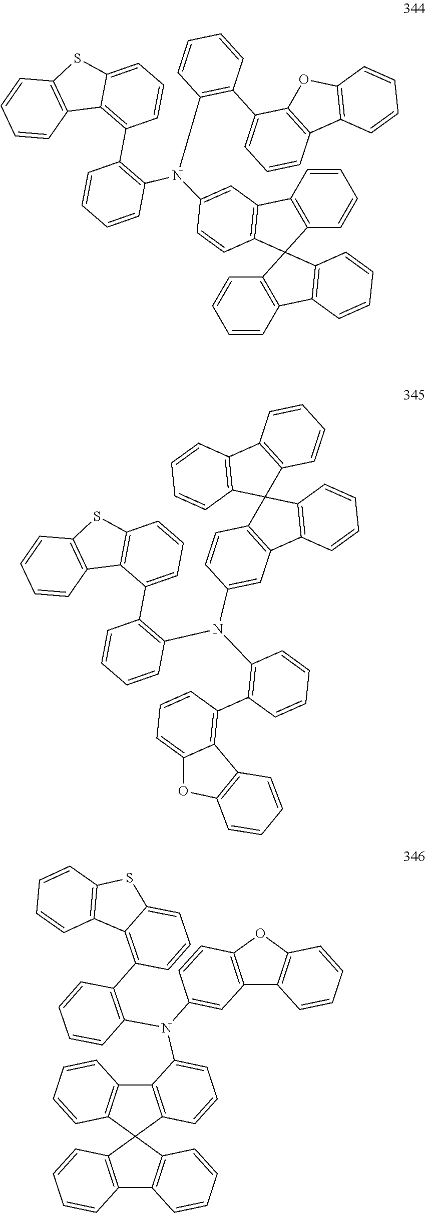 [SMILES: c1ccc(N(c2ccc3c(c2)-c2ccccc2C32c3ccccc3-c3ccccc32)c2ccccc2-c2cccc3sc4ccccc4c23)c(-c2cccc3c2oc2ccccc23)c1 ; c1ccc(N(c2ccc3c(c2)-c2ccccc2C32c3ccccc3-c3ccccc32)c2ccccc2-c2cccc3sc4ccccc4c23)c(-c2cccc3oc4ccccc4c23)c1 ; c1ccc(N(c2ccc3oc4ccccc4c3c2)c2cccc3c2-c2ccccc2C32c3ccccc3-c3ccccc32)c(-c2cccc3sc4ccccc4c23)c1]

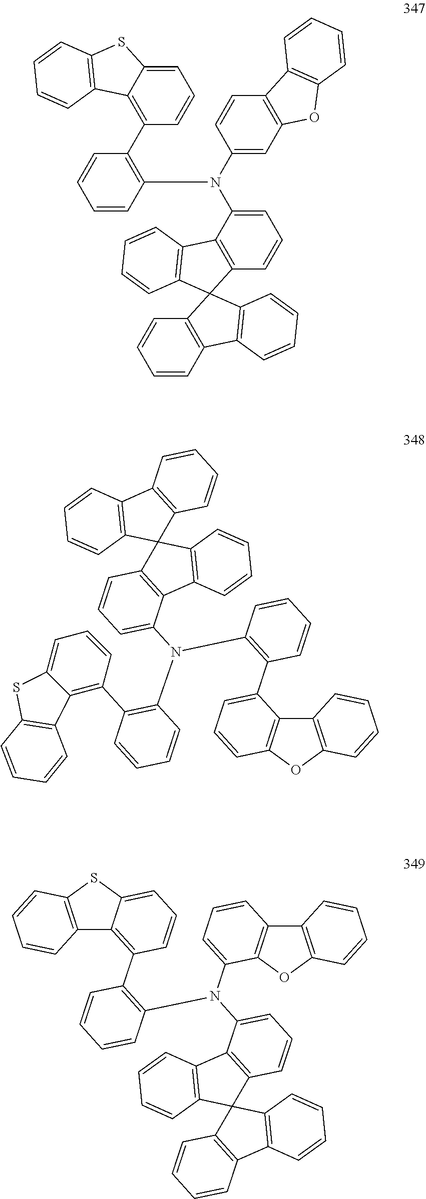 [SMILES: c1ccc(N(c2ccc3c(c2)oc2ccccc23)c2cccc3c2-c2ccccc2C32c3ccccc3-c3ccccc32)c(-c2cccc3sc4ccccc4c23)c1 ; c1ccc(N(c2ccccc2-c2cccc3sc4ccccc4c23)c2cccc3c2-c2ccccc2C32c3ccccc3-c3ccccc32)c(-c2cccc3oc4ccccc4c23)c1 ; c1ccc(N(c2cccc3c2-c2ccccc2C32c3ccccc3-c3ccccc32)c2cccc3c2oc2ccccc23)c(-c2cccc3sc4ccccc4c23)c1]

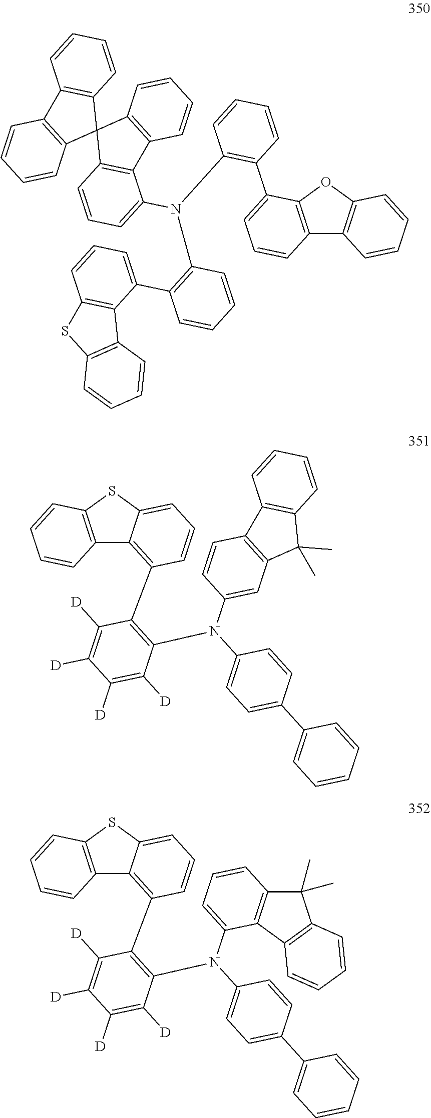 [SMILES: c1ccc(N(c2ccccc2-c2cccc3sc4ccccc4c23)c2cccc3c2-c2ccccc2C32c3ccccc3-c3ccccc32)c(-c2cccc3c2oc2ccccc23)c1 ; [2H]c1c([2H])c([2H])c(N(c2ccc(-c3ccccc3)cc2)c2ccc3c(c2)C(C)(C)c2ccccc2-3)c(-c2cccc3sc4ccccc4c23)c1[2H] ; [2H]c1c([2H])c([2H])c(N(c2ccc(-c3ccccc3)cc2)c2cccc3c2-c2ccccc2C3(C)C)c(-c2cccc3sc4ccccc4c23)c1[2H]]

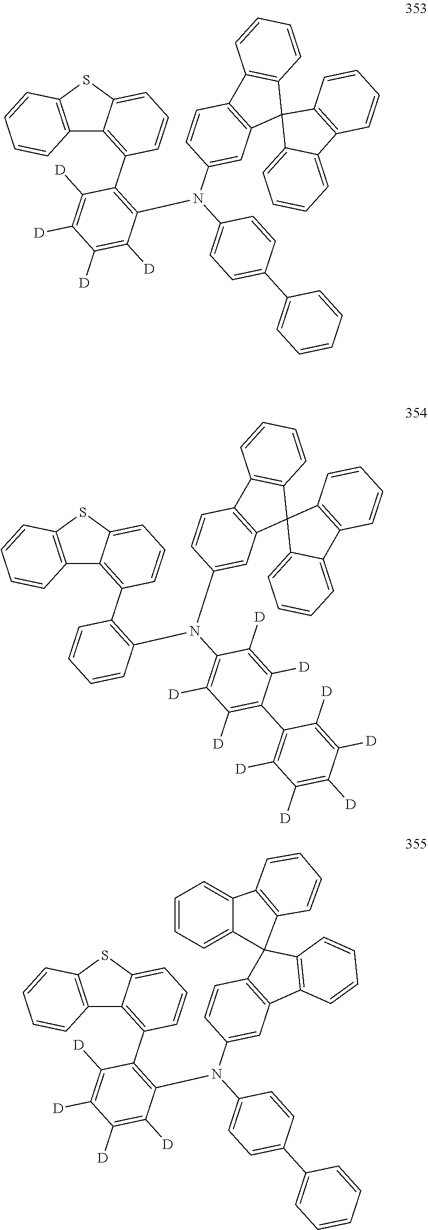

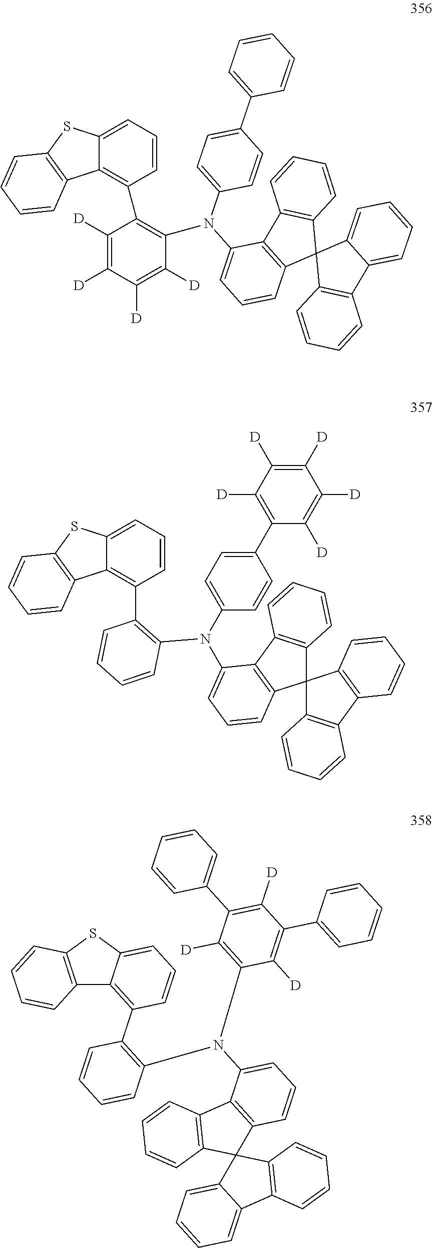 [SMILES: [2H]c1c([2H])c([2H])c(N(c2ccc(-c3ccccc3)cc2)c2cccc3c2-c2ccccc2C32c3ccccc3-c3ccccc32)c(-c2cccc3sc4ccccc4c23)c1[2H] ; [2H]c1c([2H])c([2H])c(-c2ccc(N(c3ccccc3-c3cccc4sc5ccccc5c34)c3cccc4c3-c3ccccc3C43c4ccccc4-c4ccccc43)cc2)c([2H])c1[2H] ; [2H]c1c(-c2ccccc2)c([2H])c(N(c2ccccc2-c2cccc3sc4ccccc4c23)c2cccc3c2-c2ccccc2C32c3ccccc3-c3ccccc32)c([2H])c1-c1ccccc1]

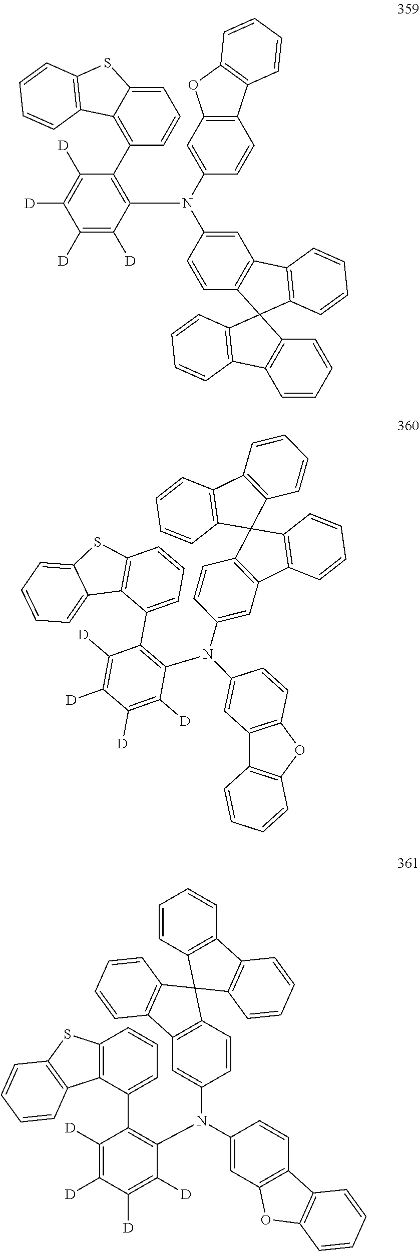

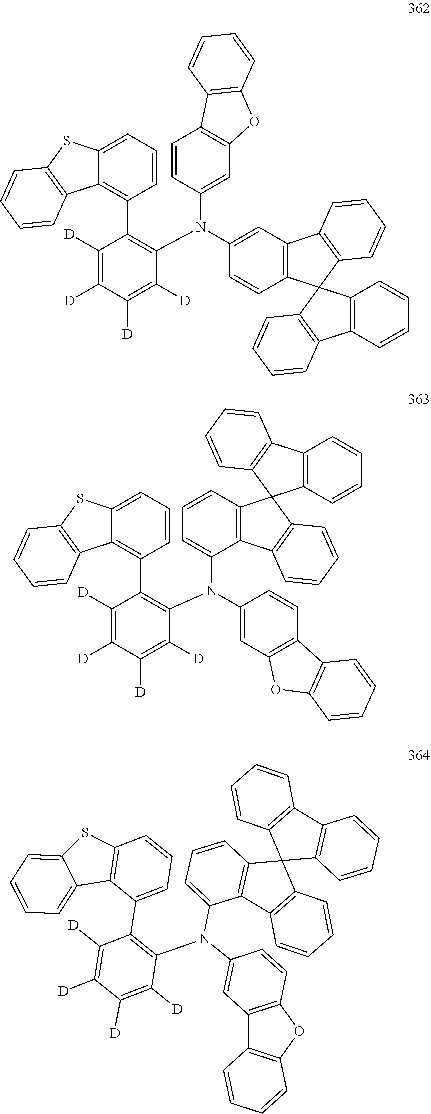

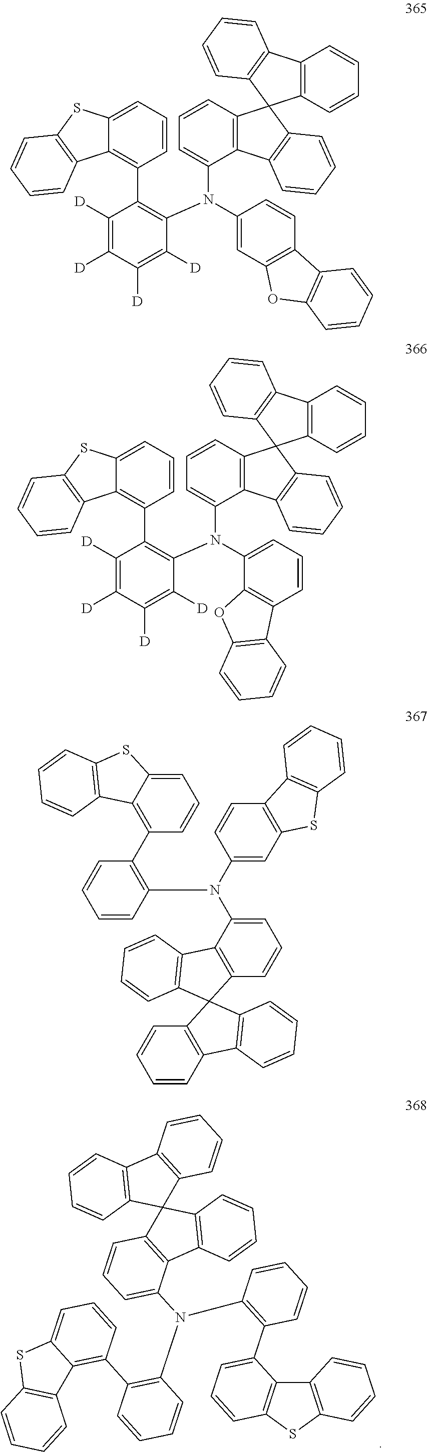

4. An organic light-emitting device comprising a first electrode, a second electrode, and one or more organic layers arranged between the first electrode and the second electrode, wherein
the one or more organic layers include a compound represented by Formula I according to claim 1.

5. The organic light-emitting device of claim 4, wherein the organic layers include one or more layers selected from a hole injecting layer, a hole transport layer, a layer having functions of both hole injection and hole transport, an electron transport layer, an electron injecting layer, a layer having functions of both electron transport and electron injection, an electron blocking layer, a hole blocking layer, and a light-emitting layer; and
the one or more layers include a compound represented by the Formula I above.

6. The organic light-emitting device of claim 5, wherein any one of the hole transport layer, the hole injecting layer, and the layer having functions of both hole injection and hole transport includes a compound represented by the Formula I above.

7. The organic light-emitting device of claim 5, wherein the electron blocking layer includes a compound represented by Formula I above.
